(19) United States
(12) Patent Application Publication
(10) Pub. No.: US 2025/0057658 A1
(43) Pub. Date: Feb. 20, 2025

(54) SYSTEMS AND METHODS OF TARGETED/FOCAL KNEE JOINT RESURFACING

(71) Applicant: Overture Resurfacing Inc. dba Overture Orthopaedics, Irvine, CA (US)

(72) Inventors: Benjamin Arnold, San Diego, CA (US); Hyun Woo Bae, Los Angeles, CA (US); James Young Kim, Irvine, CA (US); Jonathan Nielsen, Rio Rancho, NM (US); Riley J. Williams, III, New York, NY (US); Brian Bowman, Carlsbad, CA (US); Aaron Gunn, Del Mar, CA (US); Sachiye Koide, San Diego, CA (US)

(21) Appl. No.: 18/806,051

(22) Filed: Aug. 15, 2024

Related U.S. Application Data
(60) Provisional application No. 63/520,221, filed on Aug. 17, 2023.
(60) Provisional application No. 63/520,210, filed on Aug. 17, 2023.

(51) Int. Cl. A61F 2/38; A61B 17/16; A61F 2/30

(52) U.S. Cl. CPC A61F 2/389; A61B 17/1675; A61F 2/30771; A61F 2/3859; A61F 2002/30841; A61F 2002/30878; A61F 2002/3092; A61F 2002/30934

(57) ABSTRACT

The present disclosure includes examples of an implant system configured for targeted joint resurfacing in which only an area including the osteochondral defect is resected, wherein the area resected occupies an area smaller than an entire joint surface. The implant system can include a monolithic first implant with 3D printed metal and a second implant including a plastic portion overmolded onto a metal portion. The metal portion of the second implant also includes 3D printed metal. The first and/or second implants include solid metal portion(s) and porous metal portion(s). In an example, the first implant can be a femoral implant and the second implant can be a tibial implant. The first and second implants can be used on their own or in tandem.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the priority benefit of U.S. Provisional Application Nos. 63/520,221, filed Aug. 17, 2023, and U.S. Provisional Application Nos. 63/520,210, filed Aug. 17, 2023, the entirety of each of which is hereby incorporated by reference herein and should be considered part of this specification.

### BACKGROUND

### Field

The present disclosure relates generally to targeted partial replacement (also referred to as “focal-plasty” in the present disclosure) of the articulating surfaces of the knee joint, in particular, in cases where a defect (which may not be the only defect) is located on only one side of the knee joint and occupies a smaller area than an entire joint surface or half of an entire joint surface. Additional defects, if present, may be treated in parallel with a second focal-plasty or another modality.

### Description of the Related Art

Various knee prostheses and procedures are available to treat patients with knee joint deterioration, for example, caused by arthritis, injury, disease, and the like. One common procedure is knee replacement, also known as knee arthroplasty, in which the tibia is resected and replaced with a tibial component, and the femur is resected and replaced with a femoral component. The tibial and femoral components replace the weight-bearing surfaces of the knee joint to relieve pain. Knee replacement surgery can be in the form of either a total knee replacement or partial (also referred to as unicompartmental) knee replacement. One type of partial knee replacement is patellofemoral (kneecap) joint replacement, which treats the patellofemoral compartment. Other common knee surgeries can address subchondral bone defects or injuries, including subchondroplasty or subchondral intraosseous calcium phosphate injection.

### SUMMARY

Arthritis can start at a young age, for example, before age 50, as early as between 20 and 40. The cause of arthritis in the young patients may be an autoimmune condition rather than wear and tear of the joints. In early-onset arthritis, the area of cartilage lesion may be small compared to the total surface area of the joint. At the knee joint, current implants in for total or partial knee replacement are not suitable for treating such small areas of cartilage lesion because total or partial knee replacement procedures remove too much healthy bone and cartilage in the patient. The targeted or focal repair strategies disclosed herein are helpful in bridging the time between early arthritis treatment and total knee replacement. In addition, for some patients, a knee joint implant may need to be replaced at least once during his/her lifetime. Preserving as much healthy bone as possible in the first surgery can lead to better surgical outcome when another surgery is needed to replace the current implants, for example, with bigger implants.

Subchondral bone plasty is a surgical procedure performed when there are subchondral bone defects. Subchondral bone defects or injuries are also known as bone marrow lesions. Subchondral bone defect may include cartilage lesion or may not include cartilage lesion. Subchondral bone plasty usually replaces the subchondral bone that is defective with a bone substitute material.

Unicompartmental targeted or focal joint resurfacing implants for osteochondral defects (which can be bone defect and/or cartilage defect) of the articulating joint surfaces are disclosed here. The defects or injuries in the present disclosure can encompass any one or combination of a chondral defect, osteochondral defect, or early arthritic lesion. The implants disclosed herein can be used for treating early arthritis and/or subchondral bone defects. A reduced implant footprint allows for minimal resection of the articulating joint surfaces.

In a first aspect, embodiments of the present disclosure encompass unicompartmental knee resurfacing system and methods for their use and manufacture. An exemplary unicompartmental targeted knee resurfacing system can include a femoral implant and a tibial implant. The femoral implant can include a convex bearing surface portion and a subchondral surface portion. The tibial implant can include a concave bearing surface portion and a subchondral surface portion. In some cases, the concave bearing surface portion of the tibial implant has a rim that defines a proximal plane, the subchondral surface portion of the tibial implant defines a distal plane, and the proximal plane defined by the rim of the concave bearing surface portion of the tibial implant is non-parallel to the distal plane defined by the subchondral surface portion of the tibial implant. In some cases, the concave bearing surface portion of the tibial implant has a rim that defines a proximal plane, the subchondral surface portion of the tibial implant defines a distal plane, and the proximal plane defined by the rim of the concave bearing surface portion of the tibial implant is parallel to the distal plane defined by the subchondral surface portion of the tibial implant. In some cases, the femoral implant further includes at least one proximal peg. In some cases, a proximal peg of the femoral implant includes at least one barb. In some cases, the femoral implant further includes at least one anti-rotation spike. In some cases, the tibial implant further includes at least one distal peg. In some cases, a distal peg of the tibial implant includes at least one barb. In some cases, the concave bearing surface portion of the tibial implant has a rim that defines a proximal plane, the subchondral surface portion of the tibial implant defines a distal plane, the tibial implant further includes at least one distal peg, and a distal peg of the tibial implant defines an axis that is perpendicular to the distal plane defined by the subchondral surface portion of the tibial implant and that is non perpendicular to the proximal plane defined by the rim of the concave bearing surface portion of the tibial implant. In some cases, the tibial implant further includes at least one anti-rotation spike. In some cases, the convex bearing surface portion of the femoral implant includes nonporous titanium. In some cases, the subchondral surface portion of the femoral implant includes porous titanium. In some cases, a peg of the femoral implant includes multiple barbs. In some cases, the femoral implant further includes one or more anti-rotation spikes. In some cases, the femoral implant further includes one or more proximal pegs. In some cases, the concave bearing surface portion of the tibial implant includes ultra-high-molecular-weight-polyethylene (UHMWPE). In some cases, the subchondral surface portion of the tibial implant has a proximal side and a distal side, and the concave bearing surface portion of the tibial implant is compression molded with the proximal side of the subchondral surface portion of the tibial implant. In some cases, the proximal side of the subchondral surface portion of the tibial implant includes porous titanium. In some cases, the distal side of the subchondral surface portion of the tibial implant includes an irregular lattice. In some cases, a distal peg of the tibial implant includes one or more barbs. In some cases, the tibial implant further includes one or more anti-rotation spikes. In some cases, the tibial implant further includes one or more distal pegs. In some cases, the convex bearing surface portion of the femoral implant includes a round profile. In some cases, the convex bearing surface portion of the femoral implant includes an oblong racetrack profile. In some cases, the convex bearing surface portion of the femoral implant includes a three-circle profile. In some cases, a proximal peg of the femoral implant includes a trabecular porous structure. In some cases, a distal peg of the tibial implant includes a trabecular porous structure. In some cases, the convex bearing surface portion of the femoral implant includes nonporous stainless steel, nonporous cobalt chrome, nonporous ceramic, or any combination thereof. In some cases, the proximal side of the subchondral surface portion of the tibial implant includes nonporous stainless steel, nonporous cobalt chrome, nonporous ceramic, or any combination thereof. In some cases, the convex bearing surface portion of the femoral implant includes a round profile having a diameter value within a range from about 12 mm to about 20 mm. In some cases, the convex bearing surface portion of the femoral implant includes an oblong racetrack profile having a length value within a range from about 20 mm to about 35 mm. In some cases, the convex bearing surface portion of the femoral implant includes an oblong racetrack profile having a width value within a range from about 12 mm to about 20 mm. In some cases, the femoral implant has a thickness value within a range from about 5 mm to about 10 mm. In some cases, the tibial implant has a diameter value within a range from about 15 mm to about 25 mm. In some cases, the tibial implant has a thickness value that is equal to or greater than about 6.5 mm. In some cases, the femoral implant has a circular shape and further includes a bone screw fixation mechanism. In some cases, the tibial implant has a circular shape and further includes a bone screw fixation mechanism.

In another aspect, embodiments of the present disclosure encompass targeted joint resurfacing systems and methods for their use and manufacture. An exemplary joint resurfacing system can include a first implant and a second implant. The first implant can have a convex bearing surface portion and a subchondral surface portion. The second implant can have a concave bearing surface portion and a subchondral surface portion. The system can be configured for implantation into a joint of a patient. In some cases, the joint is a knee joint, a shoulder joint, a hip joint, an ankle joint, or a first metatarsal-phalangeal joint. In some cases, the first implant includes a femoral implant, the second implant includes a tibial implant, and the joint is a knee joint. In some cases, the concave bearing surface portion of the second implant has a rim that defines a proximal plane, the subchondral surface portion of the second implant defines a distal plane, and the proximal plane defined by the rim of the concave bearing surface portion of the second implant is non-parallel to the distal plane defined by the subchondral surface portion of the second implant. In some cases, the concave bearing surface portion of the second implant has a rim that defines a proximal plane, the subchondral surface portion of the second implant defines a distal plane, and the proximal plane defined by the rim of the concave bearing surface portion of the second implant is parallel to the distal plane defined by the subchondral surface portion of the second implant. In some cases, the first implant further includes at least one proximal peg. In some cases, a proximal peg of the first implant includes at least one barb. In some cases, the first implant further includes at least one anti-rotation spike. In some cases, the second implant further includes at least one distal peg. In some cases, a distal peg of the second implant includes at least one barb. In some cases, the concave bearing surface portion of the second implant has a rim that defines a proximal plane, the subchondral surface portion of the second implant defines a distal plane, the second implant further includes at least one distal peg, and a distal peg of the second implant defines an axis that is perpendicular to the distal plane defined by the subchondral surface portion of the second implant and that is non perpendicular to the proximal plane defined by the rim of the concave bearing surface portion of the second implant. In some cases, the second implant further includes at least one anti-rotation spike. In some cases, the convex bearing surface portion of the first implant includes nonporous titanium. In some cases, the subchondral surface portion of the first implant includes porous titanium. In some cases, a peg of the first implant includes one or more barbs. In some cases, the first implant further includes one or more anti-rotation spikes. In some cases, the first implant further includes one or more proximal pegs. In some cases, the concave bearing surface portion of the second implant includes UHMWPE. In some cases, the subchondral surface portion of the second implant has a proximal side and a distal side, and the concave bearing surface portion of the second implant is compression molded with the proximal side of the subchondral surface portion of the second implant. In some cases, the proximal side of the subchondral surface portion of the second implant includes porous titanium. In some cases, the distal side of the subchondral surface portion of the second implant includes an irregular lattice. In some cases, a distal peg of the second implant includes one or more barbs. In some cases, the second implant further includes one or more anti-rotation spikes. In some cases, the second implant further includes one or more distal pegs. In some cases, the convex bearing surface portion of the first implant includes a round profile. In some cases, the convex bearing surface portion of the first implant includes an oblong racetrack profile. In some cases, the convex bearing surface portion of the first implant includes a three-circle profile. In some cases, a proximal peg of the first implant includes a trabecular porous structure. In some cases, a distal peg of the second implant includes a trabecular porous structure. In some cases, the convex bearing surface portion of the first implant includes nonporous stainless steel, nonporous cobalt chrome, nonporous ceramic, or any combination thereof. In some cases, the proximal side of the subchondral surface portion of the second implant includes nonporous stainless steel, nonporous cobalt chrome, nonporous ceramic, or any combination thereof. In some cases, the convex bearing surface portion of the first implant includes a round profile having a diameter value within a range from about 10 mm to about 35 mm. In some cases, the convex bearing surface portion of the first implant includes an oblong racetrack profile having a length value within a range from about 20 mm to about 35 mm. In some cases, the convex bearing surface portion of the first implant includes an oblong racetrack profile having a width value within a range from about 12 mm to about 20 mm. In some cases, the first implant has a thickness value within a range from about 5 mm to about 10 mm. In some cases, the second implant has a diameter value within a range from about 15 mm to about 25 mm. In some cases, the second implant has a thickness value that is equal to or greater than about 6.5 mm. In some cases, the first implant has a circular shape and further includes a bone screw fixation mechanism. In some cases, the second implant has a circular shape and further includes a bone screw fixation mechanism.

In another aspect, embodiments of the present disclosure encompass systems and methods for implanting a targeted joint resurfacing system into a joint of a patient. Exemplary methods may include engaging a first implant of the resurfacing system with a distal portion of a first bone of the joint of the patient, where the first implant includes a convex bearing surface portion and a subchondral surface portion. Methods may further include engaging a second implant of the resurfacing system with a proximal portion of a second bone of the joint of the patient, where the second implant includes a concave bearing surface portion and a subchondral surface portion. In some cases, the joint is a knee joint, a shoulder joint, a hip joint, an ankle joint, or a first metatarsal-phalangeal joint. In some cases, the joint is a knee joint, the first implant includes a femoral implant, and the second implant includes a tibial implant. In some cases, the concave bearing surface portion of the second implant has a rim that defines a proximal plane, the subchondral surface portion of the second implant defines a distal plane, and the proximal plane defined by the rim of the concave bearing surface portion of the second implant is non-parallel to the distal plane defined by the subchondral surface portion of the second implant. In some cases, the concave bearing surface portion of the second implant has a rim that defines a proximal plane, the subchondral surface portion of the second implant defines a distal plane, and the proximal plane defined by the rim of the concave bearing surface portion of the second implant is parallel to the distal plane defined by the subchondral surface portion of the second implant. In some cases, the first implant further includes at least one proximal peg. In some cases, a proximal peg of the first implant includes at least one barb. In some cases, the first implant further includes at least one anti-rotation spike. In some cases, the second implant further includes at least one distal peg. In some cases, a distal peg of the second implant includes at least one barb. In some cases, the concave bearing surface portion of the second implant has a rim that defines a proximal plane, the subchondral surface portion of the second implant defines a distal plane, the second implant further includes at least one distal peg, and a distal peg of the second implant defines an axis that is perpendicular to the distal plane defined by the subchondral surface portion of the second implant and that is non perpendicular to the proximal plane defined by the rim of the concave bearing surface portion of the second implant. In some cases, the second implant further includes at least one anti-rotation spike. In some cases, the convex bearing surface portion of the first implant includes nonporous titanium. In some cases, the subchondral surface portion of the first implant includes porous titanium. In some cases, a peg of the first implant includes one or more barbs. In some cases, the first implant further includes one or more anti-rotation spikes. In some cases, the first implant further includes one or more proximal pegs. In some cases, the concave bearing surface portion of the second implant includes UHMWPE. In some cases, the subchondral surface portion of the second implant has a proximal side and a distal side, and the concave bearing surface portion of the second implant is compression molded with the proximal side of the subchondral surface portion of the second implant. In some cases, the proximal side of the subchondral surface portion of the second implant includes porous titanium. In some cases, the distal side of the subchondral surface portion of the second implant includes an irregular lattice. In some cases, the distal peg of the second implant includes one or more barbs. In some cases, the second implant further includes one or more anti-rotation spikes. In some cases, the second implant further includes one or more distal pegs. In some cases, the convex bearing surface portion of the first implant includes a round profile. In some cases, the convex bearing surface portion of the first implant includes an oblong racetrack profile. In some cases, the convex bearing surface portion of the first implant includes a three-circle profile. In some cases, a proximal peg of the first implant includes a trabecular porous structure. In some cases, a distal peg of the second implant includes a trabecular porous structure. In some cases, the convex bearing surface portion of the first implant includes nonporous stainless steel, nonporous cobalt chrome, nonporous ceramic, or any combination thereof. In some cases, the proximal side of the subchondral surface portion of the second implant includes nonporous stainless steel, nonporous cobalt chrome, nonporous ceramic, or any combination thereof. In some cases, the convex bearing surface portion of the first implant includes a round profile having a diameter value within a range from about 10 mm to about 35 mm. In some cases, the convex bearing surface portion of the first implant includes an oblong racetrack profile having a length value within a range from about 20 mm to about 35 mm. In some cases, the convex bearing surface portion of the first implant includes an oblong racetrack profile having a width value within a range from about 12 mm to about 20 mm. In some cases, the first implant has a thickness value within a range from about 5 mm to about 10 mm. In some cases, the second implant has a diameter value within a range from about 15 mm to about 25 mm. In some cases, the second implant has a thickness value that is equal to or greater than about 6.5 mm. In some cases, the first implant has a circular shape and further includes a bone screw fixation mechanism. In some cases, the second implant has a circular shape and further includes a bone screw fixation mechanism.

In some aspects, the present disclosure relates to a monolithic implant for targeted joint resurfacing to replace a defect while preserving as much healthy bone and/or cartilage as possible of a patient, the implant including: a solid metal portion including an articulating surface, the articulating surface being convex and load-bearing; and a porous metal portion including a subchondral surface configured to interface with a surgically prepared bone surface, the porous metal portion further including at least an outer portion of a fixation peg extending from the subchondral surface, wherein an inner or core portion of the fixation peg can be part of the solid metal portion.

In some aspects, a part of the implant between the articulating surface and the subchondral surface can have a round profile.

In some aspects, the round profile can have a diameter of between 17.5 mm and 27.5 mm.

In some aspects, a part of the implant between the articulating surface and the subchondral surface can have an oblong profile, the oblong profile having a length that can be greater than a width.

In some aspects, the oblong profile can be formed by three circles aligned along the length such that at least two neighboring circles can partially overlap.

In some aspects, the oblong profile can have a length between 25 mm and 40 mm.

In some aspects, the oblong profile can have a width between 17.5 mm and 27.5 mm.

In some aspects, the monolithic implant can further include a second peg, at least an outer portion of the second peg being part of the porous metal portion.

In some aspects, the solid metal portion can further include a plurality of anti-rotation spikes extending from the subchondral surface, the plurality of anti-rotation spikes being 3D printed onto the subchondral surface.

In some aspects, the solid metal portion and the porous metal portion can include titanium or a titanium alloy.

In some aspects, the porous metal portion can be 3D-printed.

In some aspects, the fixation peg can include a plurality of barbs, the plurality of barbs being part of the solid metal portion.

In some aspects, the plurality of barbs can be 3D-printed.

In some aspects, the fixation peg can include a plurality of channels each extending along a length of the peg and cutting into the plurality of barbs and a body of the peg, the plurality of channels configured to direct flow of bone cement.

In some aspects, the subchondral surface can include a well or trough around the fixation peg,

In some aspects, the well or trough can be configured to receive overflow of bone cement.

In some aspects, the bone cement can be configured to facilitate primary fixation between the peg and a hole drilled into the reamed bone surface and the porous metal portion can be configured to provide secondary fixation by osteointegration.

In some aspects, the monolithic implant can be configured to be inserted without bone cement.

In some aspects, the articulating surface can further include a titanium nitride coating.

In some aspects, the fixation peg can be perpendicular to the subchondral surface.

In some aspects, the monolithic implant can further include at least two pockets on opposite sides of the articulating surface, the at least two pockets configured to receive an insertion tool or a removal tool.

In some aspects, the monolithic implant can be a femoral implant configured for targeted knee joint resurfacing in which only an area including the defect is removed, wherein the area removed can remove an area smaller than an entire surface of a femoral condyle.

In some aspects, the present disclosure relates to a single-piece implant for targeted joint resurfacing to replace a defect while preserving as much healthy bone and/or cartilage as possible of a patient, the implant including: a metal portion including a subchondral surface configured to interface with a surgically prepared bone surface, the metal portion including solid metal parts and porous metal parts, the porous metal parts including the subchondral surface and an overmolding surface, the solid metal parts located at least between the subchondral surface and the overmolding surface, wherein the metal portion can further include a fixation peg extending from the subchondral surface, at least an outer portion of the fixation peg being one of the porous metal parts and an inner or core portion of the fixation peg being one of the porous metal parts; and a plastic portion overmolded on the metal portion such that the single-piece implant can be preassembled during manufacturing, the plastic portion including an articulating surface and an overmolding section, the articulating surface being concave and load-bearing, the overmolding section interfacing with the overmolding surface of the metal portion, wherein the single-piece implant can include a circumferential groove at an interface of the overmolding section and the overmolding surface, the circumferential groove configured to allow clamping of the metal portion during overmolding to provide a clean stop of plastic flow below the circumferential groove.

In some aspects, the fixation peg can be perpendicular to the subchondral surface.

In some aspects, the fixation peg can include a plurality of barbs, the plurality of barbs being one of the solid metal parts.

In some aspects, the plurality of barbs can be 3D-printed.

In some aspects, the fixation peg can include a plurality of channels each extending along a length of the peg and cutting into the plurality of barbs and a body of the peg, the plurality of channels configured to direct flow of bone cement.

In some aspects, the subchondral surface can include a well or trough around the fixation peg, the well or trough configured to receive overflow of bone cement.

In some aspects, the bone cement can be configured to facilitate primary fixation between the peg and a hole drilled into the reamed bone surface and the porous metal parts can be at least configured to provide secondary fixation by osteointegration.

In some aspects, the single-piece implant can be configured to be inserted without bone cement.

In some aspects, a part of the implant between the articulating surface and the subchondral surface can have a round profile.

In some aspects, the round profile can have a diameter of between 17.5 mm and 27.5 mm.

In some aspects, the articulating surface can have a rim defining a proximal plane and the subchondral surface defines a distal plane, the proximal plane and the distal plane being at a non-zero angle so as to facilitate insertion in an antegrade approach.

In some aspects, the non-zero angle can be between 10° to 15°.

In some aspects, the non-zero angle can be 10°.

In some aspects, the metal portion can further include a plurality of anti-rotation spikes extending from the subchondral surface, the plurality of anti-rotation spikes being solid and 3D printed onto the subchondral surface.

In some aspects, the techniques described herein relate to a single-piece implant, wherein the metal portion includes titanium or a titanium alloy.

In some aspects, the techniques described herein relate to a single-piece implant, wherein the porous metal parts are 3D-printed.

In some aspects, the techniques described herein relate to a single-piece implant, further including at least two pockets on opposite sides of the articulating surface, the at least two pockets configured to receive an insertion tool or a removal tool.

In some aspects, the techniques described herein relate to a single-piece implant, wherein the single-piece implant is a tibial implant configured for targeted knee joint resurfacing in which only an area including the defect is removed, wherein the area removed occupies an area smaller than an entire surface of a tibial plateau.

In some aspects, the techniques described herein relate to a single-piece implant for targeted joint resurfacing to replace a defect while preserving as much healthy bone and/or cartilage as possible of a patient, the implant including: a metal portion including a subchondral surface configured to interface with a surgically prepared bone surface, the metal portion including solid metal parts and porous metal parts, the porous metal parts including the subchondral surface and an overmolding surface, the metal portion further includes a fixation peg extending from the subchondral surface, the solid metal parts located at least between the subchondral surface and the overmolding surface, wherein the metal portion can further include a fixation peg extending from the subchondral surface, at least an outer portion of the fixation peg being one of the porous metal parts and an inner or core portion of the fixation peg being one of the porous metal parts; and a plastic portion overmolded on the metal portion, the plastic portion including an articulating surface and an overmolding section, the articulating surface being concave and load-bearing, the overmolding section interfacing with the overmolding surface of the metal portion, wherein the articulating surface has a rim defining a proximal plane and the subchondral surface defines a distal plane, the proximal plane and the distal plane being at a non-zero angle so as to facilitate insertion of the single-piece implant in an antegrade approach.

In some aspects, the techniques described herein relate to a single-piece implant, wherein the non-zero angle is between 10° to 15°.

In some aspects, the techniques described herein relate to a single-piece implant, wherein the non-zero angle is 10°.

In some aspects, the techniques described herein relate to a single-piece implant, wherein a part of the implant between the articulating surface and the subchondral surface has a round profile.

In some aspects, the techniques described herein relate to a single-piece implant, wherein the round profile has a diameter of between 17.5 mm and 27.5 mm.

In some aspects, the techniques described herein relate to a single-piece implant, wherein the metal portion further includes a plurality of anti-rotation spikes extending from the subchondral surface, the plurality of anti-rotation spikes being solid and 3D printed onto the subchondral surface.

In some aspects, the metal portion can include titanium or a titanium alloy.

In some aspects, the porous metal parts can be 3D-printed.

In some aspects, the fixation peg can include a plurality of barbs, the plurality of barbs being one of the solid metal parts.

In some aspects, the plurality of barbs can be 3D-printed.

In some aspects, the fixation peg can include a plurality of channels each extending along a length of the peg and cutting into the plurality of barbs and a body of the peg, the plurality of channels configured to direct flow of bone cement.

In some aspects, the subchondral surface can include a well or trough around the fixation peg.

In some aspects, the bone cement can be configured to facilitate primary fixation between the peg and a hole drilled into the reamed bone surface and the porous metal parts can be at least configured to provide secondary fixation by osteointegration.

In some aspects, the single-piece implant can be configured to be inserted without bone cement.

In some aspects, the single-piece implant can further include at least two pockets on opposite sides of the articulating surface, the at least two pockets configured to receive an insertion tool or a removal tool.

In some aspects, the single-piece implant can be a tibial implant configured for targeted knee joint resurfacing in which only an area including the defect is removed, wherein the area removed can occupy an area smaller than an entire surface of a tibial plateau.

In some aspects, the present disclosure relates to an implant system configured for targeted joint resurfacing in which only an area including the defect is removed, wherein the area removed can occupy an area smaller than an entire joint surface, the implant system including: a first implant, wherein the first implant can be the monolithic implant; and a second implant, wherein the second implant can be the single-piece implant.

In some aspects, the present disclosure relates to a single-piece bone implant including: at least a fixation peg made with metal, wherein the fixation peg can be generally cylindrical and including an inner or core portion and an outer or surface portion, the inner or core portion including solid metal and the outer or surface portion including porous metal; and a plurality of barbs arranged along a longitudinal axis of the fixation peg, the plurality of barbs including solid metal, wherein bone cement can be configured to facilitate primary fixation of the fixation peg and/or the plurality of barbs into a hole drilled into a bone and the porous metal of the outer or surface portion can be configured to provide secondary fixation by osteointegration.

In some aspects, at least any porous metal portion of the implant and the plurality of barbs can be 3D printed.

In some aspects, the fixation peg can include a plurality of channels each extending along a length of the peg and cutting into the plurality of barbs and a body of the peg, the plurality of channels configured to direct flow of the bone cement.

In some aspects, the single-piece bone implant can further include a cap or cover component defined between an articulating surface and a subchondral surface, the fixation peg extending from the subchondral surface and being perpendicular to the subchondral surface.

In some aspects, the subchondral surface can include a well or trough around the fixation peg.

In some aspects, the implant can be configured to be implanted on an area of a femoral condyle and the articulating surface is convex.

In some aspects, the implant can be configured to be implanted on an area of a tibial plateau and the articulating surface is concave.

In some aspects, the articulating surface can have a rim defining a proximal plane and the subchondral surface can define a distal plane, the proximal plane and the distal plane being at a non-zero angle so as to facilitate insertion in an antegrade approach.

In some aspects, the non-zero angle can be between 10° to 15°.

In some aspects, the non-zero angle can be 10°.

In some aspects, the present disclosure relates to a method of implanting a tibial implant configured for targeted knee joint resurfacing to replace a defect while preserving as much healthy bone and/or cartilage as possible of a patient, the method including: flexing a knee joint of a patient by at least 120°, wherein the patient can be in a supine position; making a longitudinal incision on skin of the patient at the knee joint to expose the knee joint, wherein the incision can be made on a lateral side if the osteochondral defect is on the lateral side and the incision can be made on a medial side if the osteochondral defect is on the medial side; detaching an anterior portion of a meniscus from an articulating surface of a tibia to access the tibial plateau in an antegrade approach, the meniscus remaining attached to a joint capsule; and inserting a reamer from an anterior side of the knee joint, wherein a reamer head of the reamer can include a cutout, the reamer can be oriented such that the cutout can be facing a femur condyle during insertion and removal of the reamer.

In some aspects, the tibial implant can include an articulating surface and a subchondral surface, the articulating surface can be configured to be load bearing, the subchondral surface can be configured to interface with a reamed bone surface on the tibial plateau, the articulating surface can have a rim defining a proximal plane and the subchondral surface defines a distal plane, the proximal plane and the distal plane being at a non-zero angle so as to facilitate insertion in the antegrade approach.

In some aspects, the non-zero angle can be between 10° to 15°.

In some aspects, the non-zero angle can be 10°.

In some aspects, the articulating surface or the subchondral surface can have a round profile with a diameter of between 17.5 mm to 27.5 mm.

In some aspects, the tibial implant can have a single piece component that may not require in-surgery assembly.

In some aspects, insertion of the reamer can be guided by a guide pin drilled into the tibial plateau, the method further including placing a tibial pin guide onto the tibial plateau prior to inserting the reamer, a base of the tibial pin guide defining a distal engagement plane, a longitudinal axis of a shaft of the tibial pin guide can be at a non-zero angle relative to a plane perpendicular to the distal engagement plane.

In some aspects, the non-zero angle can be between 10° to 15°.

In some aspects, the non-zero angle can be 10°.

In some aspects, the base of the tibial pin guide can include a cutout, the tibial pin guide can be oriented such that the cutout can be facing the femur condyle during insertion and removal of the tibial pin guide.

In some aspects, the method can further include using the reamer to resect only an area including the osteochondral defect, wherein the area resected can occupy an area smaller than an entire surface of a tibial plateau.

In some aspects, the method can further include scoring cartilage from the area using a scoring tool prior to inserting the reamer.

In some aspects, the reamer can be coupled to a motor and a power source to provide powered reaming.

In some aspects, the method can further include inserting a second reamer after removing the reamer, a reamer head of the second reamer can include a cutout, the second reamer can be oriented such that the cutout can be facing the femur condyle during insertion and removal of the second reamer.

In some aspects, the second reamer can be coupled to a T-handle for manual reaming.

In some aspects, a top surface of the reamer head of the second reamer can matches an articulating surface of the tibial implant, and a height of the reamer head of the second reamer can be configured to aid a user in determining a depth of a reamed hole such that when the tibial implant is inserted into the reamed hole, the articulating surface of the tibial implant can be recessed below an adjacent cartilage surface by a distance.

In some aspects, the distance can be between 0.5 mm to 1.0 mm.

In some aspects, the distance can be 0.5 mm.

In some aspects, the method can further include reattaching the anterior portion of the meniscus to the tibial plateau after inserting the tibial implant.

In some aspects, the method can further include externally rotating the knee if the osteochondral defect is on the medial side or internally rotating the knee if the osteochondral defect is on the lateral side to improve visualization.

In some aspects, the method can further include using a curved impactor to fully insert the implant into a reamed hole, the curved impactor including a curved body to clear the femoral condyle.

In some aspects, the present disclosure relates to a surgical tool configured to serve a dual purpose of reaming a bone and acting as a trial for an implant, the implant including an articulating surface and a subchondral surface, a thickness of the implant being defined as a distance between the articulating surface and the subchondral surface, the surgical tool including: a shaft with an elongate body, wherein a proximal end of the shaft can be configured to be coupled to a handle; and a reamer head coupled to a distal end of the shaft, the reamer head including a plurality of cutting blades, wherein a top surface of the reamer head can match the articulating surface of the implant, and a height of the reamer head can be configured relative to the thickness of the implant to aid a user in determining a depth of a reamed hole such that when the implant is inserted into the reamed hole, the articulating surface of the implant can be recessed below an adjacent cartilage surface by a distance.

In some aspects, the distance can be between 0.5 mm to 1.0 mm.

In some aspects, the distance can be 0.5 mm.

In some aspects, the height of the reamer head can be the same as the thickness of the implant.

In some aspects, the height of the reamer head can be greater than the thickness of the implant.

In some aspects, the implant can be a femoral implant configured for targeted knee joint resurfacing in which only an area including a defect is removed, wherein the area removed can occupy an area smaller than an entire surface of a femoral condyle, the top surface of the reamer head can be convex.

In some aspects, the implant can be a tibial implant configured for targeted knee joint resurfacing in which only an area including a defect is removed, wherein the area removed can occupy an area smaller than an entire surface of a tibial plateau, the top surface of the reamer head can be concave.

In some aspects, the surgical tool can be configured to be used in a nonpowered manner.

In some aspects, the surgical tool can be configured to be single use, the plurality of cutting blades being made of metal and at least a portion of the surgical tool being made of plastic.

In some aspects, the single use surgical tool can include molded plastic with the plurality of cutting blades embedded.

In some aspects, the single use surgical tool can further include a metal hypotube within a plastic outer tube.

In some aspects, the present disclosure relates to a method of fixing an implant into a surgically prepared hole at a joint, the implant including an articulating surface and a subchondral surface, the subchondral surface configured to interface with a bottom surface of the reamed hole, the implant further including at least one peg extending from the subchondral surface, the method including: drilling a smaller hole than the surgically prepared hole on the bottom surface, a longitudinal axis of the smaller hole being parallel with a longitudinal axis of the surgically prepared hole; applying bone cement into the smaller hole only; inserting the implant into the surgically prepared hole while aligning the at least one peg with the smaller hole, wherein the bone cement can be configured to facilitate primary fixation of the at least one peg with the smaller hole and uncemented surfaces of the implant can be configured to provide secondary fixation by osteointegration.

In some aspects, the uncemented surfaces can include porous metal.

In some aspects, the uncemented surfaces can include at least the subchondral surface and an outer surface of the at least one peg.

In some aspects, the at least one peg can include a plurality of barbs, the plurality of barbs configured to aid in the primary fixation.

In some aspects, the at least one peg can include a plurality of channels each extending along a length of the at least one peg and cutting into the plurality of barbs and a body of the at least one peg, the plurality of channels configured to direct flow of the bone cement.

In some aspects, the subchondral surface of the implant can include a well or trough around the at least one peg, the well or trough configured to receive overflow of the bone cement.

In some aspects, the implant can include two pegs, the method further including drilling a second smaller hole than the surgically prepared hole on the bottom surface and inserting the implant into the surgically prepared hole while aligning each of the two pegs with the smaller hole and the second smaller hole respectively.

In some aspects, the techniques described herein relate to a targeted joint resurfacing implant or implant system including one or more features of the foregoing description.

In some aspects, the techniques described herein relate to a method of targeted joint surfacing including one or more features of the foregoing description.

Embodiments of the present disclosure also relate generally to arthroplasty systems and methods, and in particular encompass minimal resurfacing patellofemoral implant systems and methods for osteochondral defects.

Patellofemoral joint resurfacing implants for osteochondral defects of the articulating joint surfaces are disclosed here. A reduced implant footprint allows for minimal resection of the articulating joint surfaces. Porous in-growth and barbed peg features allow for strong, cement-less fixation. A prosthetic for the saddle-shaped trochlear groove surface is offered in shapes and sizes that can be implanted along the transverse plane or sagittal plane of the trochlear groove.

In a first aspect, embodiments of the present disclosure encompass patellofemoral arthroplasty resurfacing system and methods for their use and manufacture. An exemplary patellofemoral arthroplasty resurfacing system can include a femoral implant and a patellar implant. The femoral implant can include a bearing surface portion that matches the geometry of the trochlear groove and a subchondral surface portion. The patellar implant can include domed bearing surface portion and a subchondral surface portion. In some cases, the femoral implant further includes at least one proximal peg. In some cases, a proximal peg of the femoral implant includes at least one barb. In some cases, the femoral implant further includes at least one anti-rotation spike. In some cases, a peg of the femoral implant includes multiple barbs. In some cases, the femoral implant further includes one or more anti-rotation spikes. In some cases, the femoral implant further includes one or more proximal pegs. In some cases, a proximal peg of the femoral implant includes a trabecular porous structure. In some cases, the subchondral surface portion of the femoral implant includes porous titanium. In some cases, the subchondral surface portion of the femoral implant includes an irregular lattice. In some cases, the bearing surface portion of the femoral implant includes nonporous stainless steel, nonporous cobalt chrome, nonporous ceramic, or any combination thereof. In some cases, the bearing surface portion of the femoral implant includes a round geometry. In some cases, the bearing surface portion of the femoral implant includes an oblong geometry that is longer in the plane transverse to the trochlear groove. In some cases, the bearing surface portion of the femoral implant includes an oblong geometry that is longer in the sagittal plane (i.e., parallel to the trochlear groove). In some cases, the bearing surface portion of the oblong femoral implant includes a three-circle profile. In some cases, the three-circle profile includes a middle circle that is perpendicular to the transverse and sagittal planes of the trochlear groove and two outer circles that are perpendicular to the transverse plane and angle away from the sagittal plane of the trochlear groove. In some cases, the three-circle profile includes a middle circle that is perpendicular to the transverse and sagittal planes of the trochlear groove and two outer circles that are perpendicular to the transverse plane and perpendicular to the sagittal plane. In some cases, the bearing surface portion of the femoral implant includes a round profile having a diameter value within a range from about 20 mm to about 40 mm. In some cases, the bearing surface portion of the femoral implant includes an oblong profile having a length value within a range from about 25 mm to about 40 mm. In some cases, the femoral implant has a three-circle oblong racetrack profile having a circle diameter of about 20 mm to about 35 mm. In some cases, the femoral implant has a thickness value within a range from about 4 mm to about 15 mm. In some cases, the femoral implant is a monolithic unit. In some cases, the patellar implant further includes at least one distal peg. In some cases the patellar implant peg further includes a titanium pin for X-ray visualization. In some cases, the patellar implant further includes a pocket for a cement mantle. In some cases the patellar implant includes highly crosslinked ultra-high molecular weight polyethylene. In some cases, the patellar implant includes vitamin E diffused highly crosslinked ultra-high molecular weight polyethylene. In some cases, the patellar implant includes a round geometry. In some cases, the patellar implant has a diameter value within a range from about 25 mm to about 30 mm. In some cases, the patellar implant has a thickness value that is greater than about 7.5 mm. In some cases, the patellar implant is a monolithic unit.

In another aspect, embodiments of the present disclosure encompass arthroplasty resurfacing systems and methods for their use and manufacture. An exemplary arthroplasty resurfacing system can include a first implant and a second implant. The first implant can have a bearing surface portion that matches the geometry of the distal groove of the bone and a subchondral surface portion. The second implant can have a domed bearing surface portion and a subchondral surface portion. In some cases, the joint is a knee joint, a shoulder joint, a hip joint, an ankle joint, or a first metatarsal-phalangeal joint. In some cases, the joint is a knee joint, the first implant includes a femoral implant, and the second implant includes a patellar implant. In some cases, the femoral implant further includes at least one proximal peg. In some cases, a proximal peg of the femoral implant includes at least one barb. In some cases, the femoral implant further includes at least one anti-rotation spike. In some cases, a peg of the femoral implant includes multiple barbs. In some cases, the femoral implant further includes one or more anti-rotation spikes. In some cases, the femoral implant further includes one or more proximal pegs. In some cases, a proximal peg of the femoral implant includes a trabecular porous structure. In some cases, the subchondral surface portion of the femoral implant includes porous titanium. In some cases, the subchondral surface portion of the femoral implant includes an irregular lattice. In some cases, the bearing surface portion of the femoral implant includes nonporous stainless steel, nonporous cobalt chrome, nonporous ceramic, or any combination thereof. In some cases, the bearing surface portion of the femoral implant includes a round geometry. In some cases, the bearing surface portion of the femoral implant includes an oblong geometry that is longer in the plane transverse to the trochlear groove. In some cases, the bearing surface portion of the femoral implant includes an oblong geometry that is longer in the sagittal plane (i.e., parallel to the trochlear groove). In some cases, the bearing surface portion of the oblong femoral implant includes a three-circle profile. In some cases, the three-circle profile includes a middle circle that is perpendicular to the transverse and sagittal planes of the trochlear groove and two outer circles that are perpendicular to the transverse plane and angle away from the sagittal plane of the trochlear groove. In some cases, the three-circle profile includes a middle circle that is perpendicular to the transverse and sagittal planes of the trochlear groove and two outer circles that are perpendicular to the transverse plane and perpendicular to the sagittal plane. In some cases, the bearing surface portion of the femoral implant includes a round profile having a diameter value within a range from about 20 mm to about 40 mm. In some cases, the bearing surface portion of the femoral implant includes an oblong profile having a length value within a range from about 25 mm to about 40 mm. In some cases, the femoral implant has a three-circle oblong racetrack profile having a circle diameter of about 20 mm to about 35 mm. In some cases, the femoral implant has a thickness value within a range from about 4 mm to about 15 mm. In some cases, the femoral implant is a monolithic unit. In some cases, the patellar implant further includes at least one distal peg. In some cases the patellar implant peg further includes a titanium pin for X-ray visualization. In some cases, the patellar implant further includes a pocket for a cement mantle. In some cases the patellar implant includes highly crosslinked ultra-high molecular weight polyethylene. In some cases, the patellar implant includes vitamin E diffused highly crosslinked ultra-high molecular weight polyethylene. In some cases, the patellar implant includes a round geometry. In some cases, the patellar implant has a diameter value within a range from about 25 mm to about 30 mm. In some cases, the patellar implant has a thickness value that is greater than about 7.5 mm. In some cases, the patellar implant is a monolithic unit.

In another aspect, embodiments of the present disclosure encompass systems and methods for implanting an arthroplasty resurfacing system into a joint of a patient. Exemplary methods may include engaging a first implant of the resurfacing system with a portion of a bone of the joint of the patient, where the first implant includes a bearing surface portion that matches the geometry of the distal groove of the bone and a subchondral surface portion. Methods may further include engaging a second implant of the resurfacing system with a portion of a second bone of the joint of the patient, where the second implant includes a domed bearing surface portion and a subchondral surface portion. In some cases, the joint is a knee joint, a shoulder joint, a hip joint, an ankle joint, or a first metatarsal-phalangeal joint. In some cases, the joint is a knee joint, the first implant includes a femoral implant, and the second implant includes a patellar implant. In some cases, the femoral implant further includes at least one proximal peg. In some cases, a proximal peg of the femoral implant includes at least one barb. In some cases, the femoral implant further includes at least one anti-rotation spike. In some cases, a peg of the femoral implant includes multiple barbs. In some cases, the femoral implant further includes one or more anti-rotation spikes. In some cases, the femoral implant further includes one or more proximal pegs. In some cases, a proximal peg of the femoral implant includes a trabecular porous structure. In some cases, the subchondral surface portion of the femoral implant includes porous titanium. In some cases, the subchondral surface portion of the femoral implant includes an irregular lattice. In some cases, the bearing surface portion of the femoral implant includes nonporous stainless steel, nonporous cobalt chrome, nonporous ceramic, or any combination thereof. In some cases, the bearing surface portion of the femoral implant includes a round geometry. In some cases, the bearing surface portion of the femoral implant includes an oblong geometry that is longer in the plane transverse to the trochlear groove. In some cases, the bearing surface portion of the femoral implant includes an oblong geometry that is longer in the sagittal plane (i.e., parallel to the trochlear groove). In some cases, the bearing surface portion of the oblong femoral implant includes a three-circle profile. In some cases, the three-circle profile includes a middle circle that is perpendicular to the transverse and sagittal planes of the trochlear groove and two outer circles that are perpendicular to the transverse plane and angle away from the sagittal plane of the trochlear groove. In some cases, the three-circle profile includes a middle circle that is perpendicular to the transverse and sagittal planes of the trochlear groove and two outer circles that are perpendicular to the transverse plane and perpendicular to the sagittal plane. In some cases, the bearing surface portion of the femoral implant includes a round profile having a diameter value within a range from about 20 mm to about 40 mm. In some cases, the bearing surface portion of the femoral implant includes an oblong profile having a length value within a range from about 25 mm to about 40 mm. In some cases, the femoral implant has a three-circle oblong racetrack profile having a circle diameter of about 20 mm to about 35 mm. In some cases, the femoral implant has a thickness value within a range from about 4 mm to about 15 mm. In some cases, the femoral implant is a monolithic unit. In some cases, the patellar implant further includes at least one distal peg. In some cases the patellar implant peg further includes a titanium pin for X-ray visualization. In some cases, the patellar implant further includes a pocket for a cement mantle. In some cases the patellar implant includes highly crosslinked ultra-high molecular weight polyethylene. In some cases, the patellar implant includes vitamin E diffused highly crosslinked ultra-high molecular weight polyethylene. In some cases, the patellar implant includes a round geometry. In some cases, the patellar implant has a diameter value within a range from about 25 mm to about 30 mm. In some cases, the patellar implant has a thickness value that is greater than about 7.5 mm. In some cases, the patellar implant is a monolithic unit.

For purposes of summarization, certain aspects, advantages and novel features are described herein. Of course, it is to be understood that not necessarily all such aspects, advantages or features need to be present in any particular embodiment.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIGS. 1A to 1C depict aspects of example targeted joint resurfacing implants in a knee joint.

FIG. 1D illustrates aspects of an example joint resurfacing method.

FIGS. 2A-6 illustrate aspects of a round first implant of an example targeted joint resurfacing system.

FIGS. 7A-10B illustrate aspects of oblong first implants of an example targeted joint resurfacing system.

FIGS. 11A-22 illustrate aspects of a second implant of an example targeted joint resurfacing system.

FIGS. 23A-24B illustrate aspects of an example first femoral reamer for inserting an example joint resurfacing system.

FIGS. 25A-27C illustrate aspects of a femoral reamer for an example joint resurfacing system.

FIGS. 28A-29 illustrate aspects of a tibial pin guide for an example joint resurfacing system.

FIGS. 30A-31B illustrate aspects of a first tibial reamer for an example joint resurfacing system.

FIGS. 32A and 32B illustrate aspects of a second tibial reamer for an example joint resurfacing system.

FIGS. 33A and 33B illustrate aspects of a tibial pin guide with the second tibial reamer of FIGS. 32A and 32B for an example joint resurfacing system.

FIGS. 34A to 34C depict aspects of an exemplary tibial implant of an example joint resurfacing system.

FIGS. 35A to 35C depict aspects of an exemplary oblong femoral oblong implant of an example joint resurfacing system.

FIGS. 36A and 36B depict aspects of an exemplary round femoral round implant of an example joint resurfacing system.

FIGS. 37A to 37D depict aspects of an exemplary tibial implant of an example joint resurfacing system.

FIGS. 38A to 38D depict aspects of an exemplary round femoral implant of an example joint resurfacing system.

FIGS. 39A-40E depict aspects of an exemplary oblong femoral implant of an example joint resurfacing system.

FIGS. 41A to 41E depict aspects of an exemplary round femoral implant of an example joint resurfacing system.

FIGS. 42A-42E depict aspects of an exemplary tibial implant of an example joint resurfacing system.

FIGS. 43A to 43E illustrate aspects of an example first femoral reamer for inserting an example joint resurfacing system.

FIGS. 44A to 44E illustrate aspects of a femoral reamer for inserting an example joint resurfacing system.

FIGS. 45A to 45E illustrate aspects of a femoral reamer guide for an example joint resurfacing system.

FIGS. 46A to 46E illustrate aspects of an example femoral implant pin guide for inserting a femoral implant with an oblong profile.

FIGS. 47A to 47E illustrate aspects of an example femoral implant pin guide for inserting a femoral implant with a round profile.

FIGS. 48A to 48D illustrate aspects of a tibial reamer for an example joint resurfacing system.

FIGS. 49A to 49G illustrate aspects of a tibial pin guide for an example joint resurfacing system.

FIGS. 50A-50C illustrate aspects of an example round implant impactor.

FIGS. 51A-51C illustrate aspects of an example tibial implant impactor.

FIGS. 52A-52H illustrate various views of an example oblong femoral implant of an example joint resurfacing system.

FIGS. 53A-53F illustrate various views of an example oblong femoral implant of an example joint resurfacing system.

FIGS. 54A-54G illustrate various views of an example round femoral implant of an example joint resurfacing system.

FIG. 55A illustrates a side view of an example plastic portion of a tibial implant.

FIG. 55B illustrates an exploded view of an example metal portion of a tibial implant.

FIG. 55C illustrates a side view of an example metal portion of a tibial implant.

FIGS. 56A-56H illustrate various views of an example tibial implant of an example joint resurfacing system.

FIGS. 57A-57G illustrate various views of an example tibial implant of an example joint resurfacing system.

FIGS. 58A-58B illustrate example surgical steps of implant site access and sizing for a round femoral implant.

FIGS. 59A-59E illustrate aspects of an example femoral implant pin guide for inserting a femoral implant with a round profile.

FIGS. 59F and 59G illustrate the example femoral implant pin guide with a pin inserted through a central pin hole.

FIG. 60A illustrates an example surgical step of cartilage scoring for a round femoral implant.

FIGS. 60B-60G illustrate aspects of an example cartilage scoring tool.

FIGS. 61A-61C illustrate example surgical steps of powered reaming the implant site for a round femoral implant.

FIGS. 61D-611 illustrate aspects of an example first femoral reamer used for powered reaming.

FIGS. 62A-62F illustrate aspects of an example stopper component for use with the first femoral reamer.

FIGS. 63A-63C illustrate the first femoral reamer assembled with the stopper component.

FIGS. 63D and 63E illustrate an assembly of the first femoral reamer and the stopper component sliding over a guide pin.

FIG. 64A illustrates an example surgical step of removing the first femoral reamer.

FIGS. 64B and 64C illustrate an example surgical step of hand reaming the implant site for a round femoral implant.

FIGS. 64D-64I illustrate aspects of an example second femoral reamer used for hand reaming.

FIGS. 65A and 65B illustrate an example surgical step of inserting a trial to the reamed implant site.

FIG. 65C illustrates an example surgical step of drilling peg holes.

FIGS. 65D-65F illustrate example surgical steps of inserting a round femoral implant.

FIGS. 66A and 66B illustrate example surgical steps of implant site access and sizing for an oblong femoral implant.

FIGS. 66C-66G illustrate aspects of an example base of a femoral implant pin guide for inserting an oblong femoral implant.

FIGS. 66H and 66I illustrate the example femoral implant pin guide base with guide pins inserted through the pin holes.

FIGS. 67A-67E illustrate example surgical steps of reaming a first hole for an oblong femoral implant.

FIGS. 68A-68F illustrate example surgical steps of reaming a second hole in the bone by at least repeating the steps shown in FIGS. 66A, 66B, and 67A-67E.

FIGS. 69A-69E illustrate example surgical steps of reaming a third, central hole in the bone.

FIGS. 69F-60J illustrate aspects of an example central reamer guide.

FIGS. 70A-70E illustrate aspects of an example scoring tool guide.

FIGS. 70F and 70G illustrate the example scoring tool assembled with the scoring tool guide.

FIGS. 71A-71D illustrate the example first femoral reamer inserted into the example central reamer guide.

FIGS. 72A and 72B illustrate an example surgical step of inserting a trial to the reamed implantation site.

FIG. 72C illustrates an example surgical step of drilling peg holes.

FIGS. 72D-72F illustrate example surgical steps of inserting an oblong femoral implant.

FIG. 73A illustrates example surgical steps of exposing the surgical site for a tibial implant.

FIGS. 73B-73D illustrate example surgical steps of sizing the surgical site for a tibial implant and inserting a guide pin.

FIGS. 73E-731 illustrate aspects of an example tibial implant pin guide.

FIG. 73J illustrates an example surgical step of cartilage scoring for a tibial implant.

FIGS. 74A and 74B illustrate example surgical steps of providing powered reaming at a tibial implant site.

FIGS. 74C-74H illustrate aspects of an example first tibial reamer for powered reaming of the bone.

FIG. 75A illustrates example surgical steps of reaming by hand at the tibial implant site.

FIGS. 75B-75I illustrate aspects of an example second tibial reamer for hand reaming the bone.

FIGS. 76A-76F illustrate example surgical steps of trialing and inserting a tibial implant.

FIGS. 77A-77E illustrate example surgical steps for removing a round femoral implant.

FIGS. 78A-78F illustrate example surgical steps for removing an oblong femoral implant.

FIGS. 79A-79E illustrate example surgical steps for removing tibial implant.

FIGS. 80A-80D illustrate example micrographs of porous metal structures of an implant disclosed herein.

FIGS. 81A to 81C depict aspects of arthroplasty system implants, in accordance with some embodiments.

FIG. 81D illustrates aspects of arthroplasty method, in accordance with some embodiments.

FIGS. 82A to 82C illustrate aspects of a first implant of an arthroplasty system, in accordance with some embodiments.

FIGS. 83A and 83B illustrate aspects of a first implant of an arthroplasty system, in accordance with some embodiments.

FIGS. 84A to 84C illustrate aspects of first implants of arthroplasty systems, in accordance with some embodiments.

FIGS. 85A to 85H illustrate aspects of a first implant of an arthroplasty system, in accordance with some embodiments.

FIGS. 86A to 86H illustrate aspects of a first implant of an arthroplasty system, in accordance with some embodiments.

FIGS. 87A to 87H illustrate aspects of first implants of arthroplasty systems, in accordance with some embodiments.

FIGS. 88A to 88H illustrate aspects of a first implant of an arthroplasty system, in accordance with some embodiments.

FIG. 89 illustrates aspects of a second implant of an arthroplasty system, in accordance with some embodiments.

FIGS. 90A and 90B illustrate aspects of a first implant of an arthroplasty system, in accordance with some embodiments.

FIGS. 91A to 91D illustrate aspects of a first implant of an arthroplasty system, in accordance with some embodiments.

FIG. 92 illustrates aspects of a second implant of an arthroplasty system, in accordance with some embodiments.

FIGS. 93A to 93E illustrates aspects of a second implant of an arthroplasty system, in accordance with some embodiments.

FIGS. 94A to 94G illustrate aspects of a trochlear oblong pin guide system, in accordance with some embodiments.

FIGS. 95A to 95G illustrate aspects of a trochlear ooblong pin guide system, in accordance with some embodiments.

FIGS. 96A to 96F illustrate aspects of a trochlear round pin guide system, in accordance with some embodiments.

FIGS. 97A to 97F illustrate aspects of a trochlear oblong secondary reamer system, in accordance with some embodiments.

FIG. 98 illustrates aspects of a trochlear oblong secondary reamer system, in accordance with some embodiments.

FIGS. 99A and 99B illustrate aspects of a trochlear oblong secondary reamer system, in accordance with some embodiments.

FIG. 100 illustrates aspects of a trochlear oblong secondary reamer system, in accordance with some embodiments.

FIGS. 101A to 101F illustrate aspects of a trochlear ooblong and round secondary reamer system, in accordance with some embodiments.

FIGS. 102A to 102E illustrate aspects of a trochlear oblong reamer guide system, in accordance with some embodiments.

FIG. 103 illustrates aspects of a trochlear oblong reamer guide system, in accordance with some embodiments.

FIGS. 104A to 104E illustrate aspects of a trochlear ooblong reamer guide system, in accordance with some embodiments.

FIGS. 105A to 105D illustrates aspects of a trochlear ooblong reamer guide system, in accordance with some embodiments.

### DETAILED DESCRIPTION

Although certain embodiments and examples are described below, those of skill in the art will appreciate that this disclosure extends beyond the specifically disclosed embodiments and/or uses and obvious modifications and equivalents thereof. For example, although the example joint resurfacing systems are illustrated in the context of knee joint resurfacing procedures, the example systems disclosed herein may be implanted in joints other than the knee joint, for example but not limited to the shoulder joint, hip joint, ankle joint, wrist joint, hand digits, or first metatarsal-phalangeal joint. Thus, it is intended that the scope of the disclosure herein should not be limited by any particular embodiments described below.

### A. Overview of an Example Implant System & Method of Insertion

Currently available partial or unicompartmental knee replacement (also known as arthroplasty) systems often still involve large implants that require total resection and resurfacing of the affected femoral condyle and the tibial plateau (or one compartment of the tibial plateau). Existing femoral and/or tibial implants may require significant bone resection that goes through important bone surfaces, such as the anterior tibial cortex. For patients with a small cartilage defect in the knee joint, the partial knee arthroplasty implants are too invasive. Further, these implants will need to be replaced every ten to twenty years, which may be difficult due to excessive removal of the healthy bone. There may be insufficient healthy bone to provide fixation of a new implant in future revision surgeries or future surgeries using the same or different procedures and/or implants.

Moreover, some existing knee arthroplasty systems have multiple components on the femoral and/or tibial side that require in-patient assembly. Those systems also often require cementing the entire bone contacting surfaces for insertion and fixation. Additionally, certain existing femoral components are very thin and therefore do not provide adequate structural rigidity. Some existing knee arthroplasty systems include a two-component tibial implant, namely an insert and a tray. The insert may need to be made available in different thicknesses to match the femoral implant size needed and to adjust the height of the implant to accommodate the depth of saw cut made on the tibia.

The targeted or focal joint resurfacing systems and methods disclosed herein provide unique solutions that address at least some of these limitations and/or provide other advantages.

Unlike some of the existing implants (e.g., knee replacement implants) that have multi-components needing to be assembled during the surgery, the implants disclosed herein are provided as single components. The single-component implants can reduce surgery time and steps by avoiding having to assemble parts in the operating room. Having the implant manufactured as a single component can eliminate potential motion between different components (for example, the metal and plastic components in a tibial implant for knee replacement surgery), which can create wear debris, and/or eliminate the risk of dissociation of any component (for example, insert disassociation in knee replacement, which is a rare but serious complication). The single-component implant can come in the same thickness, which can reduce inventory. All sizes of the different implants disclosed herein (for example, the femoral implant and the tibial implant) can be compatible with each other.

Implant embodiments disclosed herein can be used to address osteochondral defects in early development, and/or to prevent or delay the need for a total joint replacement, which may be required if the defect worsens over time, and/or subchondral bone defects. The approach disclosed herein offers a solution between biological techniques and the joint arthroplasties. Some system embodiments include one implant per surface/defect correction. A smaller profile of the implant just sufficient for encompassing the osteochondral defect reduces the area of articulating surface that needs to be removed (e.g., resected or otherwise), thereby preserving as much healthy bone and/or tissue as possible. Porous in-growth and/or barbed peg features allow for strong fixation with cement or optionally cement-less fixation.

Exemplary arthroplasty resurfacing systems and methods disclosed herein can include the use of a first implant having a convex bearing surface portion and a subchondral surface portion, and/or a second implant having a concave bearing surface portion and a subchondral surface portion. The implant system can be configured for implantation into a joint of a patient. The joint may be, for example, a knee joint, a shoulder joint, a hip joint, an ankle joint, a first metatarsal-phalangeal joint, or the like.

In one embodiment, a joint resurfacing system can include two main implants available for the knee joint: a femoral implant; and a tibial implant with an angled articulating surface. The two implants may be inserted in combination, or individually in a patient. As noted above, the implant(s) may only be implanted on surfaces that contain osteochondral defects.

The femoral implant may be monolithic. In some embodiments, the femoral implant has a convex load bearing or articulating surface. The femoral implant may be made with metal or metal alloy. In some implementations, the implants disclosed herein are made with metal(s) or metal alloy(s) with desirable osteointegrative properties. One example of a suitable metal for the implants disclosed herein is titanium. One example of a suitable metal alloy for the implant disclosed herein is Ti-6Al-4V. In some embodiments, the femoral implant has a coating of a hard material (for example but not limited to titanium nitride (TiN) coating or diamond hardened titanium) on the articulating surface. The femoral implant can have a circular profile or an elongated (also referred to as oblong) profile. The femoral implant can have a barbed peg as well as irregular, porous 3D printed titanium sections on the subchondral surfaces to promote fixation. There can be anti-rotation spikes from the base of the implant to prevent rotational movement. The round implant can have a single peg. The oblong implant can have more than one peg.

The femoral implants may be round with a variety of diametric sizes and/or may have an oblong profile, with variable length and width. In some cases, the femoral implant may have a different profile, such as a racetrack profile or a three-circle profile. In some cases, the three circles in the oblong femoral implant have similar diameters. In some cases, the three circles have different diameters. In some cases, a profile of the femoral implant may include a first number of circle profiles of one diameter, and a second number of circle profiles having another diameter. Such profiles may require additional steps to a reaming/preparation process than a round profile.

In some embodiments, round femoral implants range in dimension diametrically from about 10 mm to about 35 mm, or about 12.0 mm to about 27.5 mm. Oblong femoral implants can range in length and width/diameter from about 10 mm×20 mm to about 35 mm×40 mm, or about 12.0 mm×20 mm to about 27.5 mm×40 mm or 27.5 mm×50 mm, or any ranges between these values. In some embodiments, the overall thickness of femoral implants can vary from 4 mm to 10 mm. The thickness of a femoral implant can refer to the distance from the inflection point on the bearing surface (maximum for the femoral implant) to the flat subchondral surface, where the porous section engages the bottom or surface of the reamed bone.

In some embodiments, the tibial implant has a plastic portion, which can include a concave, ultra-high molecular weight polyethylene (UHMWPE) load bearing or articulating surface. In some embodiments, the plastic portion can be a polyethylene cap. Although the present disclosure refers to UHMWPE as an example, a different biocompatible plastic material may be used instead of UHMWPE for the plastic portion. The plastic portion can be overmolded onto a metal portion, which can include at least a subchondral surface and a barbed peg. The tibial implant can have an angled articulating surface relative to the subchondral surface and/or the peg. The angle can facilitate an angled access of the tibial plateau and angled insertion of the tibial implant. This angle of implantation can be set early in the surgical procedure with an angled pin guide. The metal portion can be made of titanium or any other suitable metals or their alloy. In addition to the porous subchondral surface, the metal portion can include a regular (for example, diamond) porous structure on the metal portion as a binding site to the plastic portion. The porous subchondral surface can have a separate lattice structure and can be implanted deeper into the joint surface to promote bone in-growth. In some implementation, the lattice structure can be irregular (for example, similar to the trabecular bone structure). In some implementations, the lattice structure can be regular. In some implementations, the metal portion can be a titanium base. The metal portion of the implant can include anti-rotation spikes to prevent rotational movement.

The tibial implants can include various diametric sizes (and optionally different load bearing surface angles) to facilitate angled access and insertion. The tibial implant can also include optional tibial pin guides that vary in angle of pin entry. In some embodiments, tibial implants range in diameters from 15 mm to 25 mm. In some cases, the minimum overall thickness of a tibial implant is 5.0 mm. In some cases, a trough of a concave bearing surface to a subchondral surface is 5.0 mm or 5.05 mm. The thickness of a tibial implant can refer to the distance from the inflection point on the bearing surface (minimum for the tibial implant) to the subchondral surface, where the porous section engages the bottom or surface of the reamed bone.

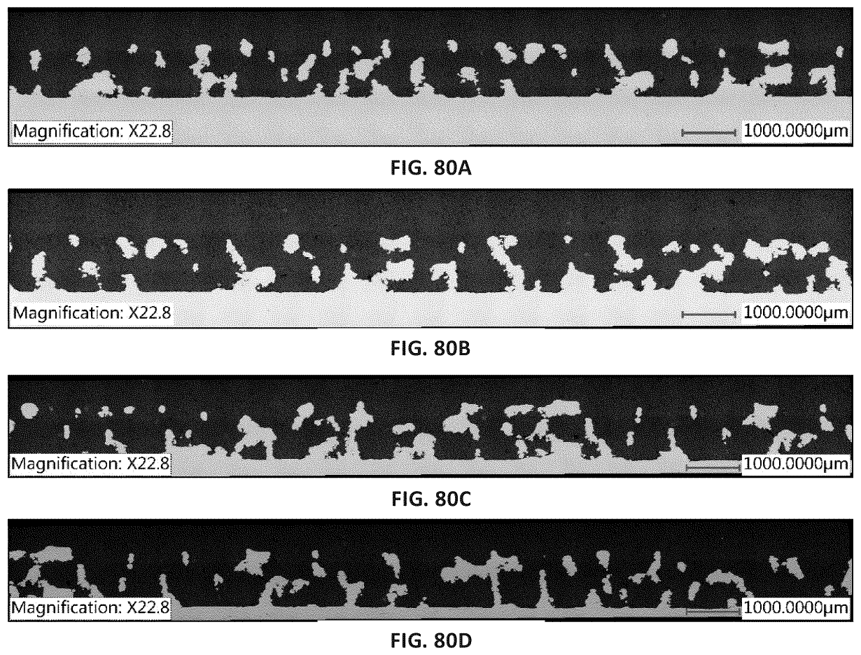

The femoral and/or tibial implants can include porous (e.g., irregular or regular) metal structures. The porous metal structures can be 3D printed. In some implementations, certain solid metal parts, such as the barbs on the peg, can also be 3D printed. One advantage of the porous metal structures is to help reduce implant thickness. The implants disclosed herein may not be designed to replace joint reconstruction. Rather, the implants disclosed herein may replace areas of the joint with bone and/or cartilage defects or injuries to alleviate joint pain. For these purposes, an implant with reduced thickness may be more preferable by preserving more healthy bone. Another advantage of the porous metal structures is to improve fixation strength. FIGS. 80A-80D illustrate micrographs of the example porous metal structures disclosed herein. Any of the porous metal structures disclosed herein can have about 30% to about 70% of porosity by volume, or about 65% to about 78% of porosity by volume. The porous metal structures can include average pore sizes of between about 100 μm to about 1,000 μm, or about 400 μm to about 550 μm, or about 600 μm. The fixation features of the implant allow for stronger, optionally cementless fixation. The stronger fixation and smaller profile compared to existing knee arthroplasty implants provide a higher level of mobility to patients, where other approaches and implants can limit patient mobility. Moreover, the implants disclosed herein can include a solid titanium peg core and solid titanium barbs with lattice structure on exterior bone-contacting surfaces of the peg, which can balance strength and bony ingrowth. In some implementations in which bone cement is used, only the peg of the implant is cemented, which combines good initial/primary fixation from cement with long term stability from bony ingrowth on uncemented surfaces (also referred to as secondary fixation).

Implant embodiments can have multi-peg or pegless versions. In a multi-peg implementation, the need for anti-rotation spikes may no longer be present such that spikes may not be included. In pegless options, the need for drilling preparation may no longer be present. A no-peg embodiment can be a helpful option to those patients with previous surgeries/hardware (e.g., interference screws). Implant embodiments encompass versions having barbless pegs that are a trabecular porous structure. Portions of an implant may include titanium, stainless steel, cobalt chrome, and the like. A bearing surface can optionally be made out of ceramic.

In exemplary embodiments, implants do not require assembly in the operating room (OR). In some embodiments, the femoral implant can be monolithic, with 3D printed titanium substrate and polished titanium nitride-coated articular surface, eliminating in situ assembly and risk of wear/corrosion between separate parts, and other disadvantages of in situ assembly as disclosed herein. In some embodiments, the connection method between the plastic and metal portions of the tibial implant can use compression molding over a porous section as a mechanical lock, which reduces the thickness of the connection site compared to other mechanical locks between similar components. A circumferential groove on the tibial implant can allow fixturing the metal portion to achieve a clean stop of plastic flow during the over molding process.

The present disclosure includes an angled, antegrade approach for the tibial implant to help protect more healthy articulating cartilage in the knee joint and provide easier access of the tibial plateau to the surgeon. In some embodiments, angled insertion of the tibial implant and the associated insertion tools allow for improved access to the tibial plateau in the presence of the femur condyles. The antegrade approach can be used in combination with partially releasing the anterior horn of the meniscus to improve access to the tibia and reattaching the meniscus after the tibial implant has been inserted.

The surgical methods disclosed herein can also facilitate the accuracy of hand reaming for fine tuning final implant fit, for example, by using both a powered reamer and a hand reamer. In some implementations, the top surface of a hand reamer can match the articular surface of an implant (for example, the height and contour of a femoral implant). This enables the surgeon to visualize where the articular surface of the implant will sit relative to the surrounding cartilage while still reaming, when adjustments to depth can be more easily made. The ability to visualize where the articular surface of the implant will sit can further allow the implant to be provided with just one thickness, even though the implant can have different diameter sizes.

In some implementations, a single-use hand reamer can be made using a combination of molded plastic with embedded metal blades, which can significantly reduce cost compared to all-metal design, while maintaining sharp cutting edges needed for reaming the bone.

Exemplary embodiments also encompass limited instrument sets and/or optionally the use of no cement, leading to reduced time for surgery and reduced risk of patient exposure. As cement can be a source of failure in orthopedic implants, the cementless application may have one less source of failure.

As noted above, the implants and methods of insertion disclosed herein may be used to treat arthritis and/or subchondral bone defects or injuries (with or without cartilage defects or injuries). The subchondral zone lies beneath the articular cartilage. This zone is made up of porous trabecular bone structure. This subchondral zone can be an independent source of pain. Lesions in the subchondral zone typically are evidenced by bony edema which increases its signal intensity on a T2-weighted MRI. The injury occurs when there are abnormal stresses that can cause microfractures into the trabecular bone. These abnormal stresses are due to local surrounding forces causing damage to the subchondral bone. These microfractures, which are thought to be a normal process of bone remodeling may heal if the rate of fractures outpaces the rate of healing or mending. These lesions, which cause pain in the subchondral zone, can be present with articular cartilage injuries, which can accelerate abnormal loading in subchondral zone and cause pain. These lesions in the subchondral zone can also be independent of any articular injury where the articular cartilage is still healthy. In order to repair the subchondral bony lesion, a cover can be placed over the subchondral zone injury. This cover can distribute the load and protect the injury site of the subchondral zone. In the present disclosure, the cover can be a portion of the femoral or tibial implant above the peg (that is, the portion between a bearing surface and a subchondral surface). In some implementations, the cover can act like a manhole cover. The cover can be made of a hard material (for example, metal or hard plastic as disclosed herein) that can interact with the articular cartilage above in this joint. The cover can also be biphasic with a hard and smooth cover on the superior surface of the joint and a porous (for example, trabecular, irregular, or regular) surface directly below (described as the subchondral surface in this disclosure) adjacent to the subchondral zone. This porous surface can not only support the subchondral zone of injury but also allow for improved rate of healing by bone ingrowth into the cover or the porous structure for support. The peg of the implant disclosed herein can be driven into the subchondral zone of injury. The peg can not only provide support for the surrounding zone of injury, but also by its size decrease the zone of injury by filling a void space so that the volume of healing required may be reduced. The peg can also include a porous metal structure (for example, mimicking trabecular bone) that allows bony ingrowth into the porous peg structure. Before insertion of the peg, the subchondral zone of injury can be prepared with a reamer. Reaming can provide access to a space that can decompress and stimulate the subchondral zone of injury. Insertion of the peg into the reamed space can allow for increased vascularization and relief of pressure in the subchondral zone of injury. This porous metal peg that is placed into the space can promote bony ingrowth and vascularity, supporting and healing the subchondral zone of injury.

Implant embodiments can also have variations to accommodate different bones and joints. Alternative embodiments can include slightly altered implants that are used in similar resurfacing procedures in other joints that frequently develop osteochondral defects, including, but not limited to, the shoulder, the hip, the ankle (talus), and the first metatarsal-phalangeal joint.

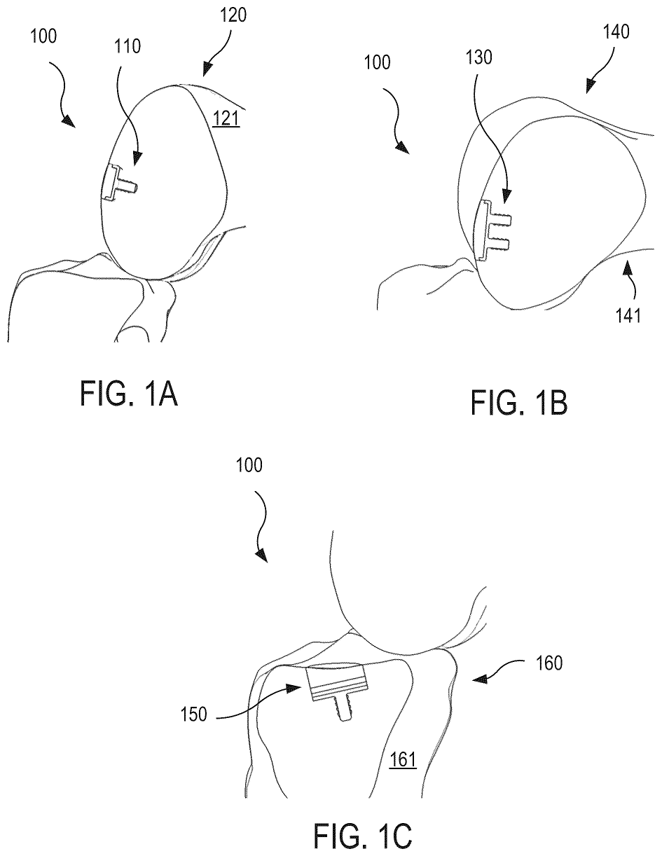

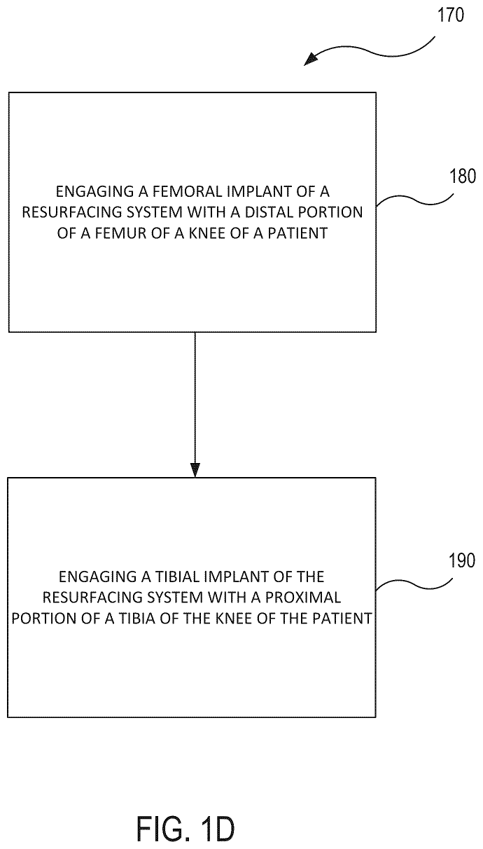

FIGS. 1A to 1C depict various aspects of an example targeted or focal knee joint resurfacing system 100, including a femoral implant and a tibial implant. FIG. 1A depicts a single-peg femoral implant 110 that is engaged with a distal portion 120 of a femur 121 of a patient. The single-peg femoral implant 110 can be any of the femoral implants with a single peg as disclosed herein. FIG. 1B depicts a multi-peg femoral implant 130 that is engaged with a distal portion 140 of a femur 141 of a patient. The multi-peg femoral implant 130 can be any of the femoral implants with two or more pegs as disclosed herein. FIG. 1C depicts a single-peg tibial implant 150 that is engaged with a proximal portion 160 of a tibia 161 of a patient. As further discussed herein, a femoral implant can have a convex bearing surface portion, the tibial implant can have a concave bearing surface portion, and the concave and convex bearing surface portions can slidingly engage one another during use of the resurfacing system, for example as the patient's knee joint undergoes flexion and extension. When implanted, the femoral implant 110 or 130 articulates against the proximal end of the tibial and/or the tibial implant 150, or the tibial implant 150 articulates against the distal end of the femur 121 and/or the femoral implant 110 or 130, in a non-constrained manner. The system 100 can be used in the targeted replacement of the articulating surfaces of the knee joint in instances where, due to compartmental degenerative disease and/or post-traumatic degenerative disease of the bone and/or cartilage, previous femoral condyle or tibial plateau fractures, deformity, or previous arthroplasty. Typically, the joint resurfacing system or surgical techniques are used when only the one side of the knee joint is affected.

The femoral and tibial implants disclosed herein can be used in any combination and/or number. A patient can receive either only one type or both types of the femoral implant and the tibial implant. In some implementations, when there are more than one small defect or injury on one side of the knee, more than one femoral or tibial implant can be inserted on the affected side. The number of femoral or tibial implants used may be the same or fewer than the number of defects or injuries, depending on the size and/or location of the defects or injuries. In some implementations, the joint resurfacing system or surgical techniques may be used on both sides of the knee joint. The femoral and tibial implants disclosed herein can also be used in any combination and/or number on one side of the knee, with partial knee arthroplasty performed on the other side of the knee. This combination can be helpful when one side of the knee has defect(s) that warrant a partial knee replacement procedure, but the defect(s) on the other side of the knee is/are relatively smaller. The femoral and tibial implants disclosed herein can also be used in any combination and/or number on one or both sides of the knee, in combination with one or more suitable trochlear implants and/or patellar implants between the distal end of the femur and the patellar. For example, the patient can receive one femoral implant on the weightbearing medial femoral condyle and a trochlear implant on the trochlear groove at the distal end of the femur.

FIG. 1D depicts aspects of a method 170 of implanting the system 100 into a compartment of a knee of a patient. As shown in this embodiment, the method 170 can include engaging a femoral implant of the resurfacing system with a distal portion of a femur of the knee of the patient, as indicated by step 180. The femoral implant can include a convex bearing surface portion and a subchondral surface portion. The method 170 can also include engaging a tibial implant of the resurfacing system with a proximal portion of a tibia of the knee of the patient, as indicated by step 190. The tibial implant can include a concave bearing surface portion and a subchondral surface portion. Alternatively, the surgical method can implant only the femoral implant or only the tibial implant.

### B. Example Femoral Implants

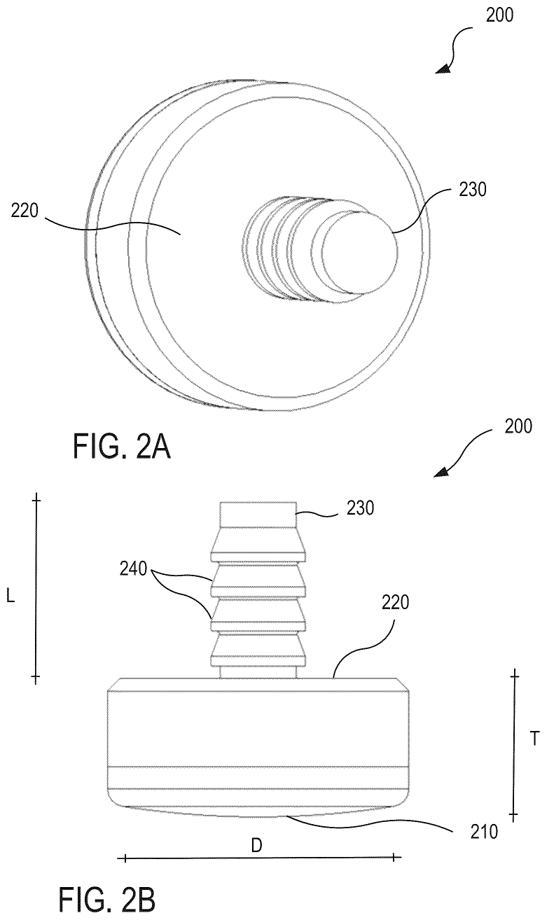

Examples of a femoral implant of a targeted knee joint resurfacing system are illustrated in FIGS. 2A-10B, 35A-36B, 38A-41E and 52A-54G. Features of any of the example femoral implants disclosed in any of these figures can be incorporated into the example femoral implants depicted in the other figures to the extent that those features are not mutually exclusive to the example femoral implants depicted in the other figures. Features with the same or similar functions are labeled with reference numbers that share the same last two digits.

The femoral implants disclosed herein can have a bearing surface portion. The bearing surface portion may include a convex bearing or articulating surface and a subchondral surface. The subchondral surface may be flat. The concave bearing surface can interface with an articulating surface of the tibial and/or a concave bearing surface of the tibial implant (which will be described in greater details below). The bearing surface may be made of solid metal and the subchondral surface may be porous. FIGS. 80A-80D illustrate micrographs of example porous metal structures disclosed herein. The convex bearing surface can further include a hard coating, for example but not limited to titanium nitride (TiN) coating or diamond hardened titanium. The femoral implant can further include one or more pegs and optionally one or more anti-rotation spikes. The peg may be made of a porous metal or its alloy. Alternatively, the peg may include a solid metal inner portion surrounded by a porous metal outer portion with surfaces that contact the bone. The solid metal inner portion may be contiguous with the solid metal portion of the bearing surface portion and optionally the spikes, if present. As another alternative, the peg may be solid throughout its structure. The peg can include barbs along at least a partial length of the peg. The barbs can be made of solid metal or its alloy. In the present disclosure, any porous metal structure can be 3D printed. In some implementations, the entire metal structure (including the porous and non-porous portions) can be 3D printed. In some implementations, the entire metal structure of the femoral implant may be manufactured as a monolithic unit such that no coupling mechanism is required to couple different parts of the metal structure together. Throughout the present disclosure, any reference to a metal may include an alloy of such metal. For example, the reference to titanium can include a titanium alloy, such as Ti-6A1-4V.

The bearing surface portion can have various shapes or profiles, for example, circular, oblong (that is, the width is smaller than the length), or otherwise. The different profiles of the bearing surface portion can provide flexibility in selecting an appropriate femoral implant that covers the entire osteochondral defect while excluding as much healthy bone and cartilage as possible. Non-limiting examples of implant kits are described below. In some implementations, a femoral implant kit can include a set of round femoral implants of different sizes from the range of sizes disclosed herein (e.g., 17.5 mm, 20.0 mm, and 22.5 mm) and a set of oblong femoral implants of different sizes from the range of sizes disclosed herein (e.g., 17.5 mm×30.0 mm, 20.5 mm×35.0 mm, and 22.5 mm×40.0 mm). In some implementations, a round femoral implant kit can include a set of round femoral implants of different sizes from the range of sizes disclosed herein (e.g., 17.5 mm, 20.0 mm, and 22.5 mm). In some implementations, an oblong implant kit can include a set of oblong femoral implants of different sizes from the range of sizes disclosed herein (e.g., 17.5 mm×30.0 mm, 20.5 mm×35.0 mm, and 22.5 mm×40.0 mm). The implant kit can include implants of any sizes disclosed herein. In some embodiments, the implant kits or surgical tool kits are sterilized before packaging. In some embodiments, the implants and single-use instruments are packaged before sterilization.

FIGS. 2A and 2B depict aspects of an exemplary femoral implant 200 in bottom perspective and side views, respectively. As shown, a femoral implant 200 can have a convex bearing surface portion 210 and a subchondral surface portion 220. The convex bearing surface portion 210 can operate as an articulating surface. The subchondral surface portion 220 can interface with a surgically prepared femoral bone surface. The femoral implant 200 can also include a proximal peg 230 extending proximally from the subchondral surface portion 220. The proximal peg 230 may include multiple barbs 240, which can improve fixation of the peg 230 in a drilled bone tunnel of the femur. In some cases, the convex bearing surface portion 210 of the femoral implant 200 can include nonporous titanium. In some cases, the convex bearing surface portion 210 can include nonporous stainless steel, nonporous cobalt chrome, nonporous ceramic, and the like. In some cases, the subchondral surface portion 220 of the femoral implant 200 can include porous titanium. In the example shown in FIGS. 2A and 2B, the convex bearing surface portion 210 of the femoral implant 200 has a round profile.

In some cases, the convex bearing surface portion 210 can provide a round profile having a diameter D with a value within a range from about 12 mm to about 20 mm, or about 12 mm to about 25 mm, or about 12 mm to about 27.5 mm. In some cases, the femoral implant 200 can have a thickness T with a value within a range from about 5 mm to about 10 mm. The thickness T of a femoral implant can refer to the distance from the inflection point on the bearing surface (maximum for the femoral implant) to the subchondral surface, where the porous titanium section engages a surface of the reamed bone of the patient. A peg length L can have a value of about 7 mm. In some cases, the proximal peg 230 can have a trabecular porous structure. In some cases, the femoral implant 200 is a monolithic unit. In some cases, the femoral implant 200 can include a bone screw fixation mechanism.

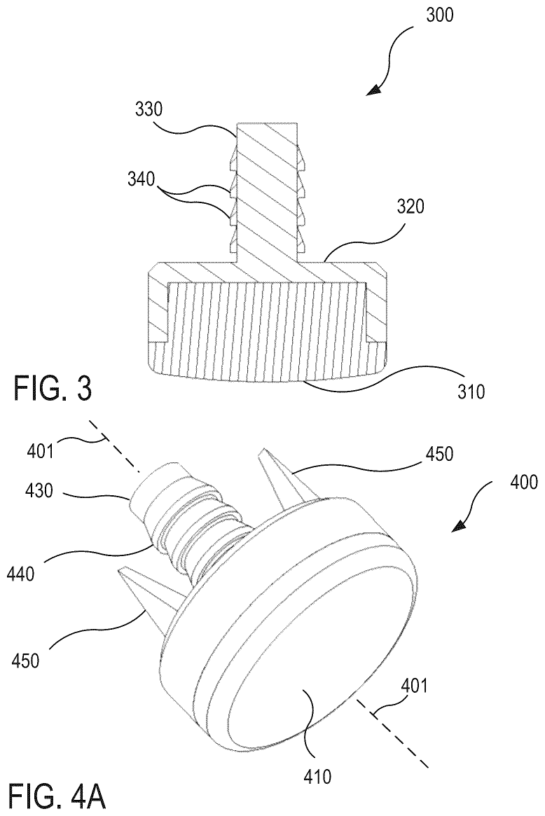

FIG. 3 depicts a cross-sectional view of an exemplary femoral implant 300. As shown, a femoral implant 300 can have a convex bearing surface portion 310 and a subchondral surface portion 320. The femoral implant 300 can also include a proximal peg 330 extending proximally from the subchondral surface portion 320. The proximal peg 330 can include multiple barbs 340. In some cases, the peg 330 and subchondral surface portion 320 can include a porous material, and the barbs 340 and the convex bearing surface portion 310 can include a solid or nonporous material. In some cases, the convex bearing surface portion 310 of the femoral implant 300 can include nonporous titanium. In some cases, the convex bearing surface portion 310 can include nonporous stainless steel, nonporous cobalt chrome, nonporous ceramic, and the like. In some cases, the barbs 340 can include a nonporous or solid material. In some cases, the subchondral surface portion 320 of the femoral implant 300 can include porous titanium. In this embodiment, the convex bearing surface portion 310 of the femoral implant 300 has a round profile. In some cases, the proximal peg 330 can have a trabecular porous structure. In some cases, the femoral implant 300 is a monolithic unit.

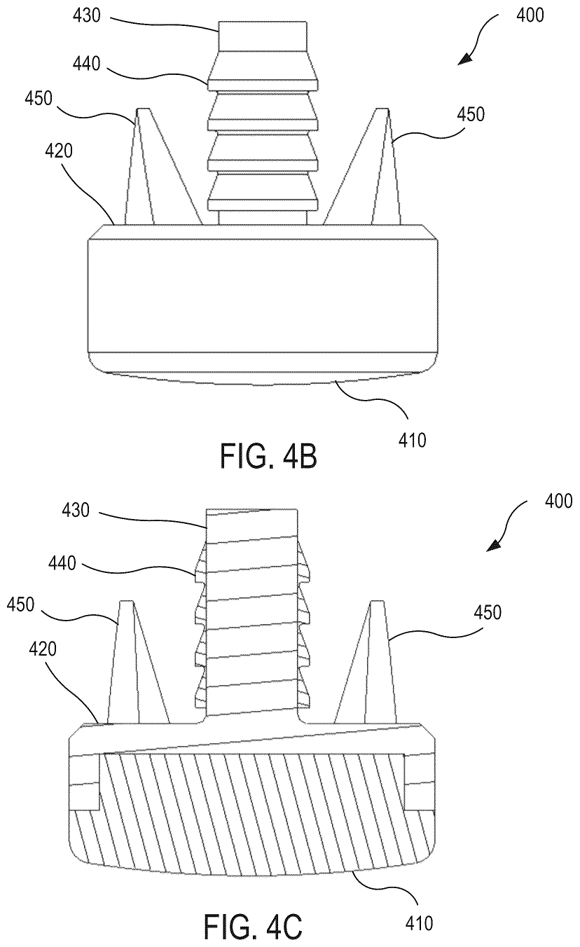

FIGS. 4A to 4G depict various views of an exemplary femoral implant 400. As shown here, a femoral implant 400 can have a convex bearing surface portion 410 and a subchondral surface portion 420. The femoral implant 400 can also include a proximal peg 430 extending proximally from the subchondral surface portion 420. The proximal peg 430 can include multiple barbs 440. In some cases, the convex bearing surface portion 410 of the femoral implant 400 can include nonporous titanium. In some cases, the convex bearing surface portion 410 can include nonporous stainless steel, nonporous cobalt chrome, nonporous ceramic, and the like. In some cases, the femoral implant 400 can include no porous structure. The convex bearing surface portion 410 of the femoral implant 400 can have a round profile. In some cases, the femoral implant 400 is a monolithic unit. In this embodiment, the femoral implant 400 can include multiple anti-rotation spikes or prongs 450. Such spikes or prongs 450 can help to prevent or inhibit the implant 400 from rotating about a central longitudinal axis 401 of the implant or proximal peg 430 when the implant is implanted in the patient's body. In some cases, the femoral implant 400 can include no porous structure (e.g., FIG. 4B). That is, the femoral implant 400 depicted in FIG. 4B is completely made of a solid or nonporous material. In other cases, such as shown in FIG. 4C, the femoral implant 400 can include a porous material in the peg 430, subchondral surface portion 420 and optionally the spikes 450, and a solid or nonporous material in the barbs 440 and the convex bearing surface portion 410.

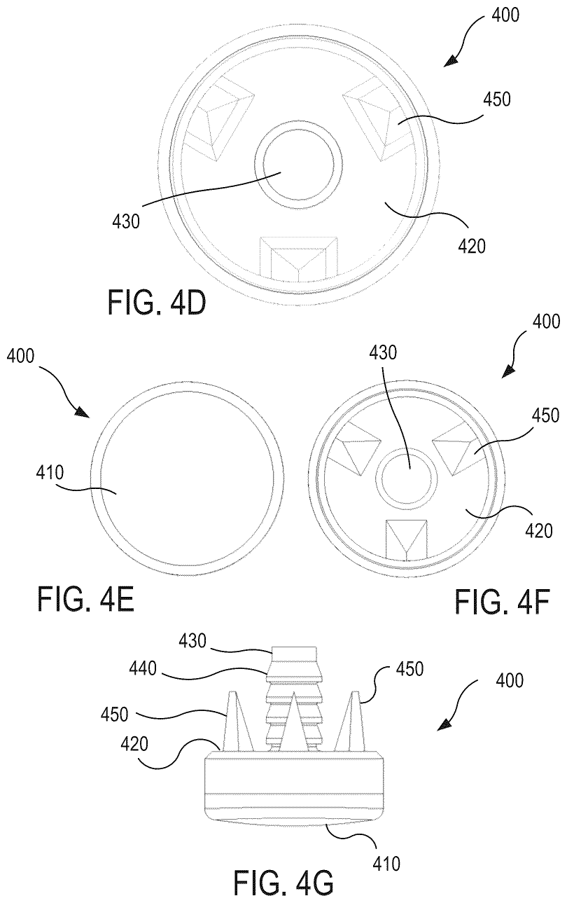

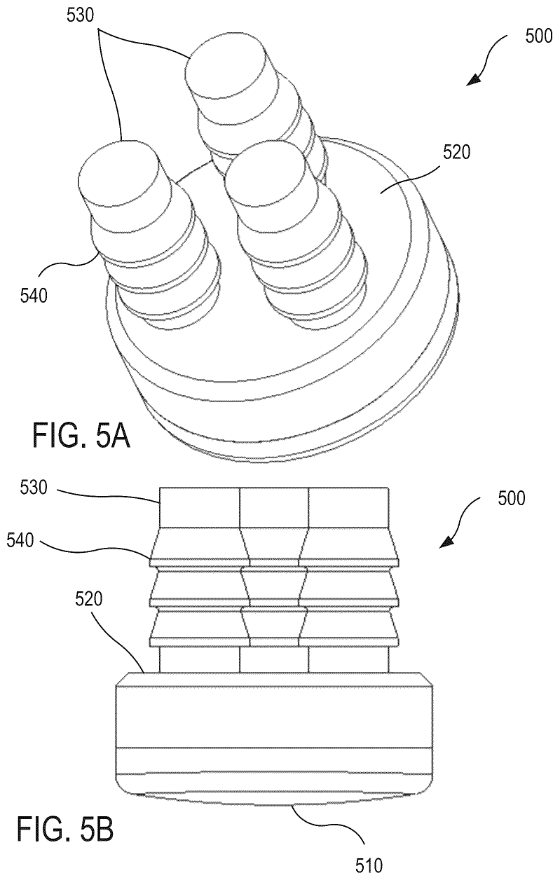

FIGS. 5A and 5B depict bottom perspective and side views of an exemplary femoral implant 500. As shown here, a femoral implant 500 can have a convex bearing surface portion 510 and a subchondral surface portion 520. The femoral implant 500 can also include multiple proximal pegs 530 extending proximally from the subchondral surface portion 520. The proximal pegs 530 can each include multiple barbs 540. In some cases, the convex bearing surface portion 510 of the femoral implant 500 can include nonporous titanium. In some cases, the convex bearing surface portion 510 can include nonporous stainless steel, nonporous cobalt chrome, nonporous ceramic, and the like. In some cases, the femoral implant can include no porous structure. In this embodiment, the convex bearing surface portion 510 of the femoral implant 500 has a round profile. In some cases, the femoral implant 500 is a monolithic unit. The femoral implant 500 can include no anti-rotation spikes. The presence of multiple pegs 530 can help to prevent or inhibit the implant 500 from rotating about a central longitudinal axis thereof when implanted in the patient's body.

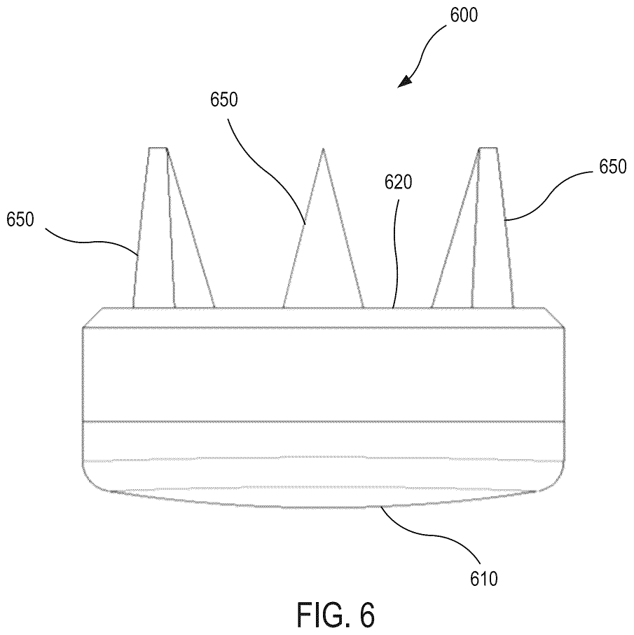

FIG. 6 depicts a side view of an exemplary femoral implant 600. As shown, a femoral implant 600 can have a convex bearing surface portion 610 and a subchondral surface portion 620. In some cases, the convex bearing surface portion 610 of the femoral implant 600 can include nonporous titanium. In some cases, the convex bearing surface portion 610 can include nonporous stainless steel, nonporous cobalt chrome, nonporous ceramic, and the like. In some cases, the femoral implant can include no porous structure. The convex bearing surface portion 610 of the femoral implant 600 can have a round profile. In some cases, the femoral implant 600 is a monolithic unit. The femoral implant 600 includes multiple anti-rotation spikes 650 and no proximal pegs. The presence of the spikes 650 can help to prevent or inhibit the implant 600 from rotating about a central longitudinal axis thereof when implanted in the patient's body.

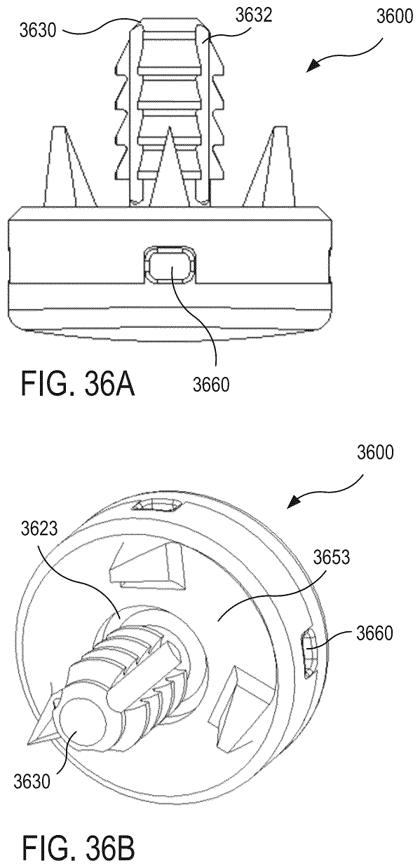

FIGS. 36A and 36B depict side and bottom perspective views of an exemplary round femoral implant 3600, that is with a round profile. The peg 3630 of the femoral implant can have one or more channels 3632 that allow for bone cement to flow along the length of the peg 3630. In some cases, the peg 3630 can be solid. In some cases, the femoral implant 3600 can include a well 3623 in a porous subchondral lattice 3653 of the subchondral surface portion at the base of the peg 3630 to receive overflowing bone cement. In some cases, the implant 3600 can include a notch 3660 just below the solid section of the convex bearing surface portion to allow for the connection of a removal tool, which will be described in greater detail herein.

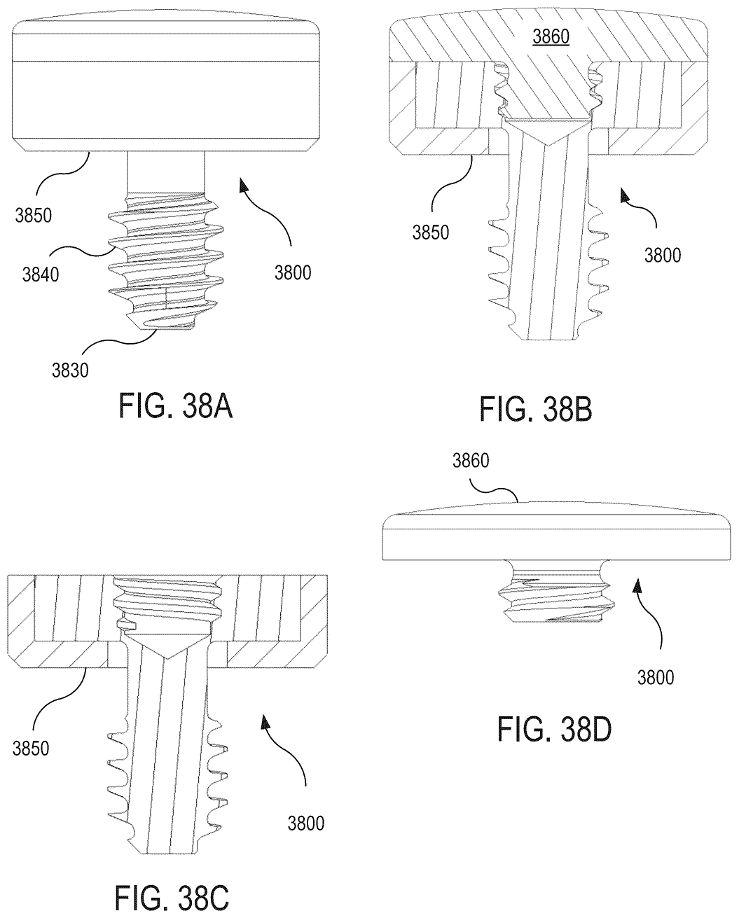

FIGS. 38A to 38D depict aspects of an exemplary femoral implant 3800 with a round profile that can be made of two parts, a bearing surface portion 3860 and a base 3850. In this example, the implant 3800 can have bone threads 3840 (e.g. on a peg 3830). A base 3850 of the femoral implant 3800 can be driven into bone by engagement of the bone with the threads 3840. The bearing surface portion 3860 of the implant 3800 can similarly be threaded into base 3850, or otherwise engage with the base 3850 via coupling.

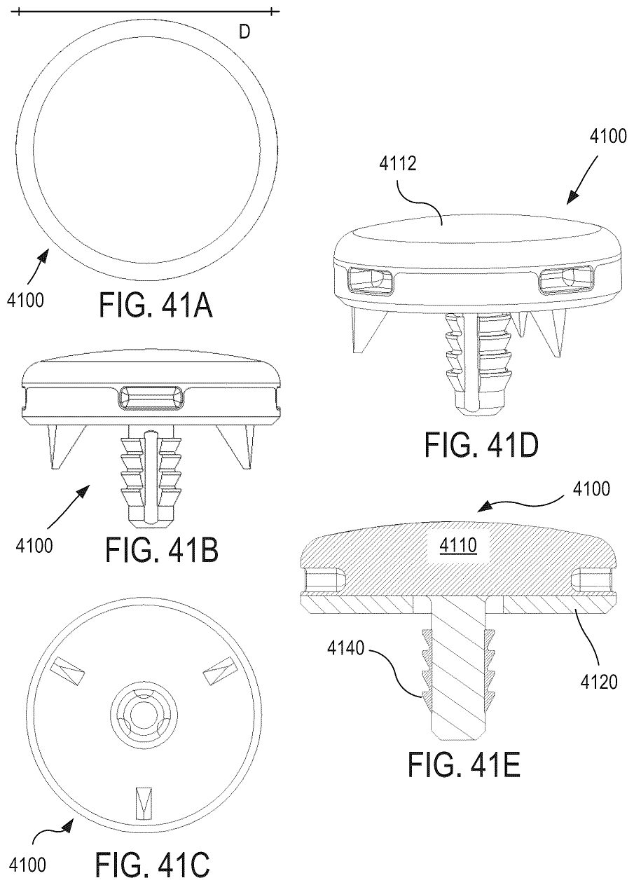

FIGS. 41A to 41E depict aspects of an exemplary femoral implant 4100 with a round profile. FIG. 41A provides a top plan view, FIG. 41B provides a side view, FIG. 41C provides a bottom plan view, FIG. 41D provides a perspective view, and FIG. 41E provides a cross-section view. In some cases, the bearing surface portion of the femoral implant 4100 can have an outer diameter D of about 17.5 mm. As shown in FIG. 41E, the peg barbs 4140 and the convex surface portion 4110 of the implant 4100 can be made of a solid material, such as solid titanium. A subchondral surface portion 4120 of the implant 4100 can be made of a non-solid material, such as a random lattice, which may include a material such as titanium. In some cases, a bearing surface 4112 can include a titanium nitride (TiN) coating.

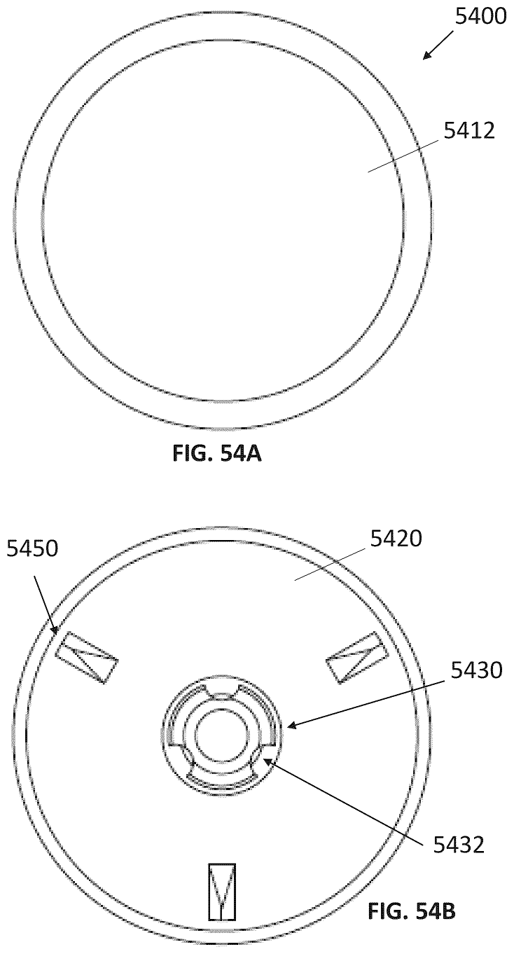

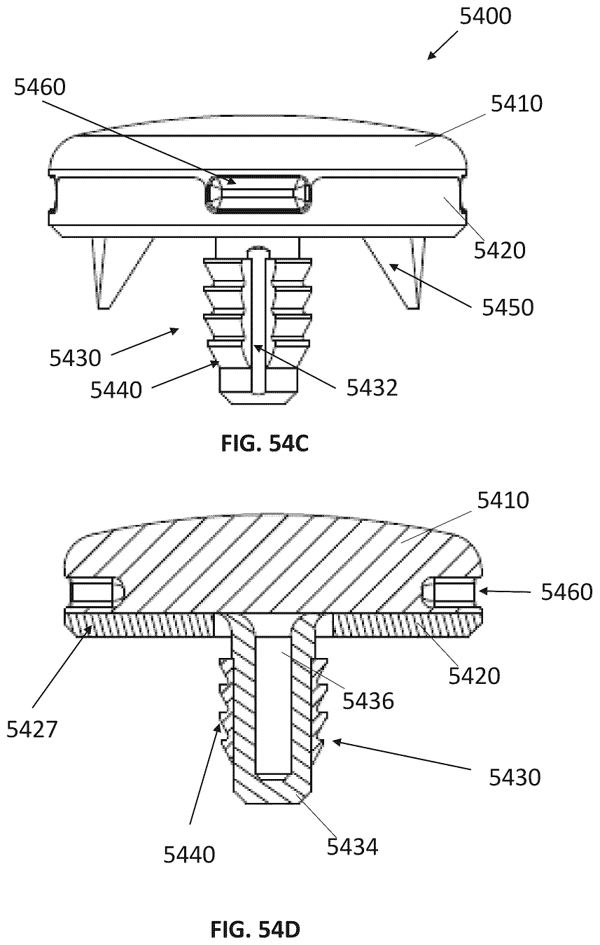

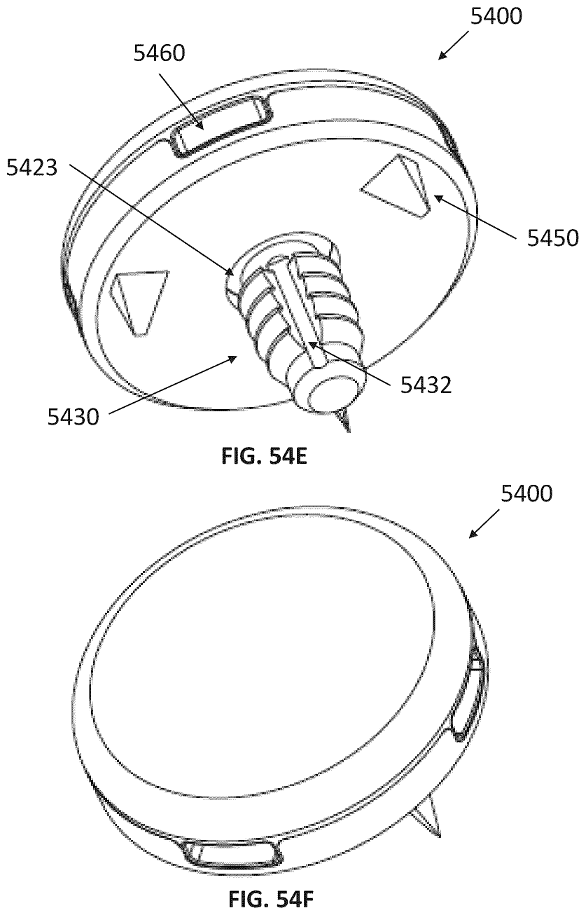

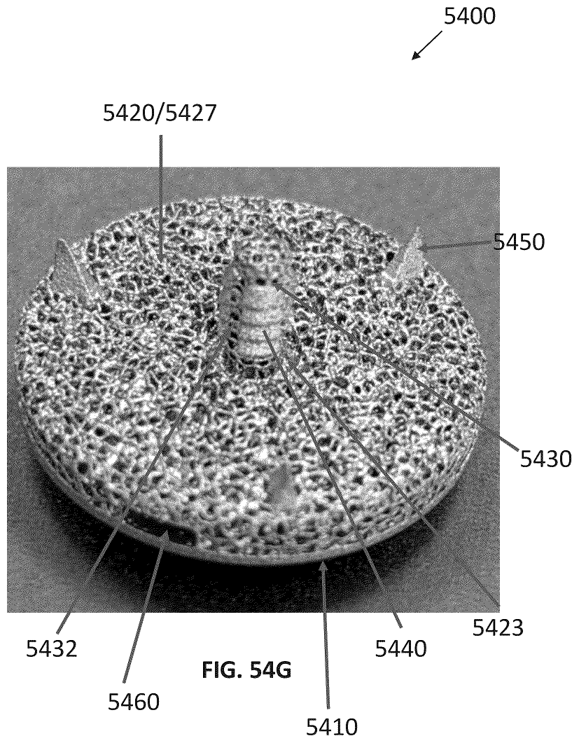

FIGS. 54A to 54G illustrate aspects of an example femoral implant 5400 with a round profile. As shown, a femoral implant 5400 can have a convex bearing surface portion 5410 and a subchondral surface portion 5420. The subchondral surface portion 5420 may have a flat bone contacting surface. The convex bearing surface 5412 can have a spherical diameter of about 60 mm to about 80 mm, for example, about 70 mm. The femoral implant 5400 can also include a proximal peg 5430 extending proximally from the subchondral surface portion 5420. The peg 5430 can have a length of about 5 mm to about 10 mm. The peg 5430 can have an outer diameter of about 2.5 mm to about 3.5 mm. The proximal peg 5430 may include multiple barbs 5440. The outer diameter of the peg 5430 at the barbed region can be between about 3.5 mm to about 4.5 mm. The barbs 5440 may be tapered, which can further improve fixation of the peg 5430 in a drilled bone tunnel of the femur (with or without bone cement). The femoral implant 5400 can include multiple anti-rotation spikes or prongs 5450 extending proximally from the subchondral surface portion 5420. For example, the femoral implant 5400 can include three anti-rotation spikes or prongs 5450 evenly spaced and at the same distance from the center of the round profile. Such spikes or prongs 5450 can help to prevent or inhibit the implant 5400 from rotating about a central longitudinal axis of the femoral implant 5400 or the proximal peg 5430 when implanted in the patient's body.

The peg 5430 of the femoral implant 5400 can have one or more channels 5432 (for example, three as shown in FIG. 54B, or two, or more) along at least a portion of the length of the peg 5430. The channels 5432 can allow for bone cement to flow along the length of the peg 5430 if bone cement is used, or allow for reduced pressure in the drilled hole in the bone if used for a cementless application. The channels 5432 can have a depth such that they cut through not only the barbs 5440 but also a part of the peg 5430. The channels 5432 can extend along the length of the peg 5430 or have any other suitable size/shape. The femoral implant 5400 can include a well 5423 to receive overflowing bone cement. The well 5423 can be in a porous subchondral lattice 5427 of the subchondral surface portion at the base of the peg 5430. The well 5423 can have a diameter of about 4.5 mm to about 5.5 mm.

The femoral implant 5400 can include two or more notches or pockets 5460 (for example, three or four) to allow for the connection of an insertion tool or a removal tool. As shown in FIG. 54D, the pockets 5460 can be located just above the porous subchondral surface portion 5420. The pockets 5460 can be located diametrically opposite each other or evenly spaced around the perimeter of the round profile. In some cases, the femoral implant 5400 may be a monolithic unit.

The femoral implant 5400 can have the same or similar features as the femoral implant 4100, but may include differences from the femoral implant 3900 as described herein with reference to FIGS. 54A to 54G. For example, as shown in FIG. 54D, the femoral implant 5400 can include a porous material in at least an outer portion 5434 of the peg 5430, subchondral surface portion 5420 (and optionally the spikes 5450), and a solid or nonporous material in the barbs 5440 and the convex bearing surface portion 5410 (and optionally the spikes 5450 and/or an inner or core portion 5436 of the peg 5430). In some implementations, the inner portion 5436 of the peg 5430 can be contiguous with the solid bearing surface portion 5410. The solid/nonporous material can include nonporous titanium or any other suitable materials disclosed herein, such as nonporous stainless steel, nonporous cobalt chrome, nonporous ceramic, and the like. The convex bearing surface 5412 can be coated with a hard material, for example, TiN or diamond hardened titanium. The porous material can include porous titanium or any other suitable materials disclosed herein. The porous material of the subchondral surface portion 5420 (and optionally an outer portion 5434 of the peg 5430) can have a trabecular porous or irregular lattice structure. The irregular lattice can facilitate osteointegration. The irregular lattice disclosed herein can have about 60-75% porosity by volume, about 600 μm average pore size, and about 225 μm strut thickness. The porous layer can be about 1.0 mm thick. The peg 5430 with the tapered barbs 5440 can provide primary fixation (optionally with bone cement) and the lattice can provide secondary fixation by osteointegration.

In some embodiments, the round femoral implant examples disclosed herein can have a diameter D having a value within a range from about 17.5 mm to about 27.5 mm. Exemplary diameter dimensions for a femoral round implant include 17.5 mm, 20 mm, 22.5 mm, 25 mm, 27.5 mm, and the like. The thickness of the round femoral implant examples (the distance from the inflection point on the bearing surface (maximum for the femoral implant) to the flat subchondral surface) can range between about 4.5 mm to about 7.5 mm, or between about 5 mm to about 7 mm.

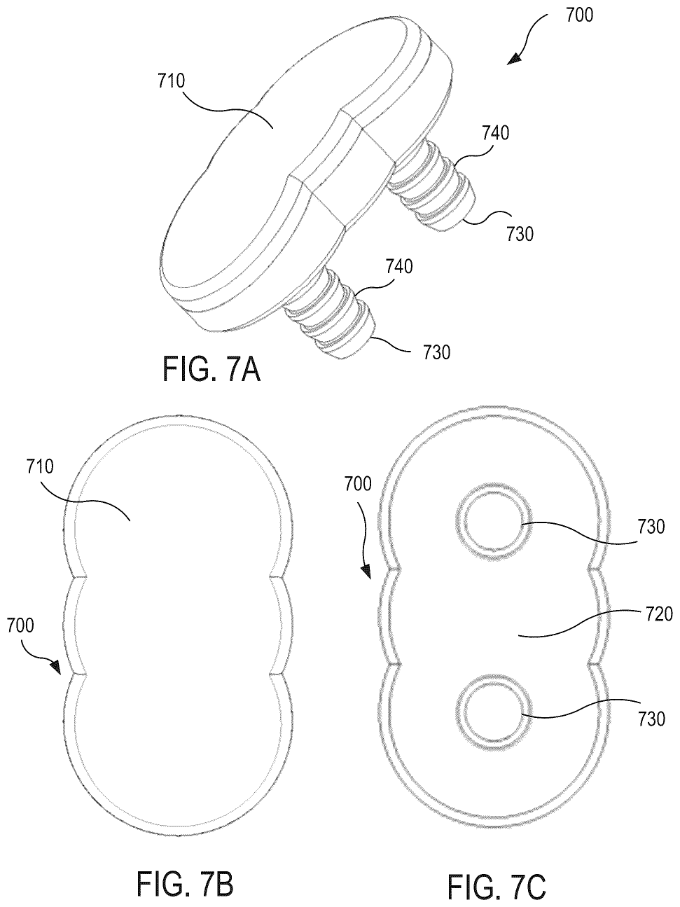

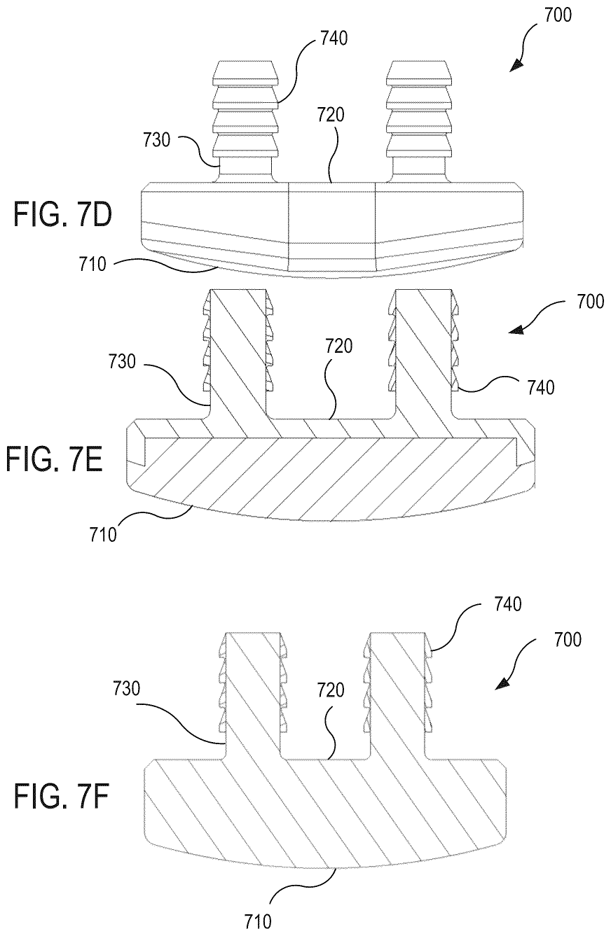

Turning to femoral implants with an oblong profile, FIGS. 7A to 7F depict aspects of such exemplary femoral implants 700. Features of the round femoral implants can be incorporated into the oblong femoral implants to the extent the features are not mutually exclusive to an oblong femoral implant. Features of the oblong femoral implants can be incorporated into the round femoral implants to the extent the features are not mutually exclusive to a round femoral implant. As shown, a femoral implant 700 can have a convex bearing surface portion 710 and a flat subchondral surface portion 720. The femoral implant 700 can also include one or more proximal pegs 730 extending from the subchondral surface portion 720.

In some cases, the subchondral surface portion 720 and/or one or more the proximal pegs 730 can include a porous material, such as porous titanium. The proximal pegs 730 include multiple barbs 740. In some cases, the convex bearing surface portion 710 of the femoral implant 700 can include nonporous titanium. In some cases, the convex bearing surface portion 710 can include nonporous stainless steel, nonporous cobalt chrome, nonporous ceramic, and the like. In some cases, the pegs 730 and subchondral surface portion 720 include a porous material, and the barbs 740 and the convex bearing surface portion 710 includes a solid or nonporous material (e.g., FIG. 7E). In some cases, the femoral implant can include no porous structure (e.g., FIG. 7F). That is, the femoral implant depicted in FIG. 7F is completely made of a solid or nonporous material. The convex bearing surface portion 710 of the femoral implant 700 has a three-circle or oblong profile. The three-circle profile provides a surface portion that has three sections formed by three overlapping circles, which allows for simple reaming with a circular reamer (e.g. in 3 adjacent reaming locations on the patient's bone, with overlapping circles). The pegs 730 may align along a longitudinal axis of the oblong profile. In FIG. 7A, the two pegs 730 may extend from the center of one of the two outer circles. In some cases, the femoral implant 700 can be a monolithic unit. In this embodiment, the femoral implant 700 includes no anti-rotation spikes, although such spikes as disclosed herein may be present in other embodiments.

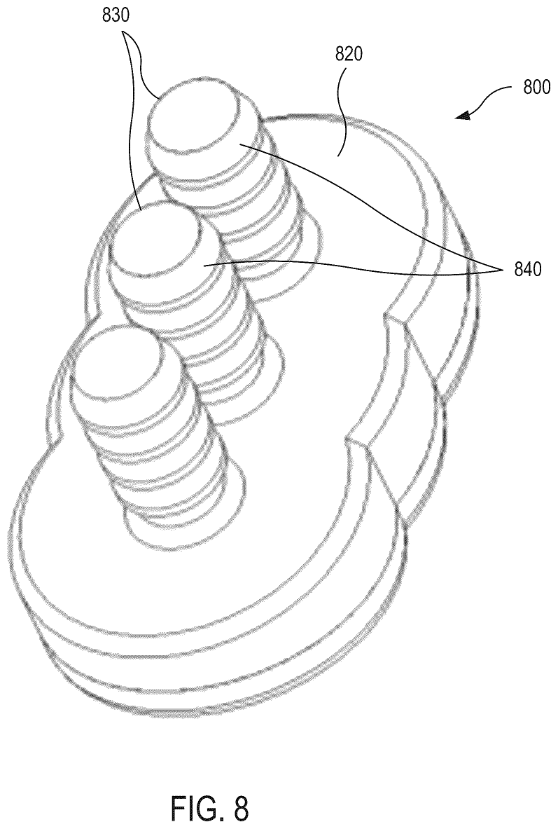

FIG. 8 depicts aspects of an exemplary femoral implant 800 with an oblong profile. As shown here, a femoral implant 800 can have a convex bearing surface portion and a subchondral surface portion 820. The femoral implant 800 can also include one or more proximal pegs 830, for example, three pegs 830 aligned with a longitudinal axis of the oblong profile as shown in FIG. 8. In some cases, each peg 830 may extend from the center of one of the three circles. In some cases, the subchondral surface portion 830 and/or one or more the proximal pegs 830 can include a porous material, such as porous titanium. In the embodiment depicted here, proximal pegs 830 include multiple barbs 840. In some cases, the convex bearing surface portion of the femoral implant 800 can include nonporous titanium. In some cases, the convex bearing surface portion can include nonporous stainless steel, nonporous cobalt chrome, nonporous ceramic, and the like. In some cases, the femoral implant can include no porous structure. The femoral implant 800 has a three-circle profile. In some cases, the femoral implant 800 is a monolithic unit. In this embodiment, the femoral implant 800 includes no anti-rotation spikes, although such spikes may be present in other embodiments.

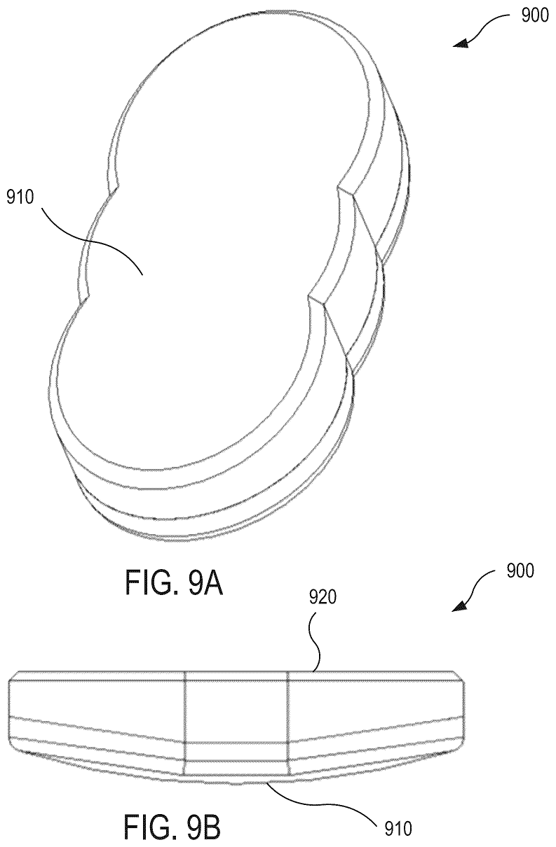

FIGS. 9A and 9B depict aspects of an exemplary femoral implant 900 with an oblong profile. As shown, a femoral implant 900 can have a convex bearing surface portion 910 and a subchondral surface portion 920. The femoral implant 900 does not include proximal pegs, although such pegs can be present in other embodiments. In some cases, the subchondral surface portion 920 can include a porous material, such as porous titanium. In some cases, the convex bearing surface portion of the femoral implant 800 can include nonporous titanium. In some cases, the convex bearing surface portion 910 can include nonporous stainless steel, nonporous cobalt chrome, nonporous ceramic, and the like. In some cases, the femoral implant can include no porous structure. The femoral implant 900 has a three-circle profile. In some cases, the femoral implant 900 is a monolithic unit. In this embodiment, the femoral implant 900 includes no anti-rotation spikes, although such spikes may be present in other embodiments.

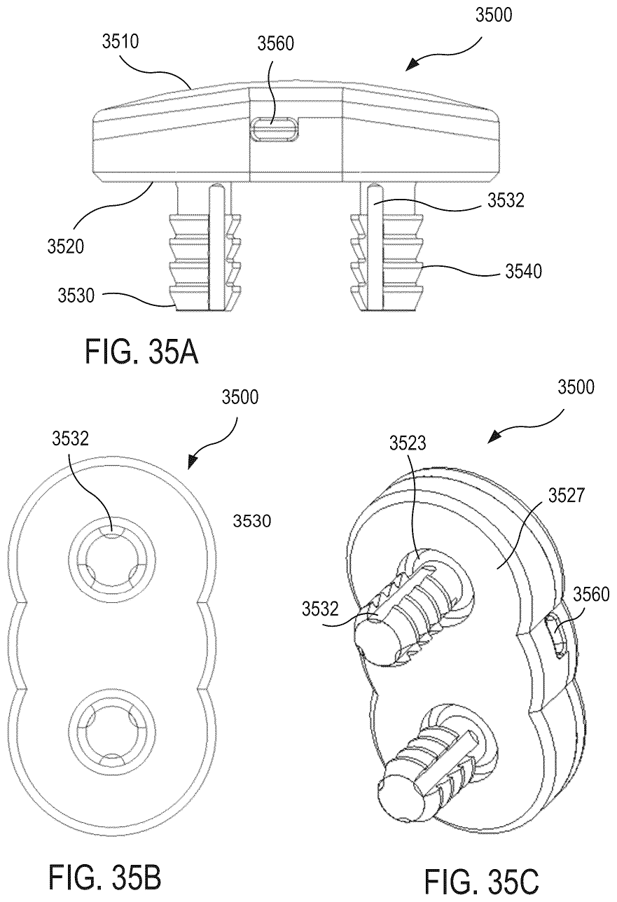

FIGS. 35A to 35C depict aspects of an exemplary femoral oblong implant 3500 with an oblong profile. The femoral implant 3500 can include a convex bearing surface portion 3510 and a subchondral surface portion 3520. The femoral implant 3500 can also include one or more (for example, two) proximal pegs 3530. In some cases, the subchondral surface portion 3520 and/or one or more the proximal pegs 3530 can include a porous material, such as porous titanium. In the embodiment depicted here, proximal pegs 3530 include multiple barbs 3540. In some cases, the convex bearing surface portion 3510 of the femoral implant 3500 can include nonporous titanium. In some cases, the convex bearing surface portion 3510 can include nonporous stainless steel, nonporous cobalt chrome, nonporous ceramic, and the like. In some cases, the pegs 3530 and subchondral surface portion 3520 can include a porous material, and the barbs 3540 and the convex bearing surface portion 3510 can include a solid or nonporous material. In some cases, the femoral implant can include no porous structure. That is, the femoral implant can be completely made of a solid or nonporous material. The convex bearing surface portion 3510 of the femoral implant 3500 has a three-circle or oblong profile. The three-circle profile provides a surface portion that has three sections formed by three overlapping circles, which allows for simple reaming with a circular reamer (e.g. in 3 adjacent reaming locations on the patient's bone, with overlapping circles). The pegs 3530 may align along a longitudinal axis of the oblong profile. In FIG. 35B, the two pegs 3530 may extend from the center of one of the two outer circles. In some cases, the femoral implant 3500 is a monolithic unit. In this embodiment, the femoral implant 3500 includes no anti-rotation spikes, although such spikes may be present in other embodiments. In this embodiment, the one or more pegs 3530 can have one or more channels 3532 (for example, three as shown in FIG. 35B, or two, or more) that allow for bone cement to flow along the length of the peg 3530. The channels 3532 can extend along the length of the peg 3530 or have any other suitable size/shape. In some cases, one or more pegs 3530 can be solid. In some cases, the implant 3500 can include one or more wells 3523 in a porous subchondral lattice 3527 of the subchondral surface portion 3520. A well 3523 can be located at the base of each of the pegs 3530 to contain overflowing cement. In some cases, the implant 3500 can include a notch or pocket 3560 just below the solid section of the convex bearing surface portion 3510 to allow for the connection of a removal tool.

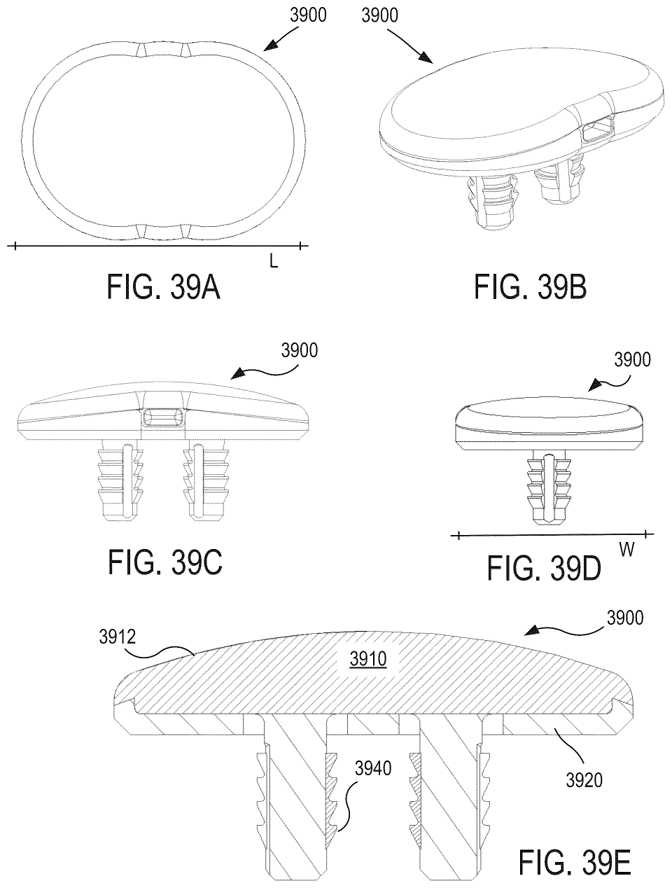

FIGS. 39A to 39E depict aspects of an exemplary femoral implant 3900 with an oblong profile. FIG. 39A provides a top plan view, FIG. 39B provides a perspective view, FIG. 39C provides a front view, FIG. 39D provides a right side view, and FIG. 39E provides a cross-section view. The oblong implant 3900 can have a length L of about 25 mm and a width W of about 17.5 mm. As shown in FIG. 39E, the peg barbs 3940 and the convex surface portion 3910 of the implant 3900 can be made of a solid material, such as solid titanium. A subchondral surface portion 3920 of the implant 3900 can be made of a non-solid material, such as a random lattice, which may include a material such as titanium. In some cases, a bearing surface 3912 includes a titanium nitride (TiN) coating.

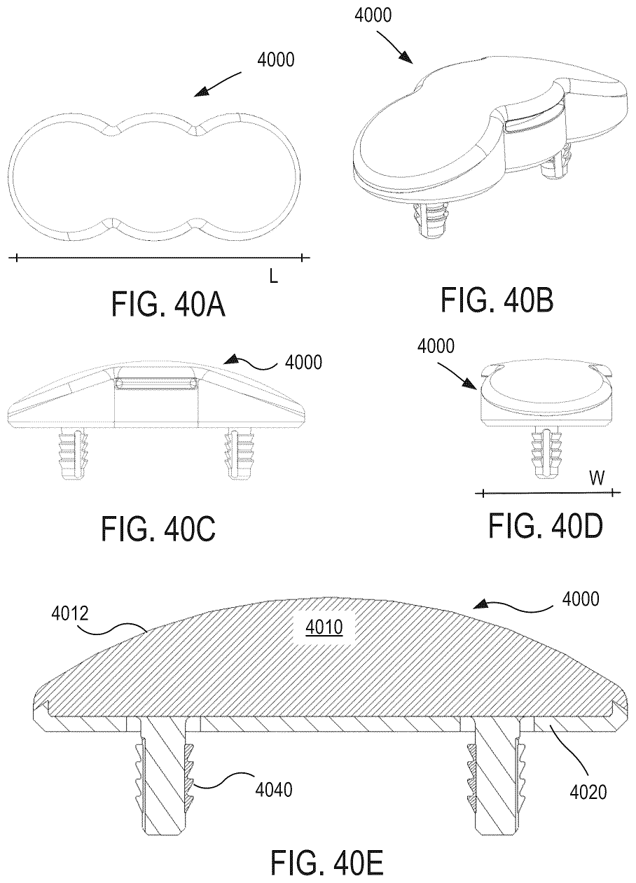

FIGS. 40A to 40E depict aspects of an exemplary femoral implant 4000 with an oblong profile. FIG. 40A provides a top plan view, FIG. 40B provides a perspective view, FIG. 40C provides a front view, FIG. 40D provides a right side view, and FIG. 40E provides a cross-section view. The femoral implant 4000 can have a length L of about 40 mm and a width W of about 17.5 mm. As shown in FIG. 40E, the peg barbs 4040 and the convex surface portion 4010 of the implant 4000 can be made of a solid material, such as solid titanium. A subchondral surface portion 4020 of the implant 4000 can be made of a non-solid material, such as a random lattice, which may include a material such as titanium. In some cases, a bearing surface 4012 includes a titanium nitride (TiN) coating.

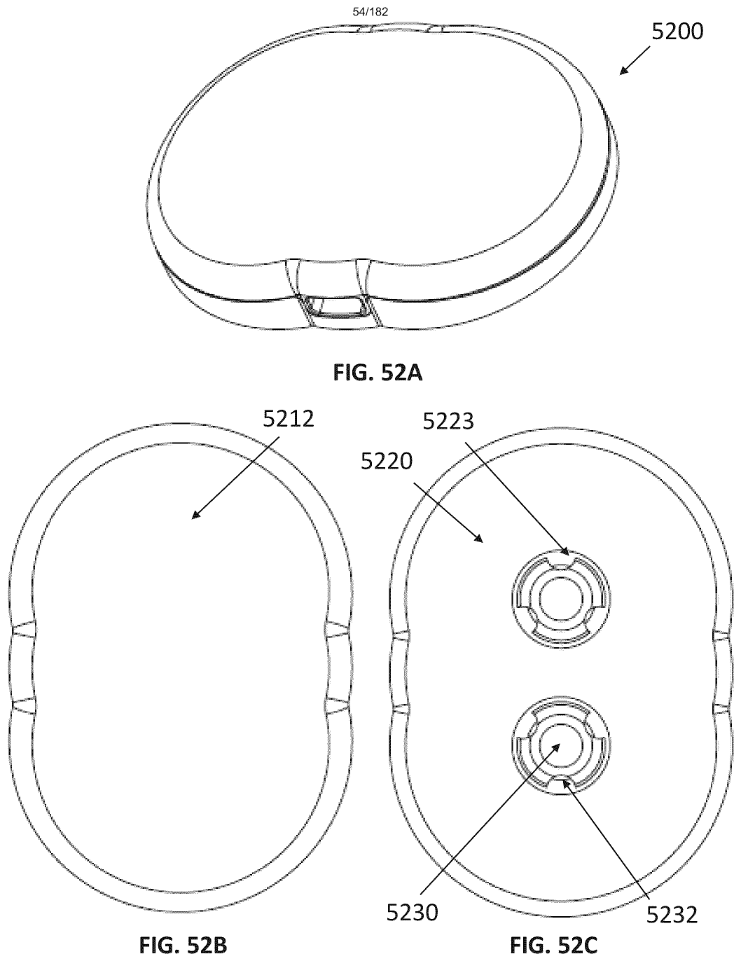

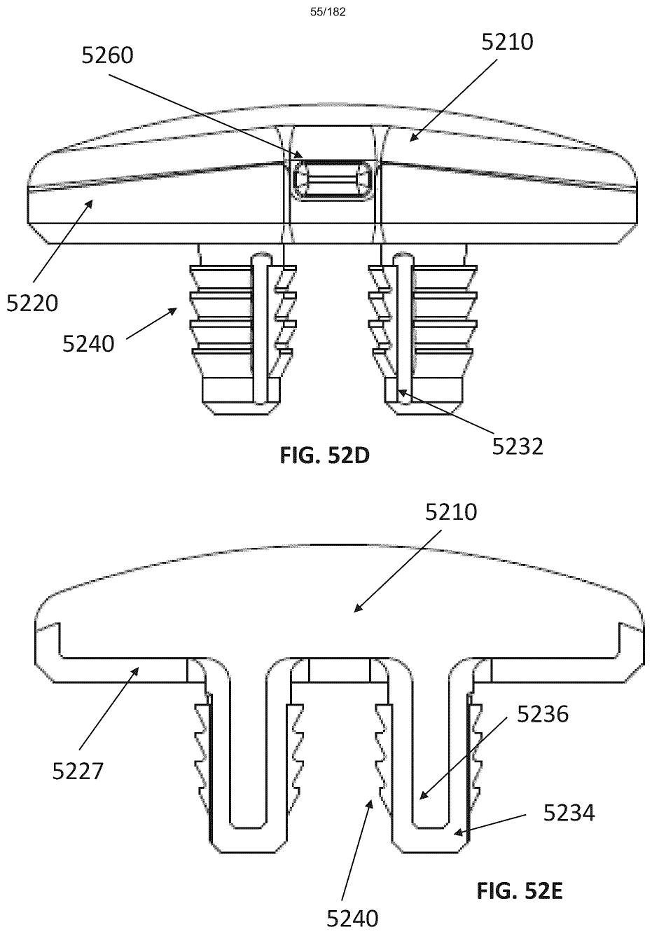

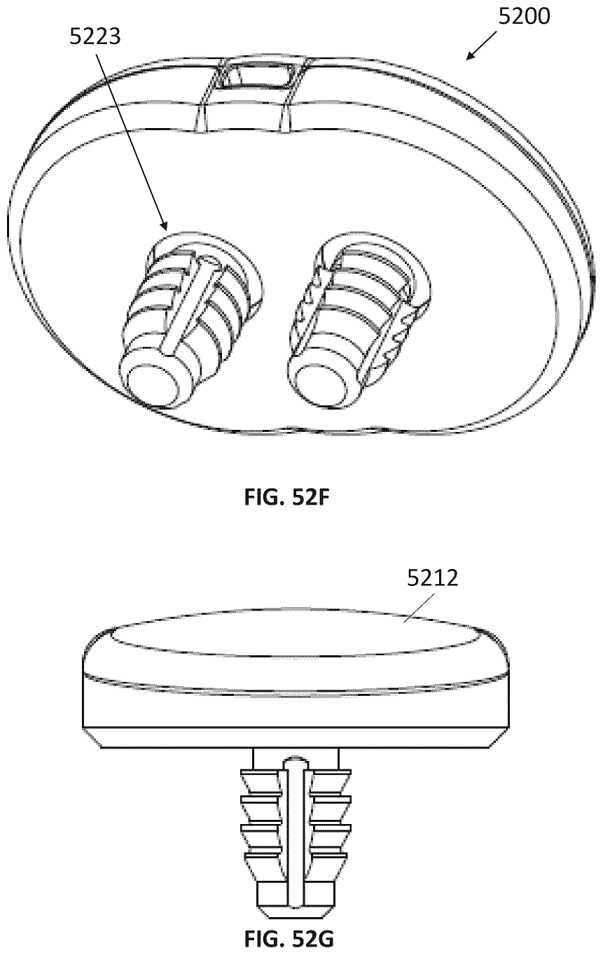

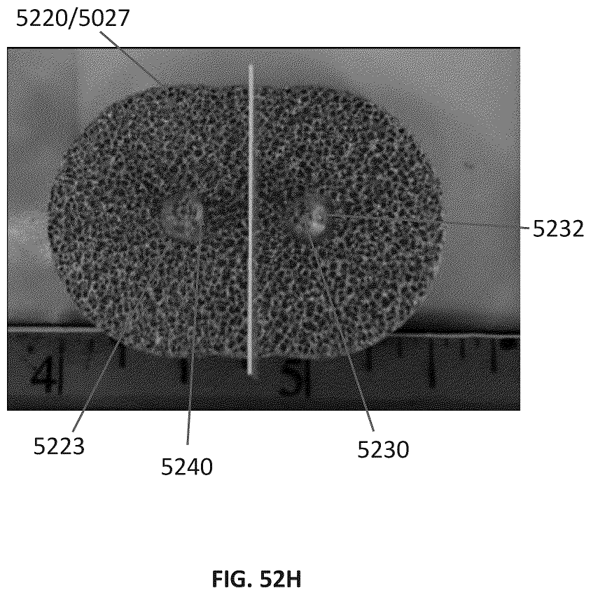

FIGS. 52A to 52H depict aspects of an exemplary femoral implant 5200 with an oblong profile. The femoral implant 5200 can include a convex bearing surface portion 5210 and a subchondral surface portion 5220, which may include a flat bone-contacting surface. The convex bearing surface 5212 can have a spherical diameter of about 60 mm to about 80 mm, for example, about 70 mm. The femoral implant 5200 can also include one or more (for example, two) proximal pegs 5230. The peg 5230 can have a length of about 5 mm to about 10 mm. The peg 5230 can have an outer diameter of about 2.5 mm to about 3.5 mm.

The femoral implant 5200 can have the same or similar features as the femoral implant 3900, but may include differences from the femoral implant 3900 as described herein with reference to FIGS. 52A to 52H. In some cases, the subchondral surface portion 5220 and/or at least an outer portion 5234 of one or more the proximal pegs 5230 can include a porous material. The porous material can include porous titanium or any other suitable materials disclosed herein. In the embodiment depicted here, proximal pegs 5230 include multiple barbs 5240. The outer diameter of the peg 5230 at the barbed region can be between about 3.5 mm to about 4.5 mm. In some cases, the barbs 5240 and the convex bearing surface portion 5210 (and optionally an inner or core portion 5236 of the peg 5230) can include a solid or nonporous material. In some implementations, the solid metal inner portion 5236 of the peg 5230 can be contiguous with the solid metal bearing surface portion 5210. The solid/nonporous material can include nonporous titanium or any other suitable materials disclosed herein, such as nonporous stainless steel, nonporous cobalt chrome, nonporous ceramic, and the like. The convex bearing surface 5212 can be coated with a hard coating material, for example but not limited to TiN or diamond hardened titanium. The porous material of the subchondral surface portion 5220 (and optionally an outer portion 5234 of the peg 5230) can have a trabecular porous or irregular lattice structure 5227. The lattice can facilitate osteointegration and/or bone ingrowth. The porous titanium backside has an irregular porous lattice with 60-75% porosity by volume, 600 μm average pore size, and 225 μm strut thickness. The porous layer is 1.0 mm thick. The peg 5230 with the tapered barbs 5240 can provide primary fixation (optionally with bone cement) and the random lattice can provide secondary fixation by osteointegration. In some cases, the femoral implant 5200 can be a monolithic unit.

The convex bearing surface portion 5210 of the femoral implant 5200 can have a three-circle or oblong profile. The three-circle profile can have three sections formed by three overlapping circles, which allows for simpler reaming with a circular reamer (e.g. in 3 adjacent reaming locations on the patient's bone, with overlapping circles) than, for example, reaming a racetrack profile or another profile with straight edges in the bone. The pegs 5230 may align along a longitudinal axis of the oblong profile. As shown in FIG. 52C, the two pegs 5230 may each extend from the center of one of the two outer circles. In this embodiment, the femoral implant 5200 includes no anti-rotation spikes, although such spikes may be present in other embodiments.

The one or more pegs 5230 can have one or more channels 5232 (for example, three as shown in FIG. 52C, or two, or more) along at least a portion of the length of the peg 5230. The channels 5232 can allow for bone cement to flow along the length of the peg 5230 if bone cement is used, or allow for reduced pressure in the drilled hole in the bone if used for a cementless application. The channels 5232 can have a depth such that they cut through not only the barbs 5240 but also a part of the peg 5230. The channels 5232 can extend along the length of the peg 5230 or have any other suitable size/shape. In some cases, the femoral implant 5200 can include one or more wells 5223 in a porous subchondral lattice 5227 of the subchondral surface portion 5220. A well 5223 can be located at the base of each of the pegs 5230 to contain overflowing cement. The well 5223 can have a diameter of about 4.5 mm to about 5.5 mm.

The femoral implant 5200 can include two or more notches or pockets 5260 to allow for the connection of an insertion tool or a removal tool. As shown in FIGS. 52A and 52D, the pockets 5260 can be located just below the convex bearing surface portion 5210 on the side wall of the middle circle. This illustrated embodiment has two pockets 5260 can be located diametrically opposite each other on the two side wall of the middle circle.

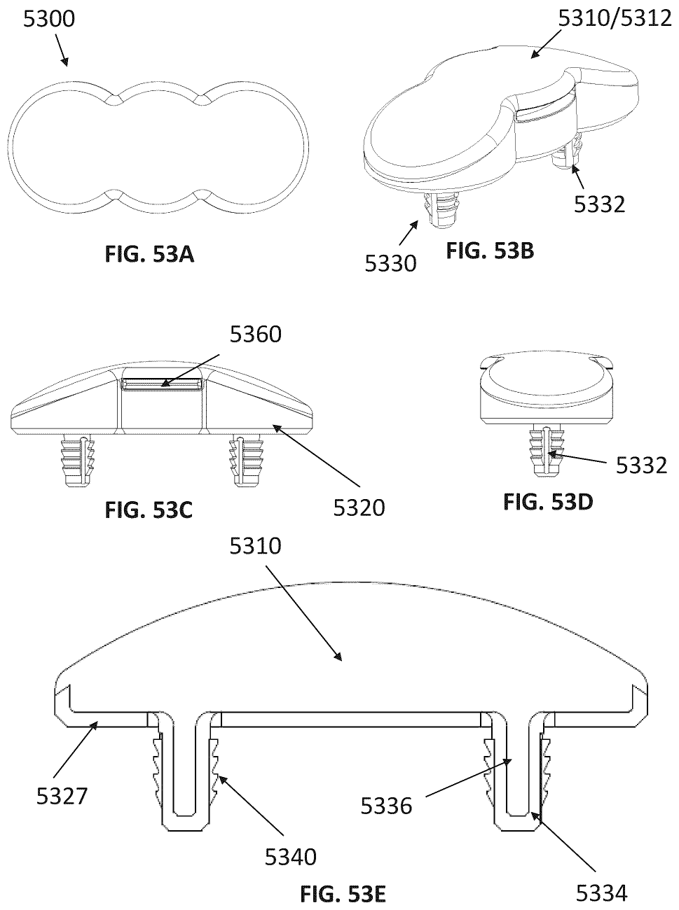

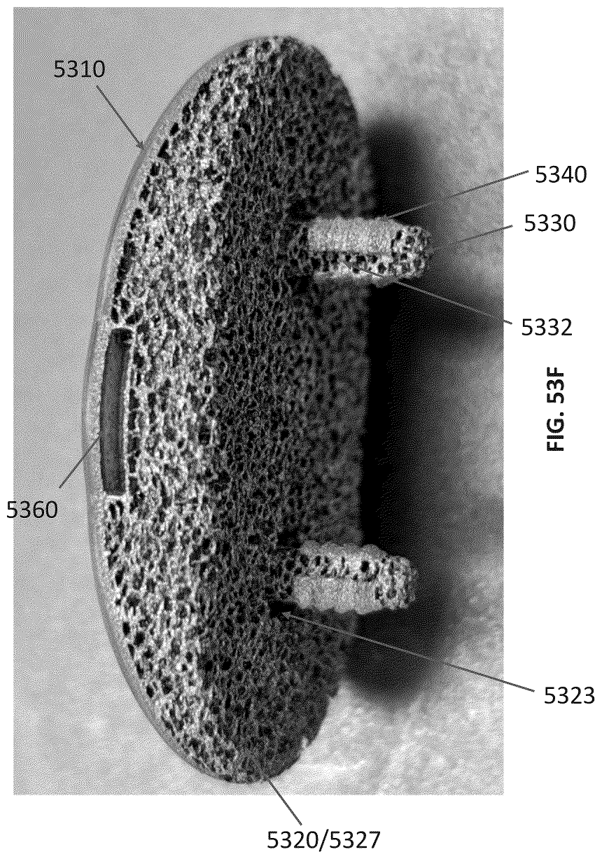

FIGS. 53A to 53F depicts aspects of an exemplary femoral implant 5300 with an oblong profile. The femoral implant 5200 can have the same or similar features as the femoral implant 3900 and/or the femoral implant 5200, but may include differences from the femoral implant 3900 and/or the femoral implant 5200 as described herein with reference to FIGS. 53A to 53F.

The femoral implant 5300 can include a convex bearing surface portion 5310 and a subchondral surface portion 5320, which may include a flat subchondral bone-contacting surface. The convex bearing surface 5312 can have a spherical diameter of about 60 mm to about 80 mm, for example, about 70 mm. The femoral implant 5300 can also include one or more (for example, two) proximal pegs 5230. The peg 5330 can have a length of about 5 mm to about 10 mm. The peg 5330 can have an outer diameter of about 2.5 mm to about 3.5 mm. In some cases, the subchondral surface portion 5320 and/or at least an outer portion 5334 of one or more the proximal pegs 5330 can include a porous material. The porous material can include porous titanium or any other suitable materials disclosed herein. In the embodiment depicted here, proximal pegs 5330 include multiple barbs 5340. The outer diameter of the peg 5330 at the barbed region can be between about 3.5 mm to about 4.5 mm. In some cases, the barbs 5340 and the convex bearing surface portion 5310 (and optionally an inner or core portion 5336 of the peg 5330) can include a solid or nonporous material. In some implementations, the solid metal inner portion 5336 of the peg 5330 can be contiguous with the solid metal bearing surface portion 5310. The solid/nonporous material can include nonporous titanium or any other suitable materials disclosed herein, such as nonporous stainless steel, nonporous cobalt chrome, non-porous ceramic, and the like. The convex bearing surface 5312 can be coated with a hard coating material, for example but not limited to TiN or diamond hardened titanium. The porous material of the subchondral surface portion 5320 (and optionally an outer portion 5334 of the peg 5330) can have a trabecular porous or irregular lattice structure 5327. The irregular lattice can facilitate osteointegration and/or bone ingrowth. The porous titanium backside has an irregular porous lattice with 60-75% porosity by volume, 600 μm average pore size, and 225 μm strut thickness. The porous layer is 1.0 mm thick. The peg 5330 with the tapered barbs 5340 can provide primary fixation (optionally with bone cement) and the random lattice can provide secondary fixation by osteointegration. In some cases, the femoral implant 5300 can be a monolithic unit.

The convex bearing surface portion 5310 of the femoral implant 5300 can have a three-circle or oblong profile. The three-circle profile can have three sections formed by three overlapping circles, which allows for simple reaming with a circular reamer (e.g. in 3 adjacent reaming locations on the patient's bone, with overlapping circles). The distances between the centers of the three circles are greater in the femoral implant 5300 than in the femoral implant 5200, resulting in the femoral implant 5300 having a greater length than the femoral implant 5200. The pegs 5330 may align along a longitudinal axis of the oblong profile. The two pegs 5330 may each extend from the center of one of the two outer circles. In this embodiment, the femoral implant 5300 includes no anti-rotation spikes, although such spikes may be present in other embodiments.

The one or more pegs 5330 can have one or more channels 5332 (for example, three, or two, or more than three) along at least a portion of a length of the peg 53230. The channels 5332 can allow for bone cement to flow along the length of the peg 5330 if bone cement is used, or allow for reduced pressure in the drilled hole in the bone if used for a cementless application. The channels 5332 can have a depth such that they cut through not only the barbs 5340 but also a part of the peg 5330. The channels 5332 can extend along the length of the peg 5330 or have any other suitable size/shape. In some cases, the femoral implant 5300 can include one or more wells 5323 in a porous subchondral lattice 5327 of the subchondral surface portion 5320. A well 5323 can be located at the base of each of the pegs 5330 to contain overflowing cement. The well 5323 can have a diameter of about 4.5 mm to about 5.5 mm.

The femoral implant 5300 can include two or more notches or pockets 5360 to allow for the connection of an insertion tool or a removal tool. As shown in FIGS. 53B and 53C, the pockets 5360 can be located just below the convex bearing surface portion 5310 on the side wall of the middle circle. This illustrated embodiment has two pockets 5360 can be located diametrically opposite each other on the two side wall of the middle circle. The pocket 5360 in the femoral implant 5300 is longer than the pocket 5260 in the femoral implant 5200.

In some embodiments, an oblong femoral implant can have a width W (see, e.g., FIG. 40D) having a value within a range from about 15 mm to about 35 mm, or from about 17.5 mm to about 27.5 mm. In some embodiments, a femoral oblong implant can have a length L (see, e.g., FIG. 40A) having a value within a range from about 25 mm to about 40 mm. Exemplary width×length dimensions for a femoral oblong implant include 17.5 mm×25 mm, 17.5×30 mm, 17.5 mm×35 mm, 17.5 mm×40 mm, 20.5 mm×30 mm, 20.5 mm×35 mm, 20.5 mm×40 mm, 22.5 mm×30 mm, 22.5 mm×35 mm, 22.5 mm×40 mm, 25 mm×35 mm, 25 mm×40 mm, 27.5 mm×35 mm, 27.5 mm×40 mm, and the like. In some implementations, for the oblong femoral implants that have the same width and/or the same diameter of the three circles, the distance between the centers of the three circles are greater and/or the extent of overlapping among the three circle are reduced for implants having a greater length. The thickness of an oblong femoral implant (the distance from the inflection point on the bearing surface (maximum for the femoral implant) to the flat subchondral surface) can range from about 4 mm to about 10 mm.

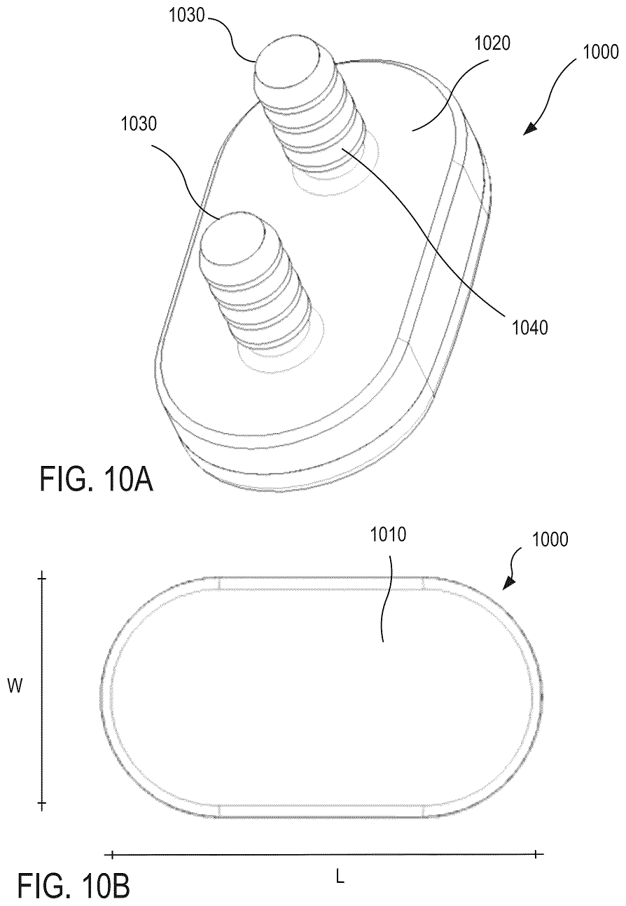

FIGS. 10A and 10B depict aspects of another exemplary femoral implant 1000 with an oblong racetrack profile, which can have any features of the oblong femoral implant examples disclosed herein, with certain differences described herein with reference to FIGS. 10A and 10B. As shown here, a femoral implant 1000 can have a convex bearing surface portion 1010 and a subchondral surface portion 1020. The femoral implant 1000 can also include one or more proximal pegs 1030. In some cases, the subchondral surface portion 1020 and/or one or more the proximal pegs 1030 can include a porous material, such as porous titanium. In the embodiment depicted here, proximal pegs 1030 include multiple barbs 1040. In some cases, the convex bearing surface portion 1010 of the femoral implant 1000 can include nonporous titanium. In some cases, the convex bearing surface portion 1010 can include nonporous stainless steel, nonporous cobalt chrome, nonporous ceramic, and the like. In some cases, the femoral implant can include no porous structure. The convex bearing surface portion 1010 of the femoral implant 1000 has an oblong racetrack profile, which includes two half circles connected by two parallel straight lines. In some cases, the oblong racetrack profile has a length L with a value within a range from about 20 mm to about 35 mm. In some cases, the oblong racetrack profile has a width W with a value within a range from about 12 mm to about 20 mm. In some cases, the femoral implant 1000 is a monolithic unit. In this embodiment, the femoral implant 1000 includes no anti-rotation spikes, although such spikes may be present in other embodiments.

### C. Example Tibial Implants

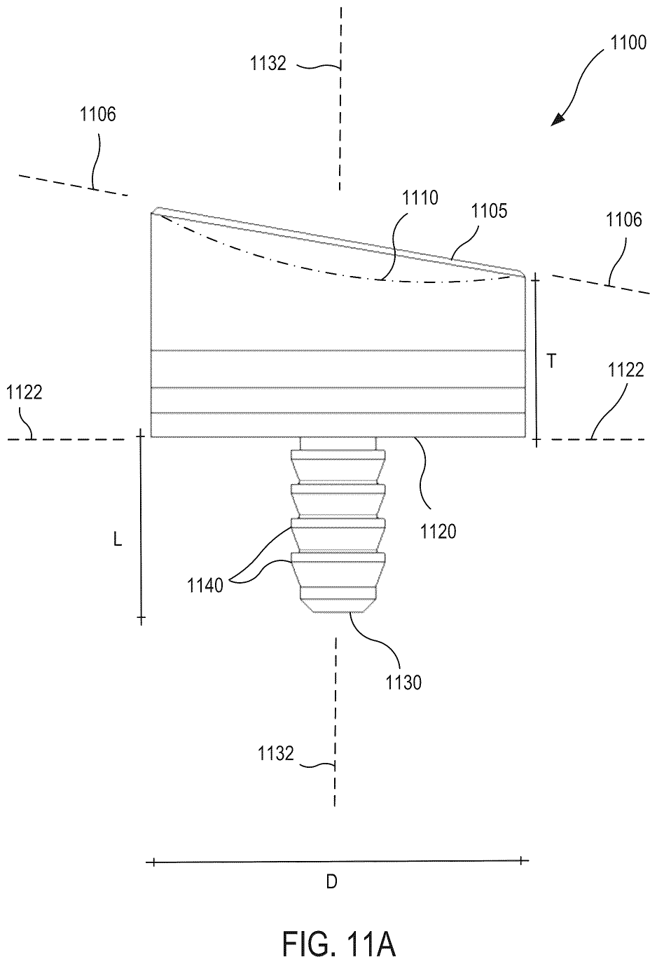

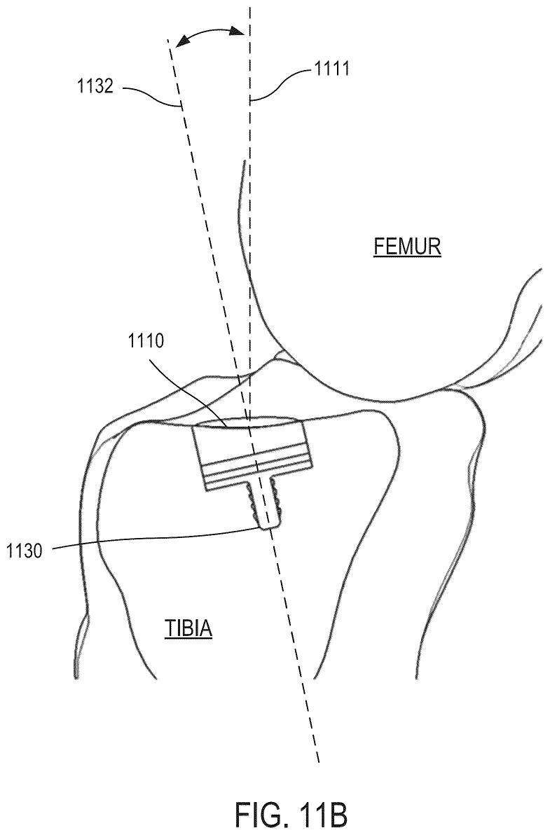

Turning to the tibial side, examples of a tibial implant of a targeted knee joint resurfacing system are illustrated in FIGS. 11A-22, 34A-34C, 37A-37D, 42A-42E, and 55A-57G. Features of any of the example tibial implants disclosed in any of these figures can be incorporated into the example tibial implants depicted in the other figures to the extent that those features are not mutually exclusive to the example tibial implants depicted in the other figures. Features with the same or similar functions are labeled with reference numbers that share the same last two digits. Moreover, any of the features of the tibial implant examples disclosed herein (for example, the peg, the barbs, the anti-rotation spikes or prongs, the channels on the peg, and/or the well at the base of the peg) can have the same dimensions, shape, composition, and/or serve the same functions as the corresponding features of the femoral implant. In some implementations, a tibial implant kit can include a set of tibial implants of different sizes from the range of sizes disclosed herein (e.g., 17.5 mm, 20.0 mm, and 22.5 mm). In some implementations, a targeted or focal knee resurfacing kit can include any of the femoral implant kit as disclosed herein and a tibial implant kit.

### 1. Manufacturing of an Example Tibial Implant

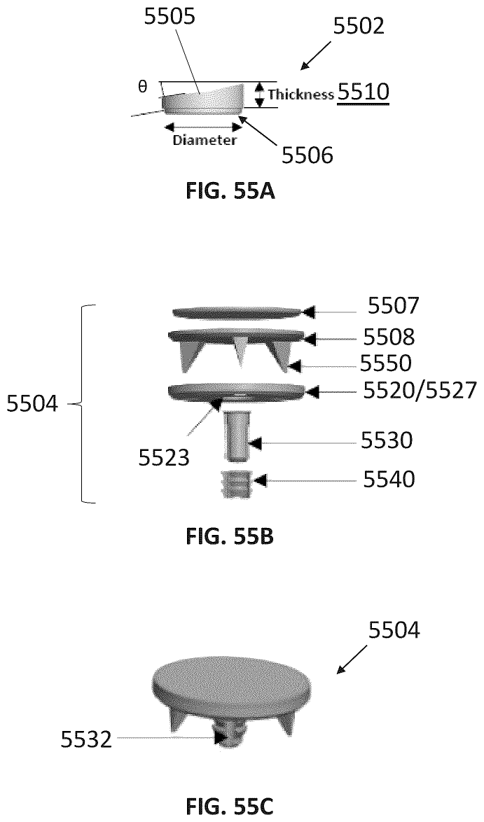

FIGS. 55A-55C illustrate the composition of an example tibial implant from a manufacturing perspective. The tibial implants disclosed herein can have a plastic portion 5502 (such as shown in FIG. 55A) and a metal portion 5504 (such as shown in FIG. 55C). The plastic portion 5502 can be overmolded (e.g., compression-molded) onto the metal portion 5504 so that the tibial implant is provided as a single unit. In other words, the plastic portion of the tibial implants disclosed herein is permanently assembled with the metal portion at the time of manufacturing such that in-situ/in patient and/or in surgery assembly is not required. The metal portion can be manufactured first, followed by molding the plastic portion onto the metal portion. As disclosed elsewhere herein, at least certain parts of the metal portion (e.g., the barbs and the porous structures) can be 3D printed. In some implementations, the metal portion may be manufactured as a monolithic unit such that no coupling mechanism is required to couple different parts of the metal portion together. Hot plastic flows into the porous metal structure of the metal portion and it becomes permanently attached to the metal portion.

The plastic portion 5502, shown in FIG. 55A, can be molded from polyethylene, such as ultra-high molecular weight polyethylene (UHMWPE). In some implementations, the plastic portion 5502 can be polyethylene cap. The plastic portion 5502 can have a polished concave surface (hidden from this view) that articulates with an articulating surface of a femur and/or the convex bearing surface of any of the femoral implant examples disclosed herein. The plastic portion 5502 can have a minimum thickness of about 2.7 mm to about 3 mm above an overmolding section 5506, i.e., a section that overlaps with a porous compression-molding section 5507 of the metal portion 5504.

The plastic portion 5502 can have a variety of diameters ranging from about 17.5 mm to about 22.5 mm. The articulating surface of the plastic portion 5502 can have a rim 5505 defining a proximal plane. The proximal plane also can have an offset angle θ (for example, from about 8° to about 15°, or about) 10° relative to a plane of the overmolding section 5506. The angle θ can allow the tibial implant to be inserted at the offset angle relative to a vertical plane of the tibia (see, e.g., FIG. 11B). The offset angle makes insertion and access easier by moving the instruments away from the femoral condyles with the knee in flexion (which will be described in greater detail below). The plastic portion 5502 is part of a concave bearing surface portion 5510 of the tibial implant as described elsewhere in the present disclosure.

As shown in FIGS. 55B and 55C, the metal portion 5504 can be made from a metal (for example, titanium, stainless steel, or any other suitable metals disclosed herein) and/or an alloy of the metal disclosed herein. In some embodiments, the metal portion 5504 can be a titanium base. The metal portion 5504 can include any of the following features: a porous compression-molding section 5507 on its proximal side, a solid metal main body including a disc section 5508 and multiple anti-rotation prongs or spikes 5550, a porous distal side 5520, a porous peg 5530 extending from the porous distal side, and/or solid barbs 5540 that can surround the porous peg 5530. The porous distal side 5520 can form at least partially the subchondral surface portion described elsewhere in the present disclosure.

The porous compression-molding section 5507 can be made up of a regular diamond lattice. The diamond lattice can include a strut length of about 1 mm and/or a strut thickness of about 225 μm. The compression-molding section 5507 can be about 0.075 mm thick. The solid disc section 5508 of the main body can define the depth to which the plastic portion 5502 is compression-molded into the metal portion 5504. The solid disc section 5508 can be about 3 mm thick. The porous distal side 5520 can define a distal plane. The proximal plane as defined by the rim 5505 of the plastic portion 5502 can be at the offset angle relative to the distal plane. The porous distal side 5520 can have an irregular porous lattice 5527. The porous lattice 5527 can have about 60% to about 75% porosity by volume, about 600 μm average pore size, and/or about 225 μm strut thickness. The porous distal side 5520 can have a thickness of about 1.0 mm. The peg 5530 can be perpendicular to the distal plane. The solid anti-rotation prongs 5550 can extend about 1.5 mm to about 3 mm from the porous distal side 5520. FIGS. 80A-80D illustrate micrographs of the example porous metal structures disclosed herein.

The peg 5530 can be about 3.0 mm in diameter and/or about 7.0 mm in length. The outer diameter of the peg 5530 at the barbed region can be about 4.0 mm. The peg 5530 can be centrally located on the tibial implant. The peg 5530 can have one or more (for example, three) channels 5532 that cut into the barbs 5540 and the periphery of the peg 5530 to allow for the flow of cement. The channels 5532 can allow for bone cement to flow along the length of the peg 5530 if bone cement is used, or allow for reduced pressure in the drilled hole in the bone if used for a cementless application. A well 5523 recessed into the porous distal side 5520 can surrounds the peg 5530 allowing for overflow of cement out of a drilled peg hole in the tibia. The well 5523 can be about 5.0 mm in diameter.

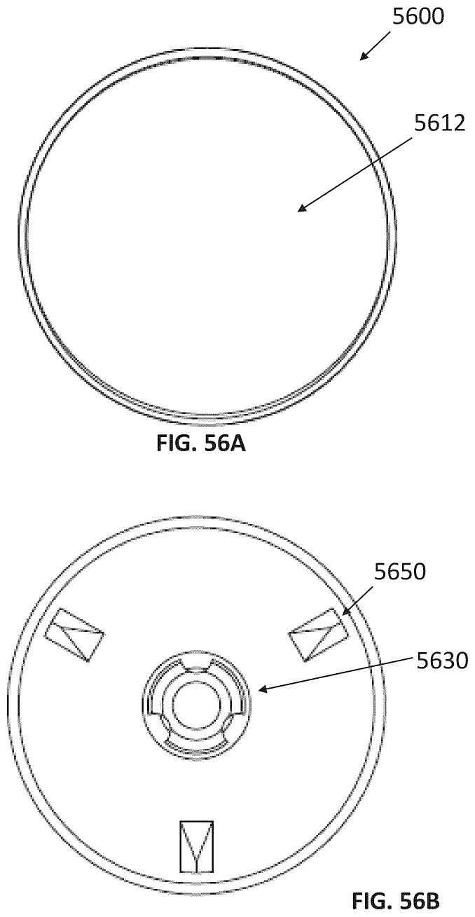

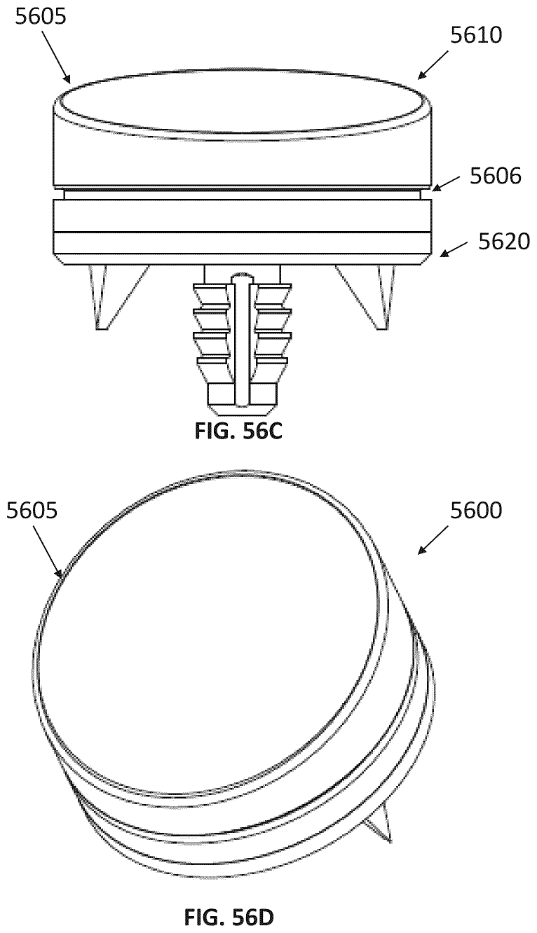

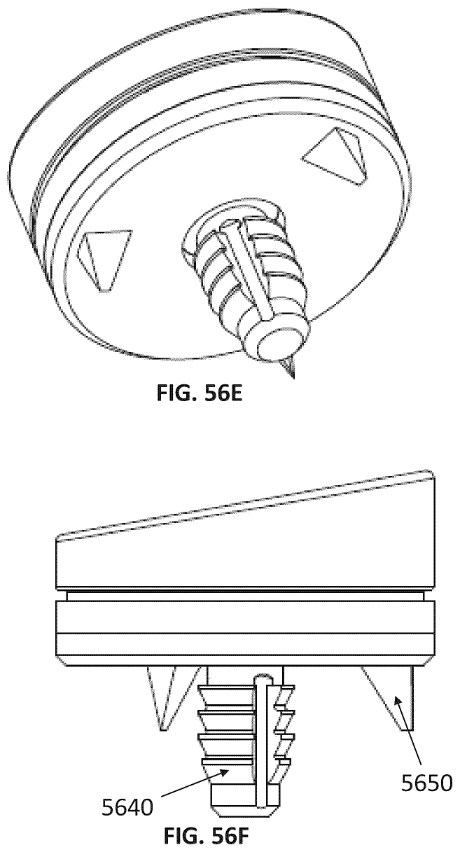

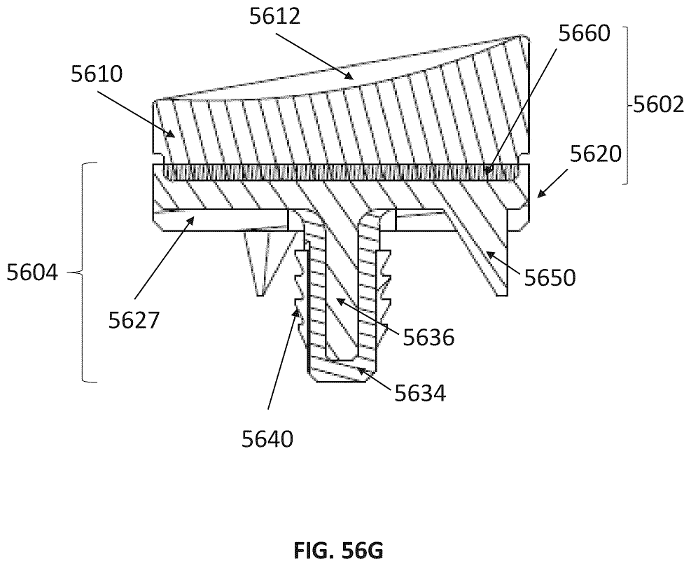

The overmolding section 5506 of the plastic portion 5502 and/or the porous compression-molding section 5507 of the metal portion 5504 can have a smaller diameter than a remainder of the plastic portion 5502 and/or the solid disc section 5508 of the main body of the metal portion 5504. During manufacturing, this reduced diameter can allow the metal portion 5502 to be fixtured or clamped at the porous compression-molding section 5507 such that the plastic is molded above the solid disc section 5508. The clamping can provide a clean stop of plastic flow during the overmolding process. This way, the plastic does not bleed into the metal portion below the solid disc section 5508. The final tibial implant can include a groove (see, e.g., groove 5606, 5706, 5806 as shown in FIGS. 56C, 57B, and 58B) where the metal portion 5504 was clamped during the overmolding process.

### 2. Various Examples of a Tibial Implant

Turning to FIGS. 11A-11B, FIG. 11A depicts aspects of an exemplary tibial implant 1100. As shown, a tibial implant 1100 can have a concave bearing surface portion 1110 (with the concave surface hidden from the side view) and a subchondral surface portion 1120. The tibial implant 1100 can also include a distal peg 1130. The subchondral surface portion 1120 and the distal peg 1130 collectively form the metal portion. In the embodiment depicted here, distal peg 1130 can include one or more barbs 1140. In some cases, the concave bearing surface portion 1110 (i.e. the plastic portion) of the tibial implant 1100 can include UHMWPE. In some cases, the proximal side of the subchondral surface portion 1120 of the tibial implant 1100 includes porous titanium. In some cases, the proximal side of the subchondral surface portion 1120 of the tibial implant 1100 includes nonporous stainless steel, nonporous cobalt chrome, and/or nonporous ceramic. In some cases, the distal side of the subchondral surface portion 1120 of the tibial implant 1100 includes an irregular lattice. In this embodiment, the concave bearing surface portion 1110 of the femoral implant 1100 has a round profile. In some cases, the concave bearing surface portion 1110 can provide a round profile having a diameter D with a value within a range from about 15 mm to about 25 mm. In some cases, the tibial implant 1100 can have a thickness T with a value that is equal to or greater than about 6.5 mm. The thickness T of a tibial implant can refer to the distance from the inflection point on the bearing surface (minimum for the tibial implant) to the subchondral surface, where the porous section engages the bottom or surface of the reamed bone. A peg length L can have a value of about 5.5 mm. In some cases, the distal peg 1130 can have a trabecular porous structure. In some cases, the tibial implant 1100 is a monolithic unit. In this embodiment, the tibial implant 1100 includes no anti-rotation spikes, although such spikes may be present in other embodiments. In some cases, the tibial implant 1100 includes a bone screw fixation mechanism.

As shown in FIG. 11A, the concave bearing surface portion 1110 of the tibial implant 1100 can have a rim 1105 that defines a proximal plane 1106, and the subchondral surface portion 1120 of the tibial implant 1100 can define distal plane 1122. In some embodiments, the proximal plane 1106 defined by the rim 1105 of the concave bearing surface portion 1110 of the tibial implant 1100 is non-parallel to the distal plane 1122 defined by the subchondral surface portion 1120 of the tibial implant 1100. The distal peg 1130 can define a longitudinal axis 1132. In some cases, the longitudinal axis 1132 defined by the distal peg 1130 can be perpendicular to the distal plane 1122 defined by the subchondral surface portion 1120 of the tibial implant 1100 and non-perpendicular to the proximal plane 1106 defined by the rim 1105 of the concave bearing surface portion 1110 of the tibial implant 1100. As shown in FIG. 11B, upon implantation, the longitudinal axis 1132 defined by the distal peg 1130 is at an angle A from an axis 1111 that is normal to the concave bearing surface portion 1110. In some cases, angle A has a value of about 10 degrees. As shown here, the bottom or distal surface of the metal portion can be perpendicular to the peg.

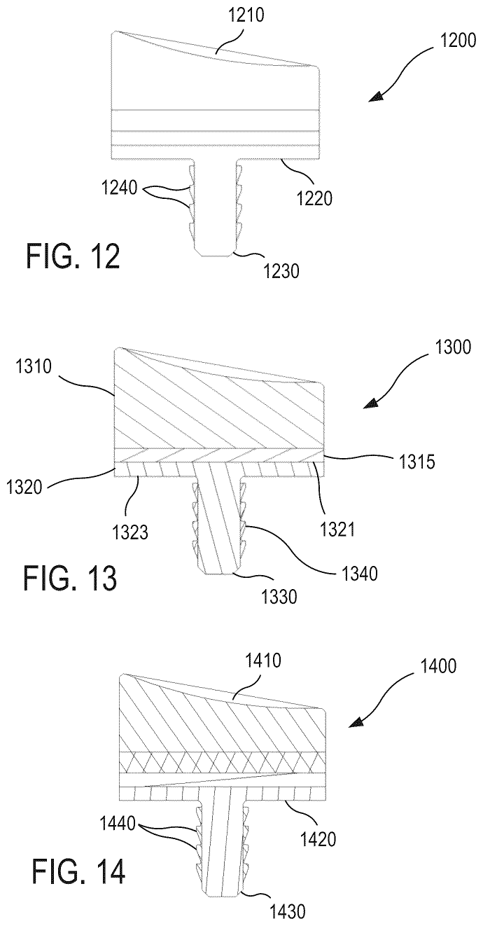

FIG. 12 depict aspects of an exemplary tibial implant 1200. Tibial implant 1200 includes a concave bearing surface 1210, a subchondral surface 1220, and a distal peg 1230 having multiple barbs 1240.

FIG. 13 depict aspects of an exemplary tibial implant 1300. In the cross-section view provided here, the implant 1300 includes a concave bearing surface portion 1310 (which may include a nonporous polymeric material), a subchondral surface portion 1320 (which may include a porous metal material) having a proximal side 1321 and a distal side 1323, and multiple barbs 1340 (which may include a nonporous metal material) on a distal peg 1330 (which may include a porous metal material). The concave bearing surface portion 1310 of the tibial implant can be compression molded with the proximal side 1321 of the subchondral surface portion 1320 of the tibial implant 1300. In some cases, due to the compression molding process, there may be an overlap area 1315 (also referred to as the overmolding section on the plastic portion and the porous compression-molding section on the metal portion). In some cases, the concave bearing surface portion 1310 includes UHMWPE, the subchondral surface portion 1320 and distal peg 1330 include a porous metal material, and the barbs 1340 include a solid metal material. The overlap area or portion 1315 can include both UHMWPE and porous metal, and can be present as a result of compression molding. For instance, the concave bearing surface portion 1310 of the tibial implant can be compression molded with the proximal side 1321 of the subchondral surface portion of the tibial implant.

FIG. 14 depict aspects of an exemplary tibial implant 1400. In the cross-section view provided here, it can be seen that tibial implant 1400 includes a concave bearing surface 1410, a subchondral surface 1420, and a distal peg 1430 having multiple barbs 1440.

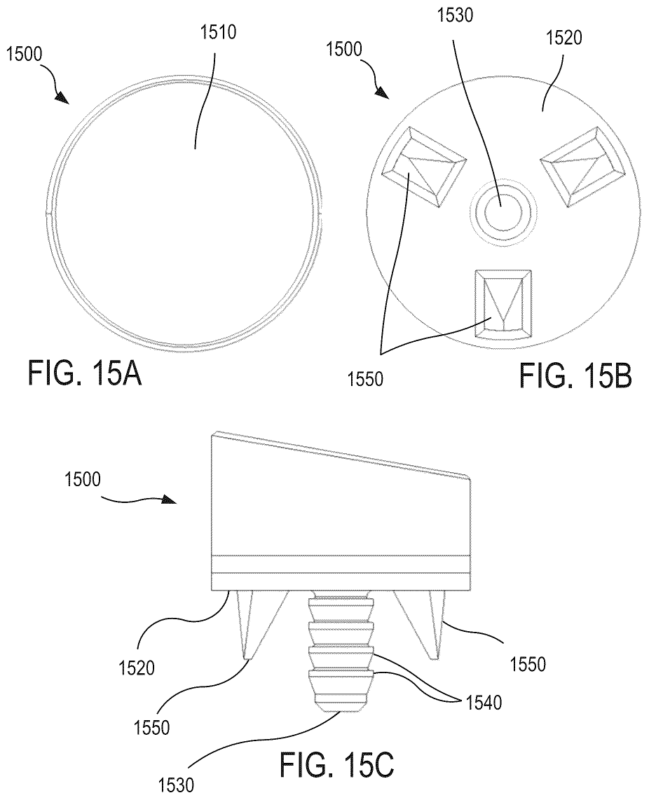

FIGS. 15A to 15C depict aspects of an exemplary tibial implant 1500. As shown, a tibial implant 1500 can have a concave bearing surface portion 1510 and a subchondral surface portion 1520. The tibial implant 1500 can also include a distal peg 1530. In the embodiment depicted here, distal peg 1530 can include one or more barbs 1540. In some cases, the concave bearing surface portion 1510 of the tibial implant 1500 can include UHMWPE. In some cases, the proximal side of the subchondral surface portion 1530 of the tibial implant 1500 includes porous titanium. In some cases, the proximal side of the subchondral surface portion 1520 of the tibial implant 1500 includes nonporous stainless steel, nonporous cobalt chrome, and/or nonporous ceramic. In some cases, the distal side of the subchondral surface portion 1520 of the tibial implant 1500 includes an irregular lattice. In this embodiment, the concave bearing surface portion 1510 of the femoral implant 1500 has a round profile. In some cases, the distal peg 1530 can have a trabecular porous structure. In some cases, the tibial implant 1500 is a monolithic unit. In some cases, the tibial implant 1500 can include one or more anti-rotation spikes 1550, although such spikes may be absent in other embodiments.

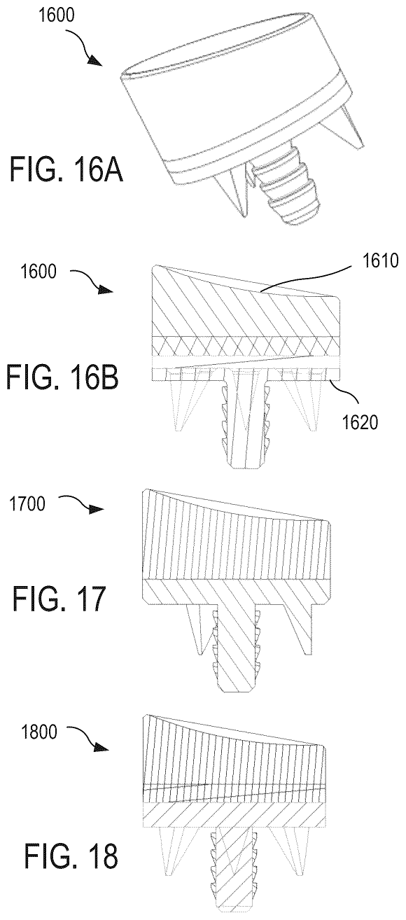

FIGS. 16A and 16B depict aspects of an exemplary tibial implant 1600. As shown, a tibial implant 1600 can have a concave bearing surface portion 1610 and a subchondral surface portion 1620. FIG. 16B provides a cross-section view of the implant depicted in FIG. 16A. FIG. 17 depict aspects of an exemplary tibial implant 1700. FIG. 18 depict aspects of an exemplary tibial implant 1800. As shown here, the tibial implant 1800 includes no trabecular bottom porous structure.

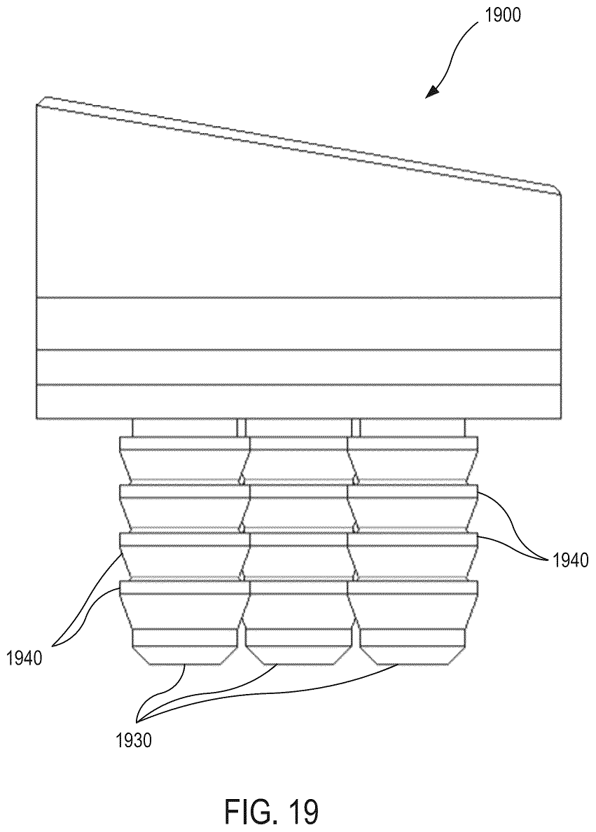

FIG. 19 depicts aspects of an exemplary tibial implant 1900. The tibial implant 1100 can include multiple distal pegs 1930 (for example, three or more). In the embodiment depicted here, distal pegs 1930 can include one or more barbs 1940.

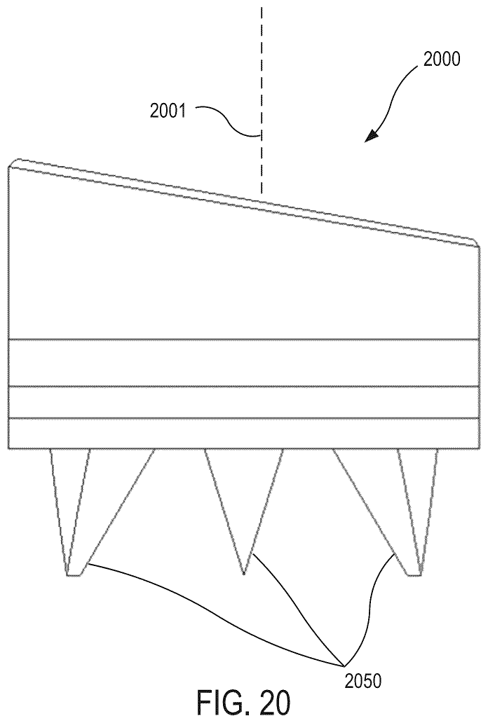

FIG. 20 depicts aspects of an exemplary tibial implant 2000. The tibial implant 2000 can include one or more anti-rotation spikes 2050. Such spikes 2050 can help to prevent or inhibit the implant 2000 from rotating about a central longitudinal axis 2001 thereof when implanted in the patient's body.

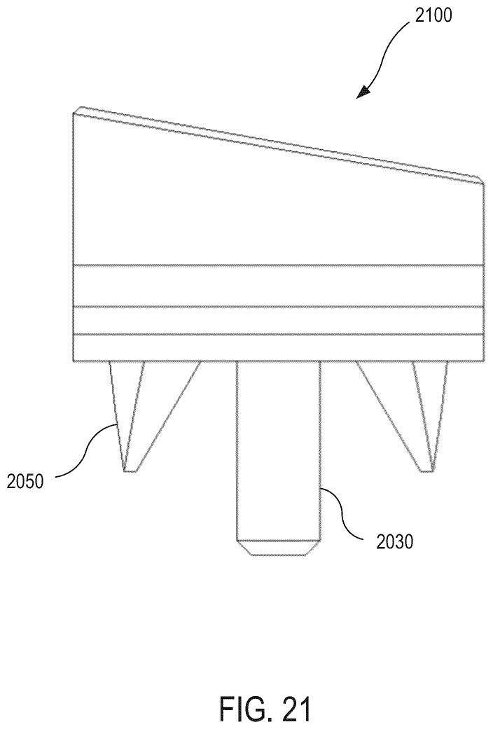

FIG. 21 depicts aspects of an exemplary tibial implant 2100. The tibial implant 2100 can include one or more anti-rotation spikes 2150. The tibial implant 2100 can also include one or more distal pegs, such as distal peg 2030. As shown here, the distal peg 2030 does not have any barbs, although barbs may be present on distal pegs in other embodiments.

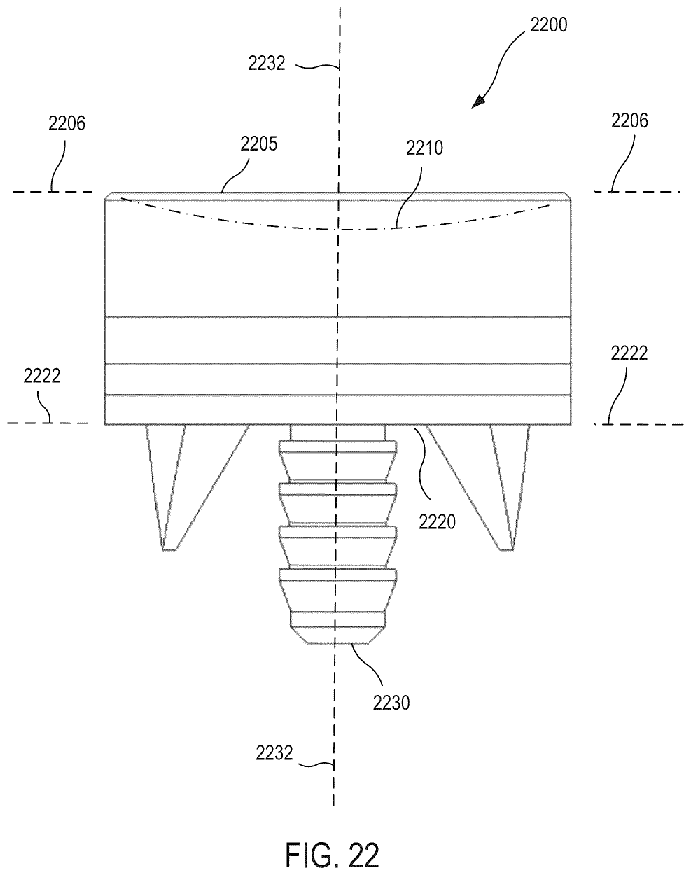

FIG. 22 depicts aspects of an exemplary tibial implant 2200. As shown here, a tibial implant 2200 can have a concave bearing surface portion 2210 (with the concave surface hidden from view) and a subchondral surface portion 2220. The tibial implant 2200 can also include a distal peg 2230. The concave bearing surface portion 2210 of the tibial implant 2200 can have a rim 2205 that defines a proximal plane 2206, and the subchondral surface portion 2220 of the tibial implant 2200 can define distal plane 2222. In some embodiments, the proximal plane 2206 defined by the rim 2205 of the concave bearing surface portion 2210 of the tibial implant 2200 is parallel to the distal plane 2222 defined by the subchondral surface portion 2220 of the tibial implant 2200. The distal peg 2230 can define a longitudinal axis 2232. In some cases, the longitudinal axis 2232 defined by the distal peg 2230 can be perpendicular to the distal plane 2222 defined by the subchondral surface portion 2220 of the tibial implant 2200 and also perpendicular to the proximal plane 2206 defined by the rim 2205 of the concave bearing surface portion 2210 of the tibial implant 2200. Embodiments of the present disclosure also encompass tibial implants where the longitudinal axis 2232 is not perpendicular to the distal plane 2222 and/or the proximal plane 2206. In some cases, tibial implant 2200 can provide a zero degree or variable angle embodiment.

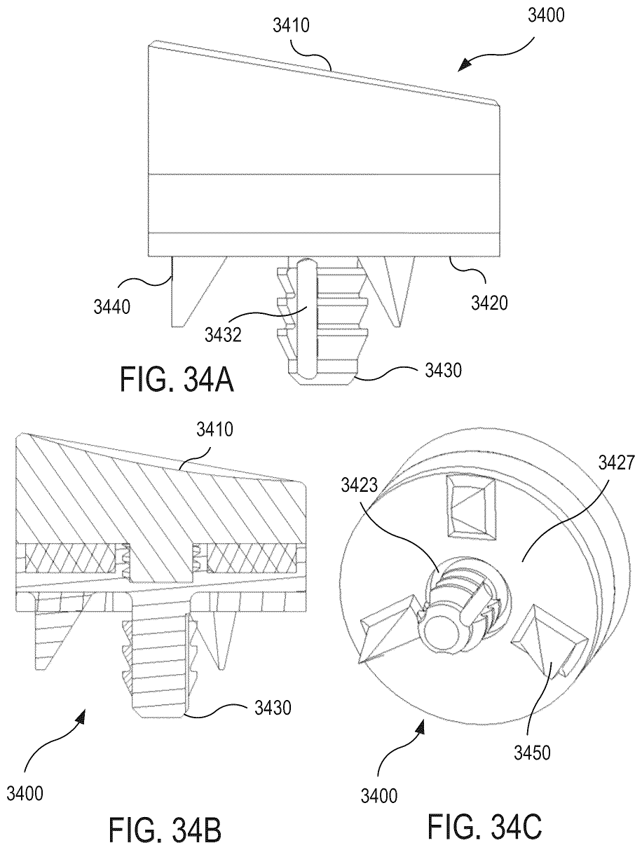

FIGS. 34A to 34C depict aspects of an exemplary tibial implant 3400. According to some embodiments, the tibial implant 3400 includes a concave bearing surface portion 3410 and a subchondral surface portion 3420. Tibial implant 3400 can also include a peg 3430 and one or more anti-rotation spikes 3440. In this embodiment, a solid portion can surround a porous overmold section. In some cases, the solid portion can include one or features such as a solid disc portion, a spike 3450, and/or a post barb 3440. The implant can have a center thread 3456 to connect to a slap hammer removal tool. The peg 3430 can have one or more channels 3432, optionally which operate to allow for cement to flow. In some embodiments, cement may not be used. The peg 3430 can be shorter and solid. The implant can include a well 3423 in a porous subchondral lattice 3427 at the base of the peg to contain overflowing cement.

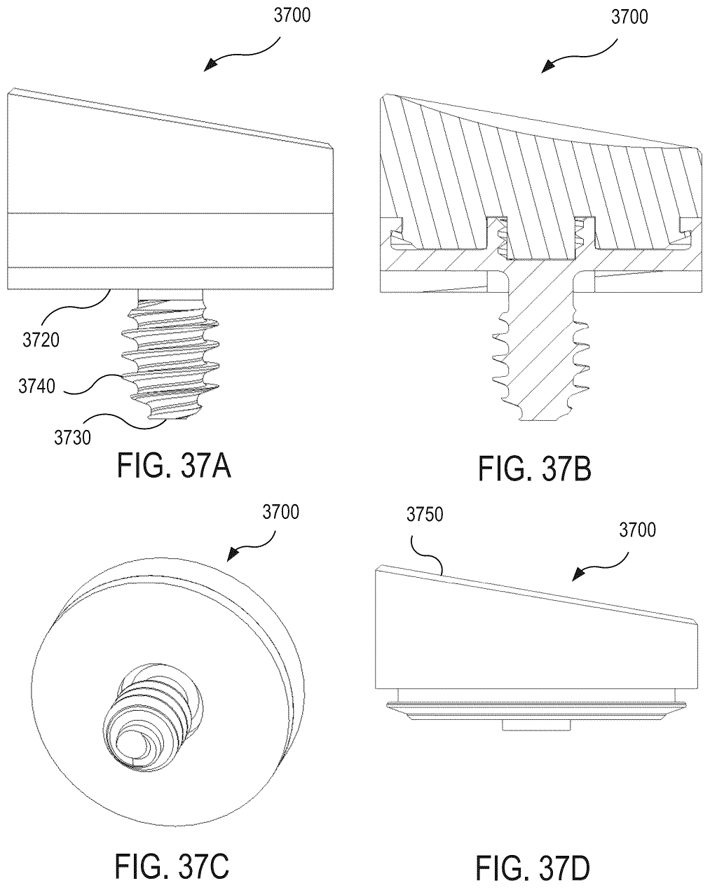

FIGS. 37A to 37D depict aspects of an exemplary tibial implant 3700. In this embodiment, the tibial implant 3700 can have bone threads 3740 (e.g. on a peg 3730). A base or metal portion 3720 of the implant 3700 can be driven in as one component by engaging with the threads 3740. For example, the threads 3740 can engage the bone of the patient. A plastic portion 3750 of the tibial implant 3700 can snap into the base after being driven in.

FIGS. 42A to 42E depict aspects of an exemplary tibial implant 4200. FIG. 42A provides a cross-section view, FIG. 42B provides a front side view, FIG. 42C provides a right side view, FIG. 42D provides a bottom plan view, and FIG. 42E provides a perspective view. As shown, tibial implant 4200 has a round shape (when viewed from the top or bottom). In some cases, implant 4200 can have a diameter D with a value within a range from about 15.0 mm to about 25.0 mm. As shown in FIG. 42A, the barbs 4240 and the anti-rotation spikes 4250 of the implant 4200 can be made of a solid material, such as solid titanium. A concave bearing surface portion or the plastic portion 4210 of the tibial implant 4200 can be made of a plastic or polymer material, such as UHMWPE. A subchondral surface portion 4220 of the implant 4200 can be made of a non-solid material, such as a random lattice, which may include a material such as titanium. A proximal plane defined by a rim around the concave bearing surface is at an angle relative to a distal plane defined by a distal surface of the subchondral surface portion 4220. The peg of implant 4200 is perpendicular to the distal plane. The implant 4200 can include an intermediate portion 4260 disposed between the concave surface portion 4210 and the subchondral surface portion 4220. In some cases, the intermediate portion 4260 can be made of a non-solid material, such as a diamond lattice, which may include a material such as titanium. In some embodiments, a tibial implant can have a diameter D having a value within a range from about 17.5 mm to about 25.0 mm. Exemplary diameter dimensions for a tibial implant include 17.5 mm, 20 mm, 25.0 mm, and the like. In some cases, the concave surface portion 4210 can be compression molded onto the subchondral surface portion 4220.

FIGS. 56A-56H depict aspects of an exemplary tibial implant 5600. The tibial implant 5600 can have any of the features of the tibial implant examples disclosed herein, for example, tibial implant 4200, with certain differences described herein with reference to FIGS. 56A-56H. As shown in FIG. 56G, the tibial implant 5600 can be manufactured by compression molding a plastic portion 5602 onto a metal portion 5604 as described herein with reference to FIGS. 55A-55C. As shown in FIGS. 56A and 56B, the tibial implant 5600 can have a round shape (when viewed from the top or bottom). A concave bearing surface portion 5610 of the tibial implant 5600 can be made of a plastic or polymer material, such as UHMWPE. The concave bearing surface portion 5610 can include a proximally facing concave bearing surface 5612.

Opposite the concave bearing surface portion 5610 on the distal side, the tibial implant 5600 can include a subchondral surface portion 5620. A proximal plane defined by a rim 5605 of the tibial implant 5600 can be at an angle (which in the present disclosure refers to a non-zero angle) relative to a distal plane defined by a distal surface of the subchondral surface portion 5620. The subchondral surface portion 5620 of the implant 5600 can include a distal layer 5627 made of a non-solid metal, such as a random or trabecular lattice, which may include a material such as titanium or titanium alloy. The implant 5600 can include a solid layer 5608 of metal (for example, titanium or its alloy) above the trabecular lattice 5627, similar to the solid disc section 5508 shown in FIG. 55B. The implant 5600 can further include a proximal layer of porous metal (for example, titanium or its alloy), which can be the porous compression-molding section 5507 shown in FIG. 55B. The tibial implant 5600 can include an intermediate portion 5660 disposed between the concave surface portion 5610 and the subchondral surface portion 5620. The intermediate portion 5660 can include the porous compression-molding section 5507 of the metal portion shown in FIG. 55B and/or a portion of the overmolding section 5506 of the plastic portion 5502 shown in FIG. 55A. In some cases, the intermediate portion 5660 can be made of a non-solid metal, such as a diamond lattice, which may include a material such as titanium or titanium alloy.

Multiple anti-rotation spikes 5650 can extend from the distal layer 5627 of the subchondral surface portion 5620. The spikes 5640 can be distributed with even spacing around a center of the circle defined by the subchondral surface portion 5620. A peg 5630 can be centrally located relative to the circle defined by the subchondral surface portion 5620 and extend distally from the subchondral surface portion 5620. The peg 5630 of implant 5600 is perpendicular to the distal plane defined by the distal surface of the subchondral surface portion 5620. The peg 5630 can be longer than the spikes 5640. At least a portion of the peg 5630 can include barbs 5640. The barbs 5640 and the anti-rotation spikes 5650 can be made of a solid material, such as solid titanium or titanium alloy. The peg 5630 can be at least partially porous. The peg 5630 can be made of metal, such as titanium or its alloy. As shown in FIG. 56G, the peg 5630 can include an outer portion 5634 that is porous and an inner portion 5636 that is solid. The solid inner portion 5634 may be contiguous with the solid disc section of the subchondral surface portion 5620 and/or the spikes 5650. The solid inner portion 5636 can provide rigidity to the peg 5630 while the porous outer portion 5634 can allow ingrowth of the bone to facilitate osteointegration.

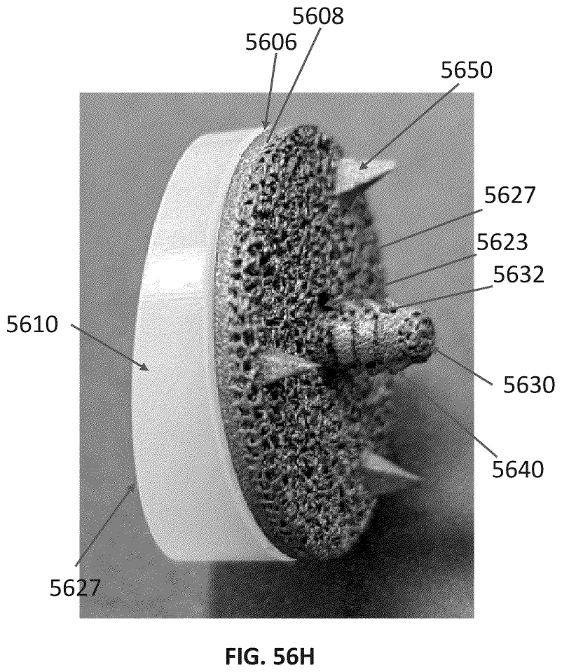

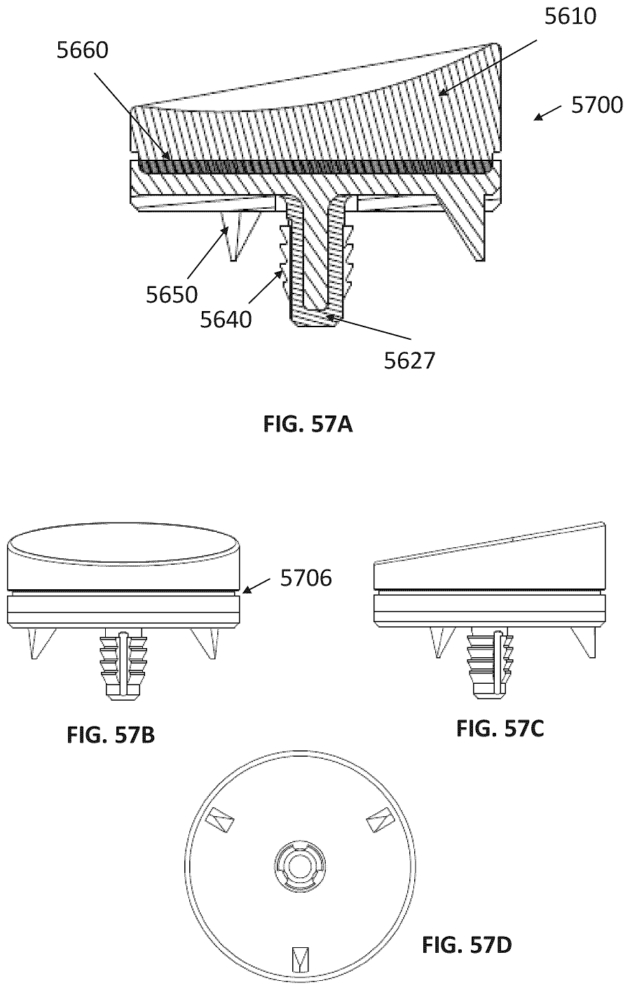

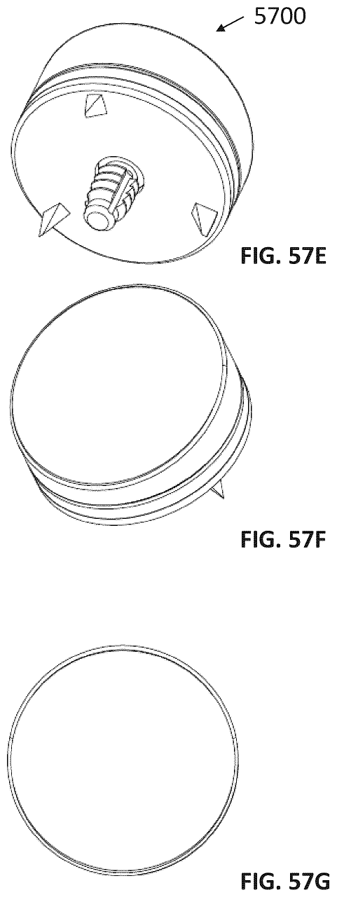

FIGS. 57A to 57G depict aspects of an exemplary tibial implant 5700. The tibial implant 5700 can have any of the features of the tibial implant examples disclosed herein, for example, the tibial implant 5600 as shown in FIGS. 56A to 56G (with the same components in FIGS. 57A-57G sharing the same reference numbers as FIGS. 56A-56H), except that the tibial implant 5700 may have a greater diameter than the tibial implant 5600.

In some cases, the tibial implant examples disclosed herein can have a diameter with a value within a range from about 15.0 mm to about 25.0 mm, or from about 17.5 mm to about 25.0 mm. Exemplary diameter dimensions for a tibial implant include 15 mm, 17.5 mm, 20 mm, 22.5 mm, 25.0 mm, and the like.

### Example Surgical Tools and Kits

A variety of insertion devices may be used for inserting one or both implants of the joint resurfacing systems disclosed herein. In some cases, surgical tools may include components such as reamers, guides, sizers, trials, drill bits, and the like. Any of the surgical tools (for either the femoral or tibial implants) can be available in a variety of sizes to make the tools suitable for inserting an implant of a particular size and/or shape. The surgical tools can be used to prepare patient tissue (e.g. bone) for receiving an implant, for positioning an implant, and/or for inserting an implant in a patient tissue or affixing an implant to a patient tissue. The surgical tools can also be used to remove an implant from the bones. Unless otherwise indicated (for example, a plastic shaft of a single-use hand reamer or a plastic handle portion of a tool), the surgical tools disclosed herein are generally made of metal, for example, stainless steel.

In some implementations, a kit of surgical tools for inserting one or both of the femoral and tibial implant examples disclosed herein, or any other suitable focal or targeted joint resurfacing implants can include a reusable tray and a disposable tray. The reusable tray can include at least one or more of a pin guide/sizer, a first reamer that may be suitable for powered reaming, and/or a second reamer that may be suitable for hand reaming, as disclosed herein. The disposable tray can include at least one or more of guide pins, bone cement, or optionally single-use hand reamer(s) as disclosed herein. The surgical tool kit may include any combinations of the surgical tools disclosed herein. The surgical tool kit can include tools for femoral implant insertion only or tibial implant insertion only such that the tool kit for femoral implants and the tool kit for tibial implants are packaged separately. Alternatively, the surgical tool kit can include tools for both femoral and tibial implant insertions. Some of the tools may be suitable for both femoral and tibial implant insertions, for example but not limited to an insertion tool, a removal tool, drill bits, guide pins, handles, etc. Certain steps of surgical procedure and the associated tools are described below.

### D. Example Femoral Implant Insertion Tools and Method of Insertion

### 1. Patient Positioning

To implant the femoral implant disclosed herein, the patient can be positioned in the supine position. A lateral knee post may be used to stabilize the knee in the flexed position. Alternatively, a dynamic lower extremity positioner may also be used. The hip and knee may be freely movable. In some implementations, the patient positioning set-up can achieve a minimum of about 120° of knee flexion.

Depending on whether the osteochondral defect is located on the medial side or the lateral side, a user or users (e.g., a surgeon and/or any other clinician) can make a longitudinal skin incision along the medial or lateral border of the patellar tendon. The user can make a medial or lateral parapatellar arthrotomy. The user may preserve the quadriceps insertion.

If the joint is exposed via the medial side, the retro-patellar fat pad can be removed for better visualization. The periosteum and soft tissues of the proximal medial tibia can be elevated and dissected medially, while preserving the superficial medial collateral ligament.

If the joint is exposed via the lateral side, the proximal portion of the arthrotomy may be extended into the iliotibial band. The retro-patellar fat pad may be removed for better visualization. The periosteum and soft tissues of the proximal lateral tibia can be elevated and dissected laterally, while preserving the insertion of the iliotibial band at Gerdy's tubercle and the lateral collateral ligament.

### 2. Femoral Implantation Site Access Round Profile

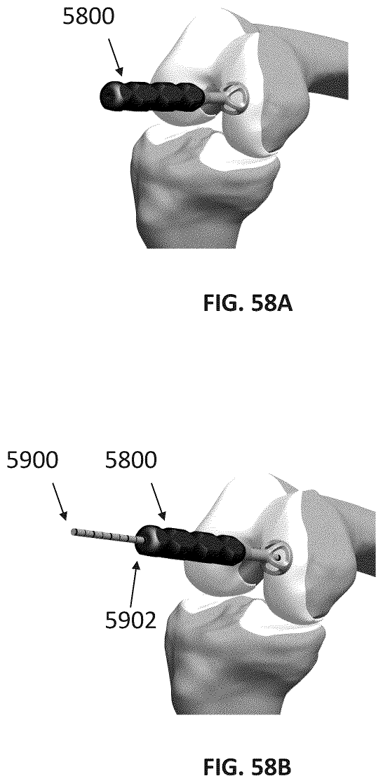

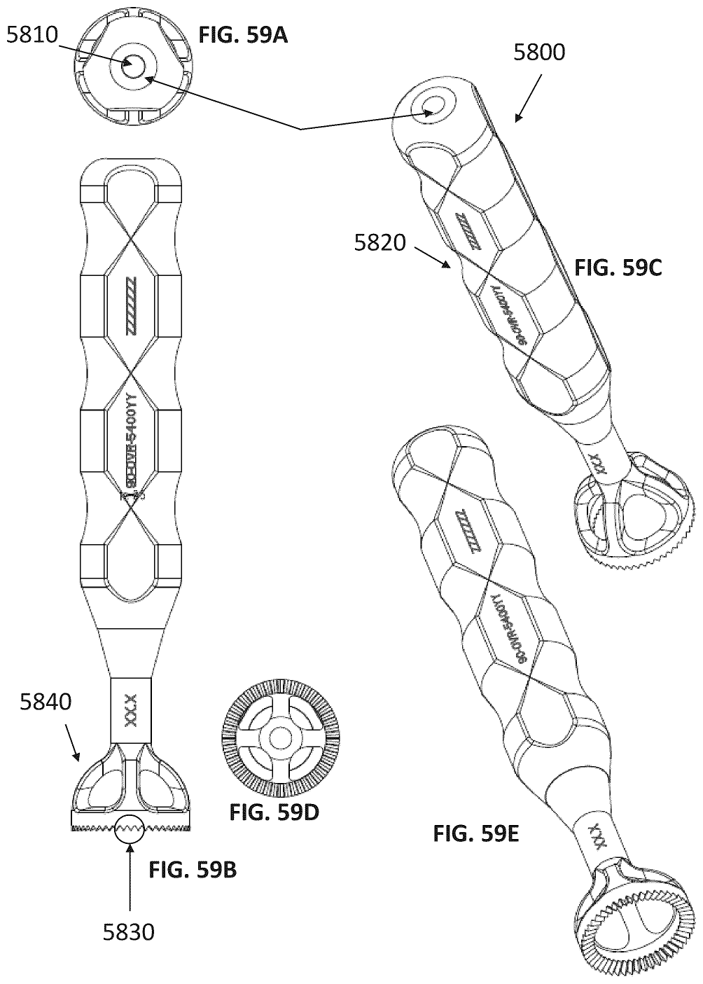

As shown in FIG. 58A, with the knee in flexion, the user can assess the proper implant size using a femoral round pin guide/sizer 5800, which can be available in a range of different sizes. The femoral round pin guide/sizer 5800 is shown in various views in FIGS. 59A-59E. The femoral round pin guide/sizer 5800 can include a base 5840 coupled to a handle portion 4720. The base 5840 can have a round profile. Different sizes of the femoral round pin guide/sizer 5800 can differ in the outer diameter of the round profile of the base 5840, for example, ranging from between 17 mm to about 25 mm. The femoral round pin guide/sizer 5800 can include guide pin hole 5810 extending along a longitudinal axis of the femoral round pin guide/sizer 5800 for receiving a guide pin 5900 such as shown in FIGS. 59F and 59G.

The user can select a femoral round pin guide/sizer with a suitable size so that the base 5840 of the femoral round pin guide/sizer 5800 fully extends beyond the osteochondral defect to ensure full removal of the affected area and proper implant coverage. The chosen implant size may be surrounded by normal/healthy articular cartilage on all sides. When using the femoral round pin guide/sizer 5800, the user may ensure the circular base 5840 is fully in full contact with the condyle cartilage on all sides. This may ensure that the femoral round pin guide/sizer 5800 is perpendicular to the femoral surface and/or the cartilage, thereby improving implant alignment.

As shown in FIG. 58B, with the femoral round pin guide/sizer 5800 flush with the surface of the native cartilage, the user may drive a guide pin 5900 into the guide pin hole 5810 on the femoral round pin guide/sizer 5800. In some implementations, the user may drive the guide pin 5900 into the condyle until a laser mark line 5902 of the pin 5900 is flush with a proximal end of the handle portion 5820 of the femoral round pin guide/sizer 5800. The base 5840 of the femoral round pin guide/sizer 5800 can include spikes 5830. The spikes can extend around a periphery of the base 5840. The spikes 5830 can stabilize the femoral round pin guide/sizer 5800 while installing pins (e.g. by engaging the bone). After the guide pin 5900 has been driven to the desired depth, the user can remove the femoral round pin guide/sizer 5800.

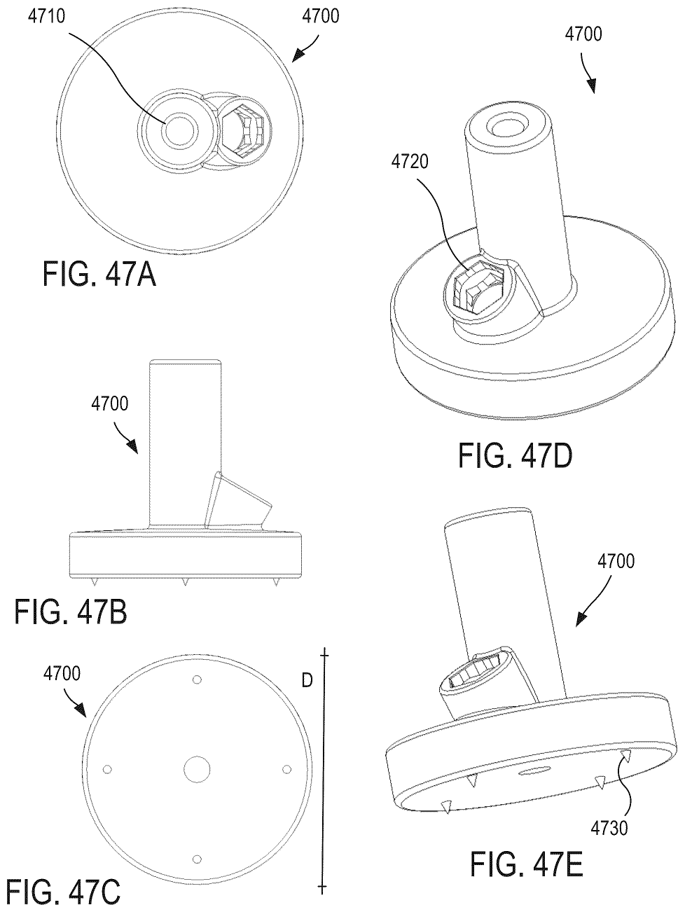

FIGS. 47A to 47E depict aspects of a pin guide sizer mechanism 4700. Any of the features of the femoral round pin guide/sizer 5800 can be incorporated into the pin guide sizer mechanism 4700 and any of the features of the pin guide sizer mechanism 4700 can be incorporated into the femoral round pin guide/sizer 5800. FIG. 47A provides a top plan view, FIG. 47B provides a right side view, FIG. 47C provides a bottom plan view, FIG. 47D provides an upper perspective view, and FIG. 47E provides a lower perspective view. As shown, the pin guide sizer mechanism 4700 can include a guide pin hole 4710, a universal instrument handle socket 4720, and spikes 4730 which can operate to stabilize the guide mechanism 4700 while installing pins (e.g. by engaging patient bone). In some cases, the pin guide mechanism 4700 can have a diameter D that can be used to determine the size of the implant.

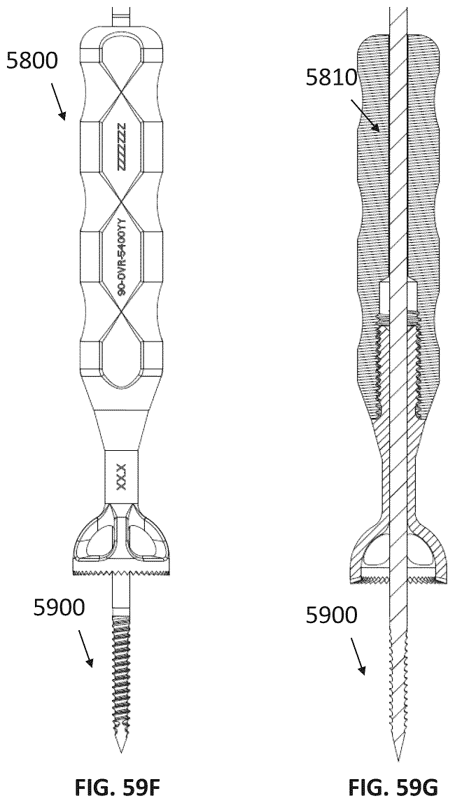

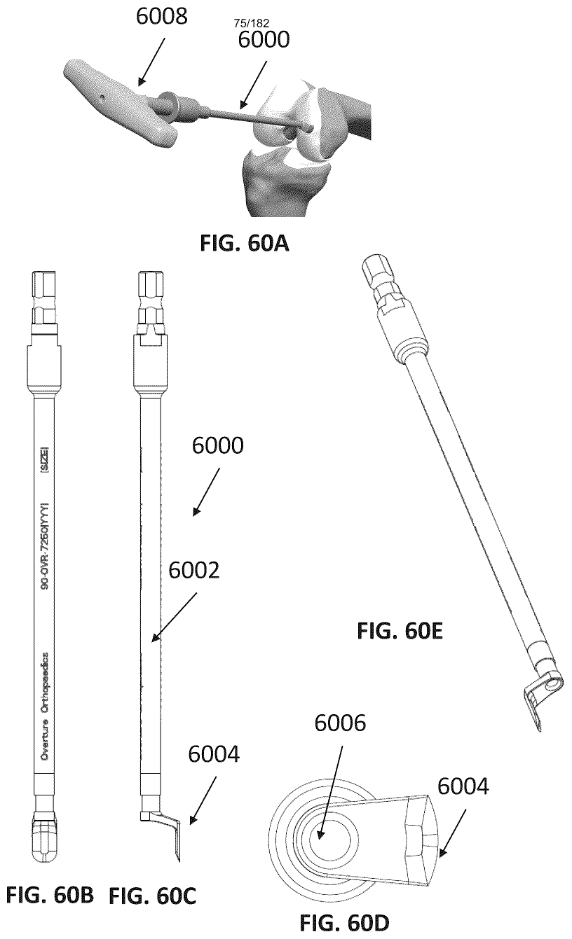

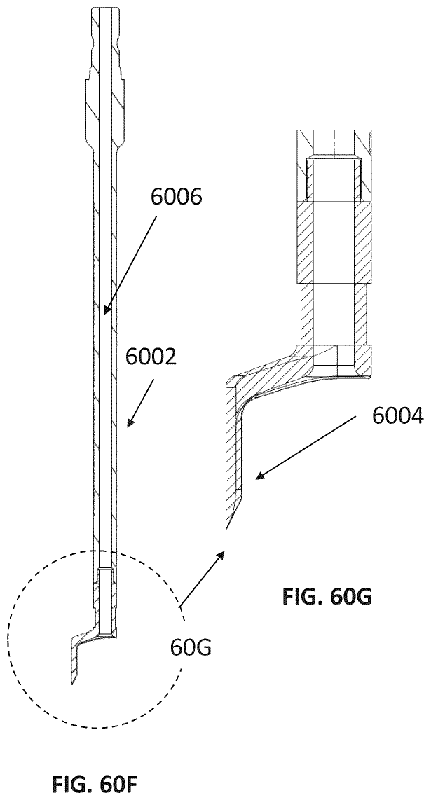

As shown in FIG. 60A, the user can place an appropriately sized cartilage scoring tool 6000 over the pin 5900. The user can create a clean cut on the edge of articular cartilage down to the subchondral bone by manually rotating the scoring tool (for example, by at least one revolution). FIGS. 60B-60G illustrate various views of the scoring tool 6000. The scoring tool 6000 can include an elongate shaft 6002 terminating at a single blade 6004 that is positioned radially offset from the elongate shaft 6002. The blade 6004 can rotate about the longitudinal axis of the shaft 6002 to cut in conformance to cartilage and underlying bone geometry. The scoring tool 6000 can include a pin hole 6006 for slidably receiving the guide pin 5900. As shown in FIGS. 60A and 60C, the elongate shaft 6002 can couple to a handle 6008 for easier rotation of the scoring tool 6000. The handle 6008 may have a universal and/or standard connection mechanism for connecting to various surgical tools disclosed herein.

The cut by the scoring tool 6000 extends beyond any existing osteochondral damage or defect. If the cut does not capture all damaged areas, a larger size implant may be needed and the user may repeat the steps shown in FIGS. 58A-B and 60A with a bigger femoral round pin guide/sizer 5800.

### 3. Femoral Implantation Site Reaming Round Profile

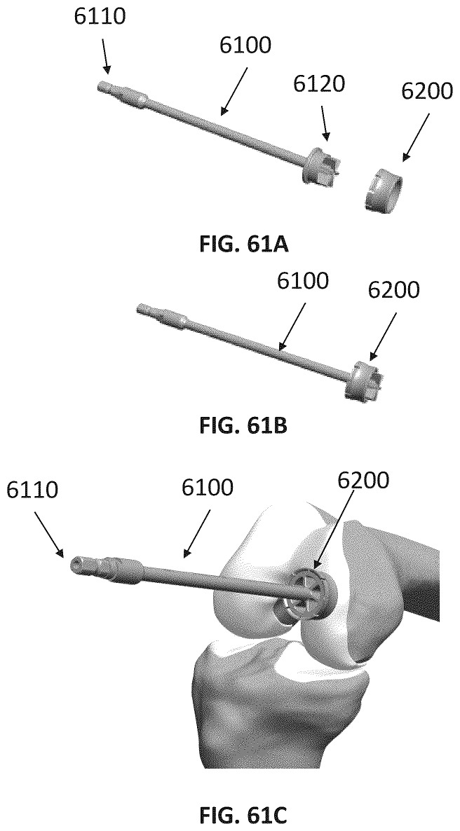

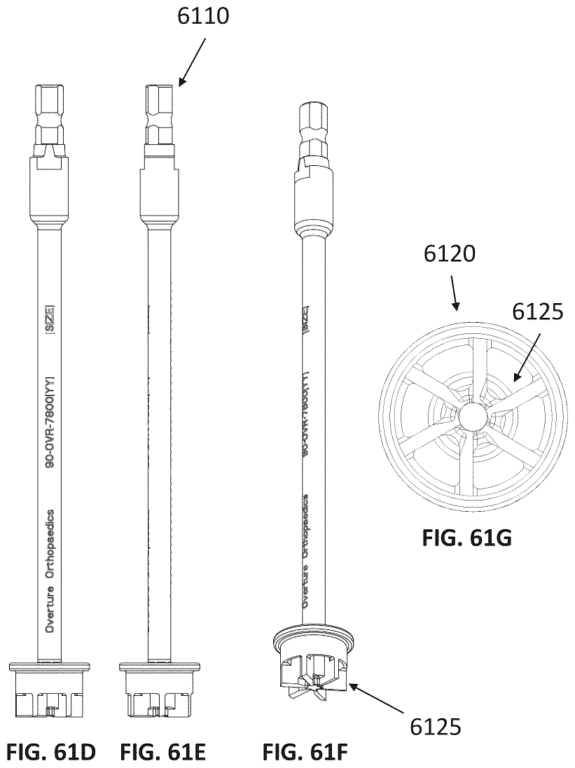

With the cartilage scoring tool 6000 removed, in FIG. 61A, the user can snap an appropriately sized stopper component 6200 onto a distal end 6120 of a first femoral reamer 6100. The distal end 6120 can include multiple cutting blades 6125, also referred to as a drill mechanism. The distal end 6120 can come in a variety of diameter sizes, depending on the size of the femoral implant that has been chosen in the step shown in FIG. 58A. As more clearly shown in FIG. 611, the distal end 6120 can include an attachment feature 6122 (such as a ledge) for snap-on or clip-on coupling of the stopper component 6200. The first femoral reamer 6100 may be powered by connecting a proximal end 6110 of the first femoral reamer 6100 to a motor and a power source. When activated, the first femoral reamer 6100 can ream the bone to produce a recess or hole in the distal portion of the femur, e.g., on a condyle.

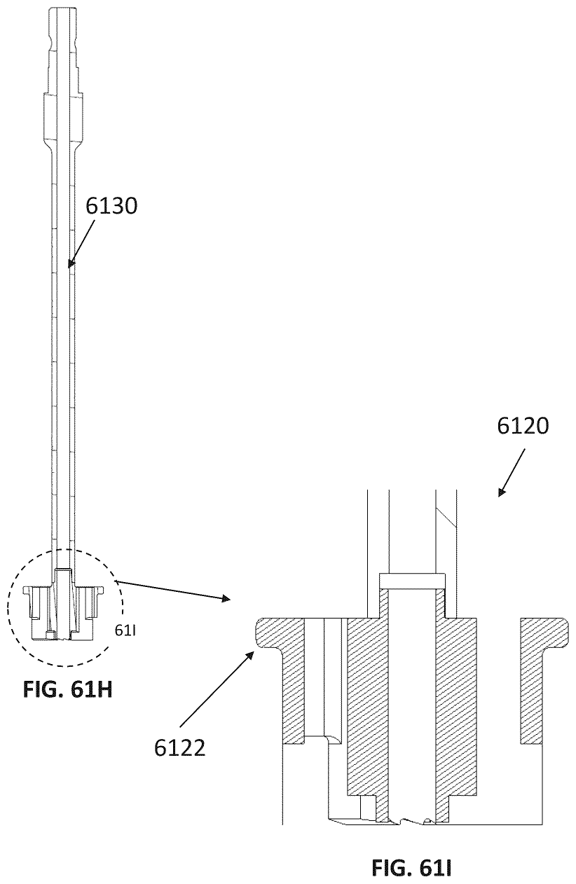

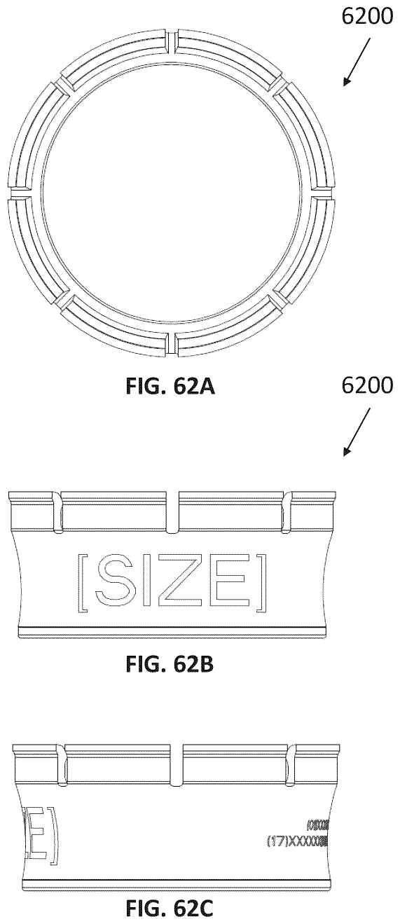

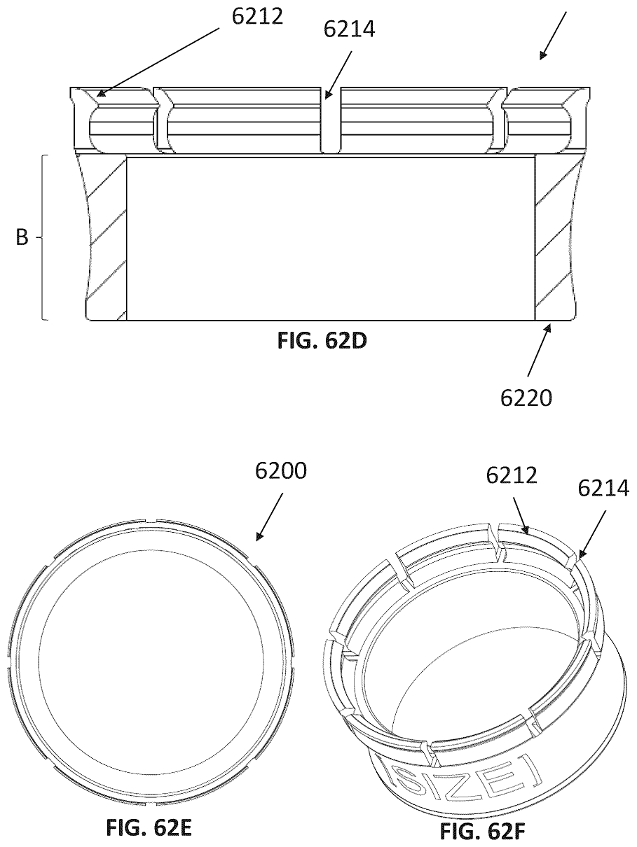

FIGS. 62A-62F illustrate aspects of an example stopper component 6200. For example, FIG. 62A illustrates a top or proximal view and FIG. 62E illustrates a bottom or distal view. The stopper component 6200 can have a size that matches the selected round femoral implant diameter. As shown, a proximal side 6210 of the stopper component 6200 can include multiple clips 6212 that are separated from one another by gaps 6214. The gaps 6214 can allow easier clipping and/or removal of the clips 6212 onto the attachment feature 6122 on the first femoral reamer 6100.

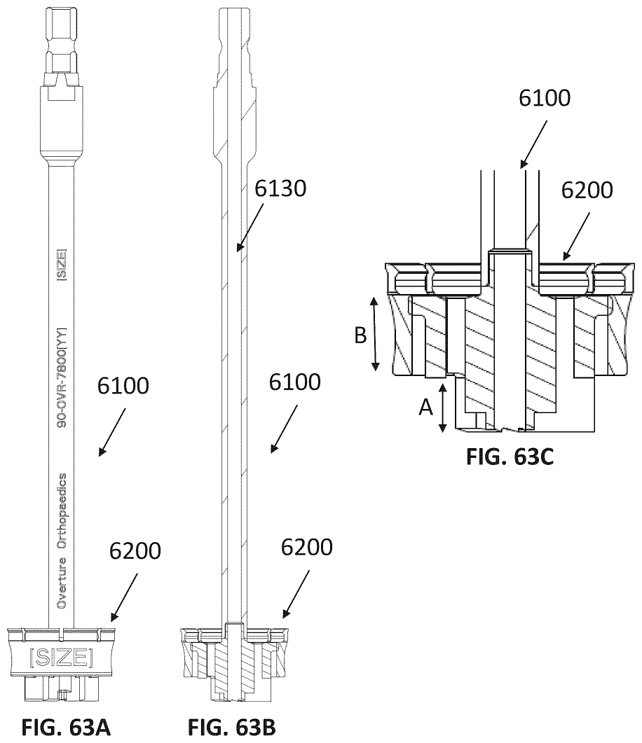

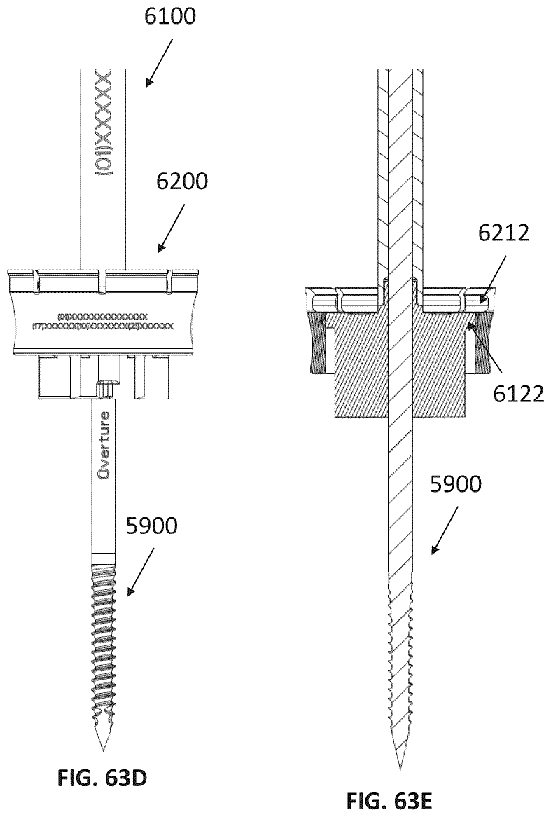

FIGS. 61B and 63A-63E illustrate the first femoral reamer 6100 with the stopper component 6200 snapped or clipped on. As illustrated in FIG. 63E, the height B of the stopper component 6200 can determine a depth A of reaming by the first femoral reamer 6100. A wide rim 6220 on a distal end of the stopper component 6200 can indicate that reaming is complete (i.e. the desired depth A has been reached) when the wide rim 6220 makes contact with the bone.

FIG. 61C illustrates the user placing the assembled first femoral reamer 6100 with the stopper component 6200 over the guide pin 5900 (hidden in this view, but illustrated in FIGS. 63C and 63D) via a guide pin hole 6130 of the first femoral reamer 6100. The distal end 6120 of the first femoral reamer is in contact with the cartilage surface. After connecting the proximal end 6110 of the first femoral reamer to a motor and a power source, the user can drive the first femoral reamer 6100 until a proper depth is achieved. The user may determine the first femoral reamer 6100 has reached the proper depth via the stopper component 6200, which can ensure that the first femoral reamer 6100 may not be able to ream past a depth as determined by the height of the stopper component 6200. As noted elsewhere in the present disclosure, the reaming depth control can facilitate having a single thickness of all the femoral implants.

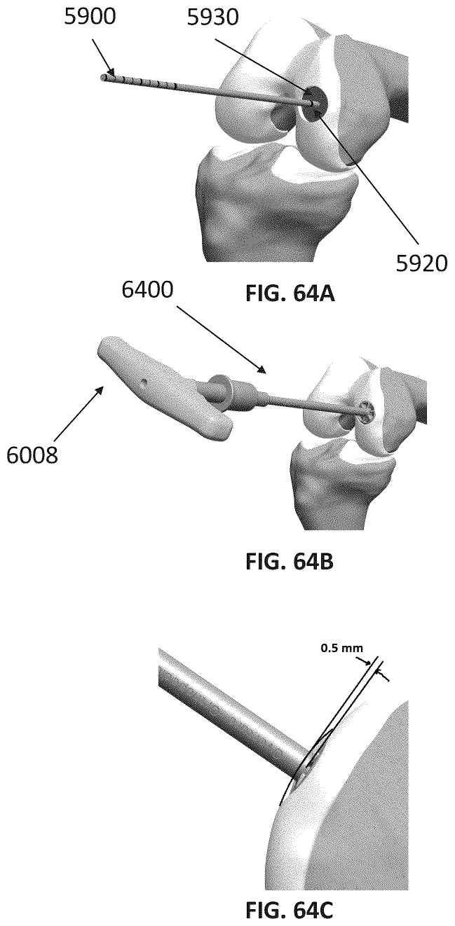

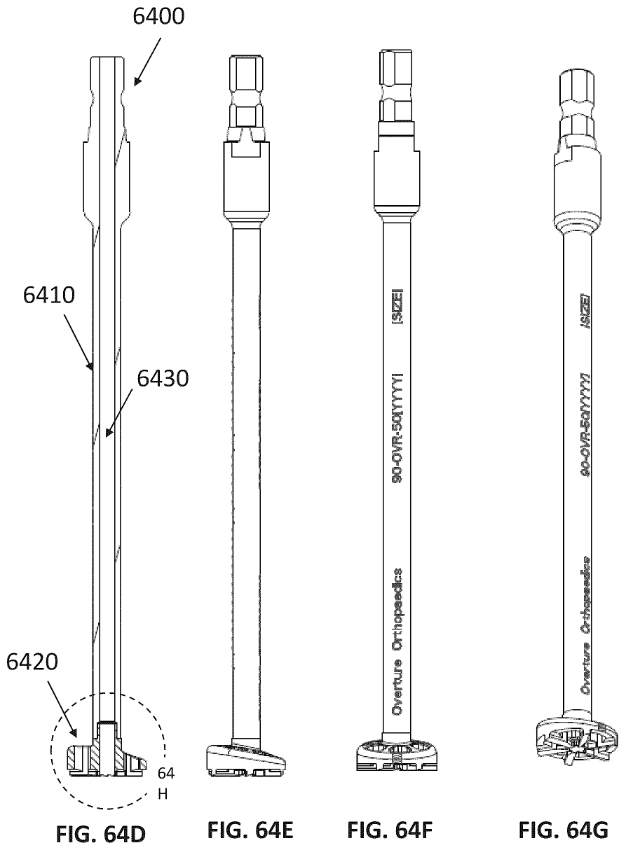

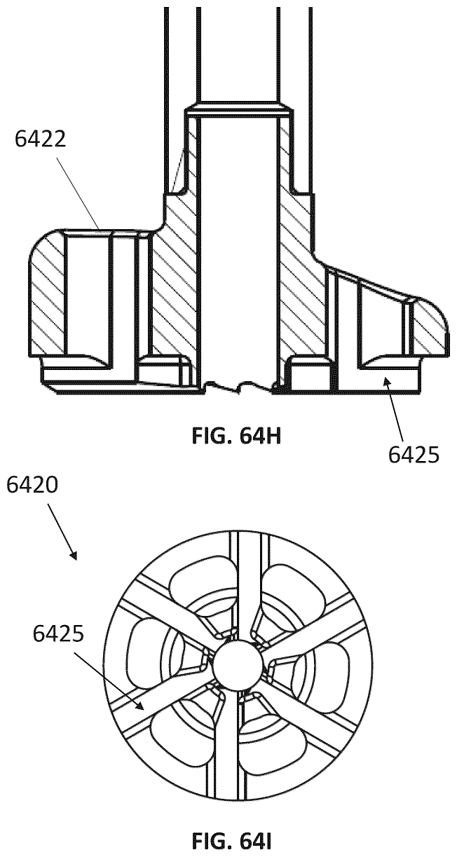

As shown in FIG. 64A, after the desired depth has been achieved, the user can then remove the first femoral reamer 6100 with the stopper component 6200, leaving behind the guide pin 5900 and a reamed bone surface 5930, which is at a distance equal to the reamed depth from a laser mark 5920 of the guide pin 5900. When the guide pin 5900 was first inserted, the laser mark 5920 was flush with the articulating bone surface. As powered reaming can generate a large amount of heat, the user can irrigate the reamed site to prevent thermal necrosis during reaming.

As shown in FIG. 64B, the user can slide a second femoral reamer 6400 over the guide pin 5900 to continue reaming by hand until a top surface of the second femoral reamer 6400 is slightly recessed below the adjacent cartilage surface. The second femoral reamer 6400 can be a hand reamer such that the user needs to rotate a handle 6008 coupled to a proximal end of the second femoral reamer 6400 in order to cut into the bone. In some implementations, the same handle 6008 can be used with a number of different tools disclosed herein (for example, the scoring tool 6000 as shown in FIG. 60A).

Using the second femoral reamer 6400 to hand ream the bone after using a powered reamer can ensure that the femoral implant will be positioned at the proper level upon final implantation. When properly inserted, the convex bearing surface of the femoral implant should sit recessed relative to the adjacent cartilage surface by a small distance. The distance can be, for example, from about 0.5 mm to about 1.0 mm, or about 0.5 mm as shown in FIG. 64C. The recess distance can take into account the deformation of cartilage during physiological loading. When the knee joint is under normal physiologic loading, the articular cartilage may elastically deform by a small distance (for example, about 0.5 mm) under such loads. Recessing the implant by a small distance can reduce or prevent premature cartilage wear on the articular surfaces of the adjacent tibia.

FIGS. 64D-64I illustrate an example second femoral reamer 6400. The second femoral reamer 6400 can include a shaft 6410 having a proximal end that defines the proximal end of the second femoral reamer 6400. The second femoral reamer 6400 can include a guide pin hole 6430 extending through a length of the second femoral reamer 6400. The shaft 6410 can be coupled to a base 6420 at a distal end of the shaft 6410. The base 6420 can have a circular profile as shown in FIG. 64H, with multiple cutting blades 6425 extending radially from a center of the circular profile of the base (when viewed from the bottom). The second femoral reamer 6400 can be available in various sizes with different diameters of the base 6420 to match the implant sizes. As more clearly shown in FIG. 64G, a top surface 6422 of the base 6420 may not be flat. Rather, the top surface 6422 of the second femoral reamer 6400 can match a shape of the selected femoral implant and/or a thickness of the base 6420 can match a thickness of the selected femoral implant. In other words, the shape and/or size of the base 6420 of the second femoral reamer 6400 can match a geometry and/or size of the femoral implant. Accordingly, the top surface 6422 of the base 6420 can be used as an indicator of the final implant position and the second femoral reamer 6400 can serve a dual function of a trial and a reamer.

Alternatively, in some implementations, the thickness of the second femoral reamer 6422 can be greater than the thickness of the femoral implant so that when a top surface 6422 of the second femoral reamer 6400 can be flush with the adjacent cartilage surface when the desired reaming depth has been reached. The user can additionally/optionally feel by hand whether the top surface 6422 of the second femoral reamer 6400 is flush with the adjacent cartilage surface.

In some implementations, the second femoral reamer 6400 may include a single-use version. The single-use second femoral reamer 6400 may have only the multiple cutting blades 6425 made in metal (e.g., stainless steel or any other suitable metal or metal alloy) and a remainder of the second femoral reamer 6400 made in plastic. The cutting blades 6425 can include stamped sheet metal with a sharp cutting edge. Optionally, the single-use second femoral reamer 6400 can also include an inner metal hypotube. The plastic portion of the single-use femoral reamer 6400 can be molded around the metal cutting blades 6425. The plastic portion can also be molded around the inner metal hypotube. The metal hypotube can increase the strength and/or rigidity of the plastic tube. The single-use femoral reamer 6400 can be more affordable than an all-metal second femoral reamer 6400 while being able to perform substantially the same function as the all-metal second femoral reamer 6400. The single-use femoral reamer 6400 also reduces the number of surgical tools for re-sterilization after the surgery.

### 4. Femoral Implant Trialing & Insertion—Round Profile

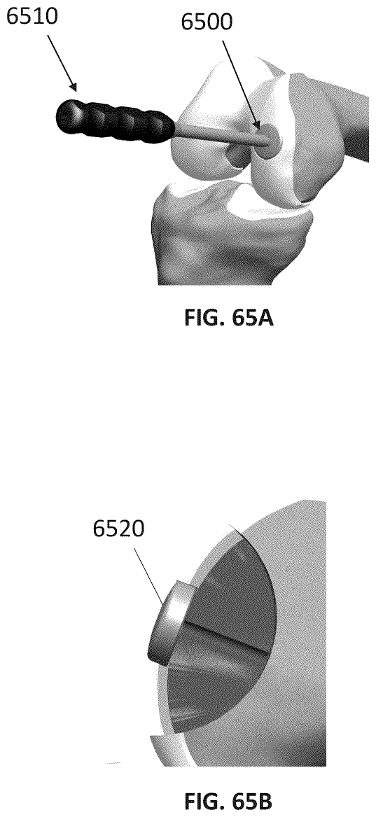

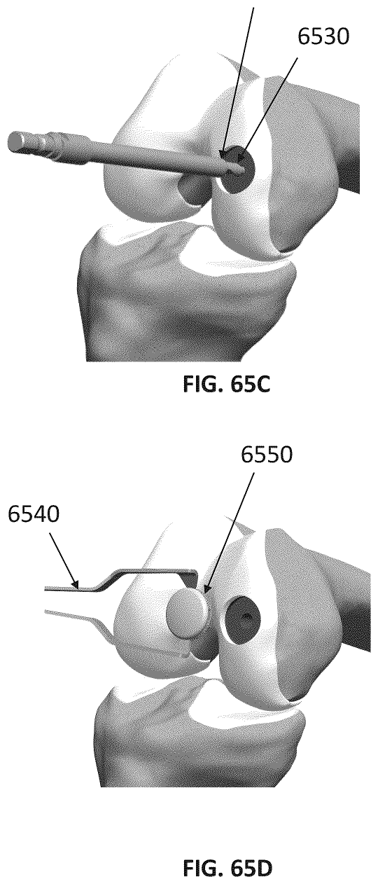

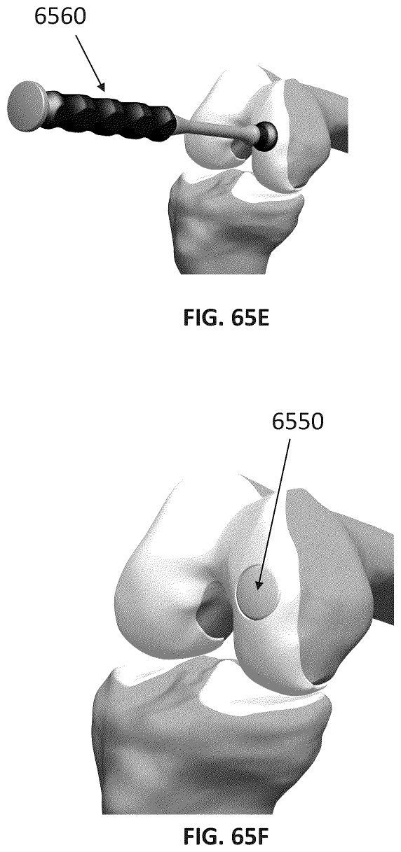

After hand reaming, the user can remove the second femoral reamer and the guide pin. The user can irrigate the reamed femoral site to remove debris. As shown in FIG. 65A, the user can insert correctly sized round femoral trial 6500 using a handle 6510. The round femoral trial 6500 can have the same size and shape as the portion of the corresponding femoral implant above the peg and/or spikes. The round femoral trial 6500 can have a central opening such that the handle 6510 can be coupled to the round femoral trial 6500 (e.g., via threaded engagement or a clip on feature). The user can visually inspect to ensure the top surface 6520 of the round femoral trial 6500 is recessed at the desired distance below the adjacent cartilage surface. Alternatively, the round femoral trial 6500 can be thicker than the corresponding femoral implant by the recessed distance disclosed herein and the user can visually and/or confirm by hand whether the round femoral trial 6500 is flush with the adjacent cartilage. When the thicker trial is flush with the cartilage, the actual femoral implant will sit recessed below the cartilage surface by the desired distance. Either way, the design of the trial and/or the inspection by the user can ensure that the final implant is correctly recessed below the surface (e.g., by about 0.5 mm). If adequate placement of the round femoral trial 6500 has not been achieved, the user can remove the round femoral trial and repeat the powered reaming and/or the hand reaming step until the proper depth is achieved. The user may need to repeat one or both of the powered reaming and hand reaming more than once to achieve the desired depth.

If the round femoral trial 6500 is properly placed, as shown in FIG. 65C, the user can remove the round femoral trial and insert the distal end of a drill bit 6530 into the hole created by the guide pin. Power can be supplied to the drill bit 6530 to drill until a shoulder 6532 on the drill bit 6530 reaches the reamed bone surface. The user can then remove the drill bit 6530. Optionally, the user can irrigate the area (e.g., with saline) to remove any loose debris and dry the bone surface (e.g., using a lap sponge). The user can apply a small amount (for example, about 1 ml to about 2 ml) of bone cement into the drilled hole. The user may ensure that the bone cement is only applied inside the drilled hole such that the porous metal subchondral surface of the femoral implant is in direct apposition to cancellous bone to facilitate integration. This way, the femoral implant can achieve hybrid fixation as disclosed herein. That is, cement fixation can be achieved at the peg/bone interface, while biologic bony in-growth fixation or osteointegration can be achieved at the porous metal subchondral surface of the femoral implant. In implementations where bone cement is used, the hole drilled by the drill bit 6530 can be slightly greater than the outer diameter of the barbs. In some implementations, additional cement may be placed under the subchondral implant surface in addition to around the peg to achieve greater initial fixation and/or to adjust the height of the implant after reaming. For example, if the bone is reamed too deep such that the implant is recessed more than a certain depth (such as 1 mm) below the native cartilage surface, the additional cement placed under the subchondral surface can add to the height to the implant. As another example, if the preparation of the hole(s) in the bone was poor, such as being on a poor angle, the cement placed under at least a portion of the subchondral surface of the implant can compensate for the poor angle.

In implementations where bone cement will not be used, the hole drilled can be slightly smaller than the outer diameter of the barbs to allow initial or primary fixation by interference between the barbs and the drilled hole before bone ingrowth into the porous metal layer. For example, the guide pin 5900 may have an outer diameter that is substantially the same as the outer diameter the peg outer wall without the barbs. The drilled hole can have an internal diameter that is about line to line with the peg outer wall without the barbs. In a cementless application, the step of drilling using the drill bit may be skipped.

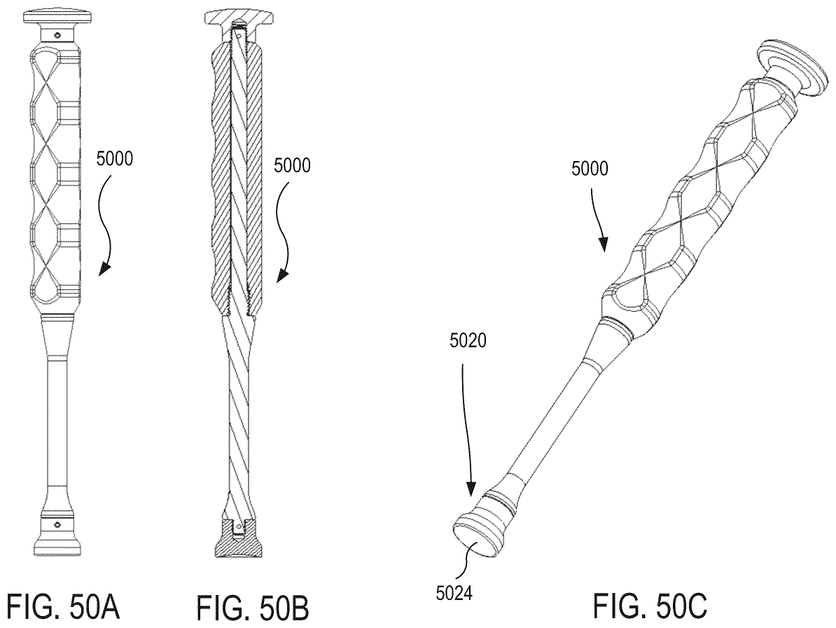

As shown in FIG. 65D, using an inserter tool 6540, the user can align the peg of the round femoral implant 6550 with the drilled hole and insert the peg into the drilled hole. The round femoral implant 6550 can be any of the example round femoral implants disclosed herein. As described elsewhere in the present disclosure, the round femoral implant can have multiple (e.g., two or four, or otherwise) pockets for interfacing with the inserter tool 6540 such that the user can grab the round femoral implant 6550 with the two prongs of the inserter tool 6540. As shown in FIG. 65E, the user can optionally use a femoral implant impactor 6560 to fully seat the round femoral implant 6550, ensuring that a periphery of the round femoral implant 6550 is recessed relative to the adjacent cartilage (for example, by about 0.5 mm to about 1.0 mm below the adjacent cartilage), such as shown in FIG. 65F. The user may center an interface surface of the femoral implant impactor 6560 over the round femoral implant 6550 to ensure that the femoral implant impactor 6560 does not contact the adjacent tissues to avoid unwanted trauma to the native articular surface. In some implementations, the interface surface of the femoral implant impactor 6560 can be concave to match the convex articulating surface of the round femoral implant 6550. The femoral implant impactor 6560 can be the impactor system 5000 shown in FIGS. 50A-50C.

FIGS. 50A-50C depict aspects of an impactor system 5000. FIG. 50A provides a side view, FIG. 50B provides a cross-section view, and FIG. 50C provides a perspective view. As shown here, an impactor system 5000 can include a distal end 5020 having a concave surface 5024, which can be shaped or configured to match or complement a convex surface of an implant (e.g. a femoral implant).

### 5. Femoral Implantation Oblong Profile (Reaming of Superior & Inferior Holes)

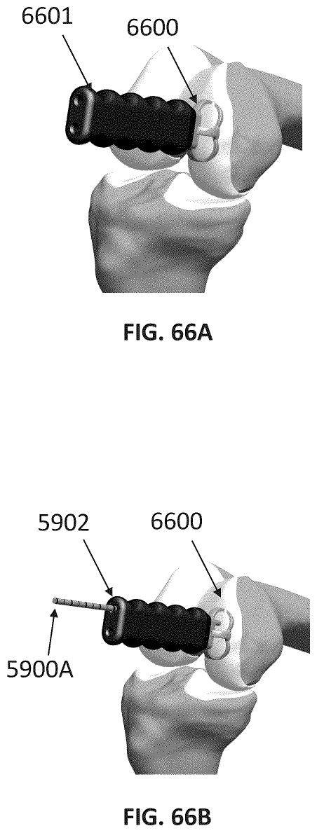

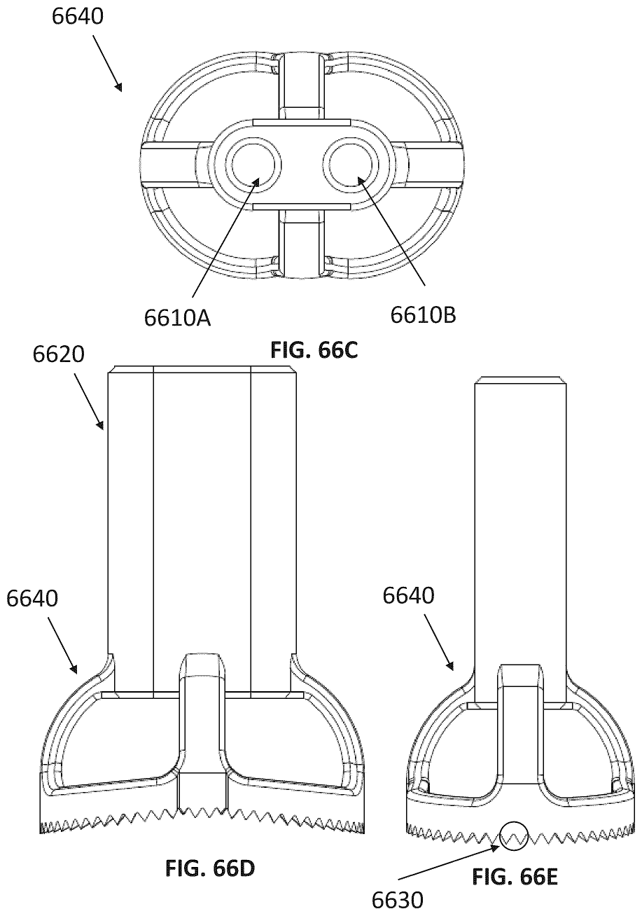

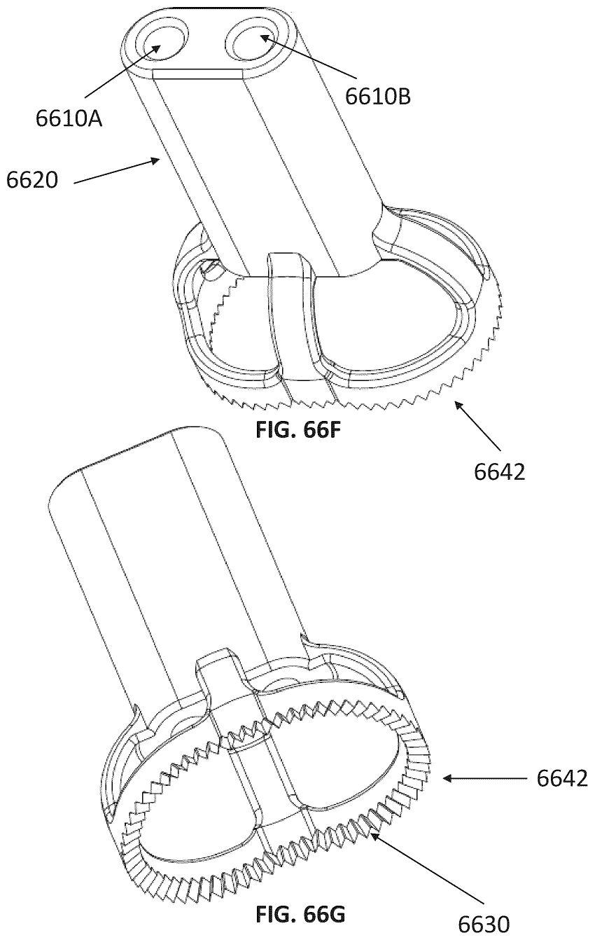

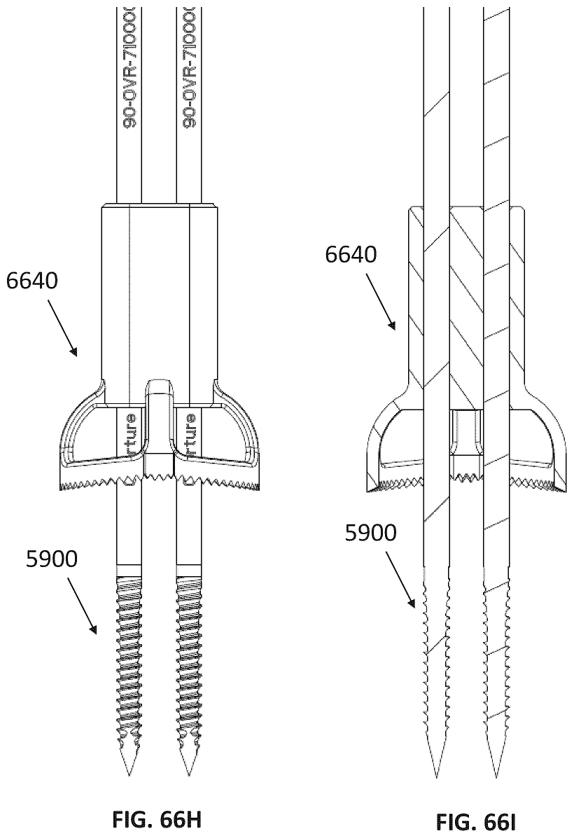

As shown in FIG. 66A, with the knee in flexion, a user can assess the proper implant size using a femoral oblong pin guide/sizer 6600, which can be available in a range of different sizes. The femoral oblong pin guide/sizer 6600 is shown in various views in FIGS. 66C-66I. The femoral oblong pin guide/sizer 6600 can include a base 6640 with a socket 6620 for coupling to a handle portion 6641. The base 6640 can have an oblong profile. Different sizes of the femoral oblong pin guide/sizer 6600 can differ in the outer diameter and/or length of the base 6640. For example, the outer diameter can range from between about 17 mm to about 22 mm. For example, the length can range from between about 30 mm to about 40 mm. The underside 6642 of the base can have a surface geometry identical to the articulating surface of the femoral implant of the corresponding size.

The femoral oblong pin guide/sizer 6600 can include guide pin holes 6610A, 6610B each extending along a longitudinal axis of the femoral round pin guide/sizer 6600. The guide pin holes 6610A, 6610B can be centered on the two outer circles of the base 6640. The user can select a femoral oblong pin guide/sizer 6600 with a suitable size so that the base 6640 of the femoral oblong pin guide/sizer 6600 fully extends beyond the osteochondral defect to ensure full removal of the affected area and proper implant coverage. The femoral oblong pin guide/sizer 6600 matching the chosen implant size may be surrounded by normal/healthy articular cartilage on all sides. When using the femoral oblong pin guide/sizer 6600, the user may ensure the base 6640 is fully in full contact with the condyle cartilage on all sides. This may ensure that the femoral oblong pin guide/sizer 6600 is perpendicular to the femoral surface and/or the cartilage, thereby improving implant alignment.

As shown in FIG. 66B, with the femoral oblong pin guide/sizer 6600 flush with the surface of the native cartilage, the user may drive a guide pin 5900A into one of the guide pin holes 6610A, 6610B. In some implementations, the user may begin with driving the guide pin hole 6610A on the femoral oblong pin guide/sizer 6600. Alternatively, the user may choose to drive the guide pin 5900A into the other guide pin hole 6610B first before driving the guide pin 5900A into the guide pin hole 6610A.

In some implementations, the user may drive the guide pin 5900A into the condyle until a laser mark line 5902 of the pin 5900A is flush with a proximal end of the handle portion 6601 of the femoral oblong pin guide/sizer 6600. The base 6640 of the femoral oblong pin guide/sizer 6600 can include spikes 6630. The spikes 5830 can stabilize the femoral round pin guide/sizer 5800 while installing guide pin(s) 5900A/5900B (e.g. by engaging the bone). After the guide pin 5900A has been driven to the desired depth, the user can remove the femoral oblong pin guide/sizer 6600.

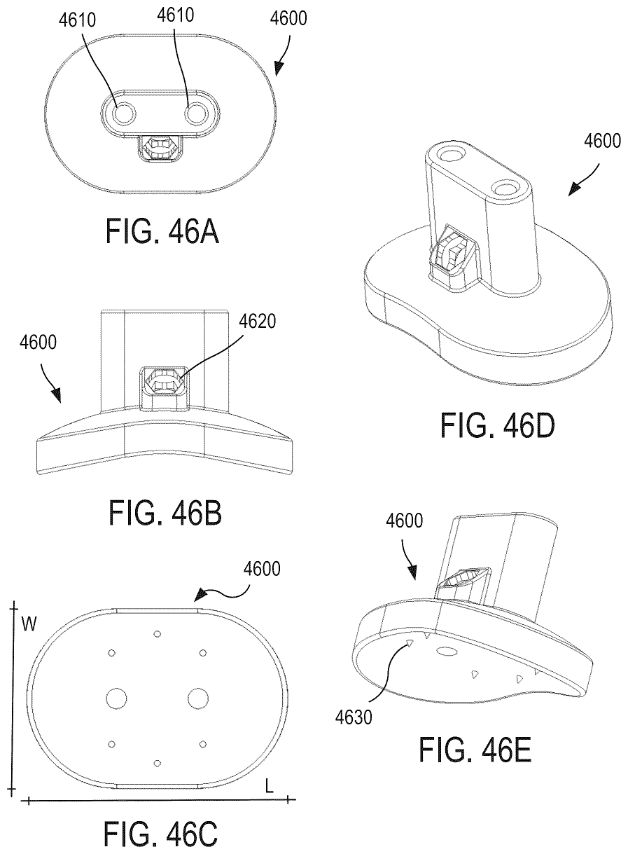

FIGS. 46A-46E depict aspects of a pin guide sizer mechanism 4600. Features of the femoral oblong pin guide/sizer 6600 can be incorporated in the pin guide sizer mechanism 4600 and vice versa. FIG. 46A provides a top plan view, FIG. 46B provides a front side view, FIG. 46C provides a bottom plan view, FIG. 46D provides an upper perspective view, and FIG. 46E provides a lower perspective view. As shown here, pin guide sizer mechanism 4600 includes guide pin holes 4610, a universal instrument handle socket 4620, and spikes 4630 which can operate to stabilize the guide mechanism 4600 while installing pins (e.g. by engaging patient bone). In some cases, the pin guide mechanism 4600 can have a width W and a length L that can be used to determine the size of the implant. In some cases, the length and width offerings are 1 to 1 with implant sizing, or otherwise mimic or match the implant sizing.

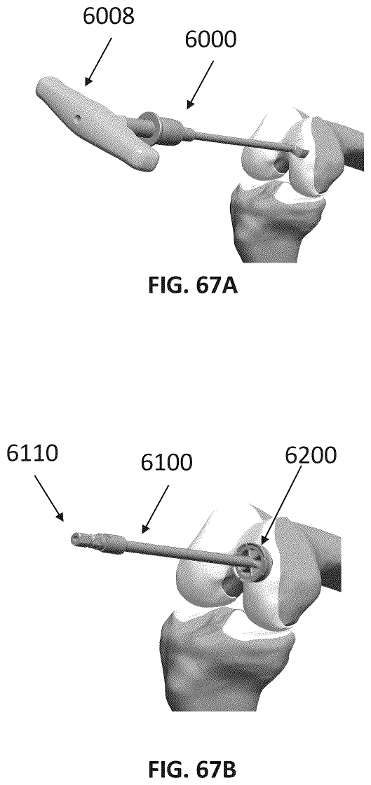

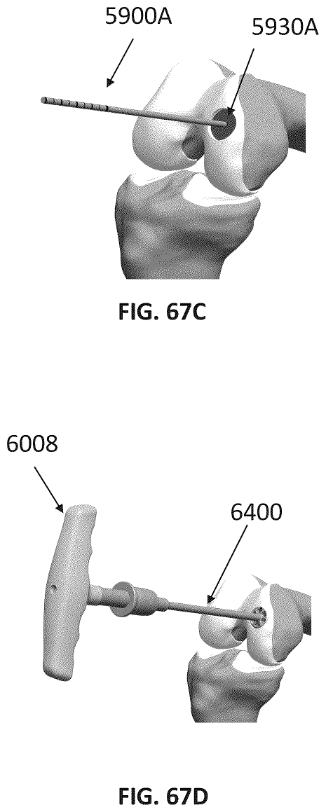

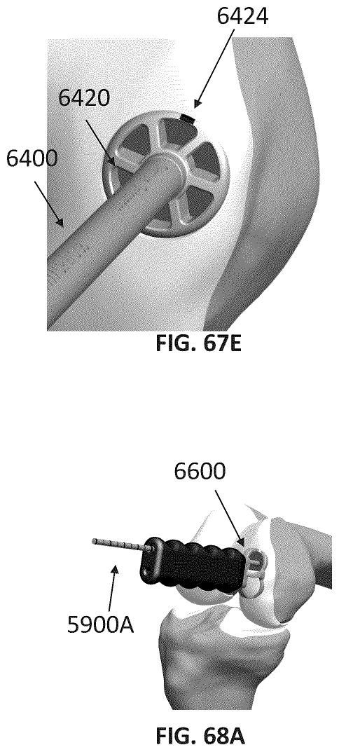

As shown in FIG. 67A, the user can place an appropriately sized cartilage scoring tool 6000 over the guide pin 5900A. The user can create a first clean cut on the edge of articular cartilage down to the subchondral bone by manually rotating the scoring tool (for example, by at least one or more revolutions) as disclosed elsewhere in the present disclosure. The cartilage scoring tool 6000 is described above with reference to FIGS. 60B-60G in the present disclosure. As will be described further below, additional cuts (e.g., second and third cuts) will be made adjacent to the first cut. The combination of all the cuts can extend beyond any existing osteochondral damage or defect. If the combination of the cuts do not capture all damaged areas (e.g., if the first cut does not extend beyond a superior defect or a superior portion of a defect in the illustrated example), a larger sized implant may be needed and the user may repeat the steps shown in FIGS. 66A, 66B and 67A with a bigger femoral oblong pin guide/sizer 6600.

With the cartilage scoring tool 6000 removed, the user can snap an appropriately sized stopper component 6200 onto a distal end 6120 of a first femoral reamer 6100. The first femoral reamer 6100 and the stopper component 6200 are described with reference to FIGS. 61D-611, 62A-62F, and 63A-63E. FIG. 67B illustrates the user placing the assembled first femoral reamer 6100 with the stopper component 6200 over the guide pin 5900A (hidden in this view but shown in FIG. 67C) via a guide pin hole 6130 of the first femoral reamer 6100. The distal end 6120 of the first femoral reamer 6100 can be in contact with the cartilage surface. After connecting the proximal end 6110 of the first femoral reamer 6100 to a motor and a power source, the user can drive the first femoral reamer 6100 until a proper depth is achieved beyond the first cut by the scoring tool 6000. The user may ensure the first femoral reamer 6100 has reached the proper depth via the stopper component 6200 as described elsewhere in the present disclosure, which can ensure that the first femoral reamer 6100 may not be able to ream past a depth as determined by the height 6220 of the stopper component 6200. As noted elsewhere in the present disclosure, the reaming depth control can facilitate an implementation of a single thickness for all the femoral implants.

As shown in FIG. 67C, after the desired depth has been achieved, the user can then remove the first femoral reamer 6100 with the stopper component 6200, leaving behind the guide pin 5900A and a reamed bone surface or hole 5930A. The reamed bone surface or hole 5930A corresponds to one of the outer circles of the selected oblong femoral implant. As powered reaming can generate a large amount of heat, the user can irrigate the reamed site to prevent thermal necrosis during reaming.

As shown in FIG. 67D, the user can slide a second femoral reamer 6400 over the guide pin 5900A and ream by hand until a top surface of the second femoral reamer 6400 is slightly recessed below the adjacent cartilage surface by a small distance. The distance can be, for example, from about 0.5 mm to about 1.0 mm, or about 0.5 mm as shown in FIG. 64C as disclosed elsewhere in the present disclosure. When properly inserted, the convex bearing surface of the femoral implant should sit recessed relative to the adjacent cartilage surface by the small distance. The second femoral reamer 6400 is described with reference to FIGS. 64D-64H elsewhere in the present disclosure.

As shown in FIG. 67E, if the first reamed bone surface or hole 5930A is a more superior hole, the base 6420 of the second femoral reamer 6400 may be oriented such that a laser marked section 6424 of the second femoral reamer 6400 is superior when checking for the depth, as this orientation of the second femoral reamer 6400 matches the shape of a superior portion of the oblong femoral implant when it is inserted into the implant site.

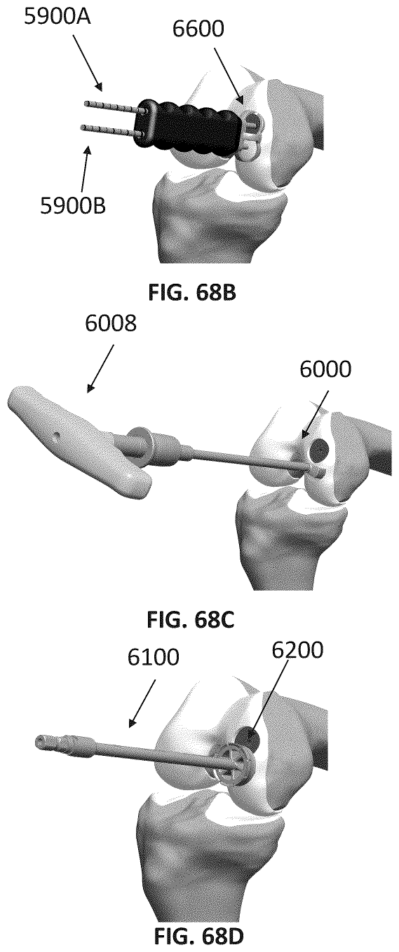

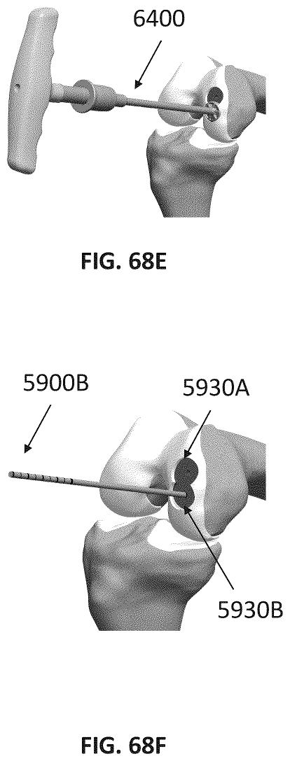

FIGS. 68A-68F illustrate example surgical steps of creating a second bone recess corresponding to the other outer circle of the selected oblong femoral implant. As shown in FIG. 68A, with the second femoral reamer 6400 removed, the femoral oblong pin guide/sizer 6600 can be reintroduced by sliding the guide pin hole 6610A over the guide pin 5900A that is still inserted into the bone. The user can orient the femoral oblong pin guide/sizer 6600 to ensure the base 6640 still covers the entire affected area (that is, area or areas with one or more osteochondral defects). As shown in FIG. 68B, the user can insert a second guide pin 5900B into the other guide pin hole 6610B. In the illustrated example, the second guide pin 5900B is inferior to the guide pin 5900A. However, as noted elsewhere in the present disclosure, the user can start the procedure with either of the two guide pin holes 6610A, 6610B. The user can then remove the guide pin 5900A and the femoral oblong pin guide/sizer 6600.

As shown in FIGS. 68C-68F, the user may repeat the steps for cartilage scoring, reaming under power, and hand reaming shown in FIGS. 67A-67D to create a second circular (or at least partially circular) reamed bone surface or hole 5930A (which is inferior to the bone surface 5930A in the illustrated example) with two differences compared to the steps of preparing the first hole 5930A. The first difference is that the scoring tool 6000, the first femoral reamer 6100 and stopper component 6200, and the second femoral reamer 6400 slide over and are guided by the guide pin 5900B instead of the guide pin 5900A. The second difference is that when performing the hand reaming step, the user may orient the laser marked section 6424 on the base 6420 of the second femoral reamer 6400 inferiorly when assessing the reaming depth, as this orientation matches the shape of the inferior portion of the oblong femoral implant when it is inserted into the implant site.

### 6. Femoral Implantation—Oblong Profile (Reaming of Central Hole)

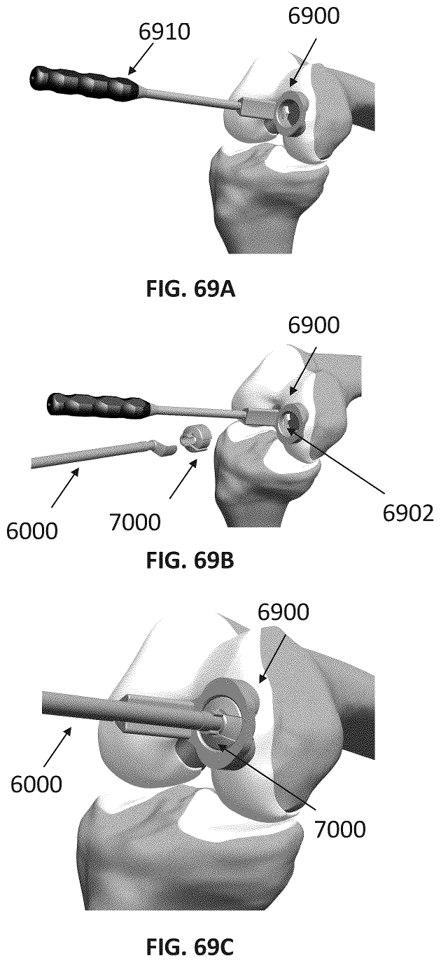

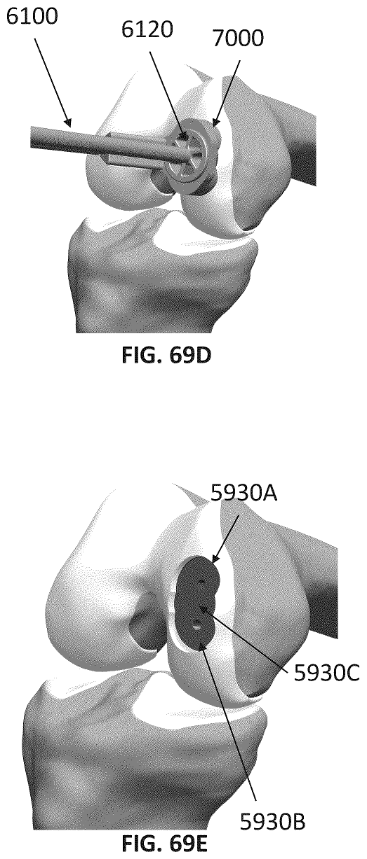

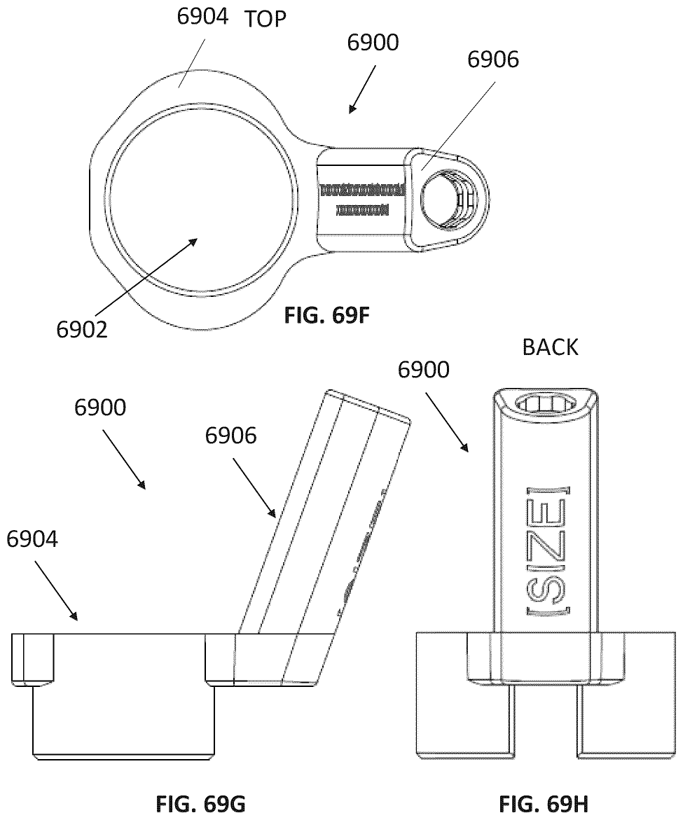

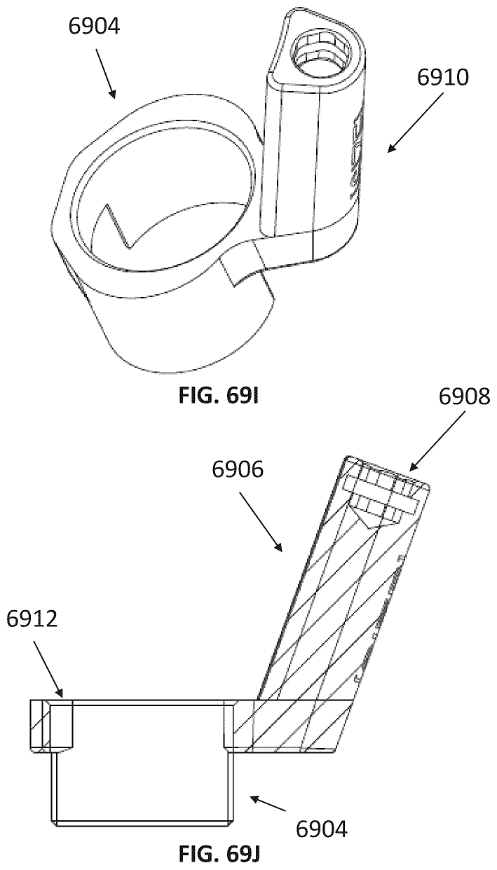

With the first or superior hole 5930A and the second or inferior hole 5930B prepared, FIGS. 69A-69C illustrate example surgical steps for creating a center hole in the bone 5930C. As shown in FIG. 69A, the user can select a correctly sized femoral reamer guide 6900 and insert the femoral reamer guide 6900 into the reamed bone (see, e.g., the partially overlapped holes 5930A, 5930B as shown in FIG. 68F) using a handle 6910. In some implementations, the handle 6910 can be the same as the handle 6510 such that a universal handle can be used for coupling with different surgical tools. The femoral reamer guide 6900 can include an opening 6902, which exposes the reamed bone surface.

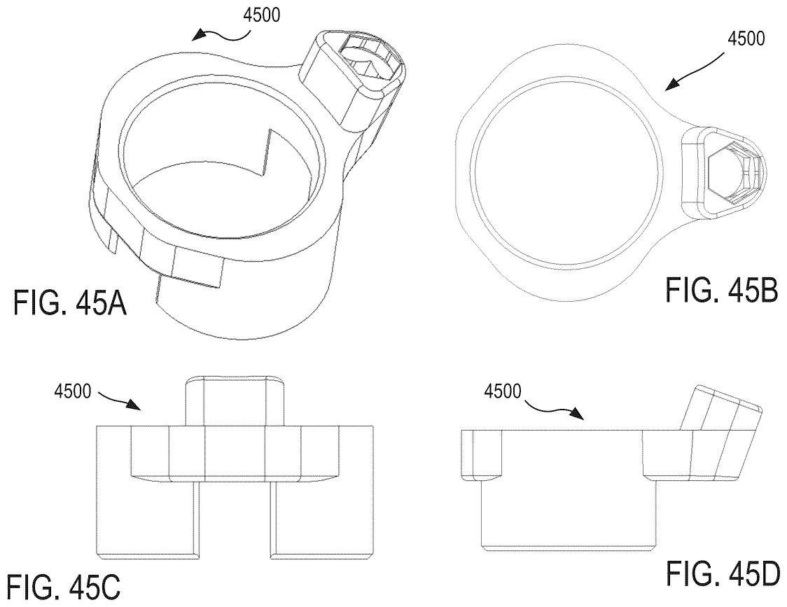

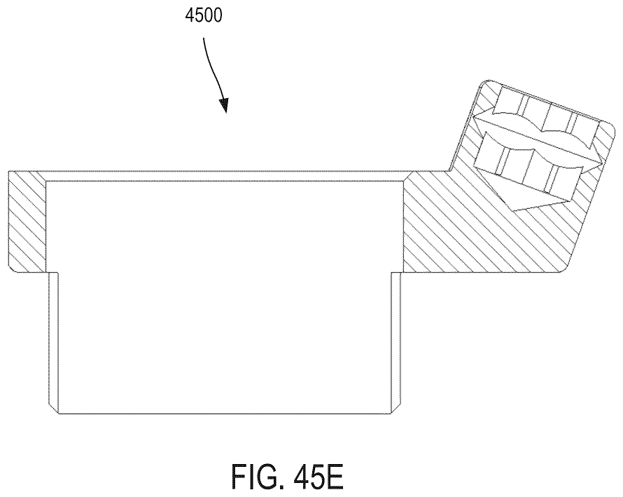

FIGS. 69F-69J illustrate various views of the femoral reamer guide 6900. The opening 6902 can be located on a body 6904 of the femoral reamer guide 6900. The body 6904 can have a footprint that generally corresponds to the size and shape of the reamed bone surface and/or the selected oblong femoral implant. As shown in FIG. 69J, the body 6904 can include a shoulder 6912 to facilitate controlling the depth of reaming of the central hole. The body 6904 can be coupled to a handle coupling portion 6906. The handle coupling portion 6906 can include coupling features 6908 for being coupled to the handle 6910, which can be any suitable mechanical coupling features, such as any frictional fit or quick release coupling features. The handle coupling portion 6906 can be slanted or non-parallel to the longitudinal axis of the opening 6902. The orientation of the handle coupling portion 6906 can ensure that the handle 6910, when coupled to the femoral reamer guide 6900, does not obstruct the path of inserting another tool into the opening 6902, for example, a scoring tool or a first and/or second femoral reamer. FIGS. 45A-45E depict aspects of a reamer guide mechanism 4500. FIG. 45A provides a perspective view, FIG. 45B provides a top plan view, FIG. 45C provides a front side view, FIG. 45D provides a right side view, and FIG. 45E provides a cross-section view. Features of the femoral reamer guide 6900 can be incorporated into the reamer guide mechanism 4500 and features of the reamer guide mechanism 4500 can be incorporated into the femoral reamer guide 6900.

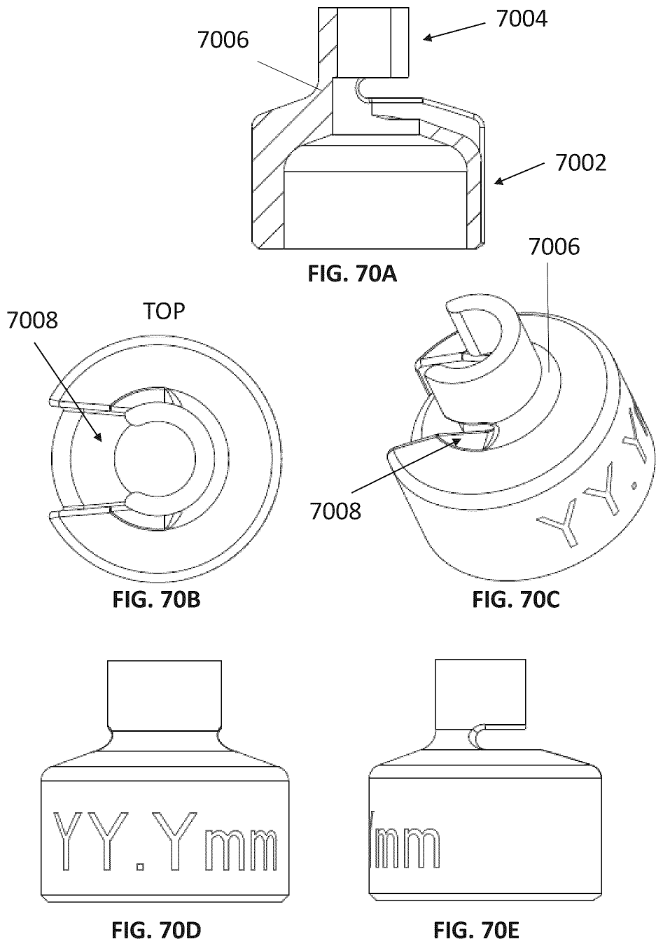

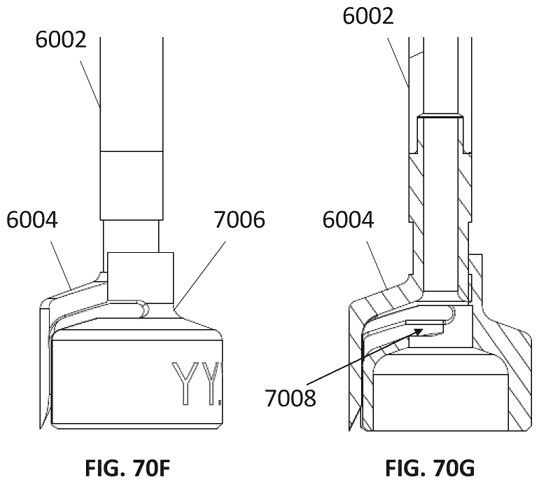

With the femoral reamer guide 6900 sized and inserted, as shown in FIG. 69B, the user can install a scoring tool guide 7000 in the opening 6902 of the femoral reamer guide 6900. The opening 6902 can be sized to slidably fit an outer diameter of a correctly sized scoring tool guide 7000. As there is no guide pin inserted in the bone for the center hole, the scoring tool guide 7000 can guide the scoring tool 6000 to cut the cartilage at the center hole. FIGS. 70A-70E illustrates various views of an example scoring tool guide 7000. The scoring tool guide 7000 can include a first portion 7002 sized to fit into the opening 6902. The scoring tool guide 7000 can also include a second, smaller portion 7004 having an opening sized to fit the shaft 6002 of the scoring tool 6000 (see FIGS. 60B-60G) by laterally inserting the shaft 6002 into the opening of the second portion 7004. The first and second portions 7002, 7004 are connected by a neck portion 7006. The neck portion 7006 and the first portion 7002 can include cutout sections 7008 such that the user can laterally insert the blade 6004 (see FIGS. 60B-60G) of the scoring tool 6000 into the cutout sections 7008 and the shaft 6002 into the opening of the second, smaller portion 7004, such as shown in FIG. 69C. FIGS. 70F and 70G illustrate inserting the scoring tool 6000 into the cut-out sections 7008 of the scoring tool guide 7000. The user can use the cartilage scoring tool 6000 to trim the cartilage by rotating the scoring tool 6000 (which causes the scoring tool guide 7000 to rotate together), for example, by one or more revolutions, as described elsewhere in the present disclosure, such as with reference to FIG. 60A.

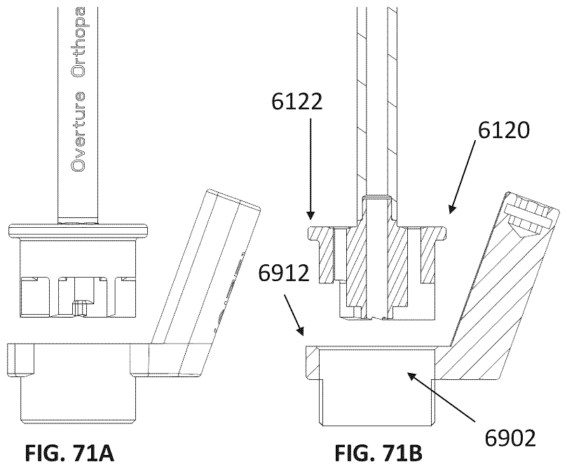

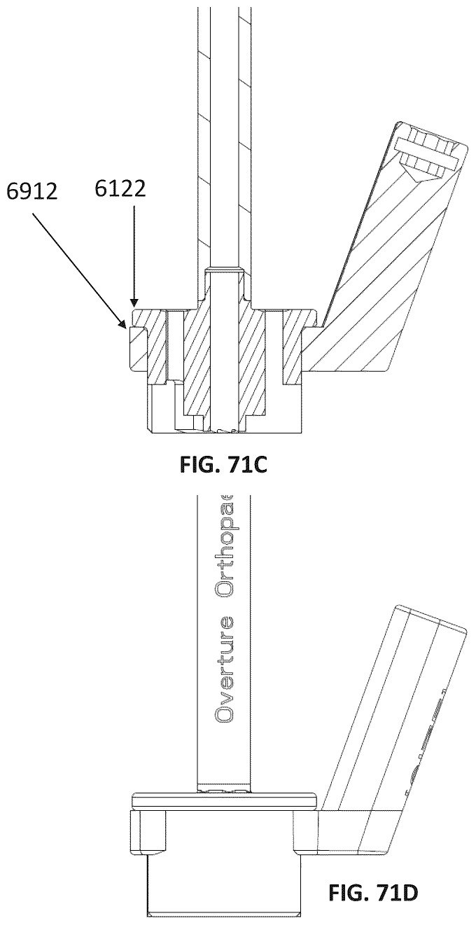

After the cartilage is trimmed, as shown in FIGS. 69D and 69E, the user can use the first femoral reamer 6100 to ream the central hole. If the first femoral reamer 6100 is still coupled with the stopper component 6200 after the powered reaming of the superior and/or inferior holes 5930A, 5930B, the user can remove the stopper component 6200 from the first femoral reamer 6100 before inserting the base of the first femoral reamer 6100 concentrically into the opening 6902 of the femoral reamer guide 6900 as shown in FIG. 69D. The user can drive the first femoral reamer 6100 to cut the central hole in the bone until the first femoral reamer 6100 reaches a built-in depth stop. As shown in FIGS. 69J and 71A-71D, the built-in depth stop can be in the form of the shoulder 6912 of the body 6904 of the femoral reamer guide 6900. The first femoral reamer 6100 (e.g., the attachment feature or ledge 6122 of the base) can bottom out at the shoulder 6912 of the femoral reamer guide 6900 during reaming of the central hole, which can determine the depth of reaming. As shown in FIG. 69E, the central hole 5930C can be centered between the first hole 5930A and the second hole 5930B. For the central hole 5930C, hand reaming using the second femoral reamer 6400 can be optional after powered reaming using the first femoral reamer 6400. Alternatively, the user can prepare the center hole 5930C by hand reaming using the second femoral reamer 6400 without using the first femoral reamer 6100.

### 7. Femoral Implant Trialing & Insertion—Oblong Profile

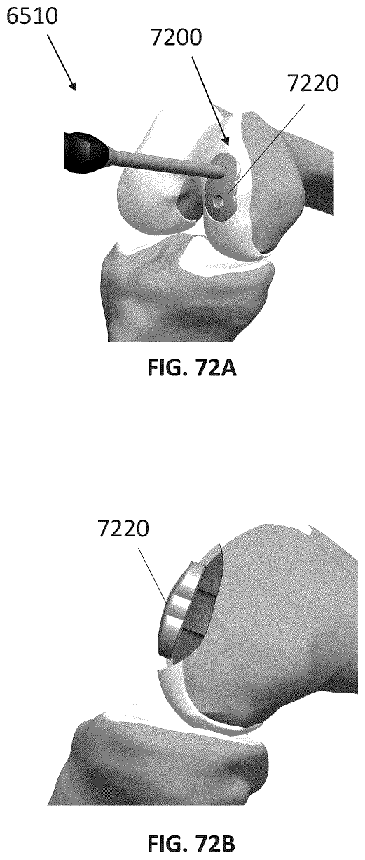

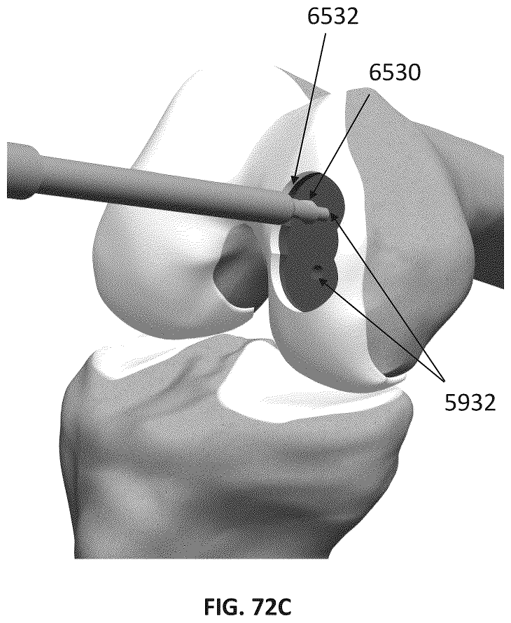

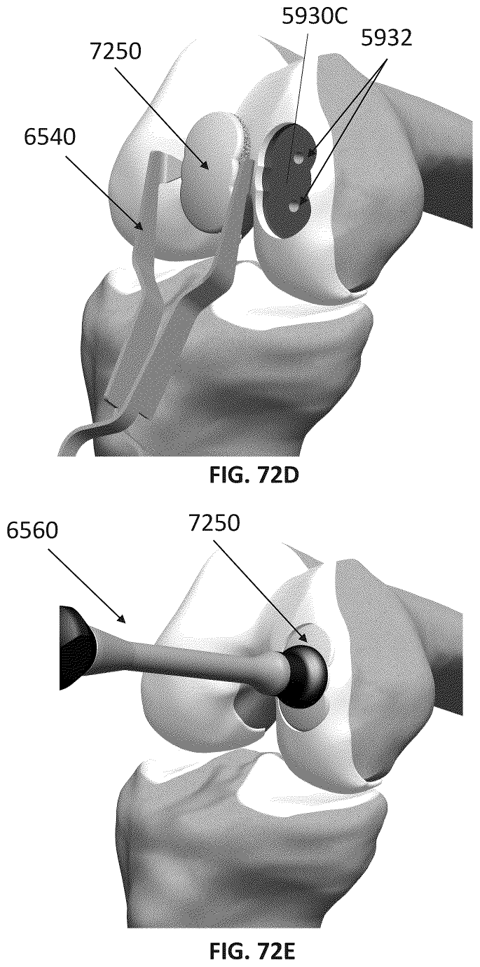

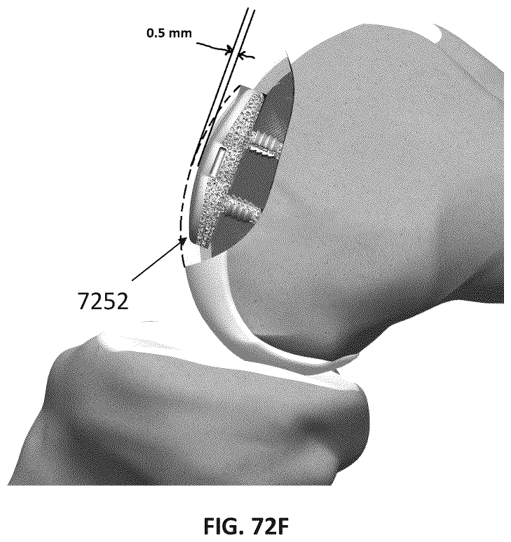

The steps of trialing and inserting the oblong femoral implant can be similar to those steps for inserting a round femoral implant as disclosed elsewhere in the present disclosure and can incorporate any of the features of the steps for trialing and inserting the round femoral implant, such as described with reference to FIGS. 65A-65E, with the differences described herein with reference to FIGS. 72A-72E. As shown in FIG. 72A, the user can insert correctly sized oblong femoral trial 7200 using the handle 6510. The oblong femoral trial 7200 can have the same size and shape as the portion of the corresponding femoral implant above the peg and/or spikes (if any), that is, between the convex bearing surface and the subchondral surface. The oblong femoral trial 7200 can have two openings centered on the two outer circles of the trial 7200 such that the handle 6510 can be coupled to the oblong femoral trial 7200 via one of the circles (e.g., via threaded engagement or a clip on feature). Alternatively, the oblong femoral trial 7200 can have a central opening. The user can visually inspect to ensure the top surface 7220 of the oblong femoral trial 7200 is recessed at the desired distance below the adjacent cartilage surface as disclosed elsewhere in the present disclosure. Alternatively, the oblong femoral trial 7200 can be thicker than the corresponding femoral implant by the recessed distance disclosed herein and the user can visually and/or confirm by hand whether the oblong femoral trial 7200 is flush with the adjacent cartilage. When the thicker trial is flush with the cartilage, the actual femoral implant will sit recessed below the cartilage surface by the desired distance. Either way, the design of the trial and/or the inspection by the user can ensure that the final implant is correctly recessed below the surface (e.g., by about 0.5 mm). If adequate placement of the oblong femoral trial 7200 has not been achieved, the user can remove the oblong femoral trial 7200 and repeat one or more of the reaming steps disclosed above until the proper depth is achieved. The user may need to repeat one or both of the powered reaming and hand reaming more than once to achieve the desired depth.

If the oblong femoral trial 7200 is properly placed, as shown in FIG. 72C, the user can remove the oblong femoral trial and insert the distal end of a drill bit 6530 into each of the two holes 5932 created by the guide pins. As noted above, each hole 5932 can be centered at the superior and/or inferior holes 5930A, 5930B. Power can be supplied to the drill bit 6530 to drill until a shoulder 6532 on the drill bit 6530 reaches the reamed bone surface. Alternatively, the user can drill to a laser mark line on the drill bit 6530. The user can then remove the drill bit 6530. Optionally, the user can irrigate the area (e.g., with saline) to remove any loose debris and dry the bone surface (e.g., using a lap sponge).

The user can apply a small amount (for example, about 1 ml to about 2 ml) of bone cement into the drilled holes. The user may ensure that the bone cement is only applied inside the drilled holes such that the porous metal subchondral surface of the femoral implant is in direct apposition to cancellous bone to facilitate integration. This way, the femoral implant can achieve hybrid fixation as disclosed herein. That is, cement fixation can be achieved at the peg/bone interface, while biologic bony in-growth fixation or osteointegration can be achieved at the porous metal subchondral surface of the femoral implant. In implementations where bone cement is used, the holes drilled by the drill bit 6530 can be slightly greater than the outer diameter of the barbs. In some implementations, additional cement may be placed under the subchondral implant surface in addition to around the peg to achieve greater initial fixation and/or to adjust the height of the implant after reaming. For example, if the bone is reamed too deep such that the implant is recessed more than a certain depth (such as 1 mm) below the native cartilage surface, the additional cement placed under the subchondral surface can add to the height to the implant. As another example, if the preparation of the hole(s) in the bone was poor, such as being on a poor angle, the cement placed under at least a portion of the subchondral surface of the implant can compensate for the poor angle.

In implementations where bone cement will not be used, the holes drilled can be slightly smaller than the outer diameter of the barbs to allow initial or primary fixation by interference between the barbs and the drilled holes before bone ingrowth into the porous metal layer. For example, the guide pin 5900 may have an outer diameter that is substantially the same as the outer diameter the peg outer wall without the barbs. The drilled hole can have an internal diameter that is about line to line with the peg outer wall without the barbs. In a cementless application, the step of drilling using the drill bit may be skipped.

As shown in FIG. 65D, using an inserter tool 6540, the user can align the two pegs of the oblong femoral implant 7250 with the two drilled holes 5932 respectively and insert the pegs into the drilled holes 5932. The oblong femoral implant 7250 can be any of the example oblong femoral implants disclosed herein. As described elsewhere in the present disclosure, the oblong femoral implant can have multiple (e.g., two or more) pockets for interfacing with the inserter tool 6540 such that the user can grab the oblong femoral implant 7250 with the two prongs of the inserter tool 6540. As shown in FIG. 72E, the user can optionally use the femoral implant impactor 6560 to fully seat the oblong femoral implant 7250, ensuring that a periphery 7252 of the oblong femoral implant 7250 is recessed relative to the adjacent cartilage (for example, by about 0.5 mm to about 1.0 mm below the adjacent cartilage), such as shown in FIG. 72F. The user may center an interface surface of the femoral implant impactor 6560 over the oblong femoral implant 7250 (e.g., by aligning the interface surface of the femoral implant impactor 6560 with the central circle of the oblong implant 7250) to avoid that the femoral implant impactor 6560 contacting the adjacent tissues, which may cause unwanted trauma to the native articular surface. The concave interface surface of the femoral implant impactor 6560 can match the convex articulating surface of the oblong femoral implant 7250.

### 8. Other Examples of Femoral Reamers

Features of the first femoral reamer 6100, and/or the second femoral reamer 6400, can be incorporated into the other example femoral reamers described below and vice versa, to the extent that the features are not mutually exclusive.

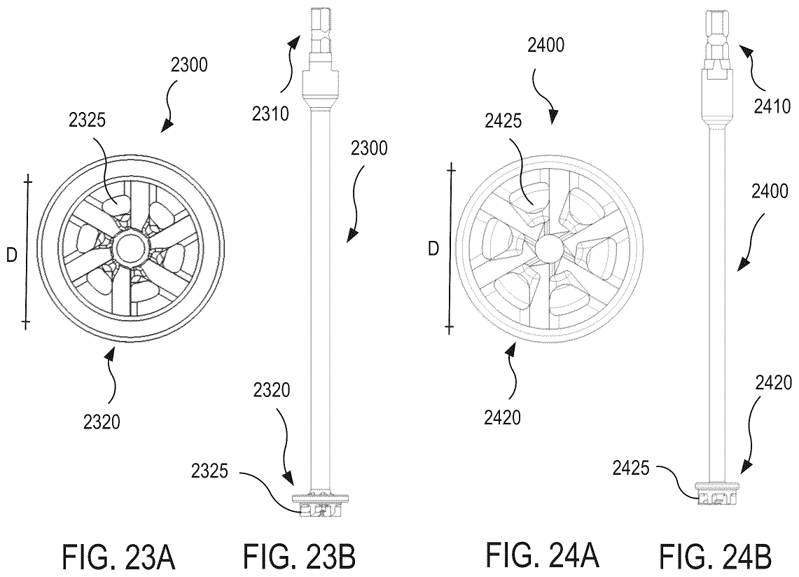

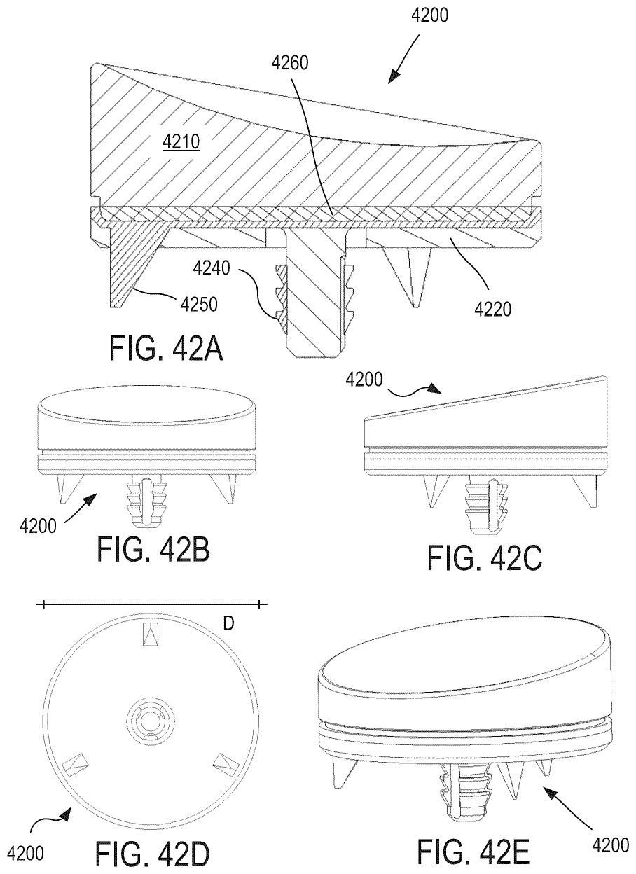

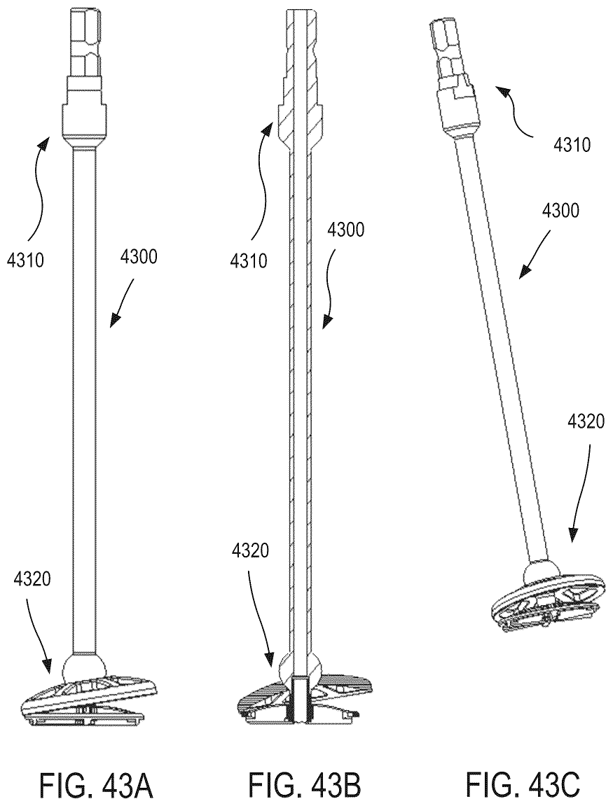

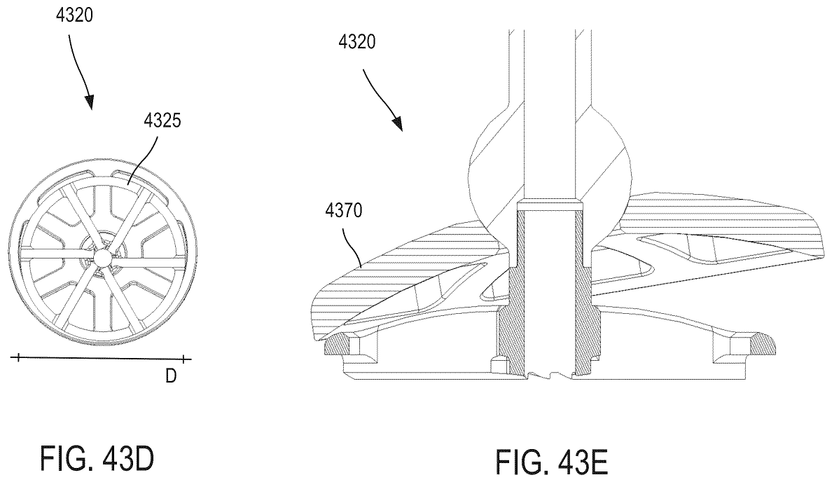

FIGS. 23A and 23B depict aspects of another first femoral reamer (primary) system 2300. FIG. 23A provides a distal end view, and FIG. 23B provides a side view. The femoral reamer (primary) system 2300 includes a proximal end 2310 and a distal end 2320, where the distal end 2320 is configured to ream bone of the patient, thereby producing a recess in the bone which is sized and/or configured to receive an implant. For example, the distal end 2320 can produce a recess or hole in the distal portion of the femur. The distal end 2320 can have a drill mechanism 2325, which may include cutting or boring elements, having a diameter D, so as to produce a recess or hole of similar diameter. According to some embodiments, FIGS. 23A and 23B depict aspects of a reamer system for a round femoral implant, and only one reamer is needed to implant a round implant. Related aspects of primary femoral reamer system embodiments are discussed elsewhere herein, for example in association with FIGS. 43A to 43E.

FIGS. 43A to 43E depict aspects of a first femoral (primary) reamer system 4300. FIG. 43A provides a side view, FIG. 43B provides a cross-section view, and FIG. 43C provides a perspective view. The primary femoral reamer system 4300 includes a proximal end 4310 and a distal end 4320, where the distal end 4320 is configured to ream bone of the patient, thereby producing a recess in the bone which is sized and/or configured to receive an implant. For example, the distal end 4320 can produce a recess or hole in the distal portion of the femur. FIG. 43D provides a bottom plan view, illustrating a distal end 4320 having a drill mechanism 4325, which may include cutting or boring elements. Drill mechanism 4325 has a diameter D, which can produce a bone recess or hole of similar diameter. FIG. 43E provides a cross-section view of the distal end 4320, illustrating a distal end 4320 having a washer mechanism 4370 that can operate to control the ream depth. In some cases, the washer mechanism 4370 can limit the ream depth by coming in contact with a condyle surface, for example located beyond a defect range. Therefore, the first femoral reamer system 4300 may not need a separate stopper component.

FIGS. 24A and 24B depict aspects of another femoral reamer (primary) system 2400. FIG. 24A provides a distal end view, and FIG. 24B provides a side view. The femoral reamer (primary) system 2400 includes a proximal end 2410 and a distal end 2420, where the distal end 2420 is configured to ream bone of the patient, thereby producing a recess in the bone which is sized and/or configured to receive an implant. For example, the distal end 2420 can produce a recess or hole in the distal portion of the femur. The distal end 2420 can have a drill mechanism 2425, which may include cutting or boring elements, having a diameter D, so as to produce a recess or hole of similar diameter.

According to some embodiments, FIGS. 24A and 24B depict aspects of a primary reamer system for an oblong implant. The system of FIGS. 23A and 23B may differ from the system of FIGS. 24A and 24B in terms of ream depth, drill depth, and/or bore depth. In some cases, a larger diameter at the top of the cutting feature can operate to limit the boring depth.

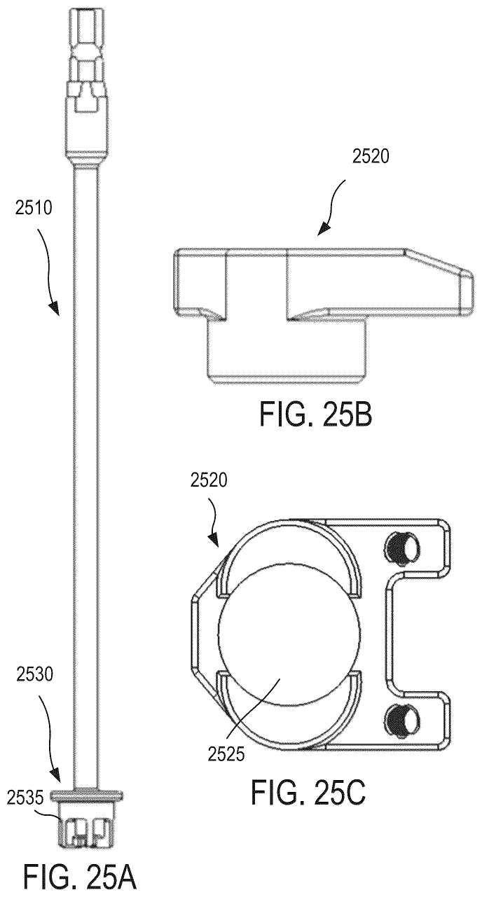

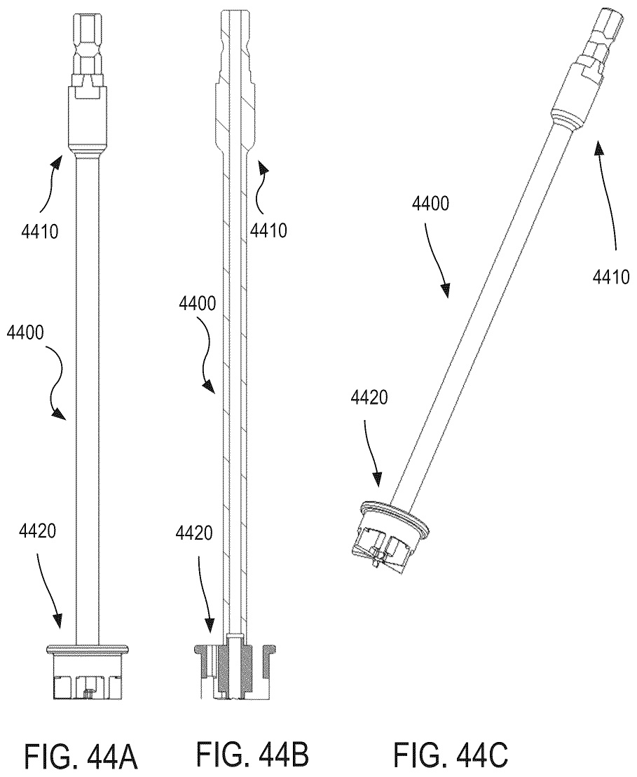

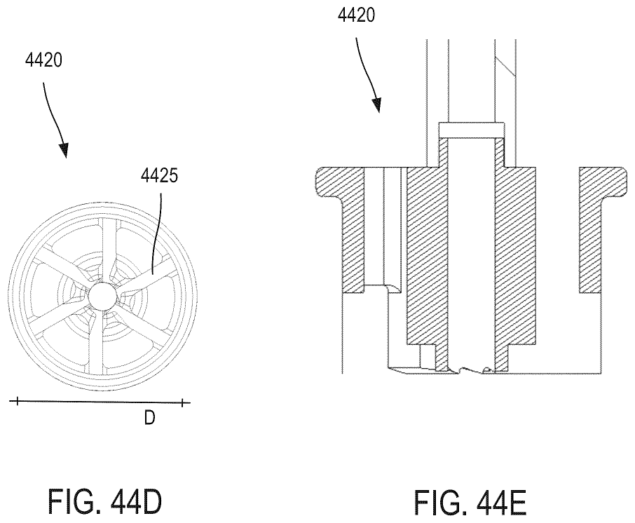

FIGS. 25A-25C depict aspects of another femoral reamer system. The system can include a drill assembly 2510 (as shown in FIG. 25A) and a guide 2520 (as shown in the side view of FIG. 25B and the plan view of FIG. 25C). In use, the guide 2520 can be engaged with the distal section of the patient's femur, and a drill mechanism 2535 of a distal end 2530 of the drill assembly 2510 can be aligned with the guide 2520, for example by placing the drill mechanism 2535 in an aperture 2525 of the guide 2520, so that the hole or bore in the distal femur bone produced by the reamer system is positioned as desired. Related aspects of secondary femoral reamer system embodiments are discussed elsewhere herein, for example in association with FIGS. 44A to 44E.

FIGS. 44A-44E depict aspects of a center femoral reamer system 4400. FIG. 44A provides a side view, FIG. 44B provides a cross-section view, and FIG. 44C provides a perspective view. The femoral reamer system 4400 includes a proximal end 4410 and a distal end 4420, where the distal end 4420 is configured to ream bone of the patient, thereby producing a recess in the bone which is sized and/or configured to receive an implant. For example, the distal end 4420 can produce a recess or hole in the distal portion of the femur. FIG. 44D provides a bottom plan view, illustrating a distal end 4420 having a drill mechanism 4425, which may include cutting or boring elements. Drill mechanism 4425 has a diameter D, which can produce a bone recess or hole of similar diameter. FIG. 44E provides a cross-section view of the distal end 4420. Related aspects of a secondary femoral reamer system embodiments are discussed elsewhere herein, for example in association with FIGS. 25A to 25E.

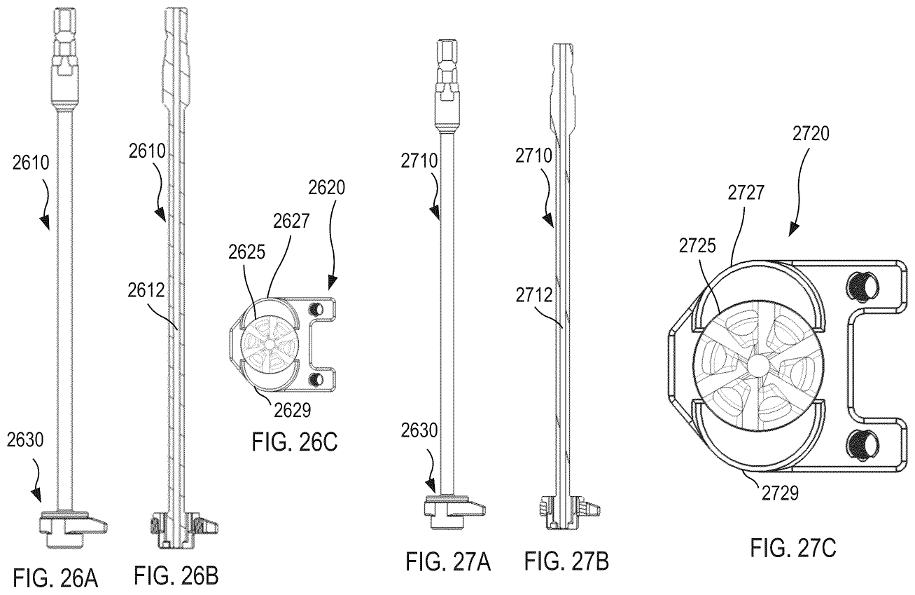

FIGS. 26A-26C depicts aspects of a femoral reamer (through guide) system. The system can include a drill support 2610 (as shown in the side view of FIG. 26A and the cross-section view of FIG. 26B) and a guide 2620 (as shown in the plan view of FIG. 26C). In use, the guide 2620 can be engaged with the distal section of the patient's femur, and a distal end 2630 of the drill support 2610 can be aligned with the guide 2620, for example by aligning the distal end 2630 with an aperture 2625 of the guide 2620, and/or with lateral sections 2627, 2629 so that one or more holes or bores can be produced in the distal femur bone by a drill (not shown) that is advanced through a central aperture 2612 of the drill support 2610. Such holes or bores can then receive respective pegs of an implant. In some embodiments, the diameter of the distal end of the reamer can vary with the implant size (e.g. larger reamer distal end diameter for larger implant size, and smaller reamer distal end for smaller implant size).

FIGS. 27A-27C depict aspects of a femoral reamer (through guide) system. The system can include a drill support 2710 (as shown in the side view of FIG. 27A and the cross-section view of FIG. 27B) and a guide 2720 (as shown in the plan view of FIG. 27C). In use, the guide 2720 can be engaged with the distal section of the patient's femur, and a distal end 2730 of the drill support 2710 can be aligned with the guide 2720, for example by aligning the distal end 2730 with an aperture 2725 of the guide 2720, and/or with lateral sections 2727, 2729 so that one or more holes or bores can be produced in the distal femur bone by a drill (not shown) that is advanced through a central aperture 2712 of the drill support 2710. Such holes or bores can then receive respective pegs of an implant. In some embodiments, the diameter of the distal end of the reamer can vary with the implant size (e.g. larger reamer distal end diameter for larger implant size, and smaller reamer distal end for smaller implant size).

If the user determines that the patient only needs a femoral implant, the incision on the skin can be closed using any suitable surgical technique. If the patient also needs a tibial implant, the example surgical tools and steps for inserting the tibial implant are described in the next section.

### E. Example Tibial Implant Insertion Tools and Method

Although certain figures referenced in this section of the disclosure illustrate a knee joint with a femoral implant already inserted, the surgical tools and steps below can be used to insert only a tibial implant and/or a tibial implant in combination with the femoral implant.

### 1. Patient Positioning

To implant the tibial implant disclosed herein, the patient can be positioned in the supine position and the joint can be exposed as disclosed elsewhere with reference to insertion of the femoral implant. The steps of patient positioning and joint exposure disclosed with reference to the insertion of the femoral implant can be performed. If the patient has already received the femoral implant, the initial exposure is complete and the patient is ready for the surgical steps on the tibial side. Alternatively, when implanting both tibial and femoral implants, the tibial implant can be inserted first followed by the femoral implant. One advantage of inserting the tibial implant first is to avoid damage to the femoral implant, which would be left exposed in the femoral condyle during the tibial procedure. However, if the exposed femoral cartilage is damaged during the tibial procedure, the damage in the cartilage can be treated later in the femoral procedure.

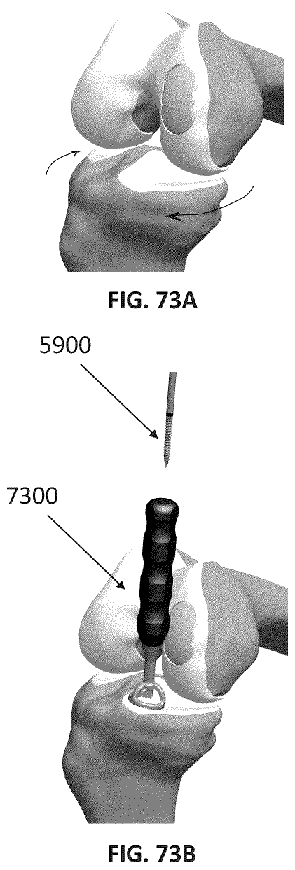

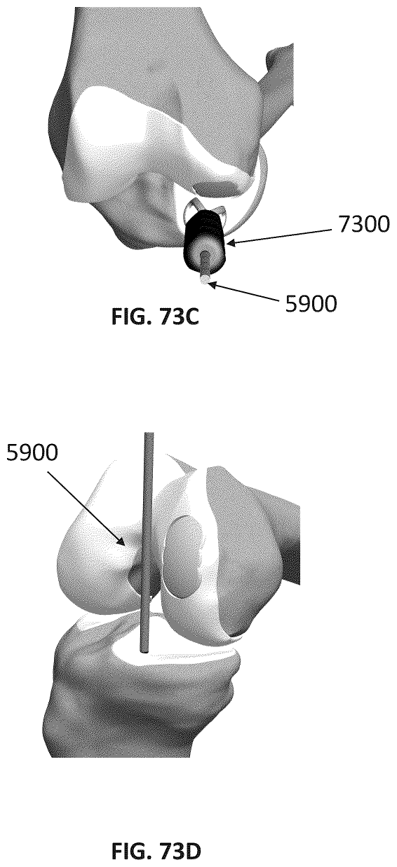

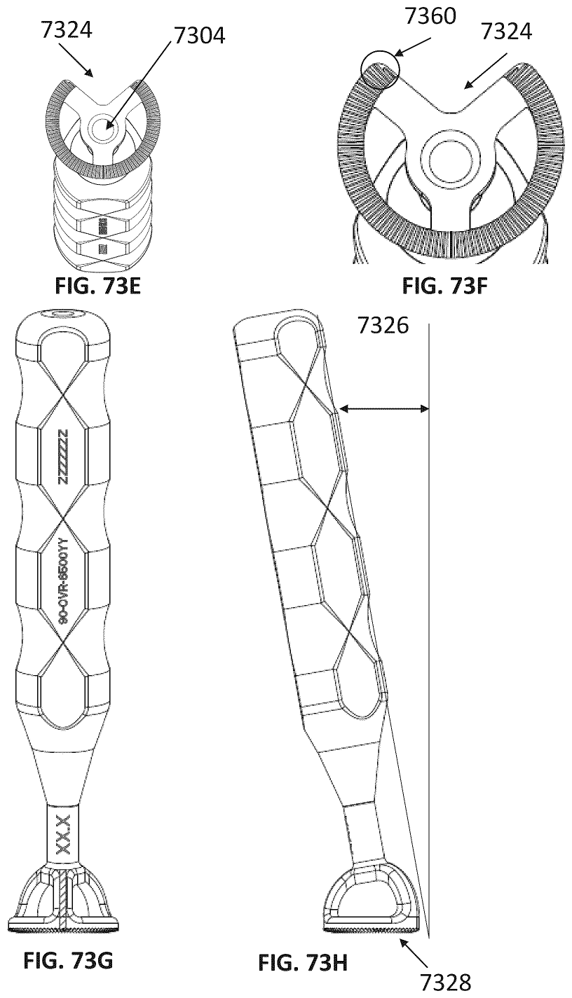

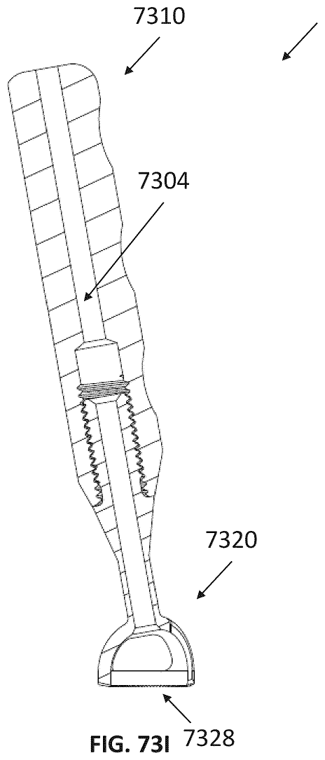

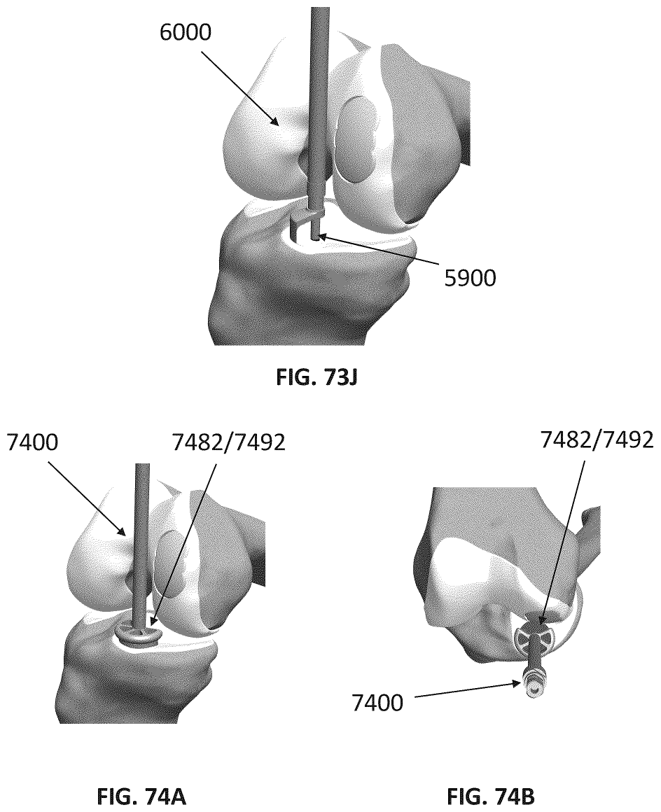

Once the initial joint exposure is complete, as shown in FIG. 73A, the user can flex the knee into deep flexion with external rotation (medial side) or internal rotation (lateral side), depending on the location of the osteochondral defect, to expose the surface of the tibial plateau. If the defect is on the medial side, the tibia can be externally rotated. If the defect is on the lateral side, the tibia can be internally rotated. To gain better visualization of the plateau surface, the user may detach the meniscus in a meniscus release procedure. In some implementations, the anterior horn of the meniscus may be detached. The meniscus can be left adherent to the joint capsule during the dissection. For a medial tibial defect, it is helpful to externally rotate the tibia for better visualization of the medial compartment. For lateral defects, internal rotation of the tibia is helpful for visualization of the affected area.

In some implementations, the tibial implant insertion technique disclosed herein can be meniscus sparing. The anterior horn insertion can be re-attached at the conclusion of the tibial implant insertion, for example, using suture anchors, anchorless attachment, or any other suitable implants/tools.

### 2. Tibial Implantation Site Access

With the knee properly positioned, as shown in FIGS. 73B and 73C, the user can assess the proper implant size using a tibial pin guide/sizer 7300, which can be available in a range of different sizes. The tibial pin guide/sizer 7300 is shown in various views in FIGS. 73E-731. The tibial pin guide/sizer 7300 include a proximal end 7310 and a distal end 7320. The proximal end 7310 can include a handle and the distal end 7320 can include a base and can define a distal engagement plane 7328. The base can have a generally round profile. Different sizes of the tibial pin guide/sizer 7300 can differ in the outer diameter of the round profile of the base, for example, ranging from between 17 mm to about 25 mm. The tibial pin guide/sizer 7300 can include a guide pin hole 7304 extending along a longitudinal axis of the tibial pin guide/sizer 7300. A guide pin delivered through the guide pin hole 7304 to the patient can enter the bone at a pin angle 7326 as shown in FIG. 73H. The pin angle can be defined as the angle between a line normal to the distal engagement plane 4928 and the longitudinal axis of the tibial pin guide/sizer 7300. The pin angle 7326 can be between about 8° to about 15°, for example, about 10°. The pin angle 7326 can match the offset angle of the tibial implant disclosed elsewhere in the present disclosure.

The base on the distal end 7320 of the tibial pin guide/sizer 7300 can include a cutout 7324, which can enable the tibial pin guide/sizer 7300 to clear a femoral condyle. The distal end 7320 can also include multiple spikes or teeth 7360. The spikes or teeth 7360 can operate to stabilize the tibial pin guide/sizer 7300 during use while installing the guide pin 5900. For example, the spikes or teeth 7360 can engage patient bone during installation of the guide pin 5900. As shown in FIGS. 73E and 73F, the spikes or teeth 7360 can extend around a periphery of the base except at the cutout 7324.

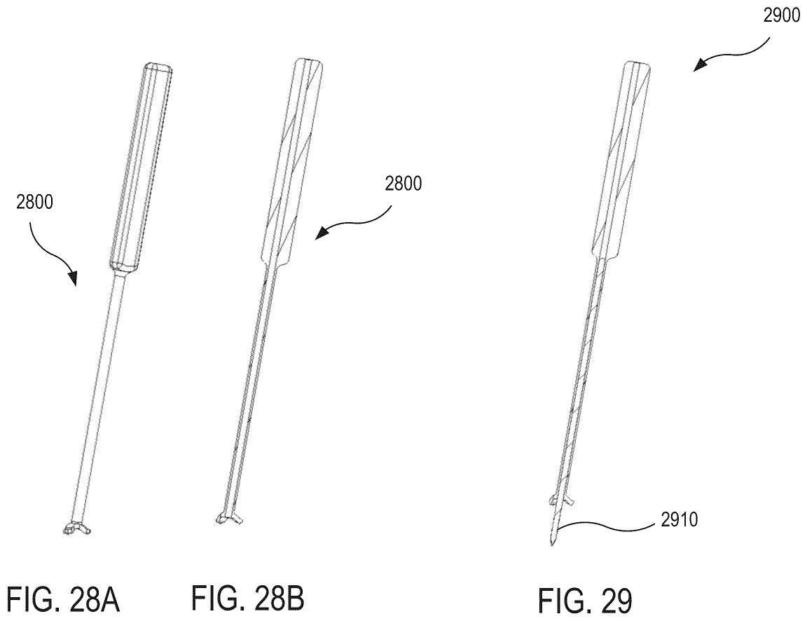

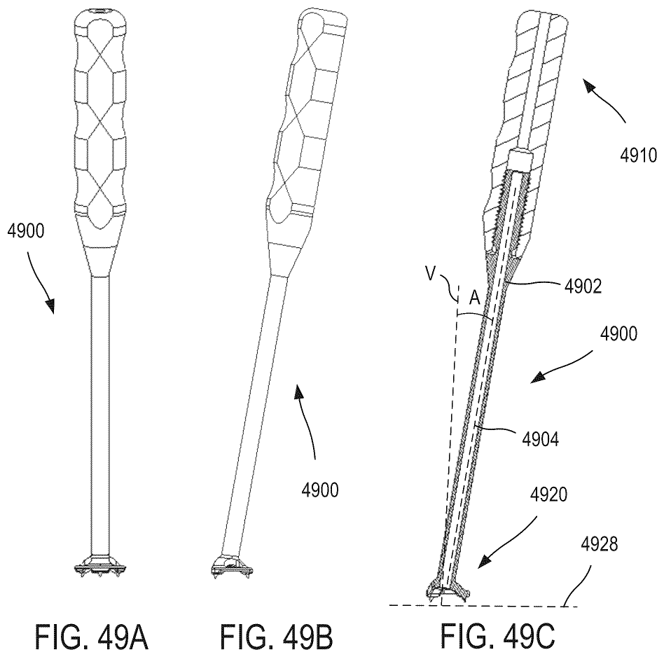

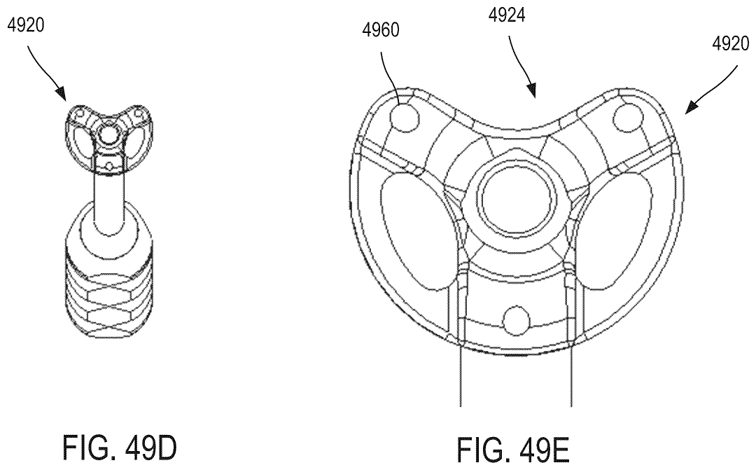

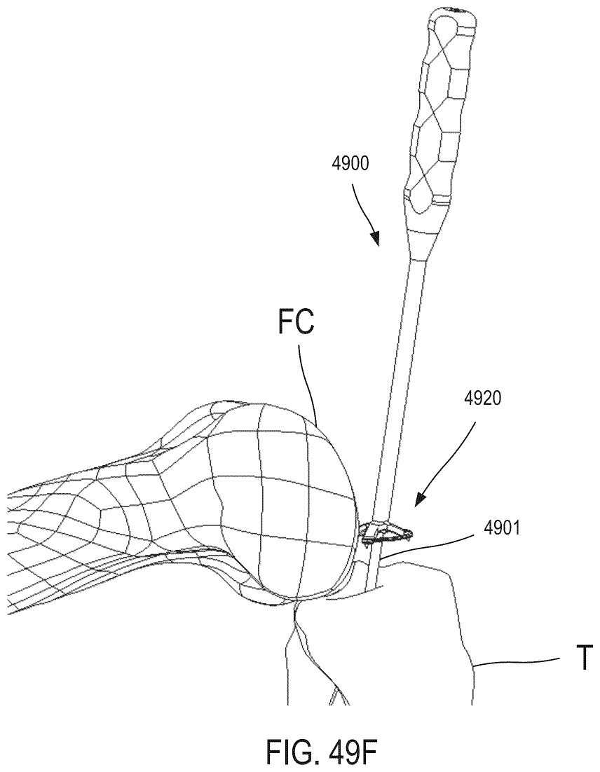

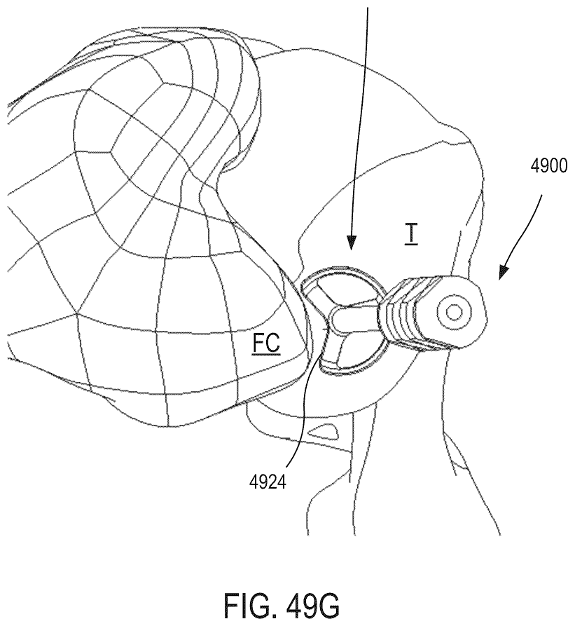

FIGS. 28A and 28B depict aspects of a tibial pin guide system 2800. Related aspects of tibial pin guide system embodiments are discussed elsewhere herein, for example in association with FIGS. 49A to 49G. FIG. 29 depicts aspects of a tibial pin device 2900. The device can include a pin 2910. In some cases, tibial pin device 2900 can provide the same functionality and/or be implemented for the same intended use as the device depicted in one or more of FIGS. 49A to 49G.

FIGS. 49A to 49G depict aspects of a tibial pin guide system 4900 and related methods of use. FIGS. 49A and 49B provide side views, and FIG. 49C provides a cross-section view. A tibial pin guide system 4900 can include a proximal end 4910 and a distal end 4920. As shown in FIG. 49C, a distal end of the tibial pin guide system 4900 can define a distal engagement plane 4928 and an interior lumen 4902 of the tibial guide system 4900 can define a central longitudinal axis 4904, such that a pin delivered through the lumen 4902 to the patient can enter the bone at a pin angle A, where angle A is the angle between a vector V normal to the plane 4928 and the central longitudinal axis 4904. As shown in FIG. 49E, the distal end 4920 of the tibial guide system 4900 includes a cutout 4924, which can enable the system 4900 to clear a femoral condyle. The distal end 4920 also includes spikes 4960 which can operate to stabilize the system 4900 during use while installing pins. For example, the spikes 4960 can engage the healthy cartilage on the tibial plateau to ensure that the entire defect is covered by the distal engagement plane 4928. FIGS. 49F and 49G illustrate aspects of a method during which system 4900 is used to deliver a pin 4901 to a patient bone (e.g. tibia T). As shown here, a cutout 4924 of the distal end 4920 of the system can enable the distal end to clear a femoral condyle FC. FIG. 49F shows a side view of a patient knee, and FIG. 49G shows a superior view of a patient knee.

The tibial pin guide/sizer 7300 can have any of the features of the tibial pin guide systems 2800, 4900; and The tibial pin guide systems 2800, 4900 can have any of the features of the tibial pin guide/sizer 7300.

Returning to FIGS. 73B and 73C, the tibial pin guide/sizer 7300 can be rotated such that the cutout 7324 in the base is rotated towards the femur. This will allow for greater clearance with the femur and easier insertion of the tibial pin guide/sizer 7300. The user may verify that the base of the tibial pin guide/sizer 7300 extends beyond the edges of the osteochondral defect to ensure adequate implant coverage and complete removal of the defect. In some implementations, the user may center the placement of the tibial pin guide/sizer 7300 on the osteochondral defect. The user may also verify that the base of the tibial pin guide/sizer 7300 is flush with the tibia plateau surface. For example, the user may verify whether all of the spikes or teeth 7360 are engaging the cartilage. When the base of the tibial pin guide/sizer 7300 is flush with the tibia cartilaginous surface, the handle will be tilted anteriorly at the pin or offset angle relative to the vertical plane perpendicular to the tibial plateau surface, which will coincide with the distal engagement plane 7328 of the tibial pin guide/sizer 7300.

With the tibial pin guide/sizer 7300 flush to the tibial plateau surface, the user can drill the guide pin into the tibia using the tibial pin guide/sizer 7300. The laser mark 5902 on the guide pin 5900 (see, e.g., FIG. 58B) may be flush with the tip of the proximal end 7310 of the tibial pin guide/sizer 7300. In FIG. 73D, the user has removed the tibial pin guide/sizer 7300, leaving behind the guide pin 5900 drilled into the tibial bone.

As shown in FIG. 73J, the user can place an appropriately sized cartilage scoring tool 6000 (see FIGS. 60B-60G) over the pin 5900. The user can create a clean cut on the edge of articular cartilage down to the subchondral bone by manually rotating the scoring tool 6000 (for example, by one or more revolutions). The cut extends beyond any existing osteochondral damage or defect. If the cut does not capture all damaged areas, a larger sized implant may be needed and the user may repeat the steps shown in FIGS. 73A-73D with a bigger tibial pin guide/sizer 7300 corresponding with the selected larger sized implant.

### 3. Tibial Implantation Site Reaming

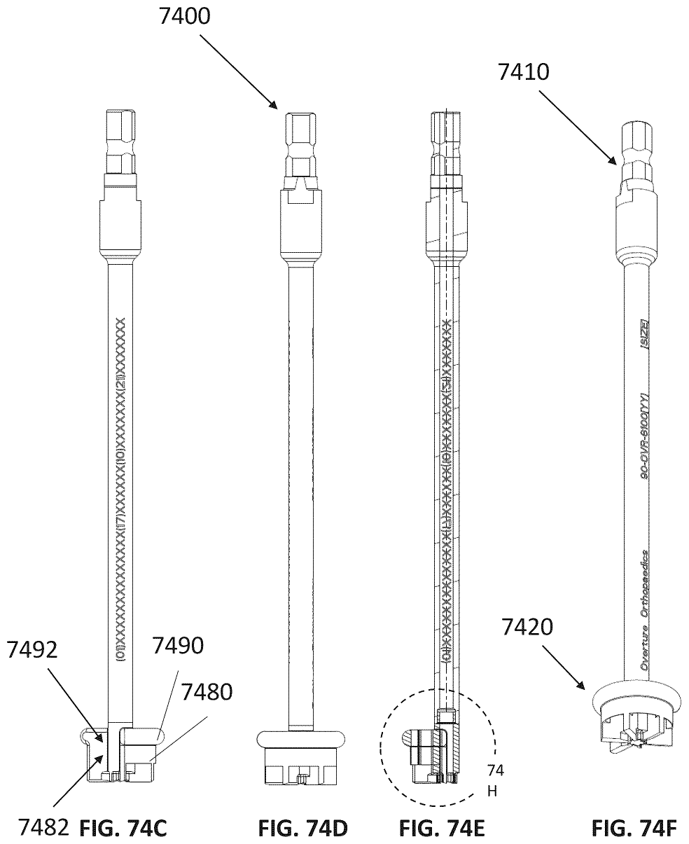

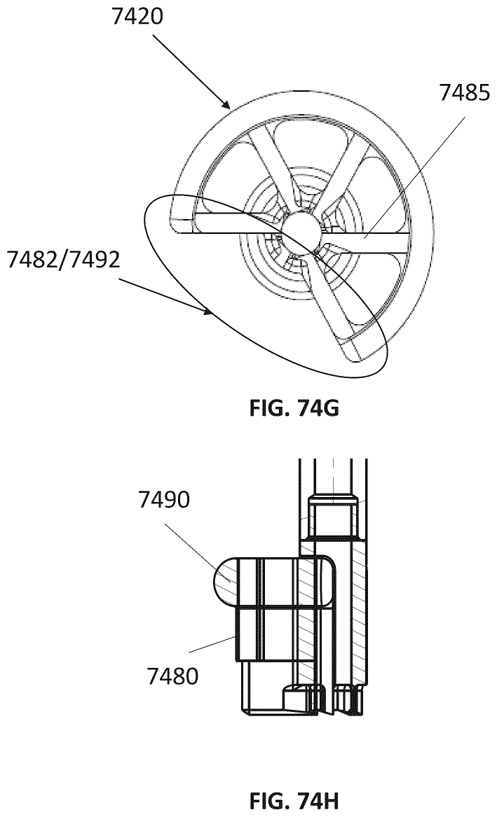

With the cartilage scoring tool 6000 removed, in FIGS. 74A and 74B, the user can place a first tibial reamer 7400 over the guide pin 5900 (not shown in these views) using power. Unlike reaming using a first femoral reamer, powered reaming using the first tibial reamer 7400 may not need to use a stopper component as described above.

FIGS. 74C-74H illustrate various views of the first tibial reamer 7400, which can include a proximal end 7410 and a distal end 7420. The first tibial reamer 7400 may be powered by connecting the proximal end 7410 to a motor and a power source. When activated, the distal end 7420 of the first tibial reamer 7400 can ream the bone to produce a recess or hole in the distal portion of the tibia. The distal end 7420 of the first tibial reamer 7400 can include a reamer head 7480 having a cutout 7482. The reamer head 7480 can include blades 7485 for reaming the bone. The distal end 7420 of the first tibial reamer 7400 can also include a depth stop washer 7490 having a cutout 7492. The cutouts 7482, 7492 can enable the distal end 7420 of the first tibial reamer 7400 to clear a femoral condyle of the patient, when the cutouts 7482, 7492 are rotated towards the femur during insertion as shown in FIGS. 74A and 74B, or removal of the first tibial reamer 7400. This will allow for additional clearance when inserting and/or removing the first tibial reamer 7400. The user can use power to ream the bone until the depth stop washer 7490 on the first tibial reamer 7400 contacts the tibial surface. In some implementations, this contact between the depth stop washer 7490 and the tibial bone surface may not need to occur along the entire periphery of the first tibial reamer 7400. When removing the first tibial reamer 7400, the user can continue orienting the first tibial reamer 7400 such that the cutouts 7482, 7492 are facing the femoral condyle to improve case of removal. As powered reaming can generate a large amount of heat, the user can irrigate the reamed site to prevent thermal necrosis during reaming.

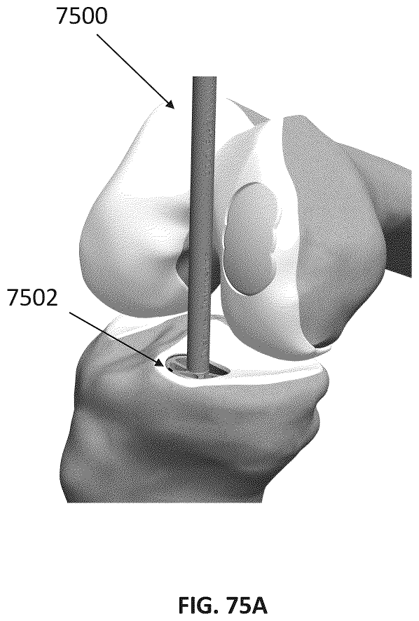

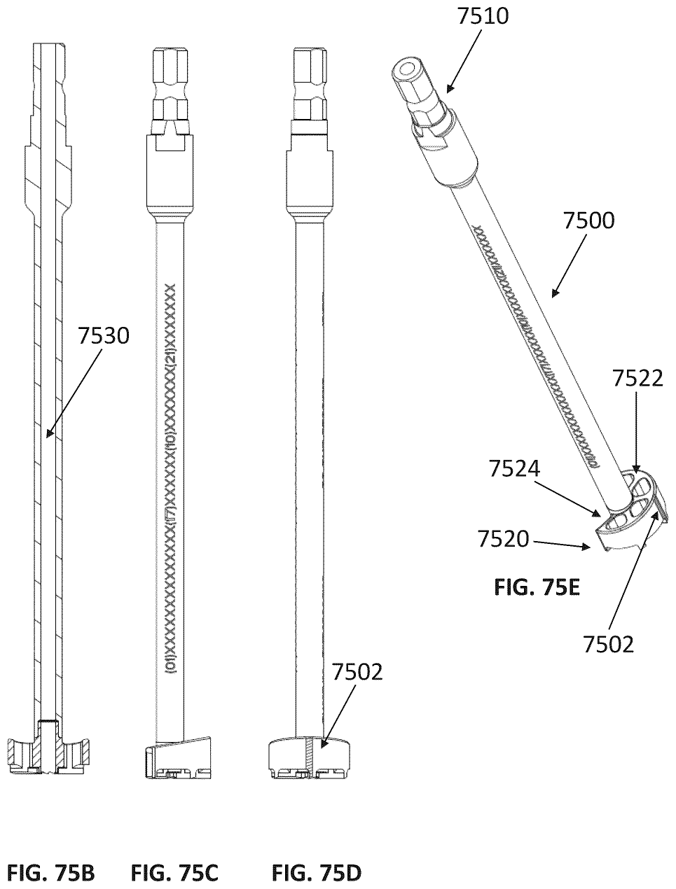

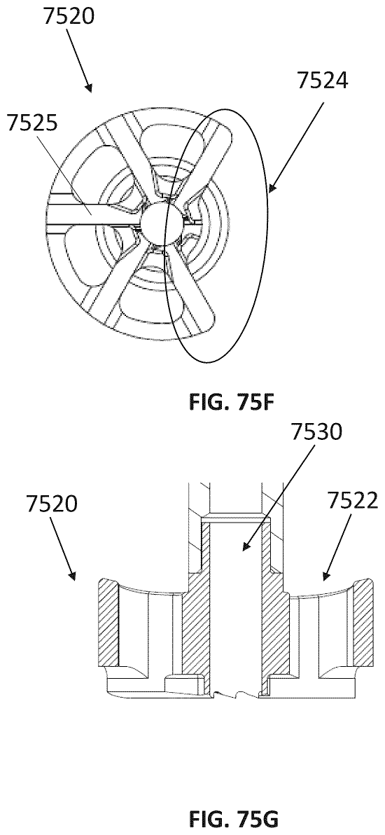

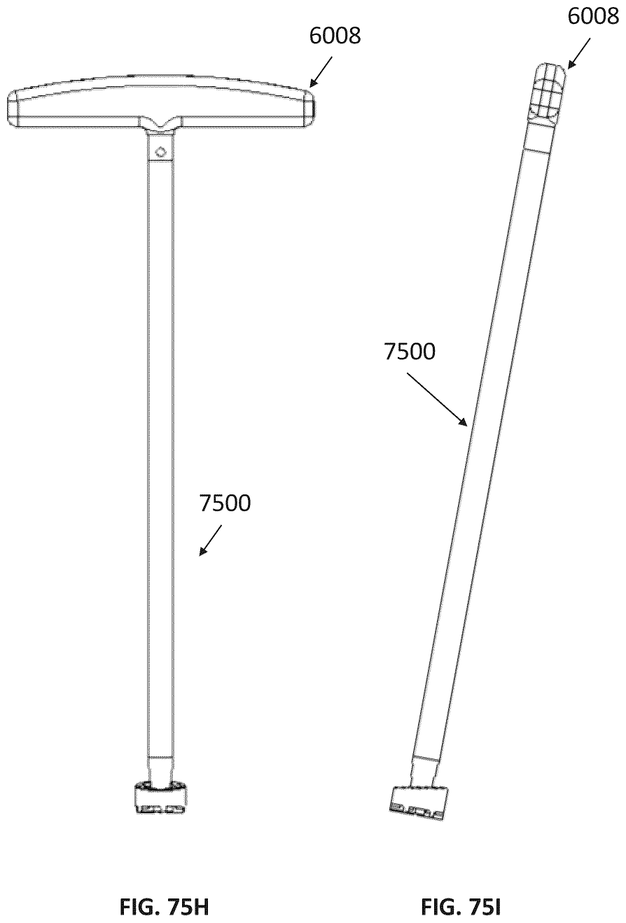

After removal of the first tibial reamer 7400, as shown in FIG. 75A, the user can slide a second tibial reamer 7500 over the guide pin 5900 and ream the bone by hand. The second tibial reamer 7500 can be a hand reamer such that the user needs to rotate a handle 6008 (see FIGS. 75H and 75I) coupled to a proximal end 7510 of the second tibial reamer 7500 in order to cut into the bone.

FIGS. 75B-75I illustrate an example second tibial reamer 7500. The second tibial reamer 7500 can include the proximal end 7510 and the distal end 7520, with a shaft extending between the proximal and distal ends 7510, 7520. The second tibial reamer 7500 can include a guide pin hole 7530 extending through a length of the second tibial reamer 7500. As noted above, the distal end 7520 can include a base. The base can have a generally circular profile as shown in FIG. 75F, with multiple cutting blades 7525 extending radially from center of the circular profile. The second tibial reamer 7500 can be available in various sizes with different diameters of the base to match the implant sizes. The base on the distal end 7520 can include a cutout 7524 to allow easier access to the tibia by clearing the femoral condyle and/or avoiding damage to the femur during insertion and/or removal (that is, when the second tibial reamer 7500 is inserted in an orientation such that the cutout 7524 faces the femur). The portion of the base where the cutout 7524 is located may not have the ability to cut. In order to ream the posterior tibial bone, the user may rotate the second tibial reamer 7500, for example, for about half to about one revolution while in contact with the tibia.

In some implementations, the second tibial reamer 7500 may include a single-use version. The single-use second tibial reamer 7500 may have only the multiple cutting blades 7525 made in metal (e.g., stainless steel or any other suitable metal or metal alloy) and a remainder of the second tibial reamer 7500 made in plastic. The cutting blades 7525 can include stamped sheet metal with a sharp cutting edge. Optionally, the single-use second tibial reamer 7500 can also include an inner metal hypotube. The plastic portion of the single-use femoral reamer 7500 can be molded around the metal cutting blades 7525. The plastic portion can also be molded around the inner metal hypotube. The metal hypotube can increase the strength and/or rigidity of the plastic tube. The single-use tibial reamer 7500 can be more affordable than an all-metal second tibial reamer 7500 while being able to perform substantially the same function as the all-metal second tibial reamer 7500. The single-use tibial reamer 7500 also reduces the number of surgical tools for re-sterilization after the surgery.

The user can hand ream the bone until a higher side (with laser mark 7502 such as shown in FIGS. 75D and 75E) of a base of the second tibial reamer 7500 is flush with the level of adjacent cartilage surface. The shape of a top surface 7522 of the second tibial reamer 7500 can match the shape of the top surface of the selected tibial implant and/or a thickness of the base at the higher side can match a thickness of the selected tibial implant. Accordingly, the top surface 7522 of the base can be used as an indicator of the final implant position and the second tibial reamer 7500 can serve a dual function of a trial and a reamer.

The user can manually make a mark on the tibial plateau to correspond with the laser mark 7502 on the second tibial reamer 7500 using a surgical marker to indicate the higher side (corresponding to a thicker side of the tibial implant). This manual marking can help to ensure the trial and final tibial implant are each properly rotated to align with the manual mark during insertion. After making the mark, the user can remove the second tibial reamer 7500 and the guide pin 5900. The user can irrigate the reamed tibial site to remove debris.

### 4. Tibial Implant Trialing & Insertion

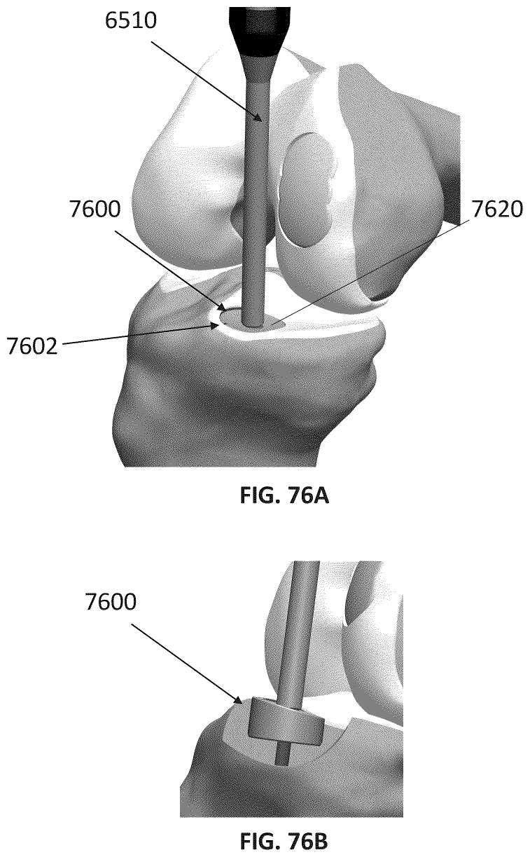

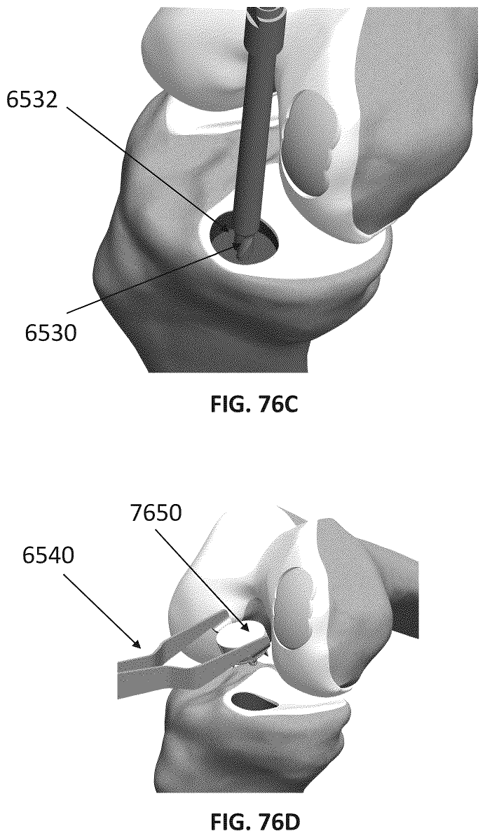

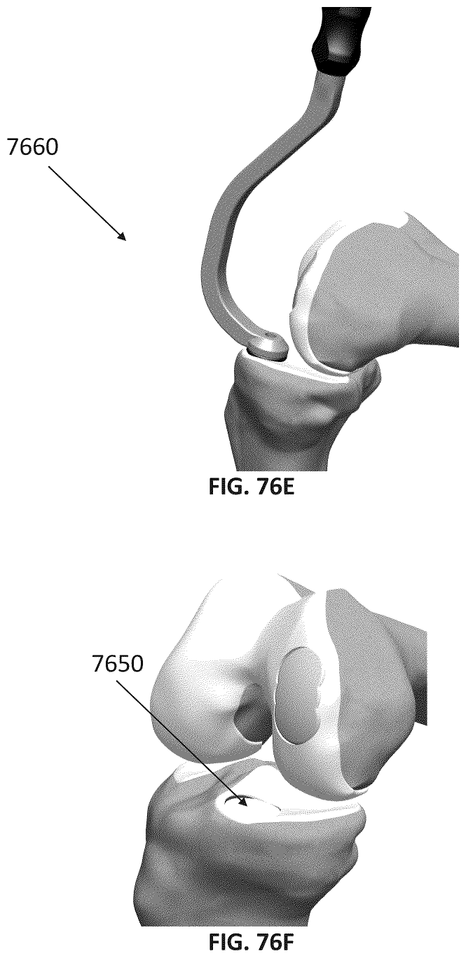

With the implant site prepared, as shown in FIG. 76A, the user can insert correctly sized tibial trial 7600 using the handle 6510 or any other suitable handle. The tibial trial 7600 can have the same size and shape as the portion of the corresponding tibial implant above the peg and/or spikes, that is, between the concave bearing surface and the subchondral surface. The tibial trial 7600 can have a central opening such that the handle 6510 can be coupled to the tibial trial 7600 (e.g., via threaded engagement or a clip on feature). Prior to insertion of the tibial trial 7600, the user can rotate the tibial trial 7600 to match the location of the laser marked line 7602 of the tibial trial 7600 with the mark manually made on the bone. The user can visually inspect to ensure the top surface 7620 of the tibial trial 7600 is recessed at the desired distance below the adjacent cartilage surface. Alternatively, the tibial trial 7600 can be thicker than the corresponding tibial implant by the recessed distance disclosed herein and the user can visually and/or confirm by hand whether the tibial trial 7600 is flush with the adjacent cartilage. When the thicker trial is flush with the cartilage, the actual tibial implant will sit recessed below the cartilage surface by the desired distance. Either way, the design of the trial and/or the inspection by the user can ensure that the final implant is correctly recessed below the surface (e.g., by about 0.5 mm to about 1.0 mm, or about 0.5 mm). As shown in FIG. 76B, if adequate placement of the tibial trial 7600 has not been achieved, for example, if the tibial trial 7600 sits proudly compared to the adjacent cartilage, the user can remove the tibial trial 7600 and repeat the powered reaming and/or the hand reaming steps until the proper depth is achieved. The user may need to repeat one or both of powered reaming and hand reaming more than once to achieve the desired depth.

If the tibial trial 7600 is properly placed, as shown in FIG. 76C, the user can remove the tibial trial and insert the distal end of a drill bit 6530 into the hole created by the guide pin. Power can be supplied to the drill bit 6530 to drill until a shoulder 6532 on the drill bit 6530 reaches the reamed bone surface. The user can then remove the drill bit 6530. Optionally, the user can irrigate the area (e.g., with saline) to remove any loose debris and dry the bone surface (e.g., using a lap sponge). The user can apply a small amount (for example, about 1 ml to about 2 ml) of bone cement into the drilled hole. The user may ensure that the bone cement is only applied inside the drilled hole such that the porous metal subchondral surface of the tibial implant is in direct apposition to cancellous bone to facilitate integration. This way, the tibial implant can achieve hybrid fixation as disclosed herein. That is, cement fixation can be achieved at the peg/bone interface, while biologic bony in-growth fixation or osteointegration can be achieved at the porous metal subchondral surface of the femoral implant. In implementations where bone cement is used, the hole drilled by the drill bit 6530 can be slightly greater than the outer diameter of the barbs. In some implementations, additional cement may be placed under the subchondral implant surface in addition to around the peg to achieve greater initial fixation and/or to adjust the height of the implant after reaming. For example, if the bone is reamed too deep such that the implant is recessed more than a certain depth (such as 1 mm) below the native cartilage surface, the additional cement placed under the subchondral surface can add to the height to the implant. As another example, if the preparation of the hole(s) in the bone was poor, such as being on a poor angle, the cement placed under at least a portion of the subchondral surface of the implant can compensate for the poor angle.

In implementations where bone cement will not be used, the hole drilled can be slightly smaller than the outer diameter of the barbs to allow initial or primary fixation by interference between the barbs and the drilled hole before bone ingrowth into the porous metal layer. For example, the guide pin 5900 may have an outer diameter that is substantially the same as the outer diameter the peg outer wall without the barbs. The drilled hole can have an internal diameter that is about line to line with the peg outer wall without the barbs. In a cementless application, the step of drilling using the drill bit may be skipped.

As shown in FIG. 76D, using an inserter tool 6540, the user can align the peg of the tibial implant 7650 with the drilled hole and insert the peg into the drilled hole. The tibial implant 7650 can be any of the example tibial implants disclosed herein. As described elsewhere in the present disclosure, the tibial implant can include a groove between its plastic portion and metal portion. The user can grab the tibial implant 7650 with the two prongs of the inserter tool 6540 around the groove.

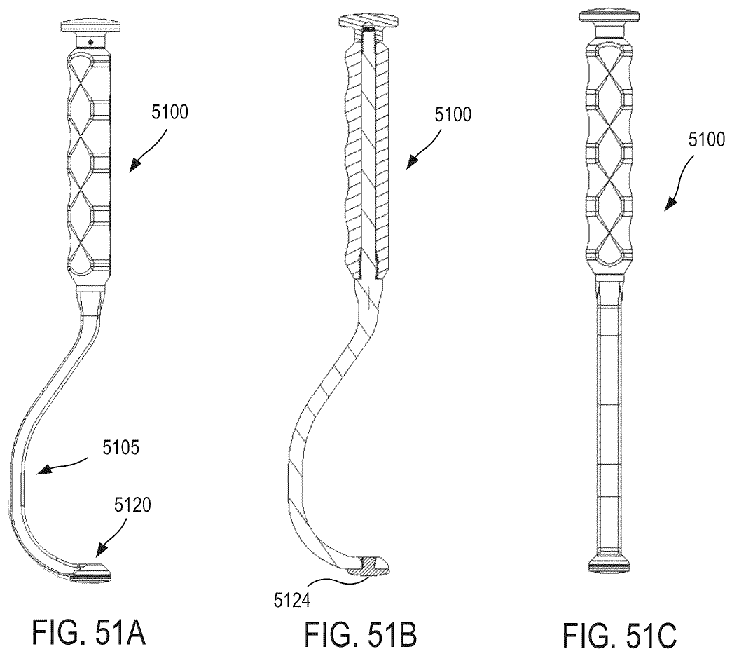

Referring back to FIG. 1C, the subchondral surface and/or the peg may be implanted at a non-zero angle relative to a plane defined by the tibial plateau (corresponding to the offset angle or the pin angle). The articulating surface of the tibial implant is coplanar with the plane defined by the tibial plateau. As noted elsewhere in the present disclosure, the angle is due to drilling hole(s) into the tibial and inserting the tibial implant anteriorly at the non-zero angle so as to clear the femoral condyle, which cannot be moved completely away from the joint space with deep flexing the knee and externally/internally rotating the foot. The rim of the concave bearing surface of the tibial implant is inserted to be generally parallel to the tibial plateau plane and recessed by the small distance from the adjacent cartilage surface. As shown in FIG. 76E, the user can optionally use a tibial implant impactor 7660 to fully seat the tibial implant 7650, ensuring that a periphery of the tibial implant 7650 is recessed relative to the adjacent cartilage (for example, by about 0.5 mm to about 1.0 mm below the adjacent cartilage), such as shown in FIG. 76F. The user may center an interface surface of the tibial implant impactor 7660 over the tibial implant 7650 to ensure that the tibial implant impactor 7660 does not contact the adjacent tissues to avoid unwanted trauma to the native articular surface. In some implementations, the interface surface of the tibial implant impactor 7660 can be convex to match the concave articulating surface of the tibial implant 7650. The tibial implant impactor 7660 can be the impactor system 5100 shown in FIGS. 51A-51C.

FIGS. 51A to 51C depict aspects of an impactor system 5100. FIG. 51A provides a side view, FIG. 51B provides a cross-section view, and FIG. 51C provides a front view. As shown here, an impactor system 5100 can include a distal end 5120 having a convex surface 5124, which can be shaped or configured to match or complement a concave surface of an implant (e.g. a tibial implant). Impactor system 5100 can also include a distal curve 5105, which can enable the impactor system 5100 to clear a femoral condyle of the patient.

### 5. Other Examples of Tibial Reamers

Features of the first tibial reamer 6100, and/or the second tibial reamer 6400, can be incorporated into the other example tibial reamers described below and vice versa, to the extent that the features are not mutually exclusive.

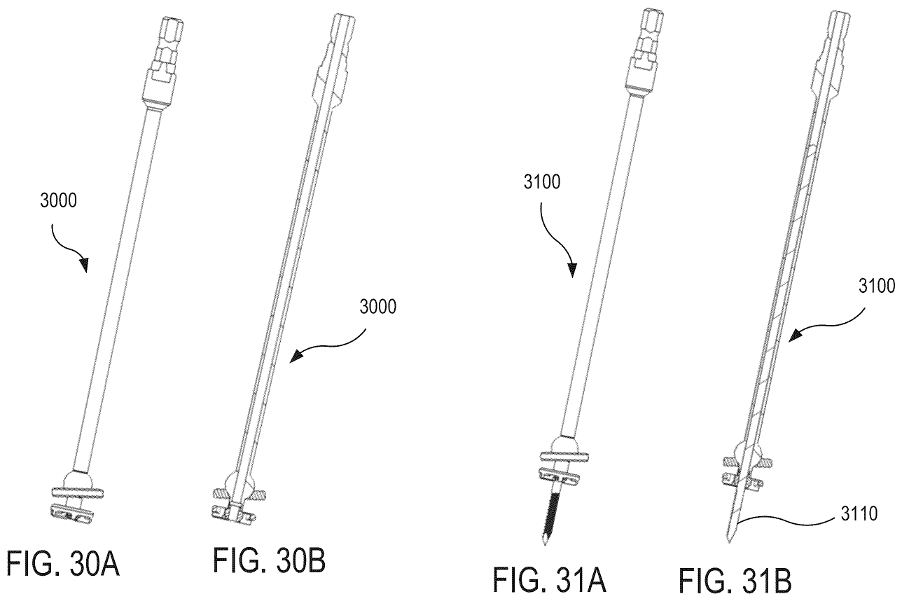

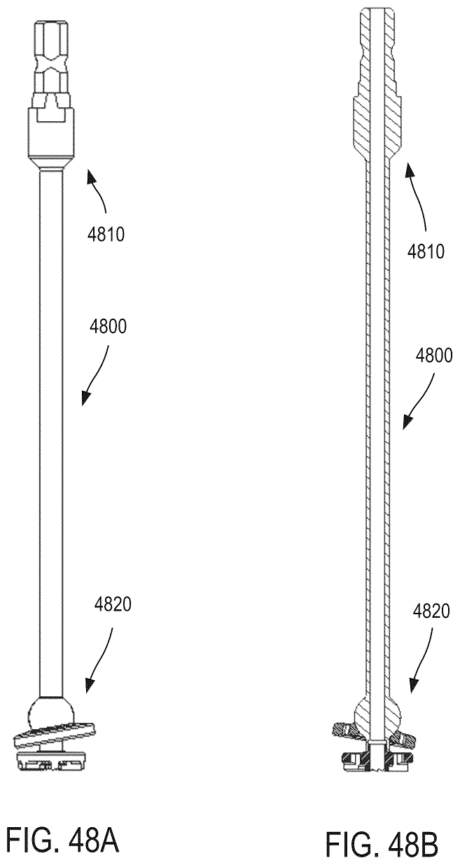

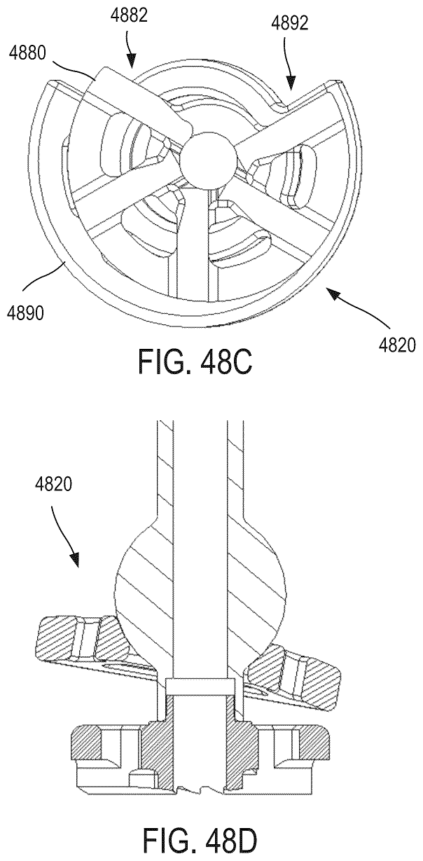

FIGS. 30A and 30B depict aspects of a tibial reamer (primary or first) system 3000. Aspects of related primary tibial reamer systems are discussed elsewhere herein, for example in association with FIGS. 48A to 48D. FIGS. 31A and 31B depicts aspects of a tibial pin device 3100. The device can include a pin 3110. In some cases, tibial pin device 3100 can provide the same functionality and/or be implemented for the same intended use as the device depicted in one or more of FIGS. 48A to 48D.

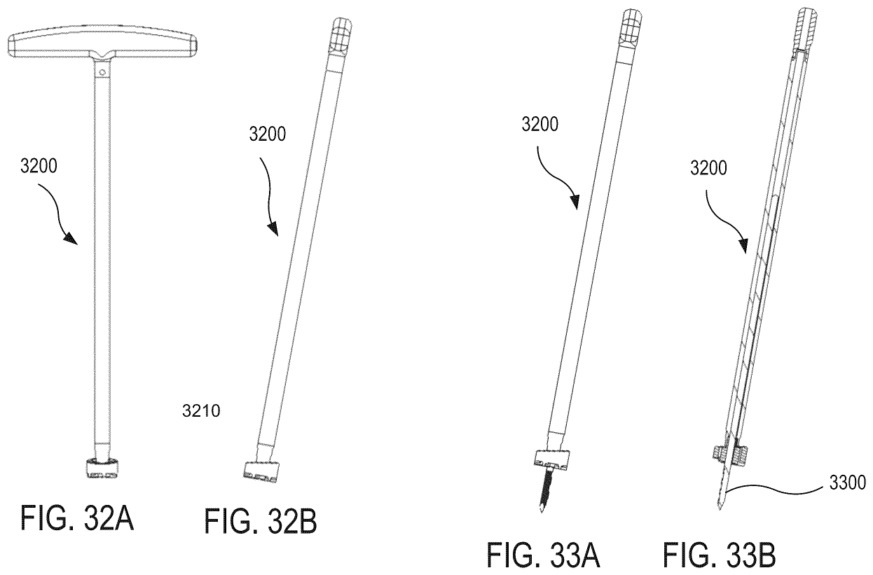

FIGS. 32A and 32B depict aspects of a tibial reamer (secondary or second) system 3200. FIGS. 33A and 33B depict aspects of a guide pin 3300 coupled to the tibial reamer system 3200. In some embodiments, a distal section 3210 of the reamer system 3200 can be configured to mimic or match the geometry of the implant, while also functioning as a reaming/drilling/boring device. In some cases, the guide pin 3300 can be used after the primary reamer is used to hand ream the bone for a precise implant fit.

FIGS. 48A to 48D depict aspects of a primary or first tibial reamer system 4800. FIG. 48A provides a side view and FIG. 48B provides a cross-section view. The primary tibial reamer system 4800 includes a proximal end 4810 and a distal end 4820, where the distal end 4820 is configured to ream bone of the patient, thereby producing a recess in the bone which is sized and/or configured to receive an implant. For example, the distal end 4820 can produce a recess or hole in the distal portion of the femur. FIG. 48C provides a bottom plan view and FIG. 48D provides a cross-section view. As depicted in FIG. 48C, the distal end 4820 of the primary tibial reamer system can include a reamer head 4880 having a cutout 4882 and a depth stop washer 4890 having a cutout 4892. The cutouts 4882, 4892 can enable the distal end 4820 to clear a femoral condyle of the patient. Aspects of such femoral clearance techniques are discussed elsewhere herein, for example in association with FIGS. 49F and 49G.

### F. Example Removal Tools and Method of Removal

An inserted implant may need to be removed for various reasons. For example, the size of the first implant may be too small and a bigger implant is needed. As another example, the patient's condition is no longer suitable for a targeted or focal-plasty so that the focal-plasty implant(s) need(s) to be removed for insertion of a partial or total knee arthroplasty implant(s). As described elsewhere in the present disclosure, the femoral implants have two or more pockets that can interface with an insertion tool or a removal tool. Similarly, the tibial implant has a groove between the metal portion and the plastic portion. A clamping device can grab the metal portion by the groove during the overmolding process. An insertion or removal tool can also grab onto the groove for insertion and removal of the tibial implant.

### 1. Femoral Removal—Round

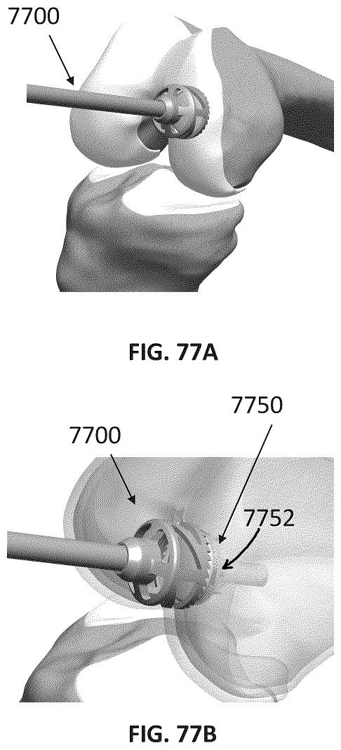

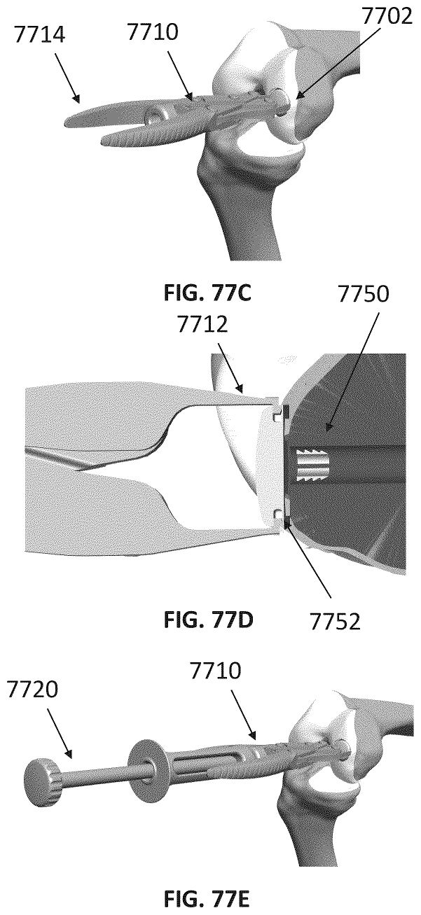

As described elsewhere in the present disclosure, the pockets 7752 on the round femoral implant 7750 can be radially opposite sides of the round femoral implant 7750. The pockets 7752 can be just below the articulating surface. As shown in FIGS. 77A and 77B, to gain access to the pockets 7752, the user can place a hole saw 7700 around the round femoral implant 7750. The hole saw 7700 can be available in different sizes to accommodate the different implant sizes. The hole saw 7700 can be powered and can drill a circumferential channel 7702 around the articulating surface of the round femoral implant 7750. The user can drill until the hole saw reaches its full depth to the surface of the round femoral implant 7750 that is contacting the bone (i.e., the subchondral surface), or at least past the depths of the pockets 7752. As shown in FIGS. 77C and 77D, the user can then remove the hole saw 7700 and place a removal tool 7710 around the round femoral implant 7750 in the circumferential channel 7702 created by the hole saw 7700. The user can align the prongs 7712 of the removal tool 7710 with the pockets 7752 of the round femoral implant 7750. If it is difficult to get both prongs 7712 connected to the pockets 7752, the user can attach the slap hammer 7720 to the removal tool 7710 and tap lightly downward on the removal tool 7710 until the user can confirm that the prongs 7712 has engaged the pockets 7752. The user can squeeze the grip 7714 of the removal tool 7710 to grab the round femoral implant 7750. In some implementations, the user can use the slap hammer 7720 to aid in the removal as shown in FIG. 77E.

### 2. Femoral Removal—Oblong

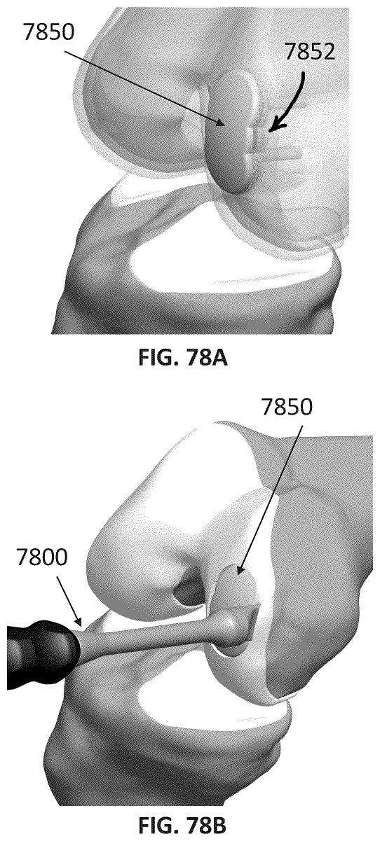

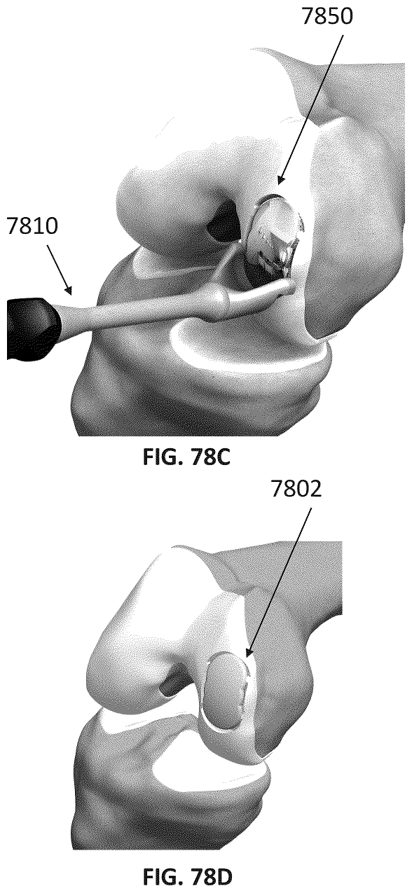

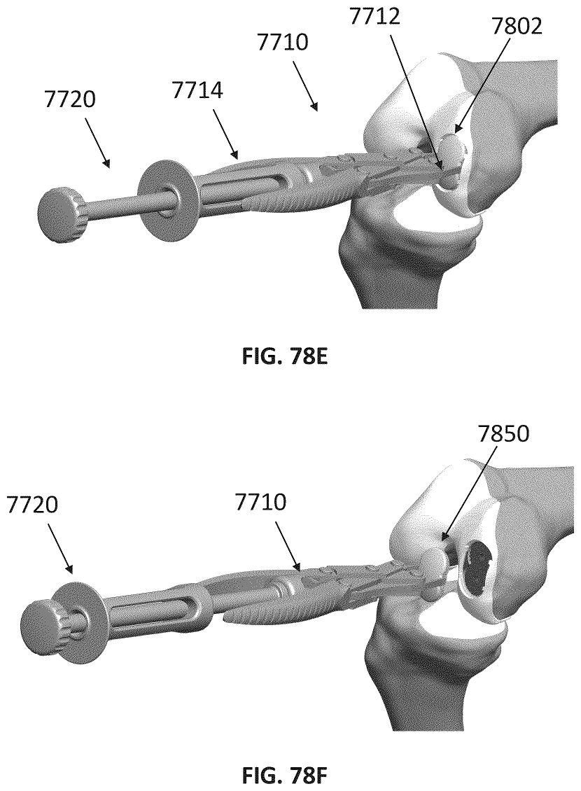

As shown in FIG. 78A, similar to the round femoral implant, the oblong femoral implant 7850 can have a set of pockets 7852 on opposite medial and lateral sides of the midline of the implant 7850 just below the articulating surface of the implant 7850. As shown in FIGS. 78B and 78C, the user can chisel around the edges of the oblong femoral implant 7850 using any one or a combination of a straight femoral removal punch 7800 and a curved femoral removal punch 7810. The chiseling can aid in freeing the oblong femoral implant 7850 from any adjacent bone. As shown in FIG. 78D, the chiseling can result in a channel 7802 around the periphery of the oblong femoral implant 7850.

As shown in FIGS. 78E and 78F, the user can then place the removal tool 7710 in the channel 7802 created by the straight femoral removal punch 7800 and/or the curved femoral removal punch 7810 around the oblong femoral implant 7850. The prongs 7712 of the removal tool 7710 can be inserted in the medial and lateral sides of the channel 7802 to gain access to the pockets 7852 of the oblong femoral implant 7850. The user can align the prongs 7712 of the removal tool 7710 with the pockets 7852 of the oblong femoral implant 7850. If it is difficult to get both prongs 7712 connected to the pockets 7852, the user can attach the slap hammer 7720 to the removal tool 7710 and tap lightly downward on the removal tool 7710 until the user can confirm that the prongs 7712 has engaged the pockets 7852. The user can squeeze the grip 7714 of the removal tool 7710 to grasp the oblong femoral implant 7850. In some implementations, the user can use the slap hammer 7720 to aid in the removal.

### 3. Tibial Removal

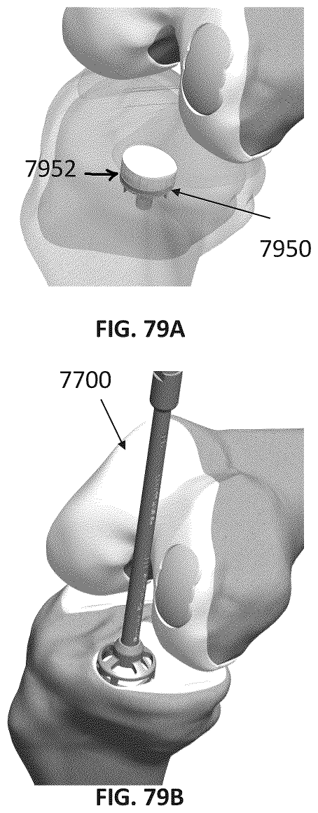

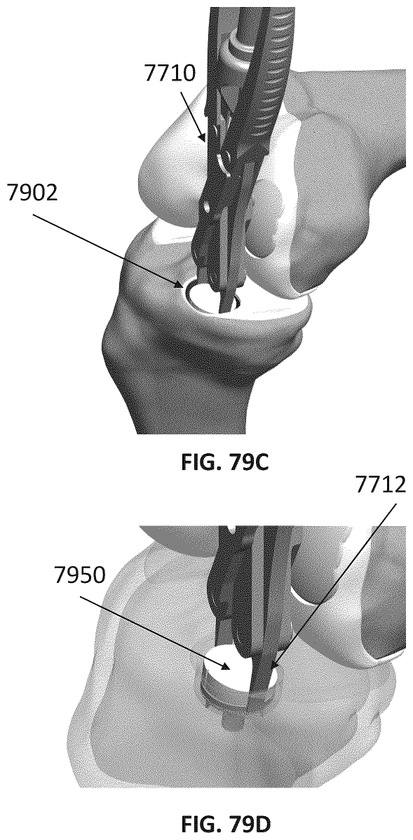

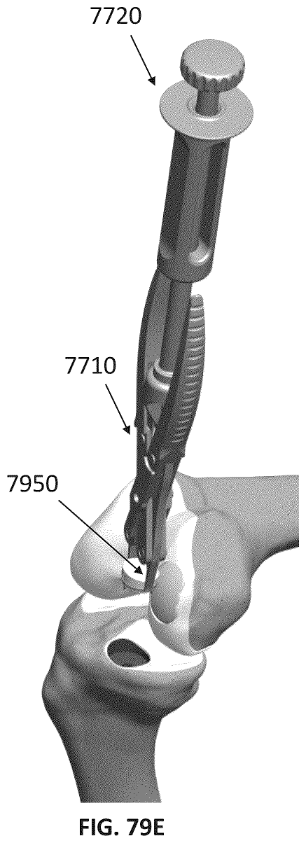

As shown in FIG. 79A, the tibial implant 7950 has a circumferential groove 7952 below the articulating surface of the implant 7905 and the groove 7952 can be used for removal. As shown in FIG. 79B, to gain access to the groove 7952, the user can place the hole saw 7700 of a selected size around the tibial implant 7950 or any other suitable hole saw. The hole saw 7700 can be powered and can drill a circumferential channel 7902 around the articulating surface of the tibial implant 7950. The user can drill until the hole saw reaches its full depth to the bone-contacting (i.e., subchondral) surface of the tibial implant 7950 or at least as deep as the groove 7952. Alternatively, a user can use manual (nonpowered) instruments to create holes adjacent to the tibial implant 7950. The holes can have a width sufficient to accommodate insertion of tips of the prongs 7712 of the removal tool 7700.

As shown in FIGS. 79C and 79D, the user can then remove the hole saw 7700 and place the removal tool 7710 around the tibial implant 7950 in the circumferential channel 7902 created by the hole saw 7700 or the manually created holes. The user can align the prongs 7712 of the removal tool 7710 with the groove 7952 of the tibial implant 7950. If it is difficult to get both prongs 7712 connected to the groove 7952, the user can attach the slap hammer 7720 to the removal tool 7710 and tap lightly downward on the removal tool 7710 until the user can confirm that the prongs 7712 has engaged the groove 7952. The user can squeeze the grip 7714 of the removal tool 7710 to grab the tibial implant 7950. In some implementations, the user can attach a slap hammer 7720 to aid in the removal as shown in FIG. 79E.

### G. Example Patellofemoral Joint Replacement

Currently available partial knee systems can involve large implants that require total resection and resurfacing of the trochlear groove of the femur and the patellar surface. The patellar implant of such systems typically includes multiple components that require in-patient assembly. In some currently available partial knee systems, the trochlear component includes multiple components that require in-patient assembly. Known systems often require cement for insertion and fixation, compromising the strength of fixation. Currently available femoral implants often require cement for fixation. Embodiments of the present disclosure provide unique solutions that address at least some of these limitations and/or other problems with current partial knee systems.

Implant embodiments disclosed herein can be used to address osteochondral defects early in their development, and to prevent or delay the need for a total joint replacement required if the defect worsens over time. This approach offers an appropriate step between biological techniques and the total joint arthroplasties. The fixation features of the implants can allow for stronger, cementless fixation. The strong fixation and small profile can provide a high level of mobility to patients, where other approaches and implants can limit patient mobility. The array of geometric implant sizing and profile options can allow for minimal bone removal and congruent surfacing between the implant and the native bone.

Exemplary arthroplasty resurfacing systems and methods disclosed herein encompass the use of a first implant having a bearing surface portion that matches the geometry of the trochlear groove in the femur and a subchondral surface portion, and a second implant having a domed bearing surface portion and a subchondral surface portion. The implant system can be configured for implantation into a joint of a patient. The joint may be, for example, a knee joint, a shoulder joint, a hip joint, an ankle joint, a first metatarsal-phalangeal joint, or the like. In some implementations, the first implant can be a trochlear implant (to be implanted on the femur side); and the second implant can be a patellar implant (to be implanted on the patellar side).

In one embodiment, a joint resurfacing system includes two main implants available for the knee joint: a round trochlear implant (also referred to as a femoral implant for the patellofemoral system); a round patellar implant. In some embodiments, the trochlear implant has a titanium bearing surface that matches the geometry of the trochlear groove. The geometry options include a minimum, nominal, and maximum geometry. In some embodiments, the trochlear implant has a titanium nitride (TiN) coating on the bearing surface. In some implementations, the entire metal structure (including the porous and non-porous portions) of the trochlear implant can be 3D printed. In some implementations, the entire metal structure of the trochlear implant may be manufactured as a monolithic unit such that no coupling mechanism is required to couple different parts of the metal structure together. The circular implant has a single peg with barbs as well as irregular, porous titanium sections on the subchondral surfaces to promote fixation. There are anti-rotation spikes from the base of the implant to prevent rotational movement.

In some cases, implants may only be implanted on surfaces that contain osteochondral defects. They can either be used individually or in tandem on articulating surfaces. Optional features and alternatives to certain round implant embodiments disclosed herein include a variety of diametric sizes and an implant with an oblong or “ooblong” profile, with variable length and width. The oblong implant is longer in the anteroposterior direction (i.e., along the trochlear groove axis). The “ooblong” implant is longer in the mediolateral direction or in the sagittal plane (i.e., across or parallel to the trochlear groove) than the oblong profile. The oblong shaped implants may also vary with directionality along the trochlear groove. The oblong shaped implants may also vary in the number of fixation pegs. In some cases, an implant may have a different profile, such as a three-circles profile. In some cases, the three circles have similar diameters. In some cases, the profile may include a center circle with a rim that creates a plane perpendicular to the transverse and sagittal planes of the trochlear groove. In some cases, the profile may include two outer circles with rims that create planes perpendicular to the sagittal plane of the trochlear groove and angled towards the transverse plane of the trochlear groove. In some cases, the profile may include two outer circles with rims that create planes parallel to the plane of the center circle.

Optional and alternative features for the patellar implants include various diametric sizes and bearing surface geometries. This includes anatomic or button bearing surface geometries.

Variations for exemplary system implants can include those without porous trabecular structures to help reduce implant thickness. Implant embodiments can also have variations to accommodate different bones and joints. Implant embodiments can have multi-peg or pegless versions. Multi-peg options can remove the need for anti-rotation spikes, and pegless options can remove the need for drilling preparation. Implant embodiments encompass versions having barbless pegs that are a trabecular porous structure.

Portions of an implant may include titanium, stainless steel, cobalt chrome, and the like. Any bearing surface can be made out of ceramic.

In some embodiments, trochlear round implants range in dimension diametrically from 20-35 mm. Trochlear oblong implants can range in length and width/diameter from 25 mm×40 mm and 20 mm to 35 mm. In some embodiments, the overall thickness of trochlear implants can vary from 4 mm to 15 mm. As explained elsewhere herein, the thickness of a trochlear implant can refer to the distance from the inflection point on the bearing surface (maximum for the femoral implant) to the subchondral surface, where the porous section engages the bottom or surface of the reamed bone. In some embodiments, the trochlear implant has a titanium bearing surface that matches the geometry of the trochlear groove. The geometry options include a minimum, nominal, and maximum geometry.

Turning to the patellar implants, some embodiments, patellar implants range in diameters from 25 mm to 30 mm. In some cases, the minimum overall thickness of a patellar implants is 7.5 mm. As explained elsewhere herein, the thickness of a patellar implant can refer to the distance from the inflection point on the bearing surface (minimum for the patellar implant) to the subchondral surface, where the porous section engages the bottom or surface of the reamed bone.

In some cases, no is cement required. Exemplary embodiments also facilitate the accuracy of hand reaming for fine tuning final implant fit. Exemplary embodiments also encompass limited instrument sets and/or the use of no cement, leading to reduced time for surgery and reduced risk of patient exposure.

Some system embodiments include one implant per surface/defect correction. In some embodiments, three-circle oblong implants include angled outer circles, which in turn reduces the depth of bone resection. In some embodiments, three-circle oblong implants include outer circles with bottom surfaces that go to a specific depth below the condylar surface, which in turn reduces the depth of bone resection. A defect-sized profile reduces the area of articulating surface that needs to the be resected. Embodiments also encompass a type of multi-/no-peg alternative. A no-peg embodiment can be a helpful option to those patients with previous surgeries/hardware (e.g., interference screws).

Alternative embodiments can include slightly altered implants that are used in similar resurfacing procedures in other joints that frequently develop osteochondral defects, including, but not limited to, the shoulder, the hip, the ankle (talus), and the first metatarsal-phalangeal joint. In some implementations, one or more implants disclosed herein can be used in combination with a femoral implant and/or a tibial implant, such as the femoral and/or tibial implants disclosed herein, and/or described in U.S. Pat. Pub. No. 2022/0249239, published on Aug. 11, 2022, the entirety of which is incorporated herein by reference. Alternatively and/or additionally to being used in combination with the femoral and/or tibial implant, one or more implants disclosed herein can be used with other treatment modalities, including but not limited to unicompartmental knee arthroscopy.

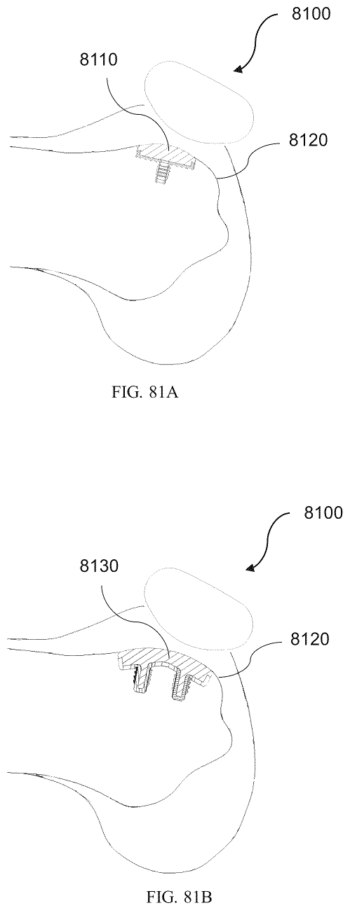

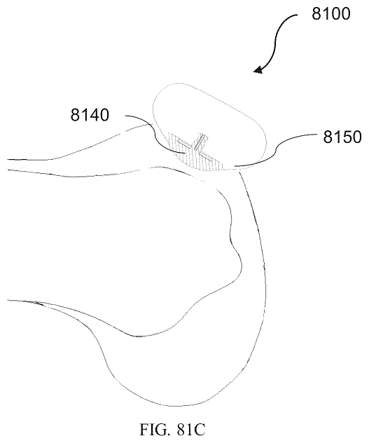

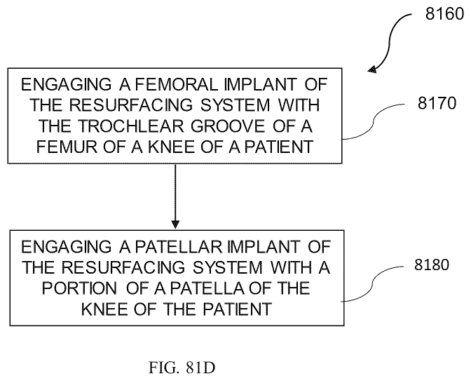

Turning now to the drawings, FIGS. 81A to 81C depict various aspects of a patellofemoral knee arthroplasty resurfacing system 8100, according to embodiments of the present disclosure. An exemplary system may include a trochlear implant and a patellar implant. For example, FIG. 81A depicts a single-peg trochlear implant 8110 that is engaged with the trochlear groove 8120 of a femur of a patient. Likewise, FIG. 81B depicts a multi-peg trochlear implant 8130 that is engaged the trochlear groove 8120 of a femur of a patient. FIG. 81C depicts a single-peg patellar implant 8140 that is engaged with a proximal portion 8150 of a patella of a patient. As further discussed herein, a trochlear implant can have a bearing surface portion that matches the geometry of the trochlear groove, the patellar implant can have a domed bearing surface portion, and the domed and bearing surface portions can slidingly engage one another during use of the resurfacing system, for example as the patient's knee joint undergoes flexion and extension. FIG. 81D depicts aspects of a method 8160 of implanting a patellofemoral knee arthroplasty resurfacing system into a compartment of a knee of a patient. As shown in this embodiment, the method 8160 can include engaging a trochlear implant of the resurfacing system with the trochlear groove of the knee of the patient, as indicated by step 8170. The trochlear implant can include a bearing surface portion and a subchondral surface portion. In some embodiments, the trochlear implant has a titanium bearing surface that matches the geometry of the trochlear groove. The geometry options include a minimum, nominal, and maximum geometry. The method 8160 can also include engaging a patellar implant of the resurfacing system with a proximal portion of a patella of the knee of the patient, as indicated by step 8180. The patellar implant can include a domed bearing surface portion and a subchondral surface portion.

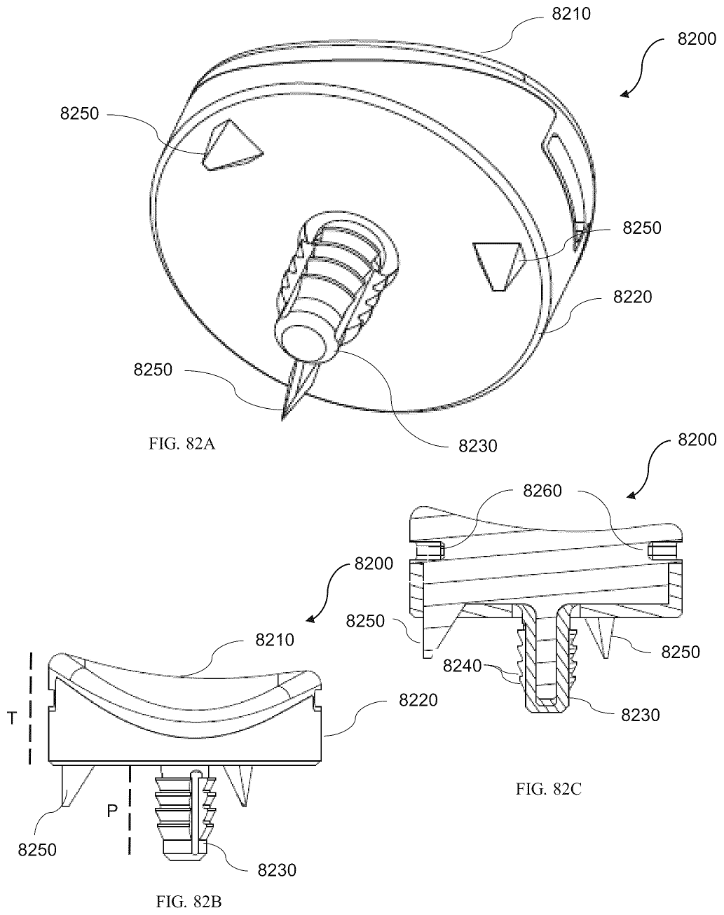

FIGS. 2A to 2C depict aspects of an exemplary trochlear implant 200 according to embodiments of the present disclosure. The trochlear implant 8200 can have a round profile. In some implementations, the trochlear implant 8200 can have an outer diameter of about 20.0 mm. As shown here, a trochlear implant 8200 can have a bearing surface portion 8210 and a subchondral surface portion 8220. The bearing surface portion 8210 can operate as an articulating surface. In some embodiments, the trochlear implant has a titanium bearing surface that matches the geometry of the trochlear groove. The geometry options include a minimum, nominal, and maximum geometry. The trochlear implant 8200 can also include a proximal peg 8230. In the embodiment depicted here, proximal peg 8230 includes multiple barbs 8240. In some cases, the bearing surface portion 8210 of the trochlear implant 8200 can include nonporous titanium. In some cases, the bearing surface portion 8210 can include nonporous stainless steel, nonporous cobalt chrome, nonporous ceramic, and the like. In some cases, the subchondral surface portion 8220 of the trochlear implant 8200 can include porous titanium. In this embodiment, the bearing surface portion 8210 of the trochlear implant 8200 has a round profile. In some cases (FIG. 82B), the trochlear implant 8200 can have a thickness T with a value within a range from about 5 mm to about 15 mm. The thickness T of a trochlear implant can refer to the distance from the inflection point on the bearing surface (maximum for the trochlear implant) to the subchondral surface, where the porous section engages the bottom or surface of the reamed bone. A peg length P can have a value of about 7 mm. In some cases, the proximal peg 8230 can have a trabecular porous structure. In some cases, the trochlear implant 8200 is a monolithic unit. In some cases, the trochlear implant 8200 includes a bone screw fixation mechanism. In this embodiment, the trochlear implant 8200 includes multiple anti-rotation spikes or prongs 250. In some cases, the trochlear implant 8200 includes removal pockets 8260 for use with a removal tool.

FIGS. 83A and 83B depict aspects of an exemplary trochlear implant 8300 according to embodiments of the present disclosure. As shown here, a trochlear implant 8300 can have a bearing surface portion 8310 and a subchondral surface portion 8320. The trochlear implant 8300 can also include a proximal peg 8330. In the embodiment depicted here, proximal peg 8330 includes multiple barbs 8340. In some cases, the peg 8330 and subchondral surface portion 8320 include a porous material, and the barbs 8340 and the bearing surface portion 8310 includes a solid or nonporous material. In some cases, the bearing surface portion 8310 of the trochlear implant 8300 can include nonporous titanium. In some cases, the bearing surface portion 8310 can include nonporous stainless steel, nonporous cobalt chrome, nonporous ceramic, and the like. In some cases, the barbs 8340 can include a nonporous or solid material. In this embodiment, the trochlear implant 8300 includes multiple anti-rotation spikes or prongs 8350. In some cases, the subchondral surface portion 8320 of the trochlear implant 8300 can include porous titanium. In this embodiment, the bearing surface portion 8310 of the trochlear implant 8300 has a round profile. In some cases, the proximal peg 8330 can have a trabecular porous structure. In some cases, the trochlear implant 8300 is a monolithic unit. In some cases, the trochlear implant 8300 includes removal pockets 8360 for use with a removal tool. In some cases, the trochlear implant can include no porous structure (e.g., FIG. 83A). That is, the trochlear implant depicted in FIG. 83A is completely made of a solid or nonporous material.

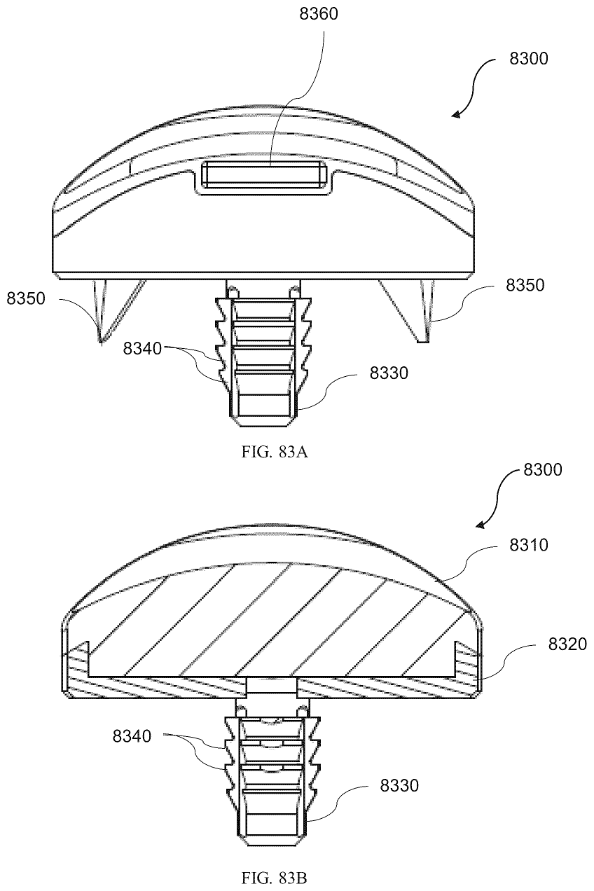

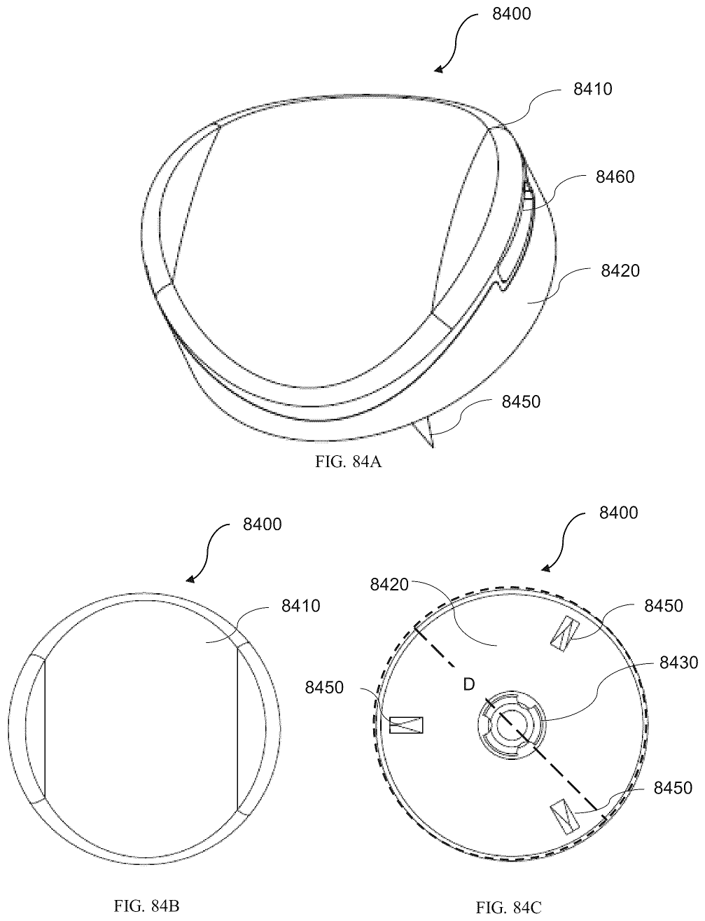

FIGS. 84A to 84C depict aspects of an exemplary trochlear implant 8400 according to embodiments of the present disclosure. As shown here, a trochlear implant 8400 can have a bearing surface portion 8410 and a subchondral surface portion 8420. The trochlear implant 8400 can also include a proximal peg 8430. In some cases, the bearing surface portion 8410 of the trochlear implant 8400 can include nonporous titanium. In some cases, the bearing surface portion 8410 can include nonporous stainless steel, nonporous cobalt chrome, nonporous ceramic, and the like. In some cases, the trochlear implant can include no porous structure. In this embodiment, the bearing surface portion 8410 of the trochlear implant 8400 has a round profile. In some cases, the trochlear implant 8400 is a monolithic unit. In this embodiment, the trochlear implant 8400 includes multiple anti-rotation spikes or prongs 8450. In some cases, the trochlear implant 8400 includes removal pockets 8460 for use with a removal tool. In some cases (FIG. 84B), the bearing surface portion 8410 can provide a round profile having a diameter D with a value within a range from about 20 mm to about 40 mm.

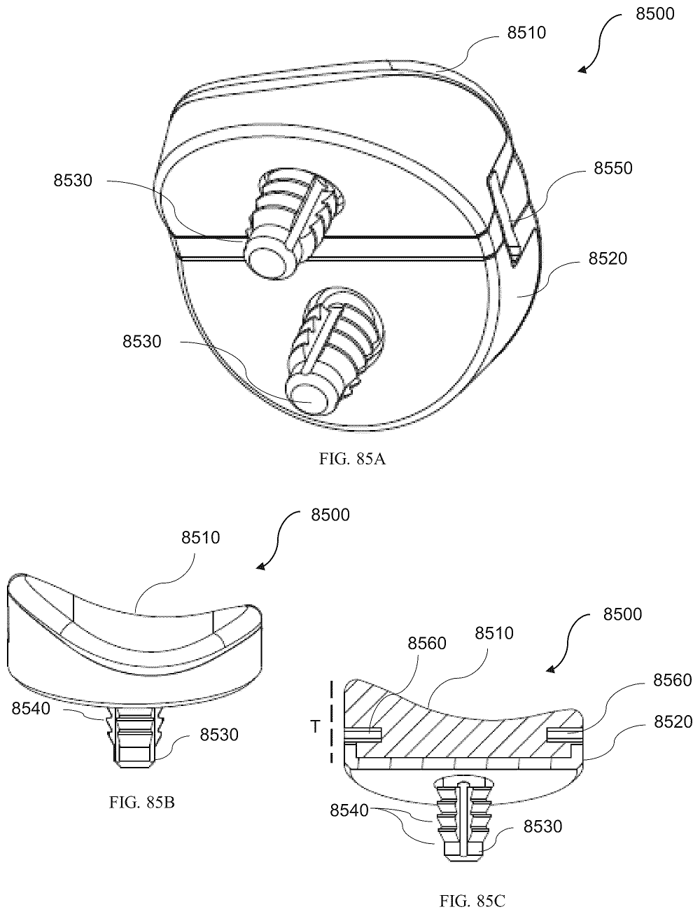

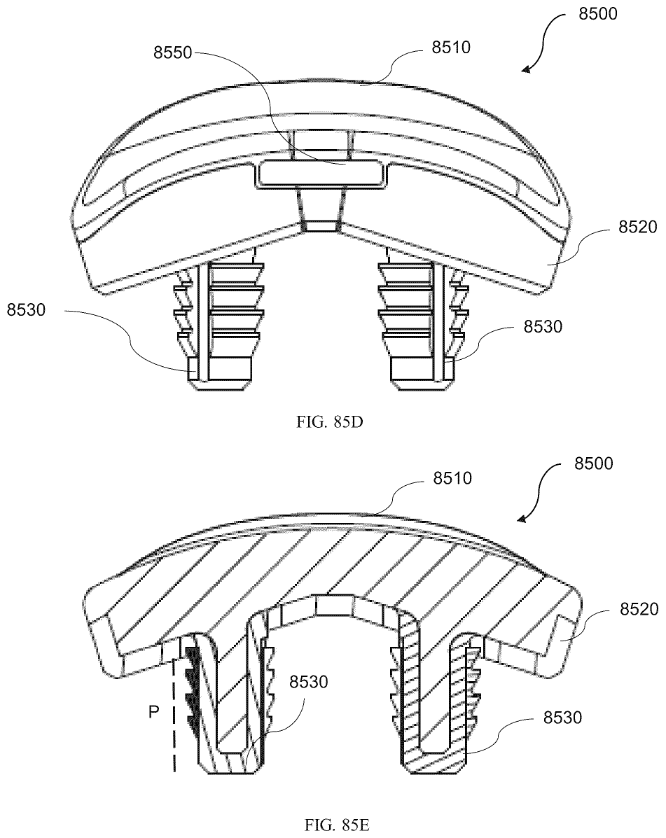

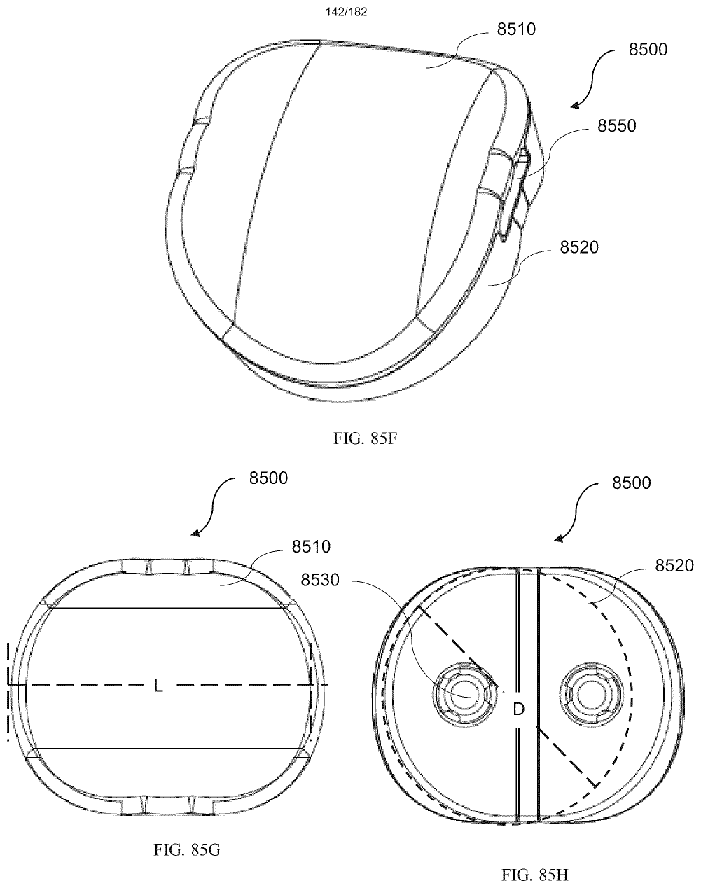

FIGS. 85A to 85H depict aspects of an exemplary trochlear implant 8500 according to embodiments of the present disclosure. The trochlear implant 8500 can have an oblong profile. In some implementations, the trochlear implant 8500 can have a dimension of about 20.0 mm×about 25.0 mm. As shown here, a trochlear implant 8500 can have a bearing surface portion 8510 and a subchondral surface portion 8520. The trochlear implant 8500 can also include one or more proximal pegs 8530. In some cases, the subchondral surface portion 8520 and/or one or more the proximal pegs 8530 can include a porous material, such as porous titanium. In the embodiment depicted here, proximal pegs 8530 include multiple barbs 8540. In some cases, the bearing surface portion 8510 of the trochlear implant 8500 can include nonporous titanium. In some cases, the bearing surface portion 8510 can include nonporous stainless steel, nonporous cobalt chrome, nonporous ceramic, and the like. In some cases, the pegs 8530 and subchondral surface portion 8520 include a porous material, and the barbs 8540 and the bearing surface portion 8510 includes a solid or nonporous material (e.g., FIG. 85E). In some cases, the trochlear implant can include no porous structure (e.g., FIG. 85D). That is, the trochlear implant depicted in FIG. 85D is completely made of a solid or nonporous material. The bearing surface portion 8510 of the trochlear implant 8500 has a three-circle or oblong profile. The three-circle profile provides a surface portion that has three sections, which allows for simple reaming with a circular reamer. In some cases, the three-circle geometry may include a center circle with a rim that creates a plane perpendicular to the transverse and sagittal planes of the trochlear groove. In some cases, the geometry may include two outer circles with rims that create planes perpendicular to the sagittal plane of the trochlear groove and angled towards the transverse plane of the trochlear groove. In some cases, the profile may include two outer circles with rims that create planes parallel to the plane of the center circle. In some cases, the trochlear implant 8500 is a monolithic unit. In some cases, the trochlear implant 8500 includes removal pockets 8550 for use with a removal tool. In this embodiment, the bearing surface portion 8510 of the trochlear implant 8500 has a nominal trochlear groove profile geometry. In this embodiment, the bearing surface portion 8510 of the trochlear implant 8500 has an oblong three-circle geometry. In this embodiment, the geometry may include two outer circles with rims that create planes perpendicular to the sagittal plane of the trochlear groove and angled towards the transverse plane of the trochlear groove. This minimizes bone resection in this orientation of the trochlear implant in the trochlear groove. In some cases (FIG. 85C), the trochlear implant 8500 can have a thickness T with a value within a range from about 5 mm to about 15 mm. The thickness T of a trochlear implant can refer to the distance from the inflection point on the bearing surface (maximum for the femoral implant) to the subchondral surface, where the porous section engages the bottom or surface of the reamed bone. In some cases (FIG. 85E), a peg length P can have a value of about 7 mm. In this embodiment, the trochlear implant 8500 includes no anti-rotation spikes, although such spikes may be present in other embodiments. In some cases (FIG. 85G), the bearing surface portion 8510 can provide a three-circle profile having a length L with a value within a range from about 25 mm to about 40 mm. In some cases (FIG. 85H), the bearing surface portion 8510 can provide a three-circle profile having a diameter D with a value within a range from about 20 mm to about 50 mm.

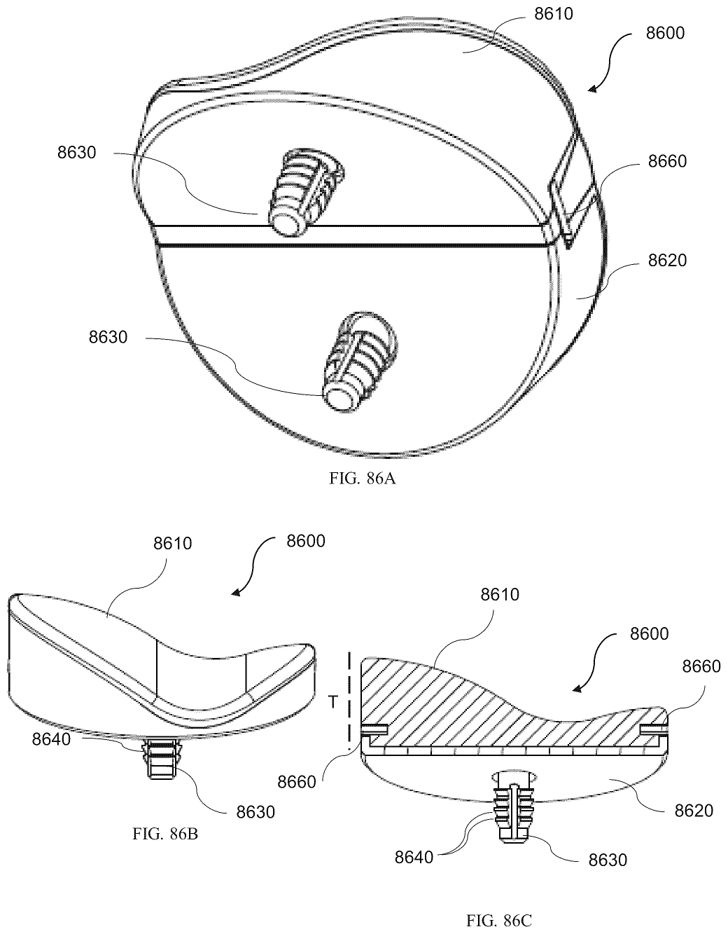

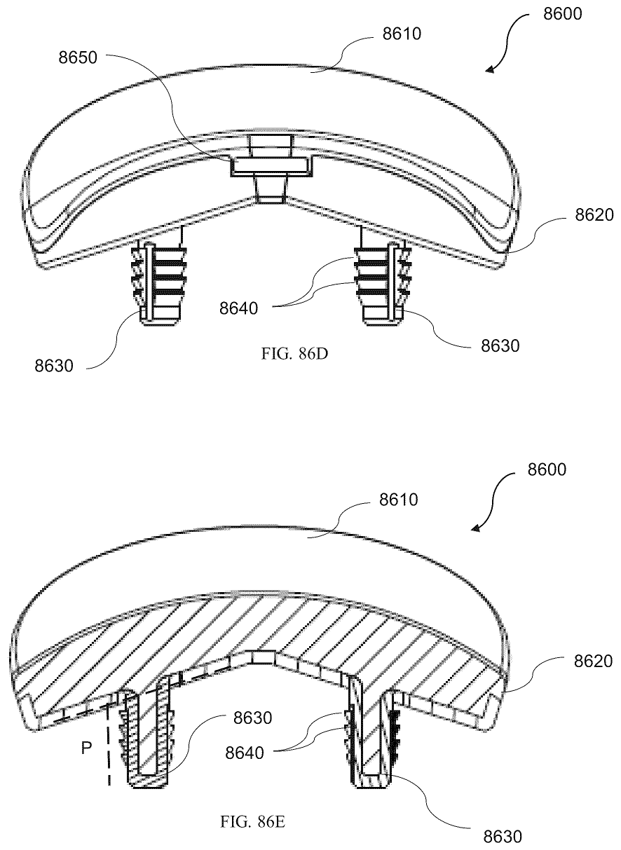

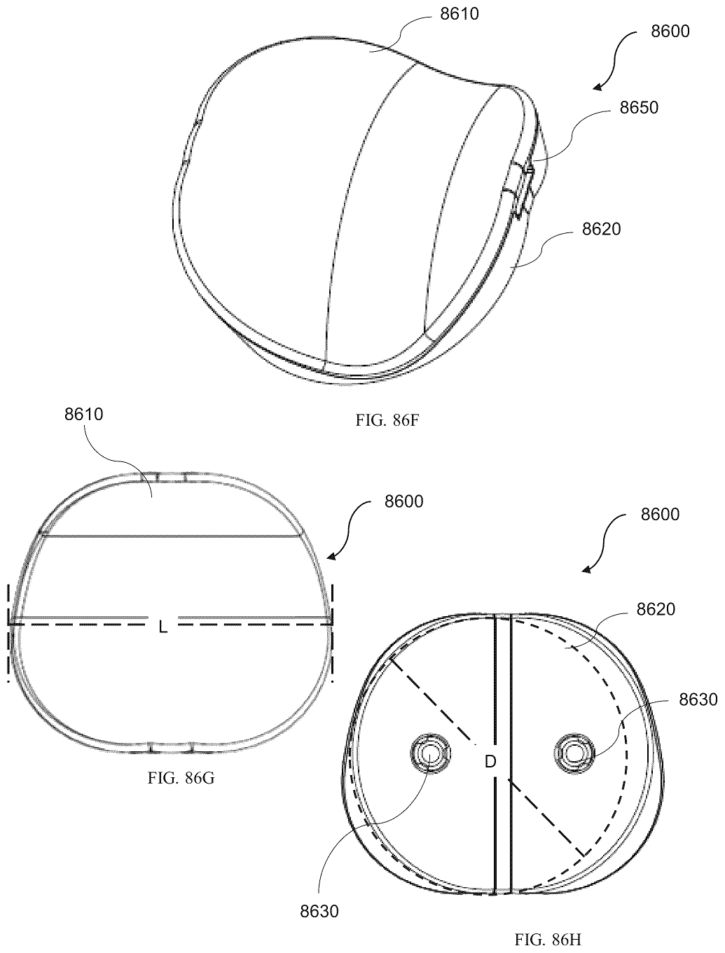

FIGS. 86A to 86H depict aspects of an exemplary trochlear implant 8600 according to embodiments of the present disclosure. The trochlear implant 8600 can have an oblong profile. In some implementations, the trochlear implant 8600 can have a dimension of about 35.0 mm×about 40.0 mm. As shown here, a trochlear implant 8600 can have a bearing surface portion 8610 and a subchondral surface portion 8620. The trochlear implant 8600 can also include one or more proximal pegs 8630. In some cases, the subchondral surface portion 8620 and/or one or more the proximal pegs 8630 can include a porous material, such as porous titanium. In the embodiment depicted here, proximal pegs 8630 include multiple barbs 8640. In some cases, the bearing surface portion 8610 of the trochlear implant 8600 can include nonporous titanium. In some cases, the bearing surface portion 8610 can include nonporous stainless steel, nonporous cobalt chrome, nonporous ceramic, and the like. In some cases, the pegs 8630 and subchondral surface portion 8620 include a porous material, and the barbs 8640 and the bearing surface portion 8610 includes a solid or nonporous material (e.g., FIG. 86E). In some cases, the trochlear implant can include no porous structure (e.g., FIG. 86D). That is, the trochlear implant depicted in FIG. 86D is completely made of a solid or nonporous material. The bearing surface portion 8610 of the trochlear implant 8600 has a three-circle or oblong profile. The three-circle profile provides a surface portion that has three sections, which allows for simple reaming with a circular reamer. In some cases, the three-circle geometry may include a center circle with a rim that creates a plane perpendicular to the transverse and sagittal planes of the trochlear groove. In some cases, the geometry may include two outer circles with rims that create planes perpendicular to the sagittal plane of the trochlear groove and angled towards the transverse plane of the trochlear groove. In some cases, the profile may include two outer circles with rims that create planes parallel to the plane of the center circle. In some cases, the trochlear implant 8600 is a monolithic unit. In some cases, the trochlear implant 8600 includes removal pockets 8650 for use with a removal tool. In this embodiment, the bearing surface portion 8610 of the trochlear implant 8600 has a nominal trochlear groove profile geometry. In this embodiment, the bearing surface portion 8610 of the trochlear implant 8600 has an oblong three-circle geometry. In this embodiment, the geometry may include two outer circles with rims that create planes perpendicular to the sagittal plane of the trochlear groove and angled towards the transverse plane of the trochlear groove. This minimizes bone resection in this orientation of the trochlear implant in the trochlear groove. In some cases (FIG. 86C), the trochlear implant 8600 can have a thickness T with a value within a range from about 5 mm to about 15 mm. The thickness T of a trochlear implant can refer to the distance from the inflection point on the bearing surface (maximum for the femoral implant) to the subchondral surface, where the porous section engages the bottom or surface of the reamed bone. In some cases (FIG. 86E), a peg length P can have a value of about 7 mm. In this embodiment, the trochlear implant 8600 includes no anti-rotation spikes, although such spikes may be present in other embodiments. In some cases (FIG. 86G), the bearing surface portion 8610 can provide a three-circle profile having a length L with a value within a range from about 25 mm to about 40 mm. In some cases (FIG. 86H), the bearing surface portion 8610 can provide a three-circle profile having a diameter D with a value within a range from about 20 mm to about 50 mm.

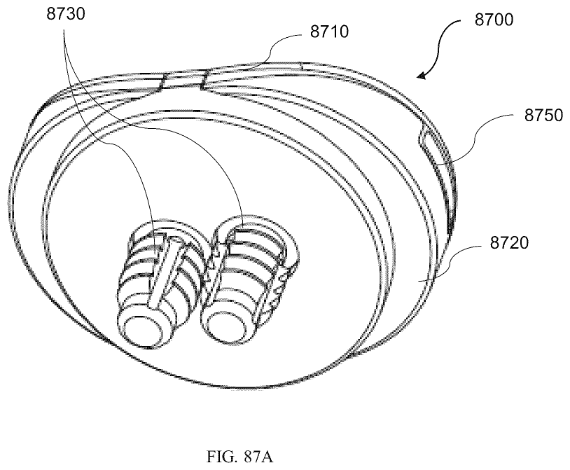

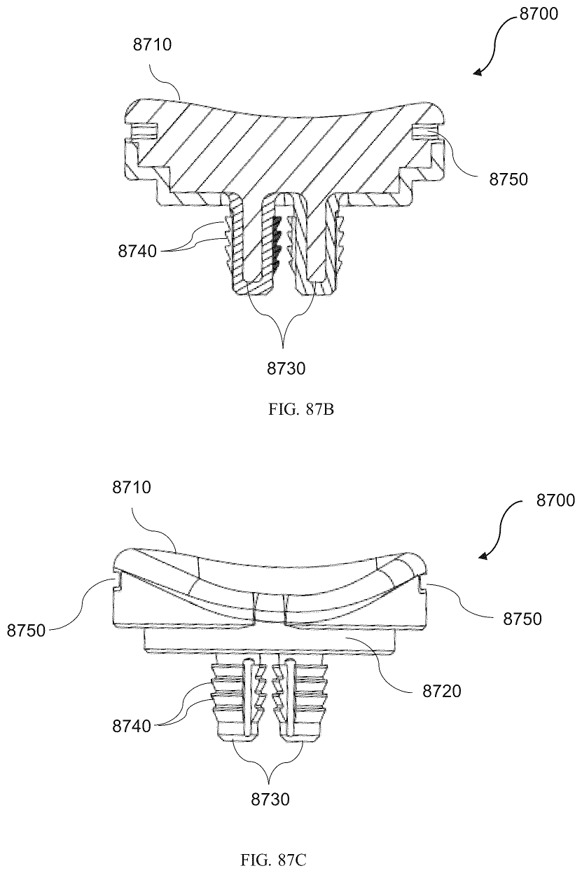

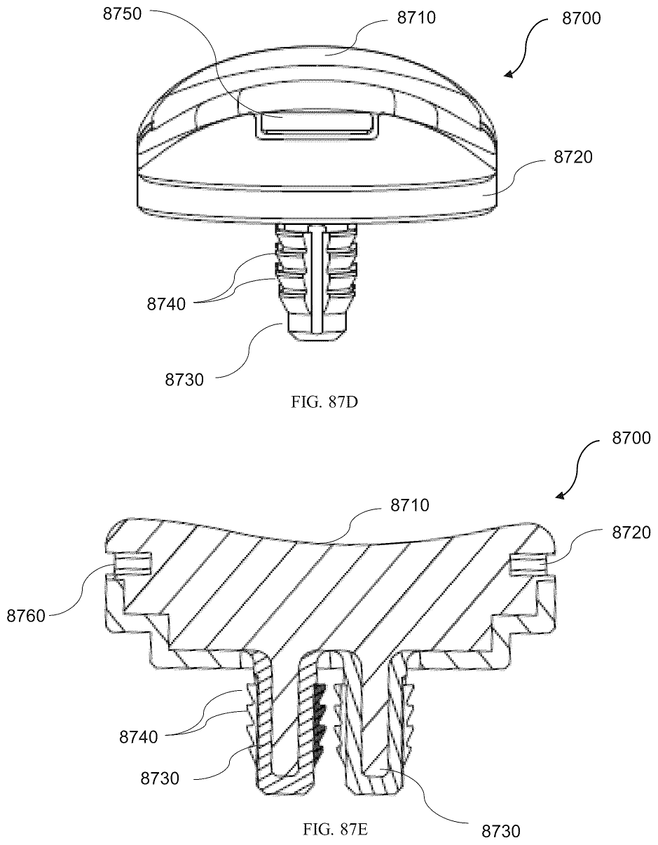

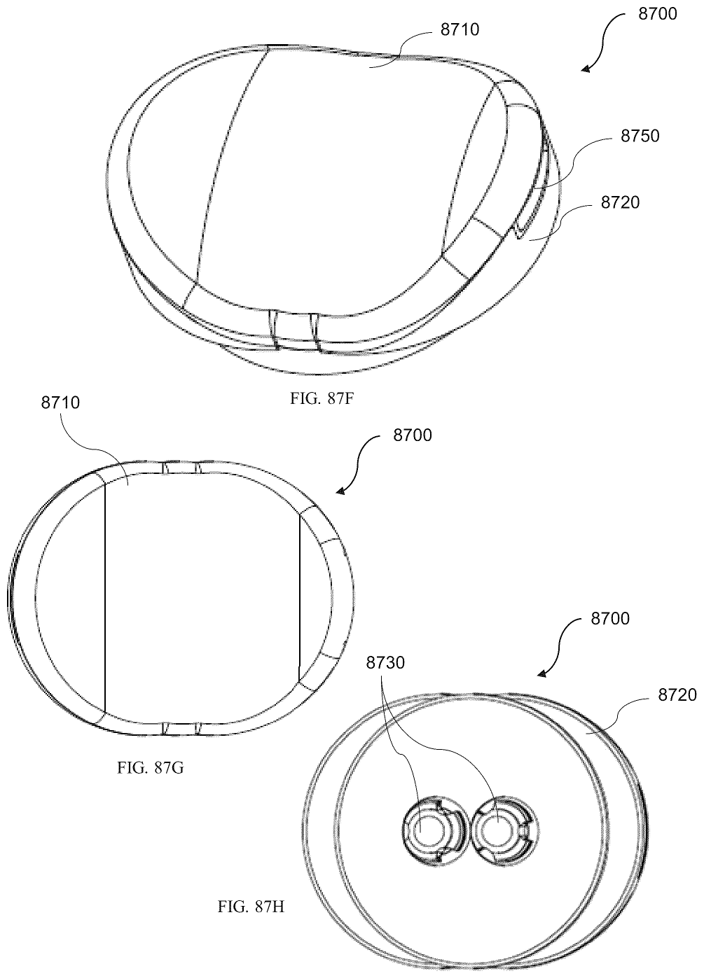

FIGS. 87A to 87H depict aspects of an exemplary trochlear implant 8700 according to embodiments of the present disclosure. The trochlear implant 8700 can include an ooblong profile. In some implementations, the trochlear implant 8700 can have a dimension of about 20.0 mm×25.0 mm. As shown here, a trochlear implant 8700 can have a bearing surface portion 8710 and a subchondral surface portion 8720. The trochlear implant 8700 can also include one or more proximal pegs 8730. In some cases, the subchondral surface portion 8720 and/or one or more the proximal pegs 8730 can include a porous material, such as porous titanium. In the embodiment depicted here, proximal pegs 8730 include multiple barbs 8740. In some cases, the bearing surface portion 8710 of the trochlear implant 8700 can include nonporous titanium. In some cases, the bearing surface portion 8710 can include nonporous stainless steel, nonporous cobalt chrome, nonporous ceramic, and the like. In some cases, the pegs 8730 and subchondral surface portion 8720 include a porous material, and the barbs 8740 and the bearing surface portion 8710 includes a solid or nonporous material (e.g., FIG. 87E). In some cases, the femoral implant can include no porous structure (e.g., FIG. 87D). That is, the trochlear implant depicted in FIG. 87D is completely made of a solid or nonporous material. The bearing surface portion 8710 of the trochlear implant 8700 has a three-circle or oblong profile. The three-circle profile provides a surface portion that has three sections, which allows for simple reaming with a circular reamer. In some cases, the three-circle geometry may include a center circle with a rim that creates a plane perpendicular to the transverse and sagittal planes of the trochlear groove. In some cases, the geometry may include two outer circles with rims that create planes perpendicular to the sagittal plane of the trochlear groove and angled towards the transverse plane of the trochlear groove. In some cases, the profile may include two outer circles with rims that create planes parallel to the plane of the center circle. In some cases, the trochlear implant 8700 is a monolithic unit. In some cases, the trochlear implant 8700 includes removal pockets 8750 for use with a removal tool. In this embodiment, the bearing surface portion 8710 of the trochlear implant 8700 has a nominal trochlear groove profile geometry. In this embodiment, the bearing surface portion 8710 of the trochlear implant 8700 has an oblong three-circle geometry. In this embodiment, the profile includes two outer circles with rims that create planes parallel to the plane of the center circle. This minimizes bone resection in this orientation of the femoral implant in the trochlear groove. In this embodiment, the trochlear implant 8700 includes no anti-rotation spikes, although such spikes may be present in other embodiments.

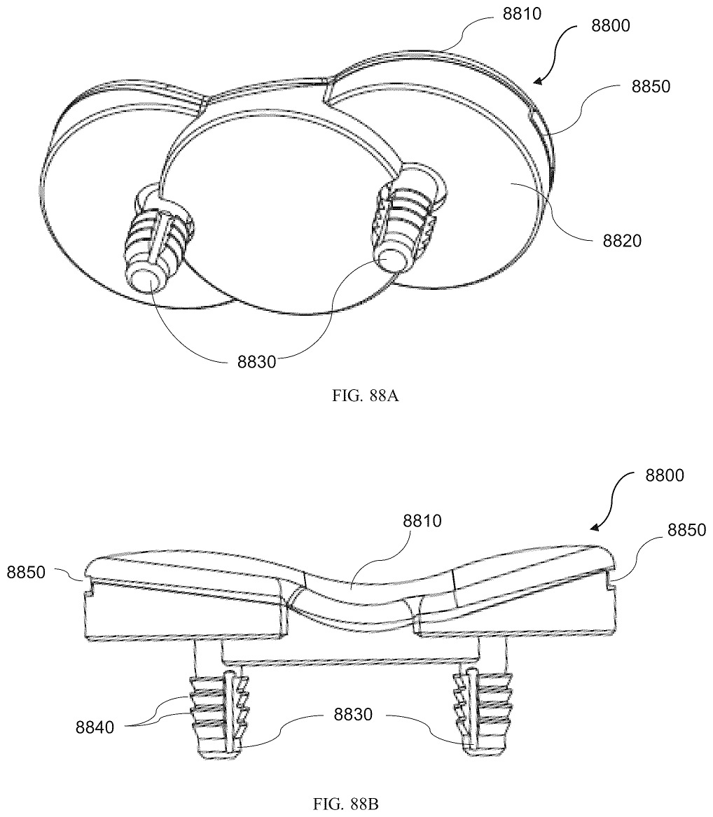

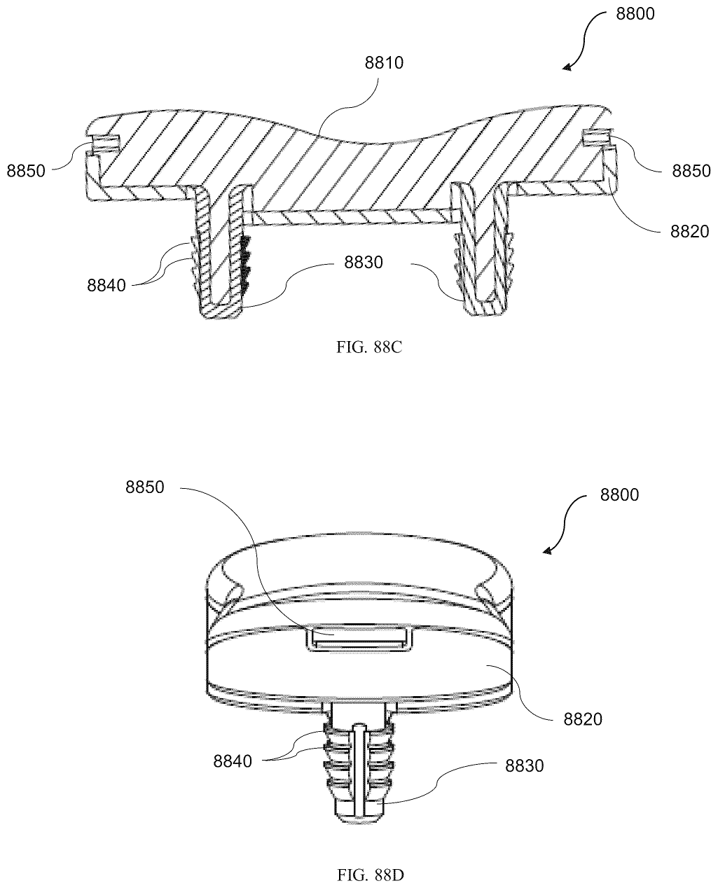

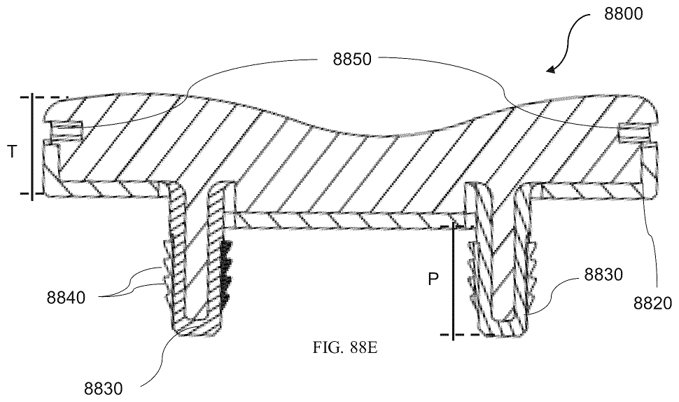

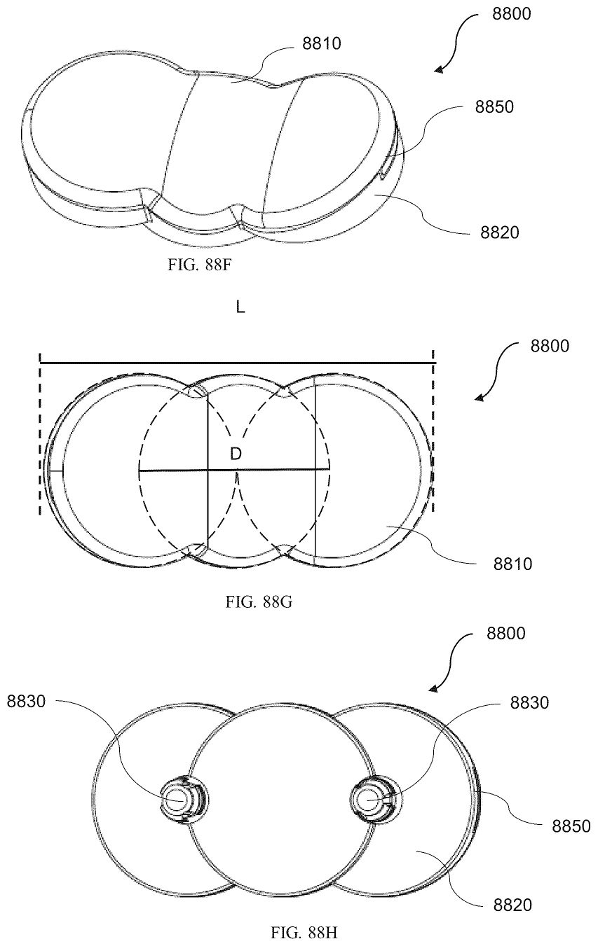

FIGS. 88A to 88H depict aspects of an exemplary trochlear implant 8800 according to embodiments of the present disclosure. The trochlear implant 8800 can include an ooblong profile. The trochlear implant 8800 can have a dimension of about 35.0 mm×40.0 mm. As shown here, a trochlear implant 8800 can have a bearing surface portion 8810 and a subchondral surface portion 8820. The trochlear implant 8800 can also include one or more proximal pegs 8830. In some cases, the subchondral surface portion 8820 and/or one or more the proximal pegs 8830 can include a porous material, such as porous titanium. In the embodiment depicted here, proximal pegs 8830 include multiple barbs 8840. In some cases, the bearing surface portion 8810 of the trochlear implant 8800 can include nonporous titanium. In some cases, the bearing surface portion 8810 can include nonporous stainless steel, nonporous cobalt chrome, nonporous ceramic, and the like. In some cases, the pegs 8830 and subchondral surface portion 8820 include a porous material, and the barbs 8840 and the bearing surface portion 8810 includes a solid or nonporous material (e.g., FIG. 88E). In some cases, the trochlear implant can include no porous structure (e.g., FIG. 88D). That is, the trochlear implant depicted in FIG. 88D is completely made of a solid or nonporous material. The bearing surface portion 8810 of the trochlear implant 8800 has a three-circle or oblong profile. The three-circle profile provides a surface portion that has three sections, which allows for simple reaming with a circular reamer. In some cases, the three-circle geometry may include a center circle with a rim that creates a plane perpendicular to the transverse and sagittal planes of the trochlear groove. In some cases, the geometry may include two outer circles with rims that create planes perpendicular to the sagittal plane of the trochlear groove and angled towards the transverse plane of the trochlear groove. In some cases, the profile may include two outer circles with rims that create planes parallel to the plane of the center circle. In some cases, the trochlear implant 8800 is a monolithic unit. In some cases, the trochlear implant 8800 includes removal pockets 8850 for use with a removal tool. In this embodiment, the bearing surface portion 8810 of the trochlear implant 8800 has a nominal trochlear groove profile geometry. In this embodiment, the bearing surface portion 8810 of the trochlear implant 8800 has an oblong three-circle geometry. In this embodiment, the profile includes two outer circles with rims that create planes parallel to the plane of the center circle. This minimizes bone resection in this orientation of the femoral implant in the trochlear groove. In some cases (FIG. 88E), the trochlear implant 8800 can have a thickness T with a value within a range from about 5 mm to about 15 mm. A peg length P can have a value of about 7 mm. The thickness T of a trochlear implant can refer to the distance from the inflection point on the bearing surface (maximum for the femoral implant) to the subchondral surface, where the porous section engages the bottom or surface of the reamed bone. In some cases (FIG. 88G), the bearing surface portion 8810 can provide a three-circle profile having a length L with a value within a range from about 25 mm to about 40 mm. In some cases, the bearing surface portion 8810 can provide a three-circle profile having a diameter D with a value within a range from about 20 mm to about 50 mm. In this embodiment, the trochlear implant 8800 includes no anti-rotation spikes, although such spikes may be present in other embodiments.

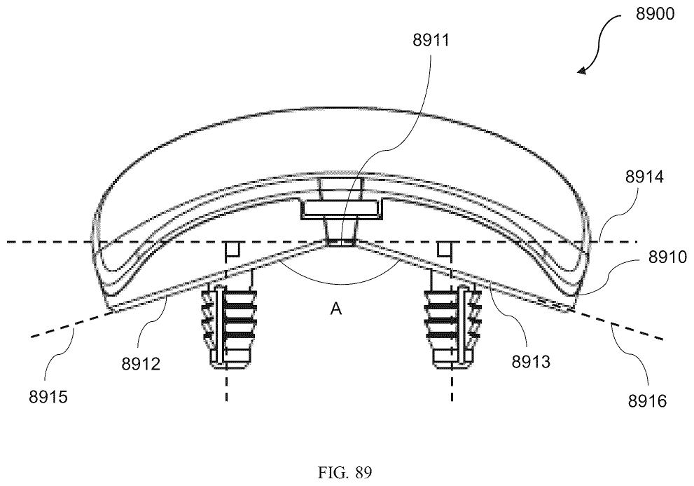

As shown in FIG. 89, the subchondral surface portion 8910 of the three-circle trochlear implant 8900 can have a rim 8911 of a center circle that defines a plane 8914, and a rim of two side circles 8912 and 8913 that define planes 8915 and 8916 at an angle A. In some cases, angle A has a value of about 35 degrees to 90 degrees. As shown here, the bottom or distal surface of the center circle can be perpendicular to the peg or pegs.

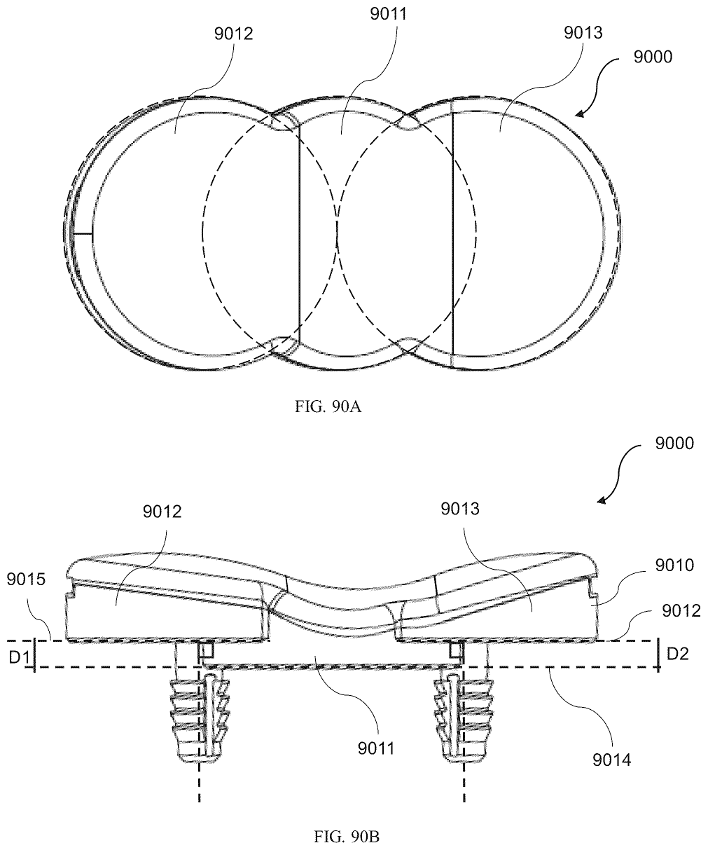

As shown in FIG. 90, the subchondral surface portion 9010 of the three-circle trochlear implant 9000 can have a rim 9011 of a center circle that defines a plane 9014, and a rim of two side circles 9012 and 9013 that define planes 9015 and 9016 that are parallel to plane. In some cases, the depth D1 between planes 9014 and 9015 and the depth D2 between planes 9014 and 9016 are equivalent. As shown here, the bottom or distal surface of the center circle and side circles can be perpendicular to the peg or pegs.

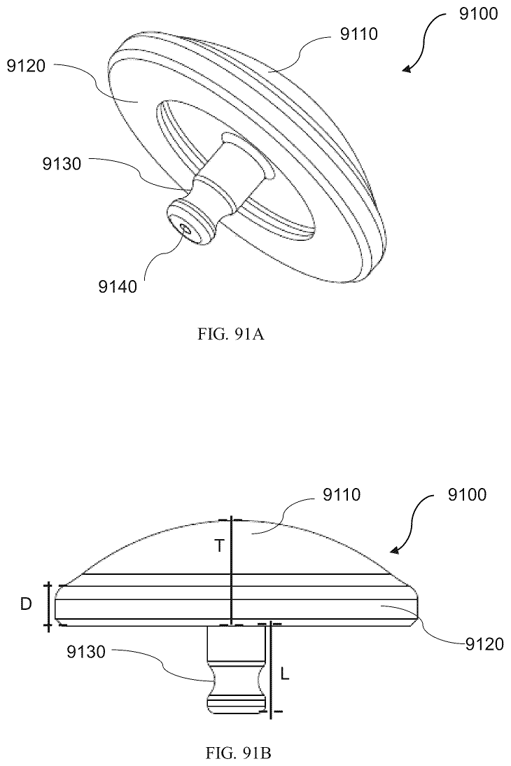

FIGS. 91A to 91D depict aspects of an exemplary patellar implant 9100 according to embodiments of the present disclosure. As shown here, a patellar implant 9100 can have a domed bearing surface portion 9110 and a subchondral surface portion 9120. The patellar implant 9100 can also include a distal peg 9130. In some cases, the distal peg 9130 includes a pin 9140 for X-ray visualization. In some cases, the domed bearing surface portion 9110 of the patellar implant 9100 can include ultra-high molecular weight (UHMWPE) polyethylene. In some cases, the domed bearing surface portion 9110 of the patellar implant 9100 can include vitamin E infused ultra-high molecular weight (UHMWPE) polyethylene. In this embodiment, the domed bearing surface portion 9110 of the femoral implant 9100 has a round profile. In some cases, the domed bearing surface portion 9110 can provide a round profile having a diameter D with a value within a range from about 25 mm to about 30 mm. In some cases, the patellar implant 9100 can have a thickness T with a value that is equal to or greater than about 7.5 mm. The thickness T of a patellar implant can refer to the distance from the inflection point on the bearing surface (minimum for the patellar implant) to the subchondral surface, where the porous section engages the bottom or surface of the reamed bone. A peg length L can have a value of about 6 mm. In some cases, the patellar implant 9100 is a monolithic unit. In this embodiment, the patellar implant 9100 includes no anti-rotation spikes, although such spikes may be present in other embodiments. In some cases, the patellar implant 9100 includes a bone screw fixation mechanism. In some cases, the patellar implant 9100 includes a pocket 9150 for a cement mantle.

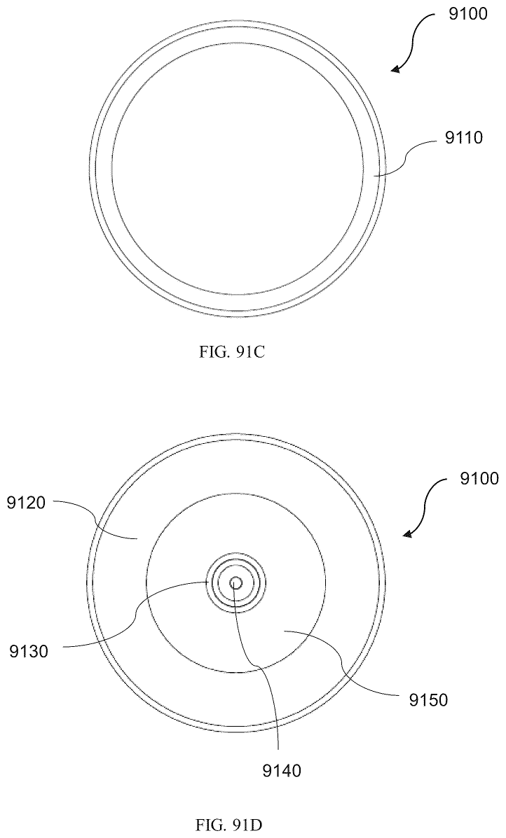

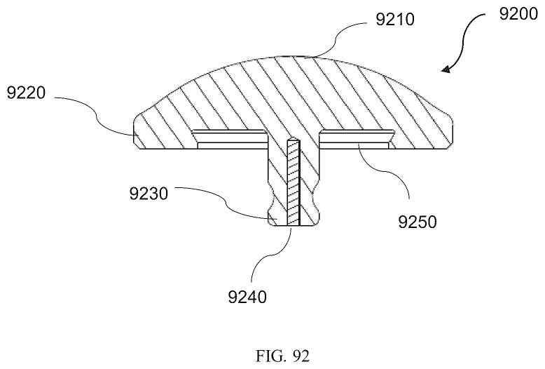

FIG. 92 depicts aspects of an exemplary patellar implant 9200 according to embodiments of the present disclosure. In the cross-section view provided here, the implant 9200 includes a domed bearing surface portion 9210 (which may include a nonporous polymeric material), a subchondral surface portion 9220 (which may include a nonporous polymeric material, and a distal peg 9230 (which may include a nonporous polymeric material). The patellar implant 9200 can be compression molded. In some cases, the domed bearing surface portion 9210 includes ultra-high molecular weight (UHMWPE) polyethylene. In some cases, the domed bearing surface portion 9210 includes vitamin E infused ultra-high molecular weight (UHMWPE) polyethylene. In some cases, a pin 9240 may be pressed into the center peg to provide X-ray visualization. In some cases the patellar implant may include a pocket around the center peg for a cement mantle.

FIGS. 93A to 93E depict aspects of an exemplary patellar implant 9300 according to embodiments of the present disclosure.

Example surgical tools for implanting the trochlear implants or the patellar implants are described below. The surgical tools for implanting the trochlear implant and/or the patellar implant can incorporate any of the features of the surgical tools for implanting the femoral implant and/or the tibial implant disclosed herein. The surgical procedure for implanting the trochlear implants and/or the patellar implants can incorporate any of the features of the surgical procedure for implanting the femoral implant and/or the tibial implant described elsewhere in the present disclosure.

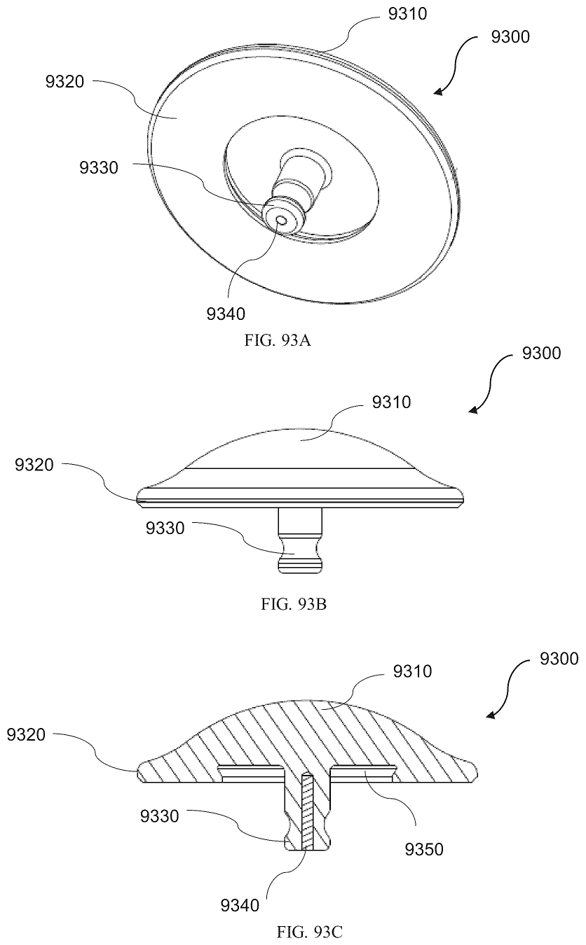

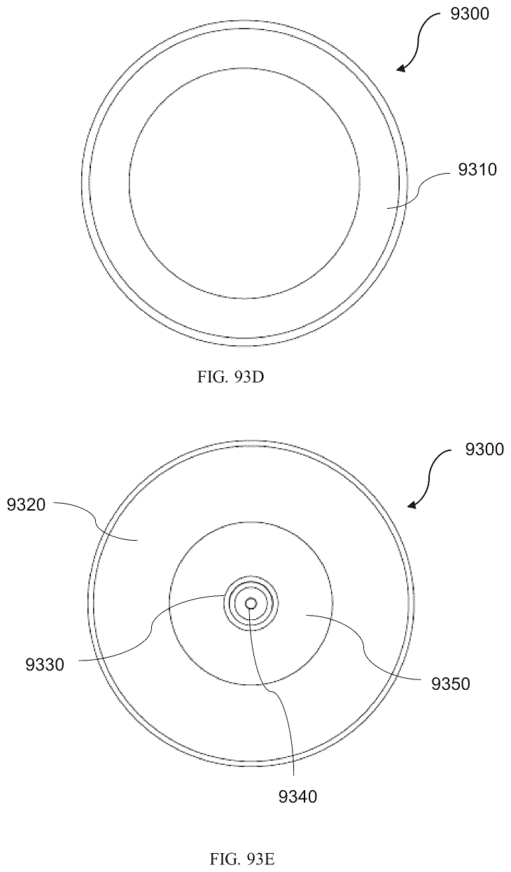

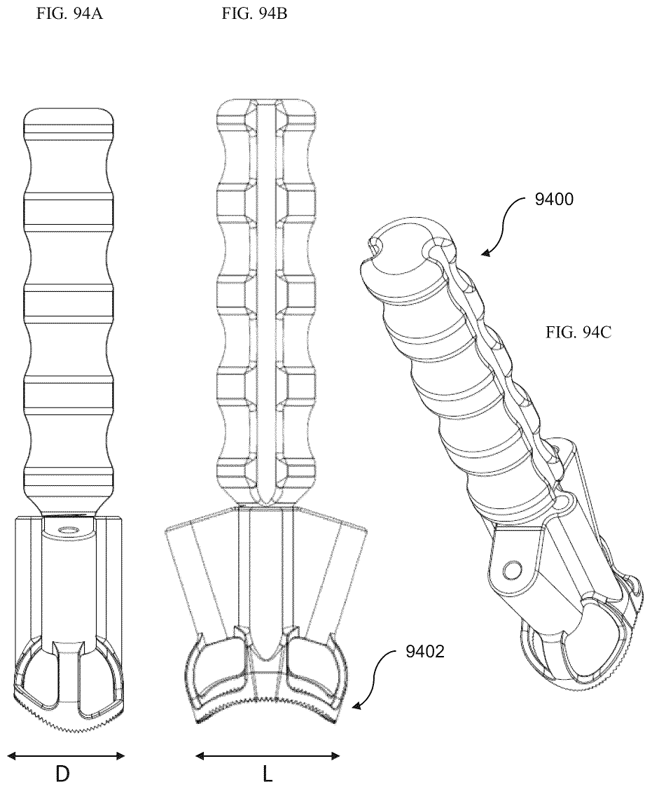

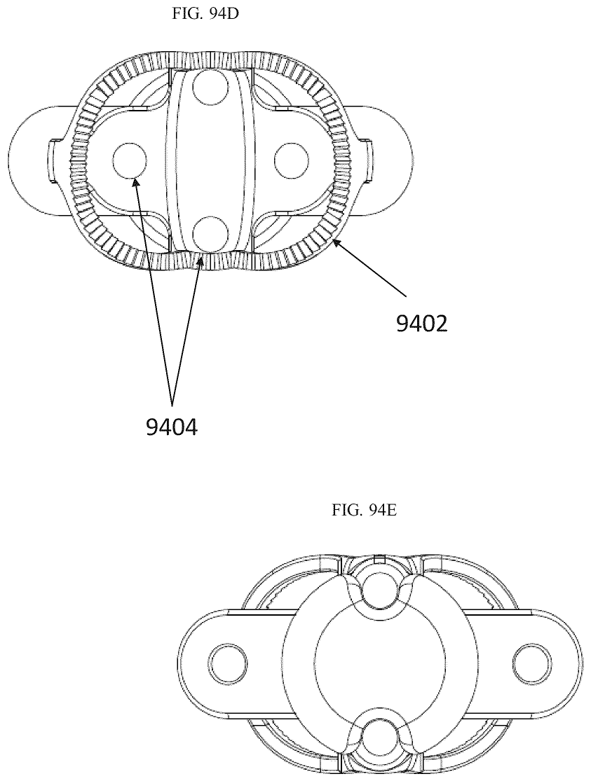

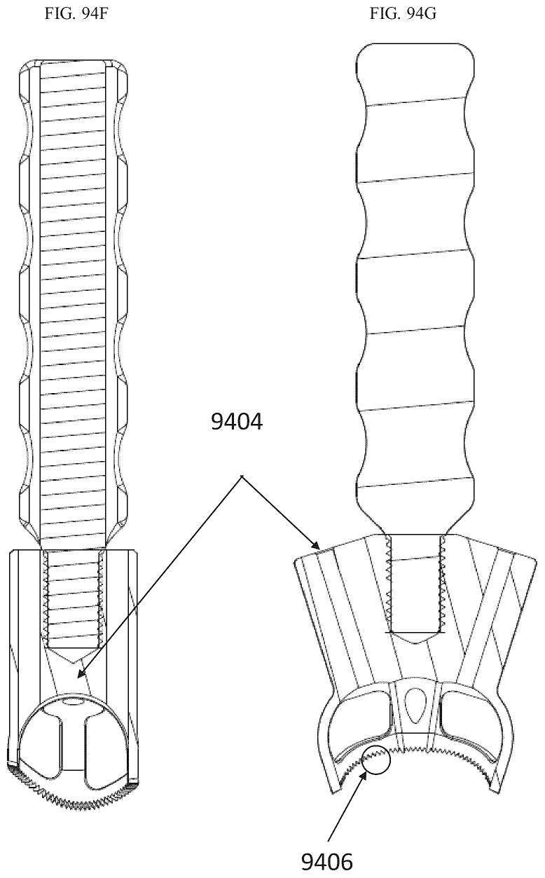

FIGS. 94A to 96F illustrate various examples of a trochlear pin guide system, which can incorporate any of the features of a pin guide system for implanting a femoral implant or a tibial implant disclosed above. FIGS. 94A to 94G depict aspects of a trochlear oblong pin guide system 9400, according to embodiments of the present disclosure. FIG. 94A illustrates a front view of the trochlear oblong pin guide system 9400, showing a diameter (D) of the pin guide 9402. FIG. 94B illustrates a right hand side view of the trochlear oblong pin guide system 9400 showing a length (L) of the pin guide 9402. FIG. 94C illustrates a perspective view of the trochlear oblong pin guide system 9400. The diameter and/or the length of the pin guide 9402 can be used to determine a size of a trochlear oblong implant for a patient. As shown in a bottom view in FIG. 94D and cross-sectional views in FIGS. 94F and 94G, the pin guide system 9400 can include multiple (for example, 4, 6, 8, or otherwise) pin guide holes 9404. An underside surface geometry of the pin guide 9402 can be identical to an articular surface of a trochlear implant, such as any trochlear oblong implants disclosed herein. The underside surface geometry of the pin guide 9402 can include multiple spikes 9406 (for example, spikes along a perimeter of the pin guide 9402) to stabilize the guide pin 9402 while installing pins. FIG. 94E illustrates a top view of the trochlear oblong pin guide system 9400.

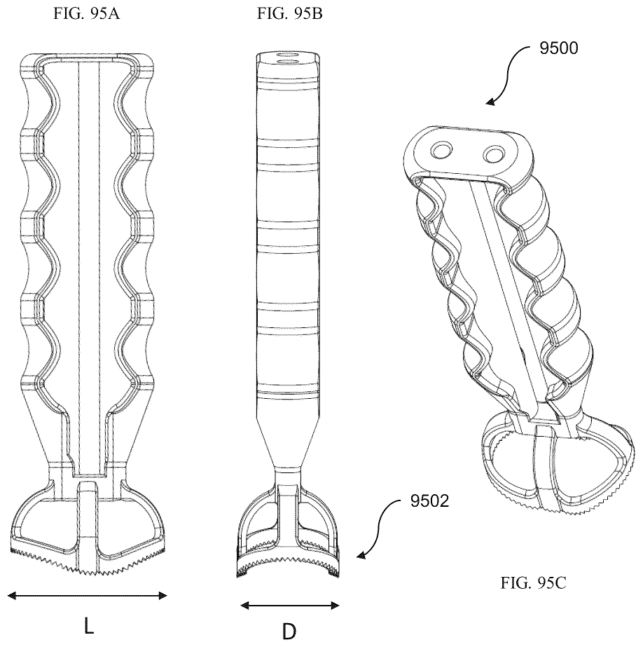

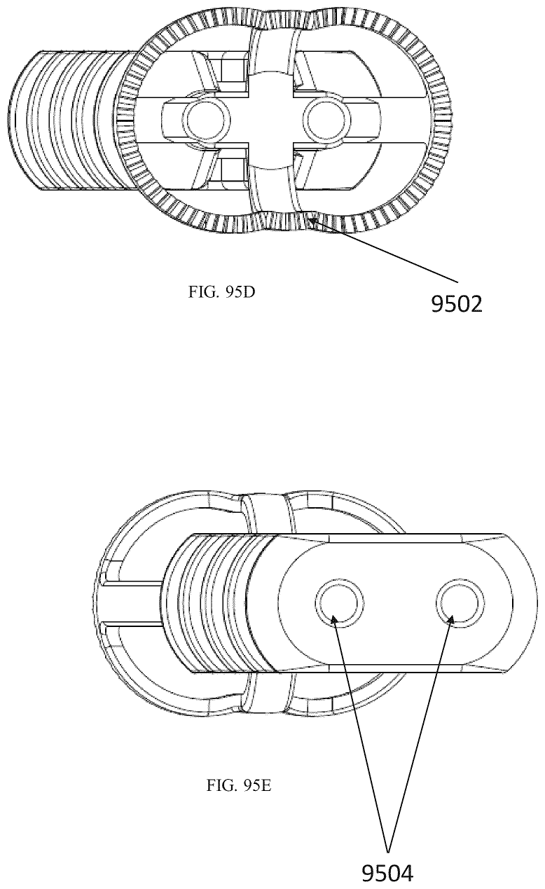

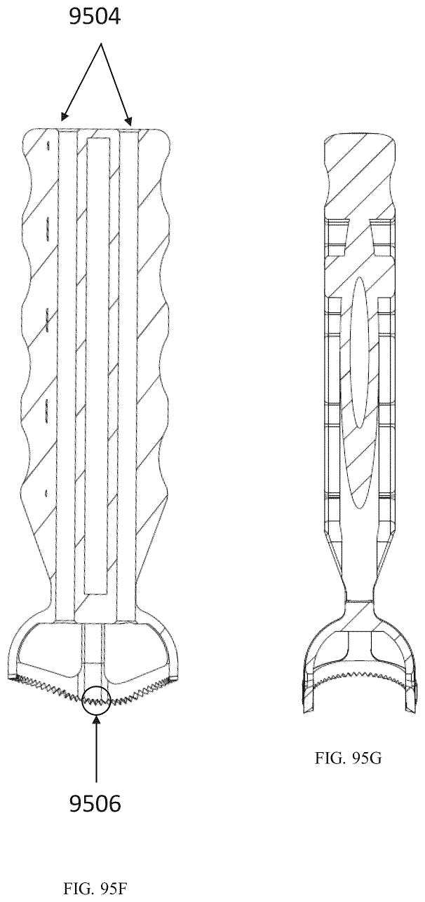

FIGS. 95A to 95G depicts aspects of a trochlear ooblong pin guide system 9500, according to embodiments of the present disclosure. FIG. 95A illustrates a right hand side view of the trochlear ooblong pin guide system 9500 showing a length (L) of the pin guide 9502. FIG. 95B illustrates a front view of the trochlear ooblong pin guide system 9500, showing a diameter (D) of the pin guide 9502. FIG. 95C illustrates a perspective view of the trochlear ooblong pin guide system 9500. The diameter and/or the length of the pin guide 9502 can be used to determine a size of a trochlear ooblong implant for a patient. As shown in a bottom view in FIG. 95D and cross-sectional views in FIG. 95F, the pin guide system 9500 can include multiple (for example, 2, 4, or otherwise) pin guide holes 9504. An underside surface geometry of the pin guide 9502 can be identical to an articular surface of a trochlear implant, such as any trochlear ooblong implants disclosed herein. The underside surface geometry of the pin guide 9502 can include multiple spikes 9506 (for example, spikes along a perimeter of the pin guide 9502) to stabilize the guide pin 9502 while installing pins. FIG. 95E illustrates a top view of the trochlear oblong pin guide system 9400. FIG. 95G illustrates another cross-sectional view of the trochlear ooblong pin guide system 9500.

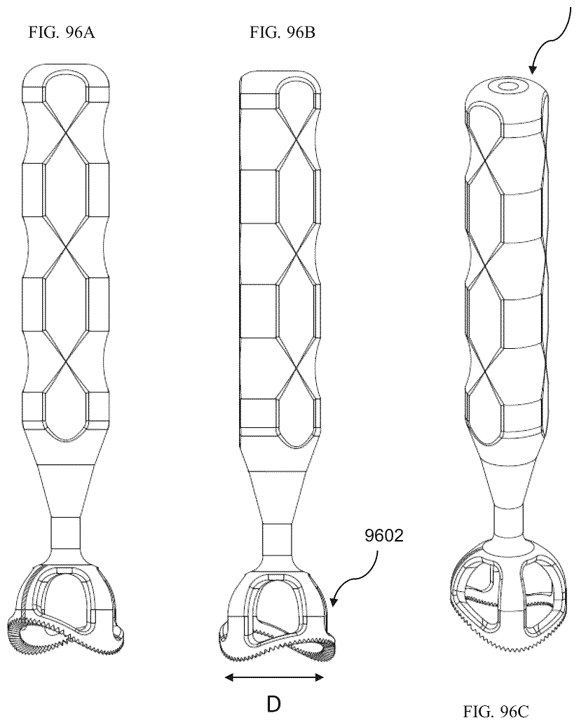

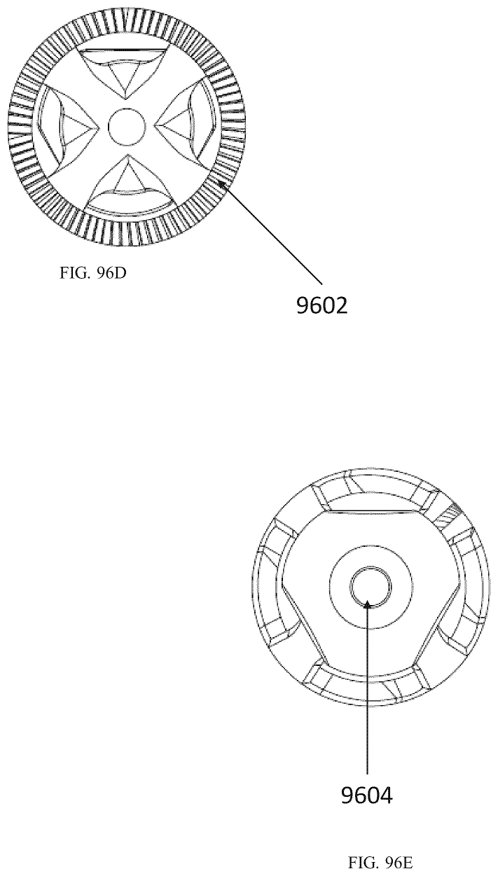

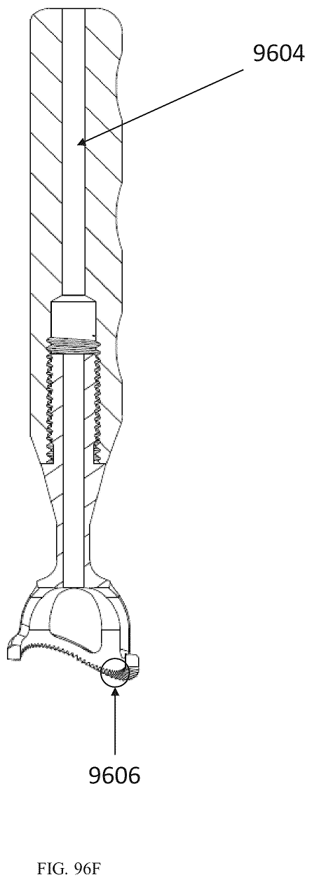

FIGS. 96A to 96F depicts aspects of a trochlear round pin guide system 9600, according to embodiments of the present disclosure. FIGS. 96A and 96B illustrate side and front views of the trochlear round pin guide system 9600 showing a diameter (D) of the pin guide 9602. FIG. 96C illustrates a perspective view of the trochlear round pin guide system 9600. The diameter of the pin guide 9602 can be used to determine a size of a trochlear round implant for a patient. As shown in FIG. 96D, an underside surface geometry of the pin guide 9602 can be identical to an articular surface of a trochlear implant, such as any trochlear round implants disclosed herein. As shown in FIG. 96F, the underside surface geometry of the pin guide 9602 can include multiple spikes 9606 (for example, spikes along a perimeter of the pin guide 9602) to stabilize the guide pin 9602 while installing pins. The pin guide system 9600 can include a central pin guide hole 9604.

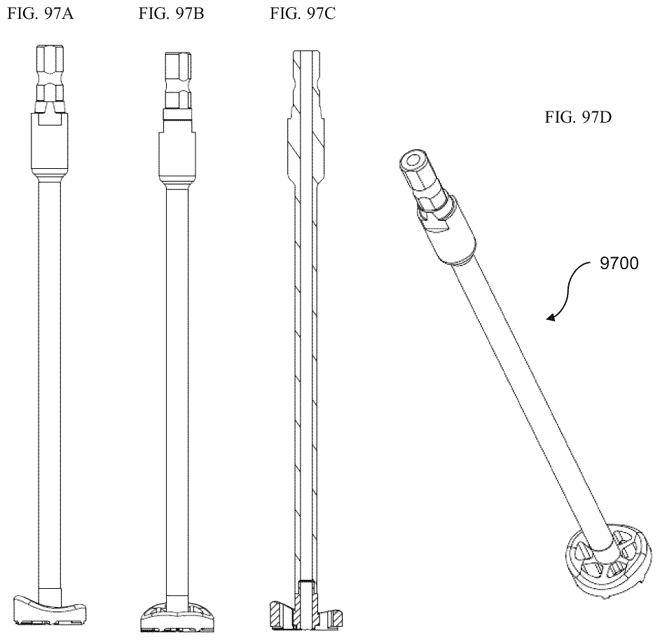

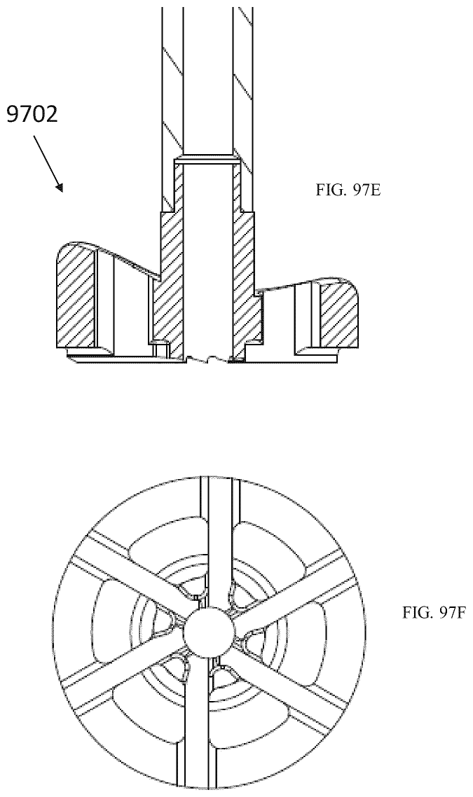

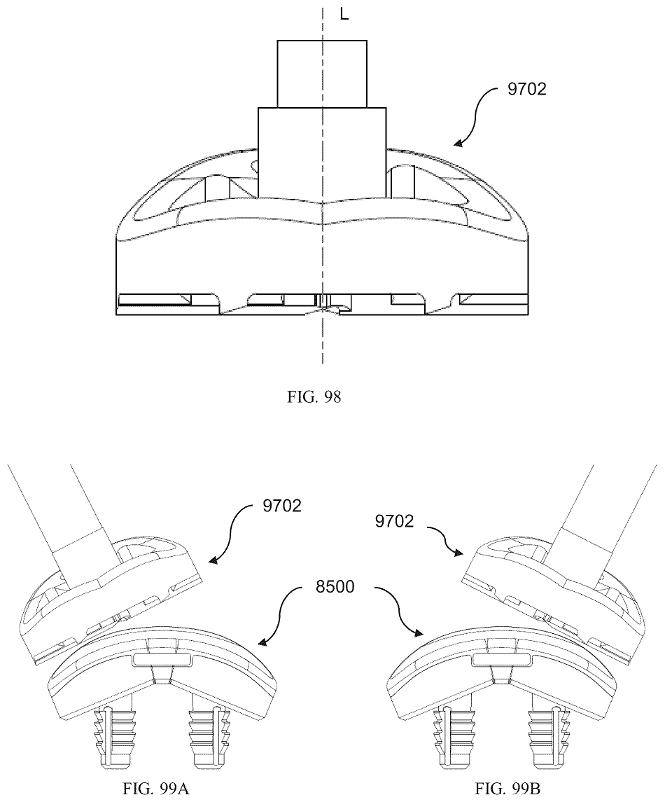

FIGS. 97A to 97F, 98, 99A, 99B, and 100 depict aspects of a trochlear oblong secondary reamer system 9700, according to embodiments of the present disclosure. FIG. 97A illustrates a front view of the trochlear oblong secondary reamer system 9700. FIG. 97B illustrates a right hand side view of the trochlear oblong secondary reamer system 9700. FIG. 97C illustrates a cross-sectional view of the trochlear oblong secondary reamer system 9700. FIG. 97D illustrates a perspective view of the trochlear oblong secondary reamer system 9700. As shown in FIG. 97E, a shape of a second reamer head 9702 of the trochlear oblong secondary reamer system 9700 can match the geometry of the trochlear oblong implant disclosed herein, allowing the trochlear oblong secondary reamer system 9700 to function as a trial while reaming. FIG. 97F illustrates a bottom view of the trochlear oblong secondary reamer system 9700. As shown in FIG. 98, which illustrates the reamer head 9702 in the right hand side view, the geometry of the reamer head 9702 is symmetrical about a longitudinal axis L of the trochlear oblong secondary reamer system 9700. The mirrored geometry can allow the reamer head 9702 to match the front and back profile of the angled reams. FIG. 99A illustrates the reamer head 9702 matching the front profile of a trochlear oblong implant (such as the implant 8500 described elsewhere in the present disclosure with reference to FIGS. 85A-85H). FIG. 99B illustrates the reamer head 9702 matching the back profile of a trochlear oblong implant (such as the implant 8500 described elsewhere in the present disclosure with reference to FIGS. 85A-85H). FIG. 100 illustrates a top perspective view of the reamer head 9702. The trochlear oblong secondary reamer system 9700 can include any features of the femoral secondary reamer 6400 described elsewhere in the present disclosure, including but not limited to being a single use system.

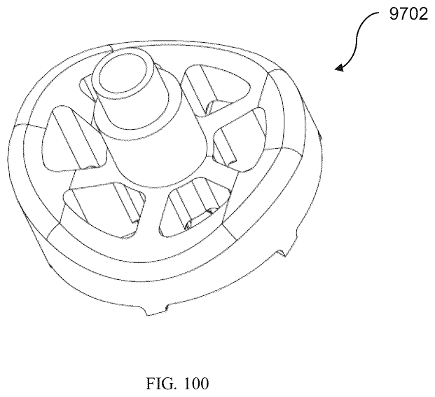

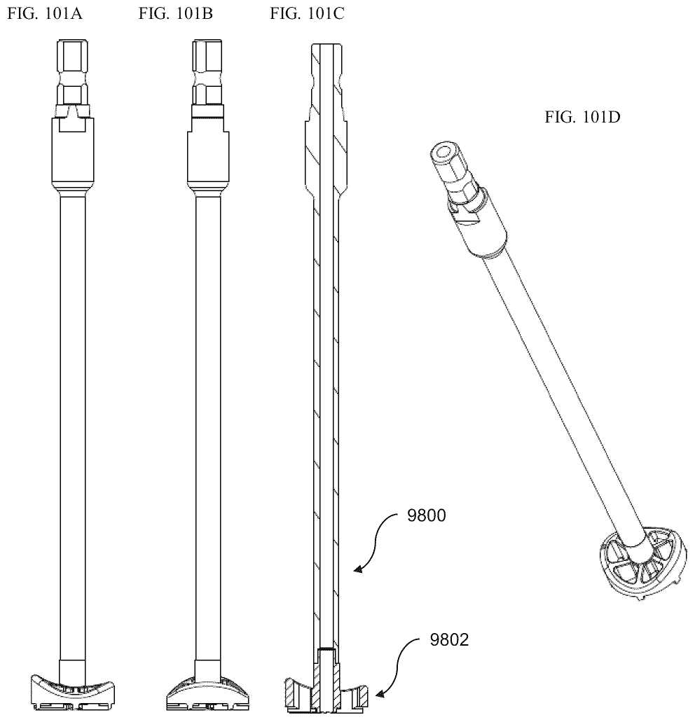

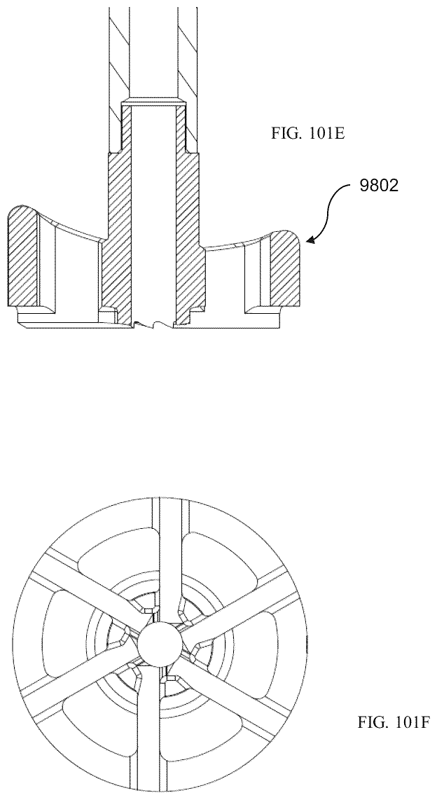

FIGS. 101A to 101F depict aspects of a trochlear ooblong and round secondary reamer system 9800, according to embodiments of the present disclosure. In other words, the secondary reamer system 9800 can be used for applications of either the trochlear ooblong implant or the trochlear round implant disclosed herein. FIG. 101A illustrates a front view of the trochlear ooblong and round secondary reamer system 9800. FIG. 101B illustrates a right hand side view of the trochlear ooblong and round secondary reamer system 9800. FIG. 101C illustrates a cross-sectional view of the trochlear ooblong and round secondary reamer system 9800. FIG. 101D illustrates a perspective view of the trochlear ooblong and round secondary reamer system 9800. As shown in FIG. 101E, a shape of a second reamer head 9802 of the trochlear ooblong and round secondary reamer system 9800 can match the geometry of the trochlear ooblong or round implant disclosed herein, allowing the trochlear ooblong and round secondary reamer system 9800 to function as a trial while reaming. FIG. 101F illustrates a bottom view of the trochlear ooblong and round secondary reamer system 9800. The trochlear ooblong and round secondary reamer system 9800 can include any features of the femoral secondary reamer 6400 described elsewhere in the present disclosure, including but not limited to being a single use system.

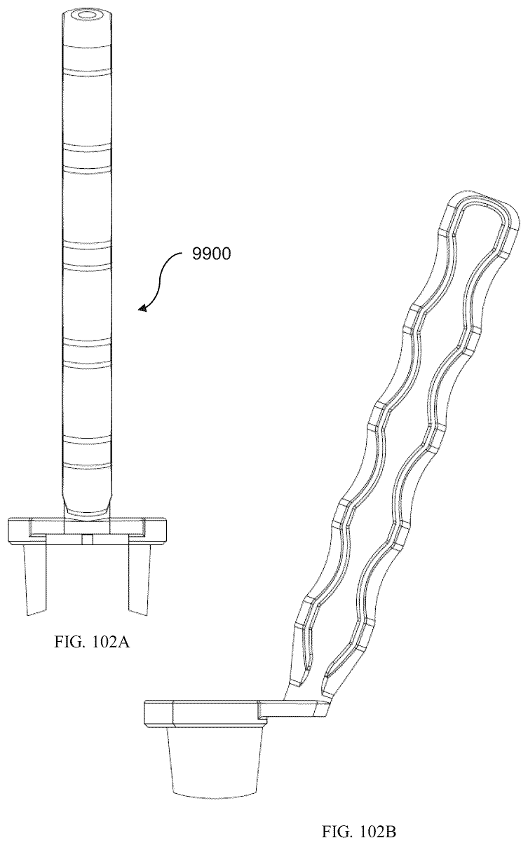

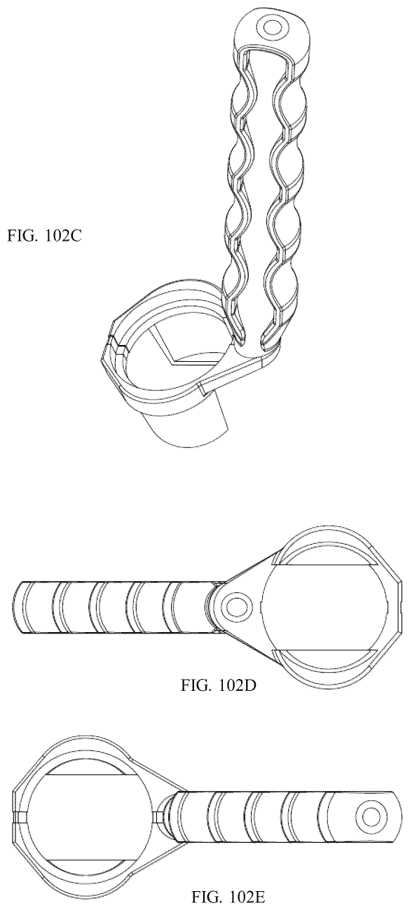

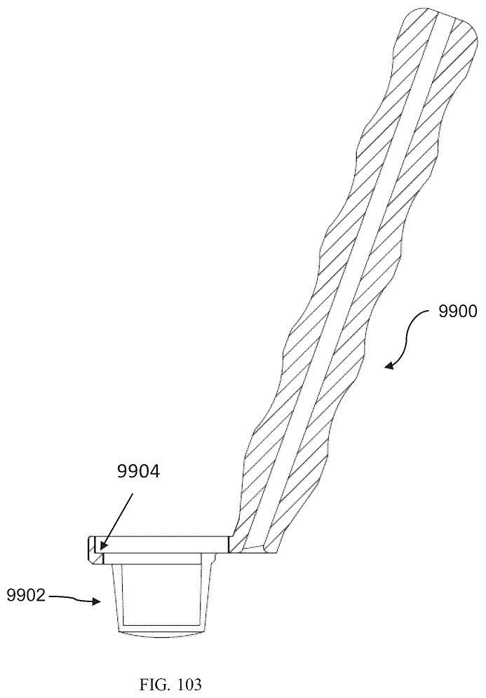

FIGS. 102A to 102E and 103 depict aspects of a trochlear oblong reamer guide system 9900, according to embodiments of the present disclosure. FIG. 102A illustrates a front view of the trochlear oblong reamer guide system 9900. FIG. 102B illustrates a right hand side view of the trochlear oblong reamer guide system 9900. FIG. 102C illustrates a perspective view of the trochlear oblong reamer guide system 9900. FIG. 102D illustrates a bottom view of the trochlear oblong reamer guide system 9900. FIG. 102E illustrates a top view of the trochlear oblong reamer guide system 9900. FIG. 103 illustrates a cross-sectional view of the trochlear oblong reamer guide system 9900. As shown, the trochlear oblong reamer guide system 9900 includes a shoulder 9904 in a reamer guide portion 9902. A trochlear oblong reamer, which may have any features of the first femoral reamer 6100 disclosed herein, can include a ledge that can bottom out at the shoulder 9904 during reaming, which can determine the depth of reaming. In some embodiments, the trochlear oblong reamer guide system 9900 can have any features of the femoral reamer guide 6900 disclosed herein.

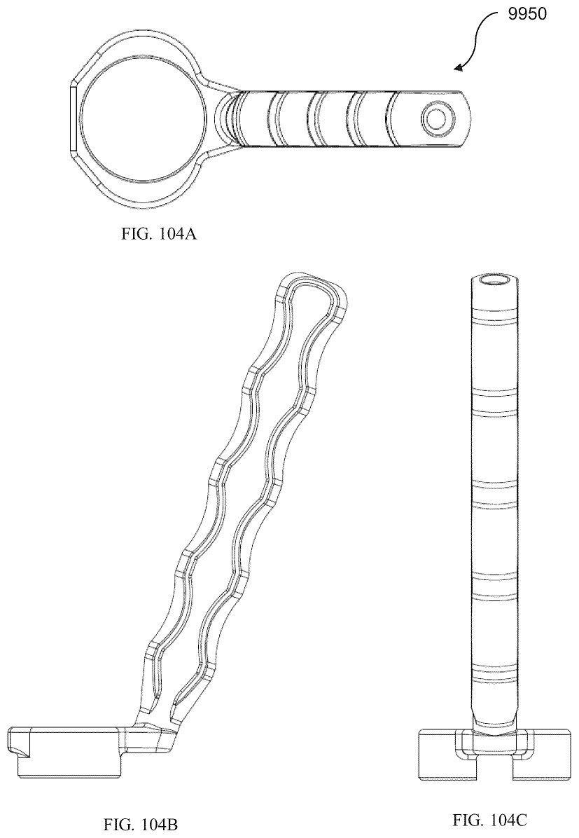

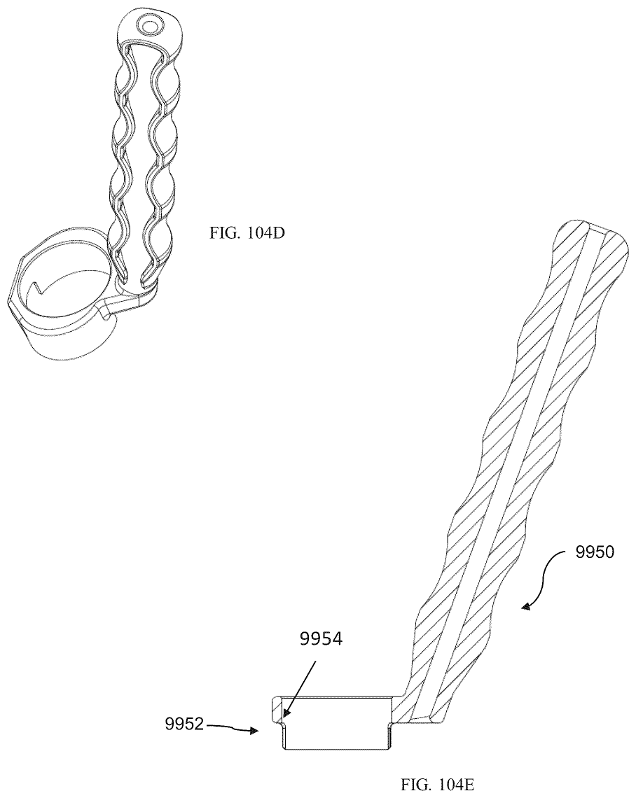

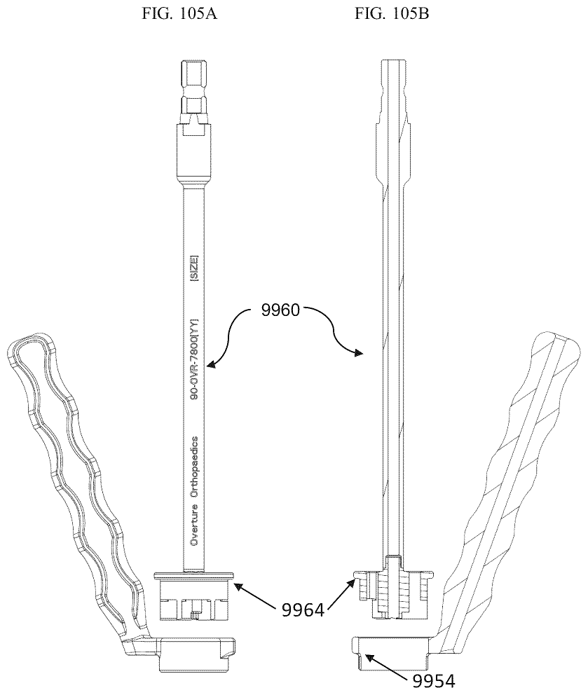

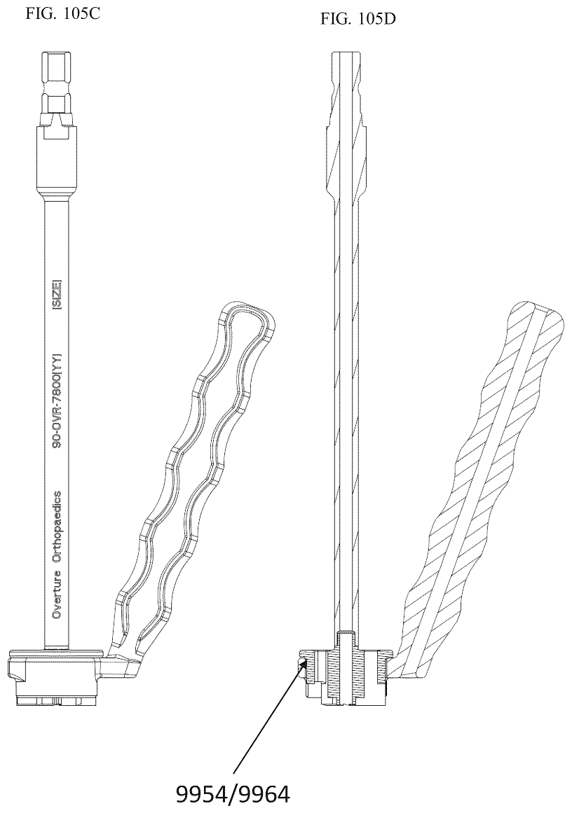

FIGS. 104A to 104E depicts aspects of a trochlear ooblong reamer guide device 9950, according to embodiments of the present disclosure. FIG. 104A illustrates a top view of the trochlear ooblong reamer guide system 9950. FIG. 104B illustrates a right hand side view of the trochlear ooblong reamer guide system 9950. FIG. 104C illustrates a back view of the trochlear ooblong reamer guide system 9950. FIG. 104D illustrates a perspective view of the trochlear oblong reamer guide system 9900. FIG. 105E illustrates a cross-sectional view of the trochlear ooblong reamer guide system 9950. the trochlear oblong reamer guide system 9900 includes a shoulder 9904 in a reamer guide portion 9902. As shown in FIGS. 105A-105D, a trochlear ooblong reamer 9960, which may have any features of the first femoral reamer 6100 disclosed herein, can include a ledge 9964 that can bottom out at the shoulder 9954 of the reamer guide portion 9952 when the reamer 9960 is inserted concentrically into the reamer guide portion 9952, which can determine the depth of reaming. In some embodiments, the trochlear ooblong reamer guide system 9950 can have any features of the femoral reamer guide 6900 disclosed herein.

### H. Conclusion

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A monolithic implant for targeted joint resurfacing to replace a defect while preserving as much healthy bone and/or cartilage as possible of a patient, the implant comprising:
a solid metal portion including an articulating surface, the articulating surface being convex and load-bearing; and
a porous metal portion including a subchondral surface configured to interface with a surgically prepared bone surface, the porous metal portion further including at least an outer portion of a fixation peg extending from the subchondral surface, wherein an inner or core portion of the fixation peg is part of the solid metal portion.

2. The monolithic implant of claim 1, wherein a part of the implant between the articulating surface and the subchondral surface has a round profile.

3. (canceled)

4. The monolithic implant of claim 1, wherein a part of the implant between the articulating surface and the subchondral surface has an oblong profile, the oblong profile having a length that is greater than a width, wherein the oblong profile is formed by three circles aligned along the length such that at least two neighboring circles partially overlap.

5. (canceled)

6. (canceled)

7. (canceled)

8. The monolithic implant of claim 1, further comprising a second peg, wherein at least an outer portion of the second peg is part of the porous metal portion.

9. The monolithic implant of claim 1, wherein the solid metal portion further comprises a plurality of anti-rotation spikes extending from the subchondral surface, the plurality of anti-rotation spikes being 3D printed onto the subchondral surface.

10. (canceled)

11. The monolithic implant of claim 1, wherein the porous metal portion is 3D-printed.

12. The monolithic implant of claim 1, wherein the fixation peg includes a plurality of barbs, the plurality of barbs being part of the solid metal portion.

13. The monolithic implant of claim 12, wherein the plurality of barbs are 3D-printed.

14.-21. (canceled)

22. The monolithic implant of claim 1, wherein the monolithic implant is a femoral implant configured for targeted knee joint resurfacing in which only an area including the defect is removed, wherein the area removed occupies an area smaller than an entire surface of a femoral condyle.

23. A single-piece implant for targeted joint resurfacing to replace a defect while preserving as much healthy bone and/or cartilage as possible of a patient, the implant comprising:
a metal portion including a subchondral surface configured to interface with a surgically prepared bone surface, the metal portion comprising solid metal parts and porous metal parts, the porous metal parts comprising the subchondral surface and an overmolding surface, the solid metal parts located at least between the subchondral surface and the overmolding surface, wherein the metal portion further includes a fixation peg extending from the subchondral surface; and
a plastic portion overmolded on the metal portion such that the single-piece implant is preassembled during manufacturing, the plastic portion including an articulating surface and an overmolding section, the articulating surface being concave and load-bearing, the overmolding section interfacing with the overmolding surface of the metal portion,
wherein the single-piece implant includes a circumferential groove at an interface of the overmolding section and the overmolding surface, the circumferential groove configured to allow clamping of the metal portion during overmolding to provide a clean stop of plastic flow below the circumferential groove.

24. (canceled)

25. The single-piece implant of claim 23, wherein the fixation peg includes a plurality of barbs, the plurality of barbs being one of the solid metal parts.

26. The single-piece implant of claim 25, wherein the plurality of barbs are 3D-printed.

27.-32. (canceled)

33. The single-piece implant of claim 23, wherein the articulating surface has a rim defining a proximal plane and the subchondral surface defines a distal plane, the proximal plane and the distal plane being at a non-zero angle so as to facilitate insertion in an antegrade approach.

34. The single-piece implant of claim 33, wherein the non-zero angle is between 10° to 15°.

35. The single-piece implant of claim 34, wherein the non-zero angle is 10°.

36. The single-piece implant of claim 23, wherein the metal portion further comprises a plurality of anti-rotation spikes extending from the subchondral surface, the plurality of anti-rotation spikes being solid and 3D printed onto the subchondral surface.

37. (canceled)

38. The single-piece implant of claim 23, wherein the porous metal parts are 3D-printed.

39. (canceled)

40. The single-piece implant of claim 23, wherein the single-piece implant is a tibial implant configured for targeted knee joint resurfacing in which only an area including the defect is removed, wherein the area removed occupies an area smaller than an entire surface of a tibial plateau.

41.-67. (canceled)

68. A method of implanting a tibial implant configured for targeted knee joint resurfacing to replace an osteochondral defect while preserving as much healthy bone and/or cartilage as possible of a patient, wherein the tibial implant comprises an articulating surface and a subchondral surface, the articulating surface configured to be load bearing, the subchondral surface configured to interface with a reamed bone surface on a tibial plateau, the articulating surface including a rim defining a proximal plane and the subchondral surface defining a distal plane, wherein the proximal plane and the distal plane are at a first non-zero angle so as to facilitate insertion of the tibial implant in an antegrade approach for better visibility of the defect, the method comprising:
flexing a knee joint of a patient by at least 120°, wherein the patient is in a supine position;
making a longitudinal incision on skin of the patient at the knee joint to expose the knee joint, wherein the incision is made on a lateral side if the osteochondral defect is on the lateral side and the incision is made on a medial side if the osteochondral defect is on the medial side;
detaching an anterior portion of a meniscus from an articulating surface of a tibia to access the tibial plateau in an antegrade approach, the meniscus remaining attached to a joint capsule; and
inserting a reamer from an anterior side of the knee joint at a second non-zero angle relative to a longitudinal axis of the tibia, wherein a reamer head of the reamer comprises a cutout, the reamer is oriented such that the cutout is facing a femur condyle during insertion and removal of the reamer.

69. (canceled)

70. The method of claim 68, wherein the first or second non-zero angle is between 10° to 15°.

71. (canceled)

72. (canceled)

73. The method of claim 68, wherein the tibial implant is a single piece component that does not require in-surgery assembly.

74.-108. (canceled)
